(19) United States
(12) Patent Application Publication
(10) Pub. No.: US 2004/0102360 A1
(43) Pub. Date: May 27, 2004

(54) Combination therapy

(76) Inventors: Stanley F. Barnett, North Wales, PA (US); Deborah D. DeFeo-Jones, Lansdale, PA (US); George D. Hartman, Lansdale, PA (US); Hans E. Huber, Lansdale, PA (US); Steven M. Stirdivant, Doylestown, PA (US); David C. Heimbrook, Coopersburg, PA (US)

Correspondence Address: MERCK AND CO INC, P O BOX 2000, RAHWAY, NJ, 070650907

(21) Appl. No.: 10/678,565

(22) Filed: Oct. 3, 2003

Related U.S. Application Data
(60) Provisional application No. 60/422,312, filed on Oct. 30, 2002.
(60) Provisional application No. 60/460,911, filed on Apr. 7, 2003.

(51) Int. Cl. A61K 31/00

(52) U.S. Cl. 514/1

(57) ABSTRACT

The present invention relates to methods of treating cancer using a combination of at least two Akt inhibitors or a compound which is an inhibitor of Akt and an inhibitor of a protein kinase, which methods comprise administering to a mammal, either sequentially in any order or simultaneously, amounts of at least two therapeutic agents selected from a group consisting of a compound(s) which are inhibitors of Akt and compound(s) which are inhibitors of protein kinases. The invention also relates to methods of preparing such compositions.

### BACKGROUND OF THE INVENTION

The present invention relates to methods of treating cancer which comprise administering to a patient in need thereof at least two inhibitors of Akt kinase or at least one inhibitor of Akt kinase and at least one inhibitor of a protein kinase. Further disclosed is a method of inhibiting Akt1 and Akt2.

The phosphatidylinositol 3′-OH kinase (PI3K)/Akt/PKB pathway appears important for regulating cell survival/cell death (Kulik et al. Mol. Cell. Biol. 17:1595-1606 (1997); Franke et al, Cell, 88:435-437 (1997); Kauffmann-Zeh et al. Nature 385:544-548 (1997) Hemmings Science, 275:628-630 (1997); Dudek et al., Science, 275:661-665 (1997)). Survival factors, such as platelet derived growth factor (PDGF), nerve growth factor (NGF) and insulin-like growth factor-1 (IGF-1), promote cell survival under various conditions by inducing the activity of PI3K (Kulik et al. 1997, Hemmings 1997). Activated PI3K leads to the production of phosphatidylinositol (3,4,5)-triphosphate (Ptdlns(3,4,5)-P3), which in turn binds to, and promotes the activation of, the serine/threonine kinase Akt, which contains a pleckstrin homology (PH)-domain (Franke et al Cell, 81:727-736 (1995); Hemmings Science, 277:534 (1997); Downward, Curr. Opin. Cell Biol. 10:262-267 (1998), Alessi et al., EMBO J. 15: 6541-6551 (1996)). Specific inhibitors of PI3K or dominant negative Akt/PKB mutants abolish survival-promoting activity of these growth factors or cytokines. It has been previously disclosed that inhibitors of PI3K (LY294002 or wortmannin) blocked the activation of Akt/PKB by upstream kinases. In addition, introduction of constitutively active PI3K or Akt/PKB mutants promotes cell survival under conditions in which cells normally undergo apoptotic cell death (Kulik et al. 1997, Dudek et al. 1997). Analysis of Akt levels in human tumors showed that Akt2 is overexpressed in a significant number of ovarian (J. Q. Cheung et al. Proc. Natl. Acad. Sci. U.S.A. 89:9267-9271(1992)) and pancreatic cancers (J. Q. Cheung et al. Proc. Natl. Acad. Sci. U.S.A. 93:3636-3641 (1996)). Similarly, Akt3 was found to be overexpressed in breast and prostate cancer cell lines (Nakatani et al. J. Biol. Chem. 274:21528-21532 (1999).

The tumor suppressor PTEN, a protein and lipid phosphatase that specifically removes the 3′ phosphate of PtdIns(3,4,5)-P3, is a negative regulator of the PI3K/Akt pathway (Li et al. Science 275:1943-1947 (1997), Stambolic et al. Cell 95:29-39 (1998), Sun et al. Proc. Natl. Acad. Sci. U.S.A. 96:6199-6204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-3663 (1997), Risinger et al. Cancer Research 57:4736-4738 (1997)).

These observations demonstrate that the PI3K/Akt pathway plays important roles for regulating cell survival or apoptosis in tumorigenesis.

Three members of the Akt/PKB subfamily of second-messenger regulated serine/threonine protein kinases have been identified and termed Akt1/PKBα, Akt2/PKBβ, and Akt3/PKBγ respectively. The isoforms are homologous, particularly in regions encoding the catalytic domains. Akt/PKBs are activated by phosphorylation events occurring in response to PI3K signaling. PI3K phosphorylates membrane inositol phospholipids, generating the second messengers phosphatidyl-inositol 3,4,5-trisphosphate and phosphatidylinositol 3,4-bisphosphate, which have been shown to bind to the PH domain of Akt/PKB. The current model of Akt/PKB activation proposes recruitment of the enzyme to the membrane by 3′-phosphorylated phosphoinositides, where phosphorylation of the regulatory sites of Akt/PKB by the upstream kinases occurs (B. A. Hemmings, Science 275:628-630 (1997); B. A. Hemmings, Science 276:534 (1997); J. Downward, Science 279:673-674 (1998)).

Phosphorylation of Akt1/PKBI occurs on two regulatory sites, Thr³⁰⁸ in the catalytic domain activation loop and on Ser⁴⁷³ near the carboxy terminus (D. R. Alessi et al. EMBO J. 15:6541-6551 (1996) and R. Meier et al. J. Biol. Chem. 272:30491-30497 (1997)). Equivalent regulatory phosphorylation sites occur in Akt2/PKB [symbol] and Akt3/PKBK. The upstream kinase, which phosphorylates Akt/PKB at the activation loop site has been cloned and termed 3′-phosphoinositide dependent protein kinase 1 (PDK1). PDK1 phosphorylates not only Akt/PKB, but also p70 ribosomal S6 kinase, p90RSK, serum and glucocorticoid-regulated kinase (SGK), and protein kinase C. The upstream kinase phosphorylating the regulatory site of Akt/PKB near the carboxy terminus has not been identified yet, but a recent report implies a role for the integrin-linked kinase (ILK-1), a serine/threonine protein kinase, or autophosphorylation.

Inhibition of Akt activation and activity can be achieved by inhibiting PI3K with inhibitors such as LY294002 and wortmannin. However, PI3K inhibition has the potential to indiscriminately affect not just all three Akt isozymes but also other PH domain-containing signaling molecules that are dependent on PdtIns(3,4,5)-P3, such as the Tec family of tyrosine kinases. Furthermore, it has been disclosed that Akt can be activated by growth signals that are independent of PI3K.

Alternatively, Akt activity can be inhibited by blocking the activity of the upstream kinase PDK1. No specific PDK1 inhibitors have been disclosed. Again, inhibition of PDK1 would result in inhibition of multiple protein kinases whose activities depend on PDK1, such as a typical PKC isoforms, SGK, and S6 kinases (Williams et al. Curr. Biol. 10:439-448 (2000).

Importantly, specific inhibition of the Akt kinases is desired in that specific Akt kinase inhibitors would not affect downsteam or upstream kinase activities, providing a more focused therapeutic attack against cancer. Compounds which selectively inhibit the various isoforms of Akt is also desired. The compounds of the instant invention are novel and selective inhibitors of Akt kinases.

Growth factor receptors bind growth factors and a cellular signal is transmitted which among other things mediates cell growth, differentiation and cell death. The cellular signal is transmitted via protein kinases which are enzymes that catalyze the phosphorylation of hydroxy groups on tyrosine, serine and threonine residues of proteins. The consequences of these seemingly simple activities are staggering; cell growth, differentiation and proliferation; i.e., virtually all aspects of cell life, in one way or another depend on signal transduction mechanisms coupled to protein kinase activity. Significantly, abnormal protein kinase activity has been related to a host of disorders, ranging from relatively non life-threatening diseases such as psoriasis to extremely virulent diseases such as glioblastoma (brain cancer). Protein kinases can be broken into two classes, the protein tyrosine kinases (PTKs) and the serine-threonine kinases (STKs).

Certain growth factor receptors exhibit PK activity and are known as receptor tyrosine kinases (RTKs). They comprise a large family of transmembrane receptors with diverse biological activity. At present, at least nineteen (19) distinct subfamilies of RTKs have been identified. One RTK subfamily contains the insulin receptor (IR), insulin-like growth factor I receptor (IGF-1R) and insulin receptor related receptor (IRR). IR and IGF-1R interact with insulin to activate a hetero-tetramer composed of two entirely extracellular glycosylated a subunits and two β subunits which cross the cell membrane and which contain the tyrosine kinase domain. The Insulin-like Growth Factor-1 Receptor (IGF-1R), and its ligands, IGF-1 and IGF-2, are abnormally expressed in numerous tumors, including, but not limited to, breast, prostate, thyroid, lung, hepatoma, colon, brain, neuroendocrine, and others.

A more complete listing of the known RTK subfamilies is described in Plowman et al., KN&P, 1994, 7(6):334-339 which is incorporated by reference, including any drawings, as if fully set forth herein.

In addition to the RTKs, there also exists a family of entirely intracellular PTKs called “non-receptor tyrosine kinases” or “cellular tyrosine kinases.” This latter designation, abbreviated “CTK”, will be used herein. CTKs do not contain extracellular and transmembrane domains. At present, over 24 CTKs in 11 subfamilies (Src, Frk, Btk, Csk, Abl, Zap70, Fes, Fps, Fak, Jak and Ack) have been identified. The Src subfamily appears so far to be the largest group of CTKs and includes Src, Yes, Fyn, Lyn, Lck, Blk, Hck, Fgr and Yrk. For a more detailed discussion of CTKs, see Bolen, Oncogene, 1993, 8:2025-2031, which is incorporated by reference, including any drawings, as if fully set forth herein.

Protein kinases include, RTKs, CTKs and STKs and all are implicated in a host of pathogenic conditions including significantly, cancer.

It is therefore an object of the instant invention to provide a method for treating cancer which offers advantages over previously disclosed methods of treatment.

### SUMMARY OF THE INVENTION

A method of treating cancer is disclosed which is comprised of administering to a patient in need of such treatment amounts of at least two inhibitors of Akt or at least one inhibitor of Akt and at least one inhibitor of a protein kinase. Further disclosed is a method of inhibiting Akt1 and Akt2.

### BRIEF DESCRIPTION OF THE FIGURES

FIG. 1: Caspase 3 Assay on LnCaP Cells +/− TRAIL Ligand

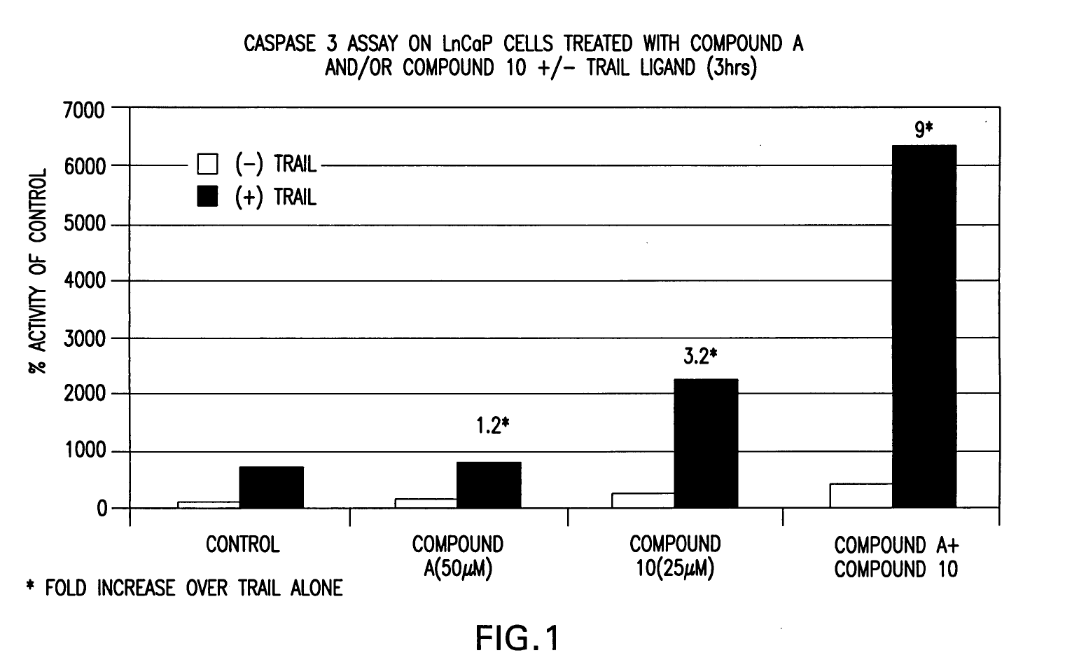

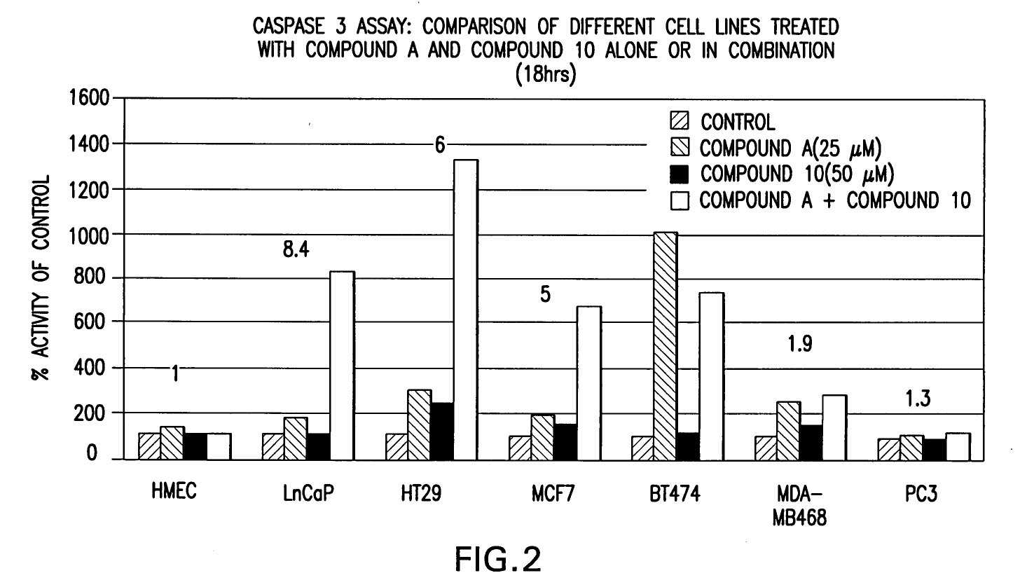

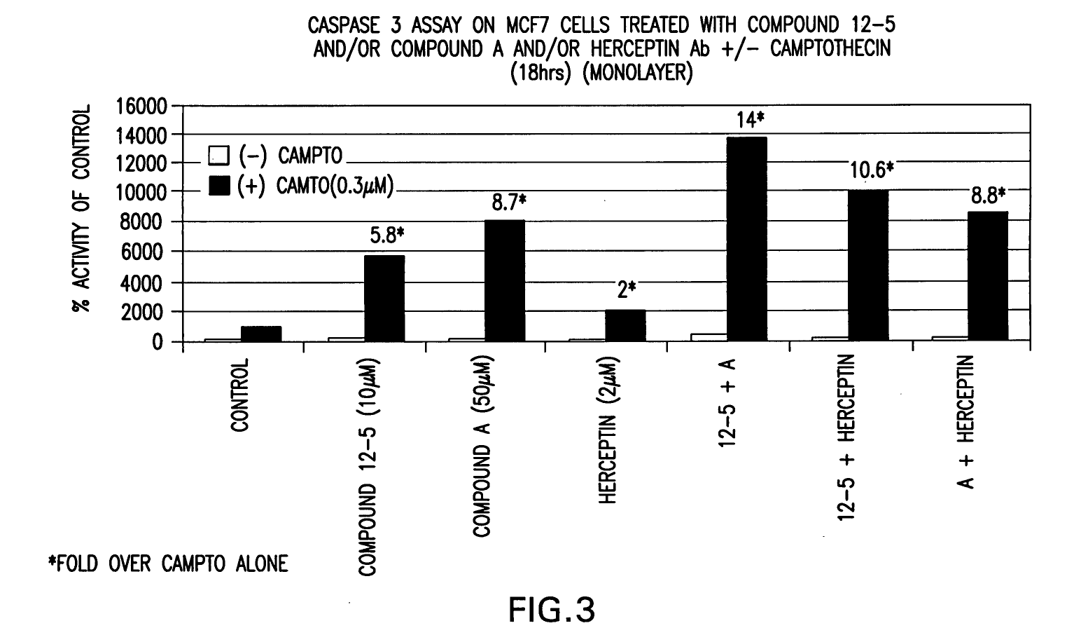

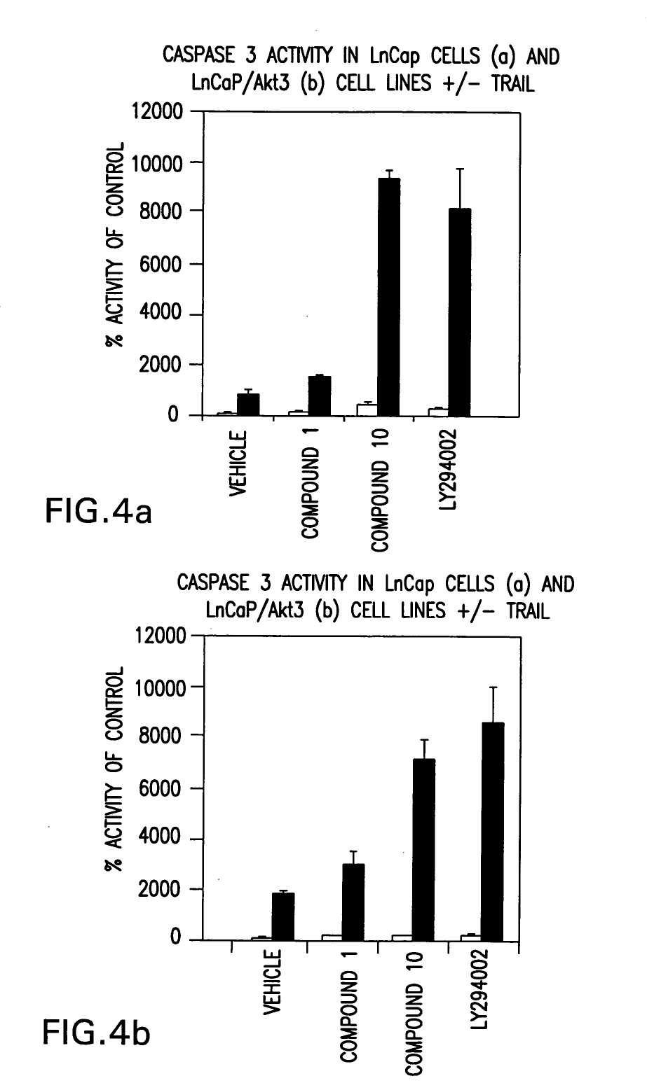

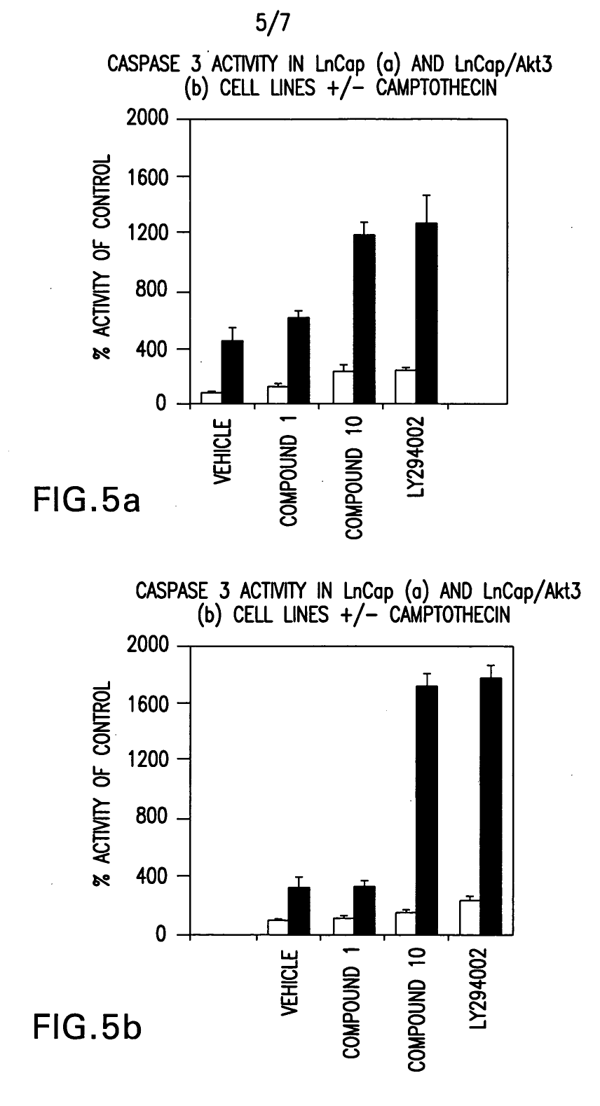

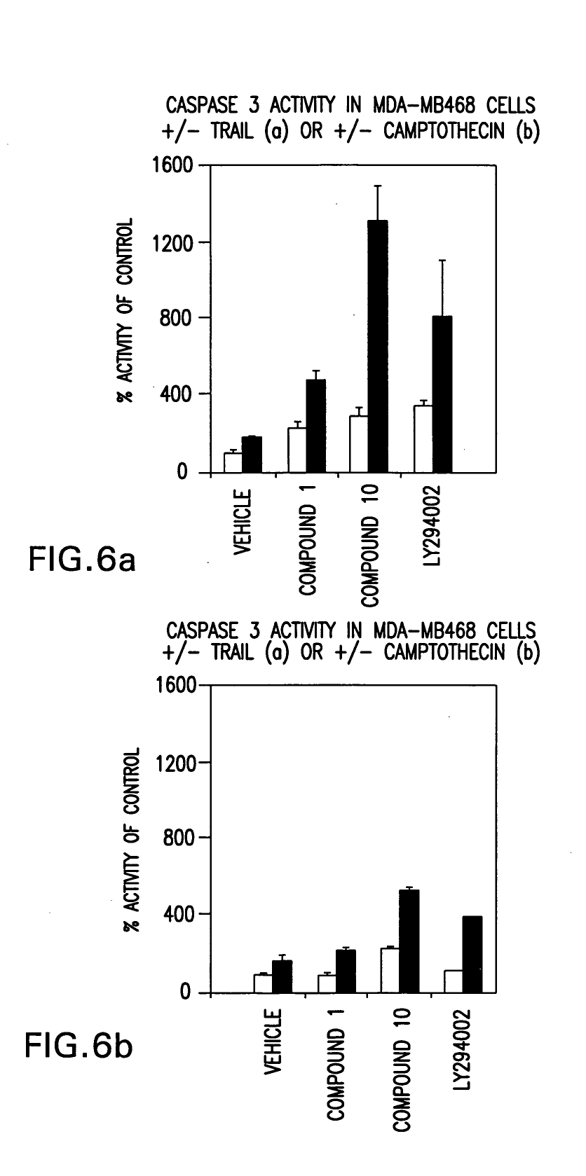

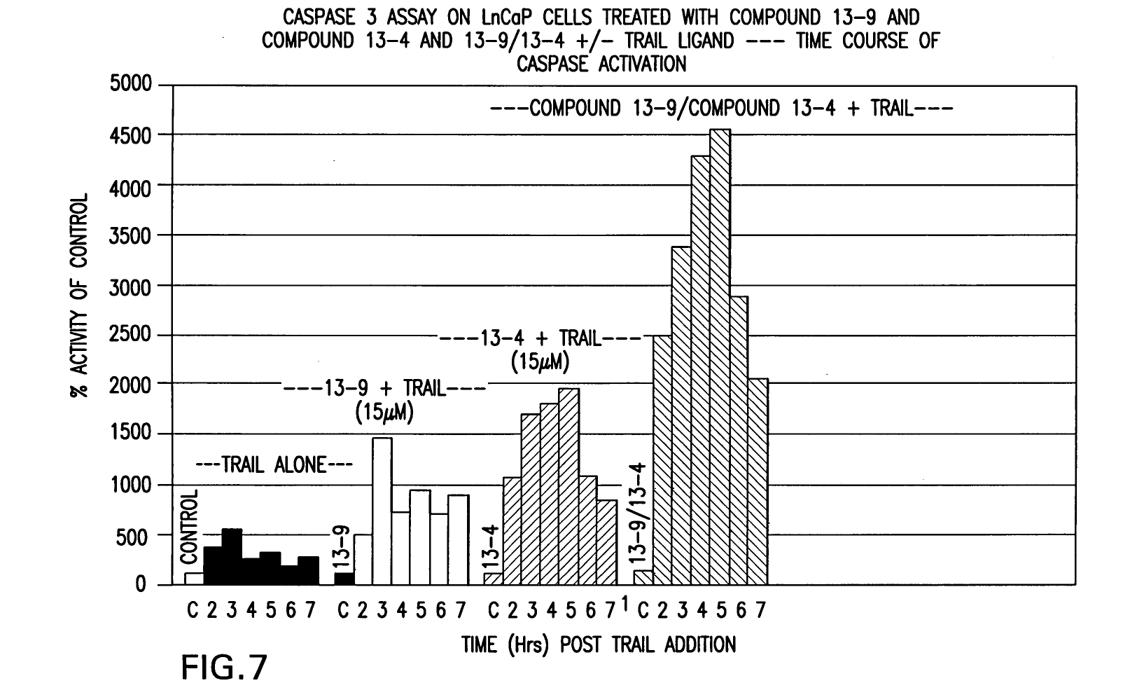

An Akt inhibitor (Compound 10, a selective Akt1 and Akt2 inhibitor) and a protein kinase inhibitor (Compound A) were tested (+/− TRAIL) for Caspase 3 activation alone or in combination in LnCaP cells. Caspase 3 activation is utilized as a marker for an increase in cell death. Compound A demonstrated a 1.2 fold increase in Caspase 3 activation over Trail alone, while Compound 10 produced a 3.2 fold increase in Caspase 3 activation over Trail alone. Combination treatment (Compounds 10 and A) demonstrated a 9 fold increase in Caspase 3 activation over Trail alone. Details of the experimental procedures are found in Example 40.

FIG. 2: Caspase 3 Assay: Comparison of Different Cell Lines

An Akt inhibitor (Compound 10, a selective Akt1 and Akt2 inhibitor) and a protein kinase inhibitor (Compound A) were tested in various cells lines for Caspase 3 activation alone or in combination. Caspase 3 activation is utilized as a marker for an increase in cell death. As demonstrated, combination treatment exhibited increased Caspase 3 activation over one test compound alone in LnCaP, HT29 and MCF7 cell lines. Details of the experimental procedures are found in Example 40.

FIG. 3: Caspase 3 Assay on MCF7 Cells +/− Camptothecin

An Akt inhibitor (Compound 12-5, a selective Akt1 and Akt2 inhibitor) and two protein kinase inhibitors (Compound A and Herceptin Ab) were tested in MCF7 cells for Caspase 3 activation alone or in combination. Caspase 3 activation is utilized as a marker for an increase in cell death. As demonstrated, co-treatment with Camptothecin and Compound 12-5, Compound A and Herceptin Ab, respectively, caused a 5.8-, 8.7- and a 2-fold increase in Caspase 3 activation over Camptothecin alone. Combinations of Compound 12-5, Compound A and Herceptin demonstrated an increase in Caspase 3 activation over the use of one compound alone. Details of the experimental procedures are found in Example 40.

FIG. 4: Caspase 3 Activity in LnCaP Cells (a) and LNCaP/Akt3 (b) Cell Lines +/− Trail

LnCaP (a) and LnCaP/Akt3 (b) cell lines were treated with vehicle, the Akt inhibitors (Compound 1, a selective Akt1 inhibitor) and (Compound 10, a selective Akt1 and Akt2 inhibitor) and LY294002 in the presence (solid bars) or in the absence (open bars) of Trail. Compounds 1, 10 and LY294002 (an inhibitor of PI3K activity) demonstrated an increase in Caspase 3 activity, which is a marker for induction of apoptosis, in the presence of Trail in LnCap cells (a). Moreover, Compounds 1, 10 and LY294002 demonstrated an increase in Caspase 3 activity, in the presence of Trail in LnCap/Akt3 cells (a) (Compound 1 has a statistically significant increase in Caspase activation in the presence of Trail). These results demonstrate that selective Akt1 inhibitors and selective Akt1 and Akt2 inhibitors increase Caspase 3 activity, a marker of cell death, in cancer cells. Moreover, as shown in (b), overexpression of Akt3 does not block the inhibitory effects of a selective Akt1 inhibitor or a selective Akt1 and Akt2 inhibitor. Details of the experimental procedures are found in Example 40.

FIG. 5: Caspase 3 Activity in LnCaP Cells (a) and LNCaP/Akt3 (b) Cell Lines +/− Camptothecin

LnCaP (a) and LnCaP/Akt3 (b) cell lines were treated with vehicle, the Akt inhibitors (Compound 1, a selective Akt1 inhibitor) and (Compound 10, a selective Akt1 and Akt2 inhibitor) and LY294002 (an inhibitor of PI3K activity) in the presence (solid bars) or in the absence (open bars) of Camptothecin. Compound 10 and LY294002 demonstrated an increase in Caspase 3 activity, which is a marker for induction of apoptosis, in the presence of Camptothecin in LnCap cells (a). Moreover, Compounds 10 and LY294002 demonstrated an increase in Caspase 3 activity, in the presence of Camptothecin in LnCap/Akt3 cells (a). These results demonstrate that selective Akt1 and Akt2 inhibition increases Caspase 3 activity, a marker of cell death, in cancer cells. Moreover, as shown in (b), overexpression of Akt3 does not block the inhibitory effects of a selective Akt1 inhibitor or a selective Akt 1 and Akt2 inhibitor. Details of the experimental procedures are found in Example 40.

FIG. 6: Caspase 3 Activity in MDA-MB468 Cells +/− Trail (a) or +/−Camptothecin (b)

MDA-MB468 cells were treated with vehicle, the Akt inhibitors (Compound 1, a selective Akt1 inhibitor) and (Compound 10, a selective Akt1 and Akt2 inhibitor) and LY294002 (an inhibitor of PI3K activity) in the presence of Trail (a) or Camptothecin (b). Solid bars represent the presence of Trail or Camptothecin while the open bars represent the absence of Trail or Camptothecin. Compound 1 demonstrated an increase in Caspase 3 activity, which is a marker for induction of apoptosis, in the presence of Trail but not Camptothecin. Compound 10 and LY294002 demonstrated an increase in Caspase 3 activity, which is a marker for induction of apoptosis, in the presence of Camptothecin or Trail. These results demonstrate that selective Akt1 inhibition increases Caspase 3 activity in the presence of Trail, and selective Akt1 and Akt2 inhibition increases Caspase 3 activity in the presence of both Trail and Camptothecin. Details of the experimental procedures are found in Example 40.

FIG. 7: Caspase 3 Assay on LnCaP Cells +/− TRAIL Ligand

Two Akt inhibitors (Compound 13-9, a selective Akt1 inhibitor) and (Compound 13-4, a selective Akt2 inhibitor) were tested, in a time course experiment, (+/− TRAIL) for Caspase 3 activation alone or in combination in LnCaP cells. Caspase 3 activation is utilized as a marker for an increase in cell death. Compound 13-9 demonstrated a 1.5-4.5 fold increase in Caspase 3 activation over Trail alone, while Compound 13-4 produced a 2.5-6 fold increase in Caspase 3 activation over Trail alone. Combination treatment (Compounds 13-9 and 13-4) demonstrated a 6.5-14 fold increase in Caspase 3 activation over Trail alone. Details of the experimental procedures are found in Example 40.

### DETAILED DESCRIPTION OF THE INVENTION

The present invention relates to a method of treating cancer which is comprised of administering to a patient in need of such treatment amounts of at least one inhibitor of Akt and at least one inhibitor of a protein kinase.

In another aspect, the invention relates to a method of treating cancer which is comprised of administering to a patient in need of such treatment amounts of at least two selective inhibitors of Akt.

In practicing the instant methods of treatment, it is understood that the individual inhibitors of Akt and the inhibitor(s) of protein kinases may be administered either simultaneously in a single pharmaceutical composition or individually in separate pharmaceutical compositions. If the individual inhibitors of Akt and the inhibitors of protein kinases are administered in separate compositions, such compositions may be administered simultaneously or consecutively.

The term “consecutively” when used in the context of administration of two or more separate pharmaceutical compositions means that administrations of the separate pharmaceutical compositions are at separate times. The term “consecutively” also includes administration of two or more separate pharmaceutical compositions wherein administration of one or more pharmaceutical compositions is a continuous administration over a prolonged period of time and wherein administration of another of the compositions occur at a discrete time during the prolonged period.

The term “inhibiting Akt/PKB activity” as used herein describes the decrease in the in vitro and in vivo biochemical modifications resulting from the phosphorylation of Akt by upstream kinases and/or the subsequent phosphorylation of downstream targets of Akt by activated Akt. Thus, the terms “inhibitor of Akt/PKB activity” and “inhibitor of Akt/PKB [isoforms]” describe an agent that, by binding to Akt, either inhibits the phosphorylation of Akt by upstream kinases (thereby reducing the amount of activated Akt) or inhibits the phosphorylation by activated Akt of protein targets of Akt, or inhibits both of these biochemical steps. In another embodiment, the inhibitor utilized in the instant methods inhibits the phosphorylation of Akt by upstream kinases (thereby reducing the amount of activated Akt) and inhibits the phosphorylation by activated Akt of protein targets of Akt.

In an embodiment, the inhibitors of Akt are selective inhibitors useful in the instant method of treatment that are selected from: a selective inhibitor of Akt1, a selective inhibitor of Akt2, a selective inhibitor of Akt3, a selective inhibitor of two of the three Akt isoforms (such as “Akt1/2, Akt 1/3 and Akt 2/3”) or a selective inhibitor of all three Akt isoforms.

In another aspect of the invention, the selective inhibitors useful in the instant method of treatment are selected from: a selective inhibitor of Akt1, a selective inhibitor of Akt2, a selective inhibitor of Akt3, a selective inhibitor of both Akt1 and Akt2 (“Akt1/2”), a selective inhibitor of both Akt1 and Akt3 (“Akt1/3”), or a selective inhibitor of both Akt2 and Akt3 (“Akt2/3”). In another embodiment, the selective inhibitors useful in the instant method of treatment are selected from: a selective inhibitor of Akt1, a selective inhibitor of Akt2 and a selective inhibitor of both Akt1 and Akt2 (“Akt1/2”). In another embodiment of the invention the selective inhibitors do not inhibit Akt3.

In another aspect of the invention, the selective Akt inhibitors useful in the instant method are small organic molecules. The term “small organic molecule”, as used herein, refers to a compound that is an organic molecule of a size comparable to those organic molecules generally used in pharmaceuticals. The term excludes biological macromolecules (e.g., proteins, nucleic acids, etc.). Preferred small organic molecules range in size up to about 2000 Da, and more preferably in size up to about 1000 Da.

The term “selective inhibitor” as used herein is intended to mean that the inhibiting compound exhibits greater inhibition against the activity of the indicated isoform(s) of Akt, when compared to the compounds inhibition of the activity of the other Akt isoform(s) and other kinases, such as PKA and PKC. In an embodiment, the selectively inhibiting compound exhibits at least about a 5 fold greater inhibition against the activity of the indicated isoform(s) of Akt. In another embodiment, the selectively inhibiting compound exhibits at least about a 50 fold greater inhibition against the activity of the indicated isoform(s) of Akt. The Akt inhibitors of the instant invention are selective inhibitors and have an IC₅₀ of <50 μM against one, two, or all three isozymes of Akt. In another embodiment, the Akt inhibitors of the instant invention are selective inhibitors and have an IC₅₀ of <50 μM against one or two of the isozymes of Akt. See WO 02/083675, WO 02/083139, WO 02/083140, WO 02/083138, WO 02/083064, U.S. S No. 60/370,833 filed on Apr. 8, 2002, U.S. S No. 60/370,842 filed on Apr. 8, 2002, U.S. S No. 60/370,847 filed on Apr. 8, 2002, U.S. S No. 60/370,827 filed on Apr. 8, 2002, U.S. S No. 60/370,846 filed on Apr. 8, 2002

In another embodiment of the invention, the methods of treating cancer and inhibiting Akt comprise administering an inhibitor whose activity is dependent on the presence of the pleckstrin homology (PH) domain, the hinge region or both the PH domain and the hinge region of Akt.

The PH domains and hinge regions of the three Akt isoforms, though presumably functionally equivalent in terms of lipid binding, show little sequence homology and are much less conserved than the catalytic domains. Inhibitors of Akt that function by binding to the PH domain, the hinge region or both are thus able to discriminate between the three Akt isozymes.

A selective inhibitor whose inhibitory activity is dependent on the PH domain exhibits a decrease in in vitro inhibitory activity or no in vitro inhibitory activity against truncated Akt/PKB proteins lacking the PH domain.

A selective inhibitor whose inhibitory activity is dependent on the hinge region, the region of the protein between the PH domain and the kinase domain (see Konishi et al. Biochem. and Biophys. Res. Comm. 216: 526-534 (1995), FIG. 2, incorporated herein by reference), exhibits a decrease in in vitro inhibitory activity or no in vitro inhibitory activity against truncated Akt proteins lacking the PH domain and the hinge region or the hinge region alone.

The method of using such an inhibitor that is dependent on either the PH domain, the hinge region or both provides a particular advantage since the PH domains and hinge regions in the Akt isoforms lack the sequence homology that is present in the rest of the protein, particularly the homology found in the kinase domains (which comprise the catalytic domains and ATP-binding consensus sequences). It is therefore observed that certain inhibitor compounds, such as those described herein, are not only selective for one or two isoforms of Akt, but also are weak inhibitors or fail to inhibit other kinases, such as PKA and PKC, whose kinase domains share some sequence homology with the kinase domains of the Akt/PKB isoforms. Both PKA and PKC lack a PH domain and a hinge region.

In another aspect of the invention that comprises administering an inhibitor whose activity is dependent on the presence of the pleckstrin homology (PH) domain, the hinge region or both the PH domain and the hinge region of Akt, the selective inhibitor is selected from: a selective inhibitor of Akt1, a selective inhibitor of Akt2 or a selective inhibitor of both Akt1 and Akt2 (“Akt1/2”).

In another aspect of the invention, the selective inhibitor(s) useful in the instant method of treatment are selected from: a selective inhibitor of Akt1, a selective inhibitor of Akt2, a selective inhibitor of Akt3 or a selective inhibitor of two of the three Akt isoforms.

In another aspect of the invention, the selective inhibitor(s) useful in the instant method of treatment are selected from: a selective inhibitor of Akt1, a selective inhibitor of Akt2, but not a selective inhibitor of Akt3.

In another embodiment, the selective inhibitor of one or two of the Akt isoforms useful in the instant method of treatment is not an inhibitor of one or both of such Akt isoforms that have been modified to delete the PH domain, the hinge region or both the PH domain and the hinge region.

In another embodiment, the selective inhibitor of all three Akt isoforms useful in the instant method of treatment is not an inhibitor of one, two or all of such Akt isoforms that have been modified to delete the PH domain, the hinge region or both the PH domain and the hinge region.

In another embodiment of the instant invention is provided a method for selectively inhibiting Akt activity in a cell which comprises the administration of one or more selective Akt inhibitors.

In another embodiment of the instant invention is provided a method for selectively inhibiting Akt activity in a cell wherein the Akt activity that is inhibited is the activity of Akt1 and Akt2. In another embodiment, the Akt activity that is inhibited is the activity of Akt1 and Akt2, but the activity of Akt3 is not inhibited.

In another embodiment of the instant invention is provided a method for selectively inhibiting Akt activity in a cell, wherein the selective inhibition of Akt activity comprises the administration of one or more selective inhibitors of Akt isoforms. In another embodiment the inhibitors of Akt isoforms are administered either simultaneously or consecutively.

In another embodiment of the instant invention is provided a method for selectively inhibiting Akt activity in a cell, wherein the selective inhibition of Akt activity comprises the administration of one or more selective inhibitors of Akt isoforms: the selective inhibitors of Akt are selected from:

a) an Akt1 selective inhibitor,

b) an Akt2 selective inhibitor,

c) an Akt3 selective inhibitor,

d) a selective inhibitor of both Akt1 and Akt2,

e) a selective inhibitor of both Akt1 and Akt3,

f) a selective inhibitor of both Akt2 and Akt3, and

g) a selective inhibitor of Akt1, Akt2 and Akt3.

In another embodiment, the selective inhibitor comprises an Akt1 selective inhibitor, an Akt2 selective inhibitor and a selective inhibitor of both Akt1 and Akt2. In another embodiment, the selective inhibitor comprises an Akt1 selective inhibitor and an Akt2 selective inhibitor but not an Akt3 selective inhibitor.

In another embodiment of the instant invention is provided a method for selectively inhibiting Akt activity in a cell, wherein the selective inhibition of Akt activity comprises the administration of one or more selective inhibitors of Akt isoforms: the selective Akt inhibitor is selected from a protein, a nucleotide and a small molecule. In another embodiment the selective inhibitor is a small molecule.

In another embodiment of the instant invention is provided a method for selectively inhibiting Akt activity in cell, wherein the selective inhibition of Akt activity comprises the administration of one or more selective inhibitors of Akt isoforms and is useful for the treatment of cancer.

Also included in the instant methods is an inhibitor of protein kinases in a mammal.

As used herein, the term “inhibit” or “inhibiting” or “inhibition” or “inhibited” with respect to an inhibitor of protein kinases refers to the inhibition of the catalytic activity of protein kinases, including but not limited to receptor tyrosine kinases (RTKs), cellular tyrosine kinases (CTKs) and serine-threonine kinases (STKs).

The term “catalytic activity” as used herein refers to the rate of phosphorylation of tyrosine under the influence, direct or indirect, of RTKs and/or CTKs or the phosphorylation of serine and threonine under the influence, direct or indirect, of STKs.

The above-referenced inhibitor of a protein kinase, that is a component of the method of this invention, inhibits a protein kinase selected from the group comprising an RTK, a CTK or an STK. In another embodiment, the protein kinase is an RTK.

Furthermore, it is an aspect of this invention that the inhibitor of a protein kinase, including RTK, is an inhibitor of a protein kinase selected from the group comprising EGF, HER2, HER3, HER4, IR, IGF-1R, IRR, PDGFRα, PDGFRβ, TrkA, TrkB, TrkC, HGF, CSFIR, C-Kit, C-fms, Flk-1R, Flk4, KDR/Flk-1, Flt-1, FGFR-1R, FGFR-1R, FGFR-3R and FGFR-4R. In another embodiment of the invention, the protein kinase, including RTK, is selected from IR, IGF-1R, or IRR.

In addition, it is an aspect of this invention that the protein kinase whose catalytic activity is inhibited by a compound that is a component of the method of this invention is selected from the group consisting of, but not limited to, Src, Frk, Btk, Csk, Abl, BCR-Abl, ZAP70, Fes, Fps, Fak, Jak, Ack, Yes, Fyn, Lyn, Lck, Blk, Hck, Fgr and Yrk.

Another aspect of this invention is that the protein kinase, including serine-threonine protein kinase, whose catalytic activity is inhibited by a compound utilized in the method of treatment of this invention, is selected from the group consisting of but not limited to CDK2, Raf, Mek, p38, Erk, JNK, and mTOR.

Furthermore, it is an aspect of this invention that the inhibitor of a protein kinase, that is a component of the method of this invention, is selected from the group comprising a small molecule compound, an antibody, or an antisense oligonucleotide.

In another aspect of the invention, the inhibitor of a protein kinase is a small molecule compound or an antibody.

In another aspect of the invention, the inhibitor of a protein kinase is a small molecule compound.

In another aspect of the invention, the inibitor of a protein kinase is a herceptin antibody.

In another aspect, this invention relates to a method for treating cancer in a mammal in need of such treatment comprising administering to the mammal a therapeutically effective amount of two or more of the compounds described herein.

The term “administration” and variants thereof (e.g., “administering” a compound) in reference to a compound utilized in the method of treatment of the invention means introducing the compound or a prodrug of the compound into the system of the animal in need of treatment. The instant methods of treatment is understood to include concurrent and sequential introduction of the compounds or prodrugs thereof and other agents.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer including the inhibition of cancerous tumor growth and regression of cancerous tumors.

The compounds of the instant invention are inhibitors of the activity of Akt and are thus useful in the treatment of cancer, in particular cancers associated with irregularities in the activity of Akt and downstream cellular targets of Akt. Such cancers include, but are not limited to, ovarian, pancreatic, breast and prostate cancer, as well as cancers (including glioblastoma) where the tumor suppressor PTEN is mutated (Cheng et al., Proc. Natl. Acad. Sci. (1992) 89:9267-9271; Cheng et al., Proc. Natl. Acad. Sci. (1996) 93:3636-3641; Bellacosa et al., Int. J. Cancer (1995) 64:280-285; Nakatani et al., J. Biol. Chem. (1999) 274:21528-21532; Graff, Expert. Opin. Ther. Targets (2002) 6(1):103-113; and Yamada and Araki, J. Cell Science. (2001) 114:2375-2382; Mischel and Cloughesy, Brain Pathol. (2003) 13(1):52-61).

Akt signaling regulates multiple critical steps in angiogenesis. Shiojima and Walsh, Circ. Res. (2002) 90:1243-1250. Moreover, effects of protein kinases in the regulation of angiogenesis are well known. The utility of angiogenesis inhibitors in the treatment of cancer is known in the literature, see J. Rak et al. Cancer Research, 55:4575-4580, 1995 and Dredge et al., Expert Opin. Biol. Ther. (2002) 2(8):953-966, for example. The role of angiogenesis in cancer has been shown in numerous types of cancer and tissues: breast carcinoma (G. Gasparini and A. L. harris, J. Clin. Oncol., 1995, 13:765-782; M. Toi et al., Japan. J. Cancer Res., 1994, 85:1045-1049); bladder carcinomas (A. J. Dickinson et al., Br. J. Urol., 1994, 74:762-766); colon carcinomas (L. M. Ellis et al., Surgery, 1996, 120(5):871-878); and oral cavity tumors (J. K. Williams et al., Am. J. Surg., 1994, 168:373-380). Other cancers include, advanced tumors, hairy cell leukemia, melanoma, chronic myelogenous leukemia, advanced head and neck, metastatic renal cell, non-Hodgkin's lymphoma, metastatic breast, breast adenocarcinoma, advanced melanoma, pancreatic, gastric, glioblastoma, lung, ovarian, non-small cell lung, prostate, small cell lung, renal cell carcinoma, various solid tumors, multiple myeloma, metastatic prostate, malignant glioma, renal cancer, lymphoma, refractory metastatic disease, refractory multiple myeloma, cervical cancer, Kaposi's sarcoma, recurrent anaplastic glioma, and metastatic colon cancer (Dredge et al., Expert Opin. Biol. Ther. (2002) 2(8):953-966). Thus, the Akt inhibitors and the inhibitors of protein kinases, disclosed in the instant application, are also useful in the treatment of these angiogenesis related cancers.

Tumors which have undergone neovascularization show an increased potential for metastasis. In fact, angiogenesis is essential for tumor growth and metastasis. (S. P. Cunningham, et al., Can. Research, 61: 3206-3211 (2001)). The Akt inhibitors and inhibitors of protien kinases disclosed in the present application are therefore also useful to prevent or decrease tumor cell metastasis.

Further included within the scope of the invention is a method of treating or preventing a disease in which angiogenesis is implicated, which is comprised of administering to a mammal in need of such treatment a therapeutically effective amount of a compound(s) of the present invention. Ocular neovascular diseases are an example of conditions where much of the resulting tissue damage can be attributed to aberrant infiltration of blood vessels in the eye (see WO 00/30651, published 2 Jun. 2000). The undesireable infiltration can be triggered by ischemic retinopathy, such as that resulting from diabetic retinopathy, retinopathy of prematurity, retinal vein occlusions, etc., or by degenerative diseases, such as the choroidal neovascularization obeserved in age-related macular degeneration. Inhibiting the growth of blood vessels by administration of the present compounds would therefore prevent the infiltration of blood vessels and prevent or treat diseases where angiogenesis is implicated, such as ocular diseases like retinal vascularization, diabetic retinopathy, age-related macular degeneration, and the like.

Further included within the scope of the invention is a method of treating or preventing a disease in which angiogenesis is implicated, including but not limited to: atherosclerosis, arthritis, psoriasis, obesity and Alzheimer's disease (Dredge et al., Expert Opin. Biol. Ther. (2002) 2(8):953-966), and other hyperproliferative disorders such as restinosis, inflamation, autoimmune diseases, allergy/asthma, and further including hyperinsulinism.

Inhibitors of the instant invention are useful in the treatment of cancer and angiogenesis related diseases, alone, in combination, or in combination with other agents as discussed herein and include administration to the patient at constant intervals at low “metronomic” dose schedules, as well as other dosing methods that are well known in the art and as discussed herein.

Included within the scope of the present invention is a pharmaceutical composition, which is comprised of a compound(s) disclosed herein and a pharmaceutically acceptable carrier. The present invention also encompasses a method of treating cancer in a mammal in need of such treatment which is comprised of administering to said mammal a therapeutically effective amount of a compound(s) disclosed herein.

In another embodiment, the types of cancers which may be treated using a pharmaceutical composition disclosed herein include, but are not limited to, breast cancer, prostate cancer, pancreatic cancer, colorectal cancer, lung cancer, ovarian cancer, renal cell carcinoma, endometrial carcinoma, glioblastoma, colon cancer and bladder cancer. In another aspect, the cancer being treated is selected from breast cancer, prostate cancer, pancreatic cancer and ovarian cancer.

A pharmaceutical composition which is useful for the treatments of the instant invention may comprise one or more inhibitors of Akt, one or more inhibitors of a protein kinase, or a combination thereof, preferably, in combination with pharmaceutically acceptable carriers, excipients or diluents, in a pharmaceutical composition, according to standard pharmaceutical practice. The composition may be administered to mammals, preferably humans. The composition can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropyl-cellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyl-eneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized.

An example of such a device is the Deltec CADD-PLUS™ model 5400 intravenous pump.

The pharmaceutical compositions may be in the form of a sterile injectable aqueous or oleaginous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conven-tionally employed as a solvent or suspending medium. For this purpose any bland fixed oil may be employed including synthetic mono- or diglycerides. In addition, fatty acids such as oleic acid find use in the preparation of injectables.

The instant compositions may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the instant compositions are employed. (For purposes of this application, topical application shall include mouth washes and gargles.)

The compositions useful in the instant invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The composition of at least two Akt inhibitors and the composition of at least one Akt inhibitor and at least one inhibitor of a protein kinase, useful in the instant methods of treatment may also be co-administered with a third therapeutic agent that is selected for a particular usefulness against the condition that is being treated.

For example, two or more selective Akt inhibitors and/or at least one selective Akt inhibitor and at least one protein kinase inhibitor are useful in combination with known anti-cancer agents and are also useful in combination with known therapeutic agents and anti-cancer agents. For example, two or more selective Akt inhibitors and/or at least one selective Akt inhibitor and at least one protein kinase inhibitor are useful in combination with known anti-cancer agents. Combinations of two or more selective Akt inhibitors and/or at least one selective Akt inhibitor and at least one protein kinase inhibitor with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, and agents that interfere with cell cycle checkpoints. Two or more selective Akt inhibitors and/or at least one selective Akt inhibitor and at least one protein kinase inhibitor are particularly useful when co-administered with radiation therapy.

In an embodiment, two or more selective Akt inhibitors and/or at least one selective Akt inhibitor and at least one protein kinase inhibitor are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites, biological response modifiers, hormonal/anti-hormonal therapeutic agents, haematopoietic growth factors, monoclonal antibody targeted therapeutic agents, topoisomerase inhibitors, proteosome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNPI350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768 and WO 01/98278, and pending U.S. Ser. Nos. 60/338,779 (filed Dec. 6, 2001), 60/338,344 (filed Dec. 6, 2001), 60/338,383 (filed Dec. 6, 2001), 60/338,380 (filed Dec. 6, 2001), 60/338,379 (filed Dec. 6, 2001) and 60/344,453 (filed Nov. 7, 2001). In an embodiment inhibitors of mitotic kinesins include, but are not limited to inhibitors of KSP, inhibitors of MKLP1, inhibitors of CENP-E, inhibitors of MCAK and inhibitors of Rab6-KIFL.

“Inhibitors of kinases involved in mitotic progression: include, but are not limited to, inhibitors of aurora kinases, inhibitors of Polo-like kinases (PLK; in particular inhibitors of PLK-1), inhibitors of bub-1 and inhibitors of bub-R¹.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

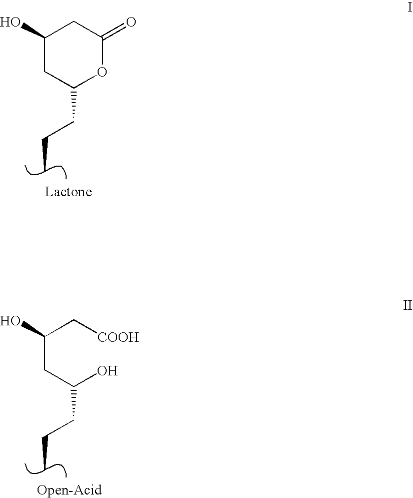

In HMG-CoA reductase inhibitors where an open-acid form can exist, salt and ester forms may be formed from the open-acid, and all such forms are included within the meaning of the term “HMG-CoA reductase inhibitor” as used herein. In an embodiment, the HMG-CoA reductase inhibitor is selected from lovastatin and simvastatin, and in a further embodiment, simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-1), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]4-(3-chlorophenyl)-1-methyl-2(1H)-quinolinone, (−)-6-[amino(4-chlorophenyl)(1-methyl-1H-imidazol-5-yl)methyl]-4-(3-chlorophenyl)-1-methyl-2(1H)-quinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)-4-[1-(4-cyanobenzyl)-5-imidazolylmethyl]-2-piperazinone, (S)-1-(3-chlorophenyl)-4-[1-(4-cyanobenzyl)-5-imidazolylmethyl]-5-[2-(ethanesulfonyl)methyl)-2-piperazinone, 5(S)-n-Butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′ ]bipyridin-5′-ylmethyl]-3H-imidazol-4-ylmethyl}benzonitrile, 4-{3-[4-(2-oxo-2H-[1,2′]bipyridin-5′-ylmethyl]-3H-imidazol-4-ylmethyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6,10:12,16-dimetheno-1H-imidazo[4,3-c][1,11,4]dioxaazacyclo-nonadecine-9-carbonitrile, (±)-19,20-dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacycloeicosine-9-carbonitrile, and (±)-19,20-dihydro-3-methyl-19-oxo-5H-18,21-ethano-12,14-etheno-6,10-metheno-22H-benzo [d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp.1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p.573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p.107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp.963-968 (October 1999); Kim et al., Nature, 362, 841-844 (1993); WO 00/44777; and WO 00/61186).

Other therapeutic agents that modulate or inhibit angiogenesis and may also be used in combination with two or more selective Akt inhibitors and/or at least one selective Akt inhibitor and at least one protein kinase inhibitor of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

As described above, the combinations with NSAID's are directed to the use of NSAID's which are potent COX-2 inhibiting agents. For purposes of this specification an NSAID is potent if it possesses an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference.

Inhibitors of COX-2 that are particularly useful in the instant method of treatment are:

3-phenyl-4-(4-(methylsulfonyl)phenyl)-2-(5H)-furanone; and

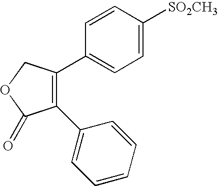

5-chloro-3-(4-methylsulfonyl)phenyl-2-(2-methyl-5-pyridinyl)pyridine;

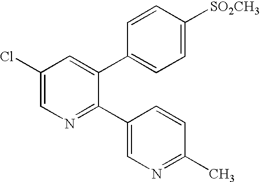

or a pharmaceutically acceptable salt thereof.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following:

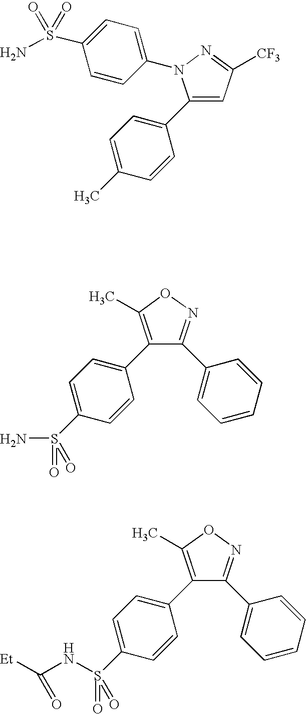

or a pharmaceutically acceptable salt thereof.

Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM 101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl-4-(4-pyridylmethyl)-1-phthalazinamine, and EMD121974.

Combinations with compounds other than anti-cancer compounds are also encompassed in the instant methods. For example, combinations of two or more selective Akt inhibitors and/or at least one selective Akt inhibitor and at least one protein kinase inhibitor with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. S No. 60/235,708 and 60/244,697).

Another embodiment of the instant invention is the use of two or more selective Akt inhibitors and/or at least one selective Akt inhibitor and at least one protein kinase inhibitor in combination with gene therapy for the treatment of cancer. For an overview of genetic strategies to treating cancer see Hall et al (Am. J. Hum. Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J. Immunol. 2000;164:217-222).

Two or more selective Akt inhibitors and/or at least one selective Akt inhibitor and at least one protein kinase inhibitor, of the instant invention, may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR⁹⁵⁷⁶, OC144-093, R¹⁰¹⁹²², VX853 and PSC833 (valspodar).

Two or more selective Akt inhibitors and/or at least one selective Akt inhibitor and at least one protein kinase inhibitor of the present invention may be employed in conjunction with anti-emetic agents to treat nausea or emesis, including acute, delayed, late-phase, and anticipatory emesis, which may result from the use of two or more selective Akt inhibitors and/or at least one selective Akt inhibitor and at least one protein kinase inhibitor, of the present invention, alone or with radiation therapy. For the prevention or treatment of emesis, two or more selective Akt inhibitors and/or at least one selective Akt inhibitor and at least one protein kinase inhibitor, of the present invention, may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. For the treatment or prevention of emesis that may result upon administration of two or more selective Akt inhibitors and/or at least one selective Akt inhibitor and at least one protein kinase inhibitor, conjunctive therapy with an antiemesis agent selected from a neurokinin-1 receptor antagonist, a 5HT3 receptor antagonist and a corticosteroid is preferred.

Neurokinin-1 receptor antagonists of use in conjunction with two or more selective Akt inhibitors and/or at least one selective Akt inhibitor and at least one protein kinase inhibitor, of the present invention, are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with two or more selective Akt inhibitors and/or at least one selective Akt inhibitor and at least one protein kinase inhibitor, of the present invention, is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

Two or more selective Akt inhibitors and/or at least one selective Akt inhibitor and at least one protein kinase inhibitor, of the instant invention, may also be administered with an agent useful in the treatment of anemia. Such an anemia treatment agent is, for example, a continuous eythropoiesis receptor activator (such as epoetin alfa).

Two or more selective Akt inhibitors and/or at least one selective Akt inhibitor and at least one protein kinase inhibitor, of the instant invention, may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

Two or more selective Akt inhibitors and/or at least one selective Akt inhibitor and at least one protein kinase inhibitor, of the instant invention, may also be administered with an immunologic-enhancing drug, such as levamisole, isoprinosine and Zadaxin.

Thus, the scope of the instant invention encompasses the use of two or more selective Akt inhibitors and/or at least one selective Akt inhibitor and at least one protein kinase inhibitor in combination with a third compound selected from:

1) an estrogen receptor modulator,

2) an androgen receptor modulator,

3) retinoid receptor modulator,

4) a cytotoxic/cytostatic agent,

5) an antiproliferative agent,

6) a prenyl-protein transferase inhibitor,

7) an HMG-CoA reductase inhibitor,

8) an HIV protease inhibitor,

9) a reverse transcriptase inhibitor,

10) an angiogenesis inhibitor,

11) PPAR-γ agonists,

12) PPAR-δ agonists,

13) an inhibitor of inherent multidrug resistance,

14) an anti-emetic agent,

15) an agent useful in the treatment of anemia,

16) an agent useful in the treatment of neutropenia,

17) an immunologic-enhancing drug,

18) an inhibitor of cell proliferation and survival signaling, and

19) an agent that interferes with a cell cycle checkpoint.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering therapeutically effective amounts of two or more selective Akt inhibitors and/or at least one selective Akt inhibitor and at least one protein kinase inhibitor, in combination with radiation therapy and/or in combination with a third compound selected from:

1) an estrogen receptor modulator,

2) an androgen receptor modulator,

3) a retinoid receptor modulator,

4) a cytotoxic/cytostatic agent,

5) an antiproliferative agent,

6) a prenyl-protein transferase inhibitor,

7) an HMG-CoA reductase inhibitor,

8) an HIV protease inhibitor,

9) a reverse transcriptase inhibitor,

10) an angiogenesis inhibitor,

11) PPAR-γ agonists,

12) PPAR-δ agonists,

13) an inhibitor of inherent multidrug resistance,

14) an anti-emetic agent,

15) an agent useful in the treatment of anemia,

16) an agent useful in the treatment of neutropenia,

17) an immunologic-enhancing drug,

18) an inhibitor of cell proliferation and survival signaling, and

19) an agent that interferes with a cell cycle checkpoint.

And yet another embodiment of the invention is a method of treating cancer that comprises administering therapeutically effective amounts of two or more selective Akt inhibitors and/or at least one selective Akt inhibitor and at least one protein kinase inhibitor, in combination with paclitaxel or trastuzumab.

The invention further encompasses a method of treating or preventing cancer that comprises administering therapeutically effective amounts of two or more selective Akt inhibitors and/or at least one selective Akt inhibitor and at least one protein kinase inhibitor, in combination with a COX-2 inhibitor.

The instant invention also includes a pharmaceutical composition useful for treating or preventing cancer that comprises therapeutically effective amounts of two or more selective Akt inhibitors and/or at least one selective Akt inhibitor and at least one protein kinase inhibitor, and a third compound selected from:

1) an estrogen receptor modulator,

2) an androgen receptor modulator,

3) a retinoid receptor modulator,

4) a cytotoxic/cytostatic agent,

5) an antiproliferative agent,

6) a prenyl-protein transferase inhibitor,

7) an HMG-CoA reductase inhibitor,

8) an HIV protease inhibitor,

9) a reverse transcriptase inhibitor,

10) an angiogenesis inhibitor,

11) a PPAR-γ agonist,

12) a PPAR-δ agonist,

13) an inhibitor of cell proliferation and survival signaling, and

14) an agent that interferes with a cell cycle checkpoint.

If formulated as a fixed dose, the compositions useful in the instant invention employ the Akt inhibitor(s) and the protein kinase inhibitor(s) within the dosage ranges described below.

When compositions according to this invention are administered into a human subject, the daily dosage will normally be determined by the prescribing physician with the dosage generally varying according to the age, weight, and response of the individual patient, as well as the severity of the patient's sysmptoms.

In one exemplary application, suitable amounts of inhibitors of Akt and a suitable amount of a protein kinase inhibitor are administered to a mammal undergoing treatment for cancer. Administration occurs in an amount of inhibitor of between about 0.1 mg/kg of body weight to about 60 mg/kg of body weight per day, preferably of between 0.5 mg/kg of body weight to about 40 mg/kg of body weight per day. A particular daily therapeutic dosage that comprises the instant composition includes from about 0.01 mg to about 1000 mg of inhibitor of Akt/PKB and an inhibitor of a growth factor or growth factor receptor. Preferably, the daily dosage comprises from about 1 mg to about 1000 mg of inhibitor.

Inhibitors of Akt kinases useful in the instant invention include the following compounds:

i) a compound of the formula I:

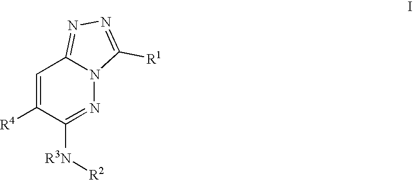

wherein

R¹ represents phenyl, furyl, thienyl or pyridinyl, any of which groups may be optionally substituted with one, two or three substituents, independently selected from:

a) halogen;

b) C₁₋₄ alkyl;

c) C₁₋₄ alkoxy;

d) cyano;

e) di(C₁₋₄ alkyl)amino;

f) hydroxy;

R² represents amino-C₁₋₆ alkyl, C₁₋₄ alkylamino-(C₁₋₆)alkyl, di(C₁₋₄ alkyl)amino-(C₁₋₆)alkyl, hydroxy-(C₁₋₆)alkyl or C₁₋₄ alkoxy-(C₁₋₆)alkyl, any of which groups may be optionally substituted;

R³ represents hydrogen or C₁₋₆ alkyl; and

R⁴ is selected from: C₃₋₇ cycloalkyl and aryl, any of which groups may be optionally substituted;

ii) a compound of the formula II:

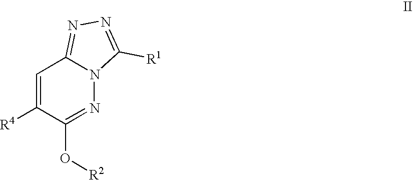

wherein

R¹ represents phenyl, furyl, thienyl or pyridinyl, any of which groups may be optionally substituted with one, two or three substituents, independently selected from:

a) halogen;

b) C₁₋₄ alkyl;

c) C₁₋₄alkoxy;

d) cyano;

e) di(C₁₋₄ alkyl)amino;

f) hydroxy;

R² represents amino-C₁₋₆ alkyl, C₁₋₄ alkylamino-(C₁₋₆)alkyl, di(C₁₋₄ alkyl)amino-(C₁₋₆)alkyl, hydroxy-(C₁₋₆)alkyl or C₁₋₄ alkoxy-(C₁₋₆)alkyl, any of which groups may be optionally substituted; and

R⁴ is selected from: C₃₋₇ cycloalkyl and aryl, any of which groups may be optionally substituted;

iii) a compound of the formula III:

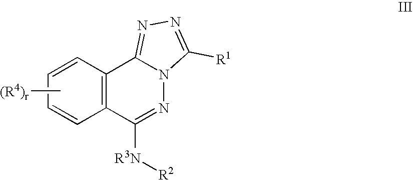

wherein

R¹ represents phenyl, furyl, thienyl or pyridinyl, any of which groups may be optionally substituted with one, two or three substituents, independently selected from:

a) halogen;

b) C₁₋₄ alkyl;

c) C₁₋₄ alkoxy;

d) cyano;

e) di(C₁₋₄ alkyl)amino;

f) hydroxy;

R² represents amino-C₁₋₆ alkyl, C₁₋₄ alkylamino-(C₁₋₆)alkyl, di(C₁₋₄ alkyl)amino-(C₁₋₆)alkyl, hydroxy-(C₁₋₆)alkyl or C₁₋₄ alkoxy-(C₁₋₆)alkyl, any of which groups may be optionally substituted;

R³ represents hydrogen or C₁₋₆ alkyl; and

R⁴ independently represents hydrogen, C₁₋₆-alkyl, halogen, HO— or C₁₋₆ alkyl-O;

r is 1 or 2;

iv) a compound of the formula IV:

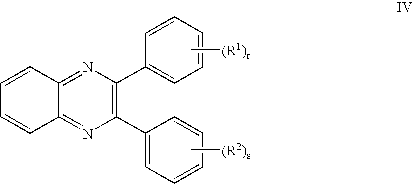

wherein

R¹ independently represents amino, C₁₋₆-alkyl amino, di-C₁₋₆-alkylamino, amino-C₁₋₆ alkyl, C₁₋₆ alkylamino-(C₁₋₆)alkyl or di(C₁₋₆ alkyl)amino-(C₁₋₆)alkyl;

R² independently represents hydrogen, amino, C₁₋₆-alkyl amino, di-C₁₋₆alkylamino, amino-C₁₋₆ alkyl, C₁₋₆ alkylamino-(C₁₋₆)alkyl or di(C₁₋₆ alkyl)amino-(C₁₋₆)alkyl;

r is 1 to 3;

s is 1 to 3;

v) a compound of the formula V:

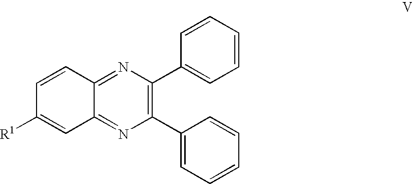

wherein

R¹ independently represents hydrogen, C₁₋₆-alkyl, halogen, HO— or C₁₋₆ alkyl-O;

or a pharmaceutically acceptable salt thereof.

vi) a compound of the formula VI:

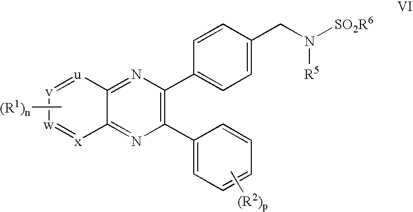

wherein:

n is 0, 1, 2 or 3;

p is 0, 1 or 2;

r is 0 or 1;

s is 0 or 1;

u, v, w and x are independently selected from: CH and N, provided that only one of u, v, w and x may be N;

R¹ is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁷R⁸,

13) NR^{c}(C═O)NR⁷R⁸,

14) S(O)ₘR^{a},

15) S(O)₂NR⁷R⁸,

16) NR^{c}S(O)ₘR^{a},

17) oxo,

18) CHO,

19) NO₂,

20) NR^{c}(C═O)O_{b}R^{a},

21) O(C═O)O_{b}C₁-C₁₀ alkyl,

22) O(C═O)O_{b}C₃-C₈ cycloalkyl,

23) O(C═O)O_{b}aryl, and

24) O(C═O)O_{b}-heterocycle,

said alkyl, aryl, alkenyl, alkynyl, heterocyclyl, and cycloalkyl optionally substituted with one or more substituents selected from R^{z};

R² is independently selected from:

1) C₁-C₆ alkyl,

2) aryl,

3) heterocyclyl,

4) CO₂H,

5) halo,

6) CN,

7) OH,

8) S(O)₂NR⁷R⁸,

said alkyl, aryl and heterocyclyl optionally substituted with one, two or three substituents selected from R^{z};

R⁵ is independently selected from:

1) H,

2) C₁-C₁₀ alkyl,

3) aryl, and

4) C₃-C₈ cycloalkyl,

said alkyl, cycloalkyl and aryl is optionally substituted with one or more substituents selected from R^{z};

R⁶ is NR⁷R⁸, (C₁-C₆)alkyl, (C₁-C₆)perfluoroalkyl, (C₃-C₆)cycloalkyl, noboranyl, aryl, 2,2,2-trifluoroethyl, benzyl or heterocyclyl, said alkyl, cycloalkyl, noboranyl, aryl, heterocyclyl and benzyl is optionally substituted with one or more substituents selected from R^{z};

R⁷ and R⁸ are independently selected from:

1) H,

2) (C═O)O_{b}C₁-C₁₀ alkyl,

3) (C═O)O_{b}C₃-C₈ cycloalkyl,

4) (C═O)O_{b}aryl,

5) (C═O)O_{b} heterocyclyl,

6) C₁-C₁₀ alkyl,

7) aryl,

8) C₂-C₁₀ alkenyl,

9) C₂-C₁₀ alkynyl,

10) heterocyclyl,

11) C₃-C₈ cycloalkyl,

12) SO₂R^{a}, and

13) (C═O)NR^{b}₂,

said alkyl, cycloalkyl, aryl, heterocylyl, alkenyl, and alkynyl is optionally substituted with one or more substituents selected from R^{z}, or

R^{z} is selected from:

1) (C═O)ᵣOₛ(C₁-C₁₀)alkyl,

2) Oᵣ(C₁-C₃)perfluoroalkyl,

3) (C₀-C₆)alkylene-S(O)ₘR^{a},

4) oxo,

5) OH,

6) halo,

7) CN,

8) (C═O)ᵣOₛ(C₂-C₁₀)alkenyl,

9) (C═O)ᵣOₛ(C₂-C₁₀)alkynyl,

10) (C═O)ᵣOₛ(C₃-C₆)cycloalkyl,

11) (C═O)ᵣOₛ(C₀-C₆)alkylene-aryl,

12) (C═O)ᵣOₛ(C₀-C₆)alkylene-heterocyclyl,

13) (C═O)ᵣOₛ(C₀-C₆)alkylene-N(R^{b})₂,

14) C(O)R^{a},

15) (C₀-C₆)alkylene-CO₂R^{a},

16) C(O)H,

17) (C₀-C₆)alkylene-CO₂H,

18) C(O)N(R^{b})₂,

19) S(O)ₘR^{a}, and

20) S(O)₂NR⁹R¹⁰

21) NR^{c}(C═O)O_{b}R^{a},

22) O(C═O)O_{b}C₁-C₁₀ alkyl,

23) O(C═O)O_{b}C₃-C₈ cycloalkyl,

24) O(C═O)O_{b}aryl, and

25) O(C═O)O_{b}-heterocycle,

said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;

R^{a} is (C₁-C₆)alkyl, (C₃-C₆)cycloalkyl, substituted or unsubstituted aryl, or heterocyclyl; and

R^{b} is H, (C₁-C₆)alkyl, substituted or unsubstituted aryl, substituted or unsubstituted benzyl, substituted or unsubstituted heterocyclyl, (C₃-C₆)cycloalkyl, (C═O)OC₁-C₆ alkyl, (C═O)C₁-C₆ alkyl or S(O)₂R^{a};

R^{c} is selected from:

1) H,

2) C₁-C₁₀ alkyl,

3) aryl,

4) C₂-C₁₀ alkenyl,

5) C₂-C₁₀ alkynyl,

6) heterocyclyl,

7) C₃-C₈ cycloalkyl,

8) C₁-C₆ perfluoroalkyl,

said alkyl, cycloalkyl, aryl, heterocylyl, alkenyl, and alkynyl is optionally substituted with one or more substituents selected from R^{z};

or a pharmaceutically acceptable salt thereof.

vii) a compound of the formula VII:

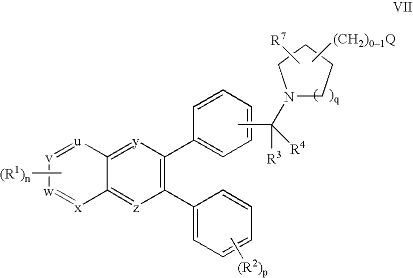

wherein:

a is 0 or 1;

b is 0 or 1;

m is 0, 1 or 2;

n is 0, 1, 2 or 3;

p is 0, 1 or 2;

q is 0, 1, 2, 3 or 4;

r is 0 or 1;

s is 0 or 1;

t is 2, 3, 4, 5 or 6;

u, v, w and x are independently selected from: CH and N;

y and z are independently selected from: CH and N, provided that at least one of y and z is N;

Q is selected from: —NR⁵R⁶, aryl and heterocyclyl, said aryl and heterocycle which is optionally substituted with one to three R^{z};

R¹ is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁵R⁶,

13) NR^{c}(C═O)NR⁵R⁶,

14) S(O)ₘR^{a},

15) S(O)₂NR⁵R⁶,

16) NR^{c}S(O)ₘR^{a},

17) oxo,

18) CHO,

19) NO₂,

20) NR^{c}(C═O)O_{b}R^{a},

21) O(C═O)O_{b}C₁-C₁₀ alkyl,

22) O(C═O)O_{b}C₃-C₈ cycloalkyl,

23) O(C═O)O_{b}aryl, and

24) O(C═O)O_{b}-heterocycle,

said alkyl, aryl, alkenyl, alkynyl, heterocyclyl, and cycloalkyl optionally substituted with one or more substituents selected from R^{z};

R² is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁵R⁶,

13) NR^{c}(C═O)NR⁵R⁶,

14) S(O)ₘR^{a},

15) S(O)₂NR⁵R⁶,

16) NR^{c}S(O)ₘR^{a},

17) CHO,

18) NO₂,

19) NR^{c}(C═O)O_{b}R^{a},

20) O(C═O)O_{b}C₁-C₁₀ alkyl,

21) O(C═O)O_{b}C₃-C₈ cycloalkyl,

22) O(C═O)O_{b}aryl, and

23) O(C═O)O_{b}-heterocycle,

said alkyl, aryl, alkenyl, alkynyl, heterocyclyl, and cycloalkyl optionally substituted with one, two or three substituents selected from R^{z};

R³ and R⁴ are independently selected from: H, C₁-C₆-alkyl and C₁-C₆-perfluoroalkyl, or

R³ and R⁴ are combined to form —(CH₂)ₜ— wherein one of the carbon atoms is optionally replaced by a moiety selected from O, S(O)ₘ, —N(R^{b})C(O)—, and —N(COR^{a})—;

R⁵ and R⁶ are independently selected from:

1) H,

2) (C═O)O_{b}R^{a},

3) C₁-C₁₀ alkyl,

4) aryl,

5) C₂-C₁₀ alkenyl,

6) C₂-C₁₀ alkynyl,

7) heterocyclyl,

8) C₃-C₈ cycloalkyl,

9) SO₂R^{a}, and

10) (C═O)NR^{b}₂,

said alkyl, cycloalkyl, aryl, heterocylyl, alkenyl, and alkynyl is optionally substituted with one or more substituents selected from R^{z}, or

R⁵ and R⁶ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};

R⁷ is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁵R⁶,

13) NR⁵(C═O)NR⁵R⁶,

14) S(O)ₘR^{a},

15) S(O)₂NR⁵R⁶,

16) NR^{s}S(O)ₘR^{a},

17) oxo,

18) CHO,

19) NO₂,

20) O(C═O)O_{b}C₁-C₁₀ alkyl, and

21) O(C═O)O_{b}C₃-C₈ cycloalkyl,

said alkyl, aryl, alkenyl, alkynyl, heterocyclyl, and cycloalkyl optionally substituted with one or more substituents selected from R^{z};

R^{z} is selected from:

1) (C═O)ᵣOS(C₁-C₁₀)alkyl,

2) Oᵣ(C₁-C₃)perfluoroalkyl,

3) (C₀-C₆)alkylene-S(O)ₘR^{a},

4) oxo,

5) OH,

6) halo,

7) CN,

8) (C═O)ᵣOₛ(C₂-C₁₀)alkenyl,

9) (C═O)ᵣOₛ(C₂-C₁₀)alkynyl,

10) (C═O)ᵣOₛ(C₃-C₆)cycloalkyl,

11) (C═O)ᵣOₛ(C₀-C₆)alkylene-aryl,

12) (C═O)ᵣOₛ(C₀-C₆)alkylene-heterocyclyl,

13) (C═O)ᵣOₛ(C₀-C₆)alkylene-N(R^{b})₂,

14) C(O)R^{a},

15) (C₀-C₆)alkylene-CO₂R^{a},

16) C(O)H,

17) (C₀-C₆)alkylene-CO₂H,

18) C(O)N(R^{b})₂,

19) S(O)ₘR^{a},

20) S(O)₂N(R^{b})₂

21) NR^{c}(C═O)O_{b}R^{a},

22) O(C═O)O_{b}C₁-C₁₀ alkyl,

23) O(C═O)O_{b}C₃-C₈ cycloalkyl,

24) O(C═O)O_{b}aryl, and

25) O(C═O)O_{b}-heterocycle,

said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂; R^{a} is substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₆)cycloalkyl, substituted or unsubstituted aryl, (C₁-C₆)perfluoroalkyl, 2,2,2-trifluoroethyl, or substituted or unsubstituted heterocyclyl; and

R^{b} is H, (C₁-C₆)alkyl, substituted or unsubstituted aryl, substituted or unsubstituted benzyl, substituted or unsubstituted heterocyclyl, (C₃-C₆)cycloalkyl, (C═O)OC₁-C₆ alkyl, (C═O)C₁-C₆ alkyl or S(O)₂R^{a};

R^{c} is selected from:

1) H,

2) C₁-C₁₀ alkyl,

3) aryl,

4) C₂-C₁₀ alkenyl,

5) C₂-C₁₀ alkynyl,

6) heterocyclyl,

7) C₃-C₈ cycloalkyl,

8) C₁-C₆ perfluoroalkyl,

said alkyl, cycloalkyl, aryl, heterocylyl, alkenyl, and alkynyl is optionally substituted with one or more substituents selected from R^{z},

or a pharmaceutically acceptable salt or a stereoisomer thereof.

viii) a compound of the formula VIII:

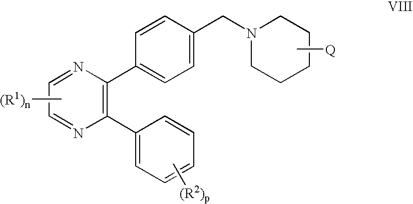

wherein:

n is 0, 1 or 2;

p is 0, 1 or 2;

r is 0 or 1;

s is 0 or 1;

Q is selected from: —NR⁷R⁸ and heterocyclyl, the heterocyclyl optionally substituted with one or two R^{z};

R¹ is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁷R⁸,

13) NR^{c}(C═O)NR⁷R⁸,

14) S(O)ₘR^{a},

15) S(O)₂NR⁷R⁸,

16) NR^{c}S(O)ₘR^{a},

17) oxo,

18) CHO,

19) NO₂,

20) NR^{c}(C═O)O_{b}R^{a},

21) O(C═O)O_{b}C₁-C₁₀ alkyl,

22) O(C═O)O_{b}C₃-C₈ cycloalkyl,

23) O(C═O)O_{b}aryl, and

24) O(C═O)O_{b}-heterocycle,

said alkyl, aryl, alkenyl, alkynyl, heterocyclyl, and cycloalkyl optionally substituted with one or more substituents selected from R^{z};

R² is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁷R⁸,

13) NR^{c}(C═O)NR⁷R⁸,

14) S(O)ₘR^{a},

15) S(O)₂NR⁷R⁸,

16) NR^{c}S(O)ₘR^{a},

17) CHO,

18) NO₂,

19) NR^{c}(C═O)O_{b}R^{a},

20) O(C═O)O_{b}C₁-C₁₀ alkyl,

22) O(C═O)O_{b}C₃-C₈ cycloalkyl,

23) O(C═O)O_{b}aryl, and

24) O(C═O)O_{b}-heterocycle,

said alkyl, aryl, alkenyl, alkynyl, heterocyclyl, and cycloalkyl optionally substituted with one, two or three substituents selected from R^{z};

R⁷ and R⁸ are independently selected from:

1) H,

2) (C═O)O_{b}C₁-C₁₀ alkyl,

3) (C═O)O_{b}C₃-C₈ cycloalkyl,

4) (C═O)O_{b}aryl,

5) (C═O)O_{b}heterocyclyl,

6) C₁-C₁₀ alkyl,

7) aryl,

8) C₂-C₁₀ alkenyl,

9) C₂-C₁₀ alkynyl,

10) heterocyclyl,

11) C₃-C₈ cycloalkyl,

12) SO₂R^{a}, and

13) (C═O)NR^{b}₂,

said alkyl, cycloalkyl, aryl, heterocylyl, alkenyl, and alkynyl is optionally substituted with one or more substituents selected from R^{z}, or

R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};

R^{z} is selected from:

1) (C═O)ᵣOₛ(C₁-C₁₀)alkyl,

2) Oᵣ(C₁-C₃)perfluoroalkyl,

3) (C₀-C₆)alkylene-S(O)ₘR^{a},

4) oxo,

5) OH,

6) halo,

7) CN,

8) (C═O)ᵣOₛ(C₂-C₁₀)alkenyl,

9) (C═O)ᵣOₛ(C₂-C₁₀)alkynyl,

10) (C═O)ᵣOₛ(C₃-C₆)cycloalkyl,

11) (C═O)ᵣOₛ(C₀-C₆)alkylene-aryl,

12) (C═O)ᵣOₛ(C₀-C₆)alkylene-heterocyclyl,

13) (C═O)ᵣOₛ(C₀-C₆)alkylene-N(R^{b})₂,

14) C(O)R^{a},

15) (C₀-C₆)alkylene-CO₂R^{a},

16) C(O)H,

17) (C₀-C₆)alkylene-CO₂H,

18) C(O)N(R^{b})₂,

19) S(O)ₘR^{a},

20) S(O)₂NR⁹R¹⁰

21) NR^{c}(C═O)O_{b}R^{a},

22) O(C═O)O_{b}C₁-C₁₀ alkyl,

23) O(C═O)O_{b}C₃-C₈ cycloalkyl,

24) O(C═O)O_{b}aryl, and

25) O(C═O)O_{b}-heterocycle,

said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;

R^{a} is (C₁-C₆)alkyl, (C₂-C₆)alkenyl, (C₂-C₆)alkynyl, (C₃-C₆)cycloalkyl, substituted or unsubstituted aryl, (C₁-C₆)perfluoroalkyl, 2,2,2-trifluoroethyl, or substituted or unsubstituted heterocyclyl; and

R^{b} is H, (C₁-C₆)alkyl, aryl, heterocyclyl, (C₃-C₆)cycloalkyl, (C═O)OC₁-C₆ alkyl, (C═O)C₁-C₆ alkyl or S(O)₂R^{a};

R^{c} is selected from:

1) H,

2) C₁-C₁₀ alkyl,

3) aryl,

4) C₂-C₁₀ alkenyl,

5) C₂-C₁₀ alkynyl,

6) heterocyclyl,

7) C₃-C₈ cycloalkyl,

8) C₁-C₆ perfluoroalkyl,

said alkyl, cycloalkyl, aryl, heterocylyl, alkenyl, and alkynyl is optionally substituted with one or more substituents selected from R^{z}, or

or a pharmaceutically acceptable salt or a stereoisomer thereof.

ix) a compound of the formula IX:

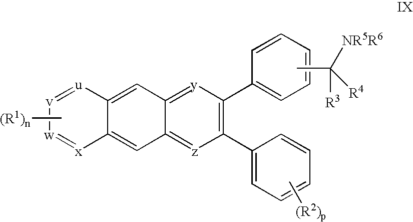

wherein:

a is 0 or 1;

b is 0 or 1;

m is 0, 1 or 2;

n is 0, 1 or 2;

p is 0, 1, 2 or 3;

r is 0 or 1;

s is 0 or 1;

t is 2, 3, 4, 5 or 6;

u, v and x are independently selected from: CH and N;

w is selected from a bond, CH and N;

y and z are independently selected from: CH and N, provided that at least one of y and z is N;

R¹ is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁷R⁸,

13) NR^{c}(C═O)NR⁷R⁸,

14) S(O)ₘR^{a},

15) S(O)₂NR⁷R⁸,

16) NR^{c}S(O)ₘR^{a},

17) oxo,

18) CHO,

19) NO₂,

20) NR^{c}(C═O)O_{b}R^{a},

21) O(C═O)O_{b}C₁-C₁₀ alkyl,

22) O(C═O)O_{b}C₃-C₈ cycloalkyl,

23) O(C═O)O_{b}aryl, and

24) O(C═O)O_{b}-heterocycle,

said alkyl, aryl, alkenyl, alkynyl, heterocyclyl, and cycloalkyl optionally substituted with one or more substituents selected from R^{z};

R² is independently selected from:

1) (C═O)ₐO_{b}C₁-C₁₀ alkyl,

2) (C═O)ₐO_{b}aryl,

3) C₂-C₁₀ alkenyl,

4) C₂-C₁₀ alkynyl,

5) (C═O)ₐO_{b} heterocyclyl,

6) (C═O)ₐO_{b}C₃-C₈ cycloalkyl,

7) CO₂H,

8) halo,

9) CN,

10) OH,

11) O_{b}C₁-C₆ perfluoroalkyl,

12) Oₐ(C═O)_{b}NR⁷R⁸,

13) NR^{c}(C═O)NR⁷R⁸,

14) S(O)ₘR^{a},

15) S(O)₂NR⁷R⁸,

16) NR^{c}S(O)ₘR^{a},

17) CHO,

18) NO₂,

19) NR^{c}(C═O)O_{b}R^{a},

20) O(C═O)O_{b}C₁-C₁₀ alkyl,

21) O(C═O)O_{b}C₃-C₈ cycloalkyl,

22) O(C═O)O_{b}aryl, and

23) O(C═O)O_{b}-heterocycle,

said alkyl, aryl, alkenyl, alkynyl, heterocyclyl, and cycloalkyl optionally substituted with one, two or three substituents selected from R^{z};

R³ and R⁴ are independently selected from: H, C₁-C₆-alkyl and C₁-C₆-perfluoroalkyl, or

R³ and R⁴ are combined to form —(CH₂)ₜ— wherein one of the carbon atoms is optionally replaced by a moiety selected from O, S(O)ₘ, —N(R^{b})C(O)—, and —N(COR^{a})—;

R⁵ and R⁶ are independently selected from:

1) H,

2) (C═O)O_{b}R^{a},

3) C₁-C₁₀ alkyl,

4) aryl,

5) C₂-C₁₀ alkenyl,

6) C₂-C₁₀ alkynyl,

7) heterocyclyl,

8) C₃-C₈ cycloalkyl,

9) SO₂R^{a}, and

10) (C═O)NR^{b}₂,

said alkyl, cycloalkyl, aryl, heterocylyl, alkenyl, and alkynyl is optionally substituted with one or more substituents selected from R^{z}, or

R⁵ and R⁶ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with Q and also optionally substituted with one or more substituents selected from R^{z};

R⁷ and R⁸ are independently selected from:

1) H,

2) (C═O)O_{b}C₁-C₁₀ alkyl,

3) (C═O)O_{b}C₃-C₈ cycloalkyl,

4) (C═O)O_{b}aryl,

5) (C═O)O_{b} heterocyclyl,

6) C₁-C₁₀ alkyl,

7) aryl,

8) C₂-C₁₀ alkenyl,

9) C₂-C₁₀ alkynyl,

10) heterocyclyl,

11) C₃-C₈ cycloalkyl,

12) SO₂R^{a}, and

13) (C═O)NR^{b}₂,

said alkyl, cycloalkyl, aryl, heterocylyl, alkenyl, and alkynyl is optionally substituted with one or more substituents selected from R^{z}, or

R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};

R^{z} is selected from:

1) (C═O)ᵣOₛ(C₁-C₁₀)alkyl,

2) Oᵣ(C₁-C₃)perfluoroalkyl,

3) (C₀-C₆)alkylene-S(O)ₘR^{a},

4) oxo,

5) OH,

6) halo,

7) CN,

8) (C═O)ᵣOₛ(C₂-C₁₀)alkenyl,

9) (C═O)ᵣOₛ(C₂-C₁₀)alkynyl,

10) (C═O)ᵣOₛ(C₃-C₆)cycloalkyl,

11) (C═O)ᵣOₛ(C₀-C₆)alkylene-aryl,

12) (C═O)ᵣOₛ(C₀-C₆)alkylene-heterocyclyl,

13) (C═O)ᵣOₛ(C₀-C₆)alkylene-N(R^{b})₂,

14) C(O)R^{a},

15) (C₀-C₆)alkylene-CO₂R^{a},

16) C(O)H,

17) (C₀-C₆)alkylene-CO₂H,

18) C(O)N(R^{b})₂,

19) S(O)ₘR^{a},

20) S(O)₂N(R^{b})₂,

21) NR^{c}(C═O)O_{b}R^{a},

22) O(C═O)O_{b}C₁-C₁₀ alkyl,

23) O(C═O)O_{b}C₃-C₈ cycloalkyl,

24) O(C═O)O_{b}aryl, and

25) O(C═O)O_{b}-heterocycle,

said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;

R^{a} is substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₆)cycloalkyl, substituted or unsubstituted aryl, (C₁-C₆)perfluoroalkyl, 2,2,2-trifluoroethyl, or substituted or unsubstituted heterocyclyl; and

R^{b} is H, (C₁-C₆)alkyl, substituted or unsubstituted aryl, substituted or unsubstituted benzyl, substituted or unsubstituted heterocyclyl, (C₃-C₆)cycloalkyl, (C═O)OC₁-C₆ alkyl, (C═O)C₁-C₆ alkyl or S(O)₂R^{a};

R^{c} is selected from:

1) H,

2) C₁-C₁₀ alkyl,

3) aryl,

4) C₂-C₁₀ alkenyl,

5) C₂-C₁₀ alkynyl,

6) heterocyclyl,

7) C₃-C₈ cycloalkyl,

8) C₁-C₆ perfluoroalkyl,

said alkyl, cycloalkyl, aryl, heterocylyl, alkenyl, and alkynyl is optionally substituted with one or more substituents selected from R^{z}, or

or a pharmaceutically acceptable salt or a stereoisomer thereof.

In another embodiment, compounds which inhibit Akt kinases and are useful in the instant method of treating cancer are represented by compounds of the formula VII.

Examples of compounds which inhibit Akt kinases include the following:

N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide;

N-[2-(diethylamino)ethyl]-2-(4-{[4-(2-oxo-2,3-dihydro-1H-benzimidazol-1-yl)piperidin-1-yl]methyl}phenyl)-3-phenylquinoxaline-6-carboxamide;

N′-(7-Cyclobutyl-3-phenyl-[1,2,4]triazolo[4,3-b]pyridazin-6-yl)-2,2,N,N-tetramethyl-propane-1,3-diamine;

N′-(7-Cyclobutyl-3-(3,5-difluoro-phenyl)-[1,2,4]triazolo[4,3-b]pyridazin-6-yl)-2,2,N,N-tetramethyl-propane-1,3-diamine;

N′-(7-Cyclobutyl-3-(3,4-difluoro-phenyl)-[1,2,4]triazolo[4,3-b]pyridazin-6-yl)-2,2,N,N-tetramethyl-propane-1,3-diamine;

N′-(7-Cyclobutyl-3-(4-fluoro-phenyl)-[1,2,4]triazolo[4,3-b]pyridazin-6-yl)-2,2,N,N-tetramethyl-propane-1,3-diamine;

N′-(7-Cyclobutyl-3-(3-fluoro-phenyl)-[1,2,4]triazolo[4,3-b]pyridazin-6-yl)-2,2,N,N-tetramethyl-propane-1,3-diamine;

2,2,N,N-tetramethyl-N-(3-phenyl-[1,2,4]triazolo[3,4-a]phthalazin-6-yl)-propane-1,3-diamine;

N′-[3-(4-Methoxy-phenyl)-[1,2,4]triazolo[4,3-a]phthalazin-6-yl)-2,2,N,N-tetramethyl-propane-1,3-diamine;

6-(2-hydroxyethyl)oxy-3,7-diphenyl-[1,2,4]triazolo[4,3-b]pyridazine;

6-(4-hydroxybutyl)oxy-3,7-diphenyl-[1,2,4]triazolo[4,3-b]pyridazine;

2-(2-aminoprop-2-ylphenyl)-3-phenylquinazoline;

1-{1-[4-(7-Phenyl-1H-imidazo[4,5-g]quinoxalin-6-yl)benzyl]piperidin-4-yl}-1,3-dihydro-2H-benzimidazol-2-one;

1-{1-[4-(6-Hydroxy-5-isobutyl-3-phenylpyrazin-2-yl)benzyl]piperidin-4-yl}-1,3-dihydro-2H-benzimidazol-2-one;

1-{1-[4-(5-Hydroxy-6-isobutyl-3-phenylpyrazin-2-yl)benzyl]piperidin-4-yl}-1,3-dihydro-2H-benzimidazol-2-one;

1-(1-{4-[5-Hydroxy-6-(1H-indol-3-ylmethyl)-3-phenylpyrazin-2-yl]benzyl}piperidin-4-yl)-1,3-dihydro-2H-benzimidazol-2-one;

1-(1-{4-[6-Hydroxy-5-(1H-indol-3-ylmethyl)-3-phenylpyrazin-2-yl]benzyl}piperidin-4-yl)-1,3-dihydro-2H-benzimidazol-2-one;

1-{1-[4-(3-Phenylquinoxalin-2-yl)benzyl]piperidin-4-yl}-1,3-dihydro-2H-benzimidazol-2-one;

3-(4-{[4-(2-Oxo-2,3-dihydro-1H-benzamidazol-1-yl)piperdin-1-yl]methyl}phenyl)-2-phenylquinaxoline-6-carboxylic acid;

2-(4-{[4-(2-Oxo-2,3-dihydro-1H-benzamidazol-1-yl)piperdin-1-yl]methyl}phenyl)-2-phenylquinaxoline-6-carboxylic acid;

N-[3-(1H-Imidazol-1-yl)propyl]-3-(4-{[4-(2-oxo-2,3-dihydro-1H-benzamidazol-1-yl)piperdin-1-yl]methyl}phenyl)-2-phenylquinaxoline-6-carboxamide;

1-{1-[4-(3-phenylpyrido[3,4-b]pyrazin-2-yl)benzyl]piperidin-4-yl}-1,3-dihydro-2H-benzimidazol-2-one;

1-{1-[4-(2-phenylpyrido[3,4-b]pyrazin-3-yl)benzyl]piperidin-4-yl}-1,3-dihydro-2H-benzimidazol-2-one;

4-cyano-N-{(3R)-1-[4-(3-phenylquinoxalin-2-yl)benzyl]pyrrolidin-3-yl}benzamide;

N-{(3R)-1-[4-(3-phenylquinoxalin-2-yl)benzyl]pyrrolidin-3-yl}-1,3-thiazole-5-carboxamide;

2-(4-{[4-(6-amino-9H-purin-9-yl)piperidin-1-yl]methyl}phenyl)-3-phenylquinoxalin-6-amine;

9-{1-[4-(3-phenylpyrido[3,4-b]pyrazin-2-yl)benzyl]piperidin-4-yl}-9H-purin-6-amine;

9-{1-[4-(3-phenylpyrido[2,3-b]pyrazin-2-yl)benzyl]piperidin-4-yl}-9H-purin-6-amine;

2-(4-{[4-(6-amino-9H-purin-9-yl)piperidin-1-yl]methyl}phenyl)-3-phenylquinoxaline-6-carboxylic acid;

1-{1-[4-(3-phenylquinolin-2-yl)benzyl]piperidin-4-yl}-1,3-dihydro-2H-benzimidazol-2-one;

1-(1-{4-[3-phenyl-6-(1H-tetrazol-5-yl)quinoxalin-2-yl]benzyl}piperidin-4-yl)-1,3-dihydro-2H-benzimidazol-2-one;

1-(1-{4-[3-phenyl-7-(1H-tetrazol-5-yl)quinoxalin-2-yl]benzyl}piperidin-4-yl)-1,3-dihydro-2H-benzimidazol-2-one;

9-(1-{4-[3-phenyl-7-(1H-tetrazol-5-yl)quinoxalin-2-yl]benzyl}piperidin-4-yl)-9H-purin-6-amine; and

9-(1-{4-[3-phenyl-6-(1H-tetrazol-5-yl)quinoxalin-2-yl]benzyl}piperidin-4-yl)-9H-purin-6-amine;

or a pharmaceutically acceptable salt or a stereoisomer thereof.

Specific examples of compounds included in this invention include:

N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide;

N-[2-(diethylamino)ethyl]-2-(4-{[4-(2-oxo-2,3-dihydro-1H-benzimidazol-1-yl)piperidin-1-yl]methyl}phenyl)-3-phenylquinoxaline-6-carboxamide;

4-cyano-N-{(3R)-1-[4-(3-phenylquinoxalin-2-yl)benzyl]pyrrolidin-3-yl}benzamide;

N-{(3R)-1-[4-(3-phenylquinoxalin-2-yl)benzyl]pyrrolidin-3-yl}-1,3-thiazole-5-carboxamide;

2-(4-{[4-(6-amino-9H-purin-9-yl)piperidin-1-yl]methyl}phenyl)-3-phenylquinoxalin-6-amine;

9-{1-[4-(3-phenylpyrido[3,4-b]pyrazin-2-yl)benzyl]piperidin-4-yl}-9H-purin-6-amine;

9-{1-[4-(3-phenylpyrido[2,3-b]pyrazin-2-yl)benzyl]piperidin-4-yl}-9H-purin-6-amine;

2-(4-{[4-(6-amino-9H-purin-9-yl)piperidin-1-yl]methyl}phenyl)-3-phenylquinoxaline-6-carboxylic acid;

1-{1-[4-(3-phenylquinolin-2-yl)benzyl]piperidin-4-yl}-1,3-dihydro-2H-benzimidazol-2-one;

1-(1-{4-[3-phenyl-6-(1H-tetrazol-5-yl)quinoxalin-2-yl]benzyl}piperidin-4-yl)-1,3-dihydro-2H-benzimidazol-2-one;

1-(1-{4-[3-phenyl-7-(1H-tetrazol-5-yl)quinoxalin-2-yl]benzyl}piperidin-4-yl)-1,3-dihydro-2H-benzimidazol-2-one;

9-(1-{4-[3-phenyl-7-(1H-tetrazol-5-yl)quinoxalin-2-yl]benzyl}piperidin-4-yl)-9H-purin-6-amine; and

9-(1-{4-[3-phenyl-6-(1H-tetrazol-5-yl)quinoxalin-2-yl]benzyl}piperidin-4-yl)-9H-purin-6-amine;

or a pharmaceutically acceptable salt or a stereoisomer thereof.

Compounds which are selective inhibitors of Akt and are useful in the present invention, and methods of synthesis thereof, can be found in the following patents, pending applications and publications, which are herein incorporated by reference:

WO 02/083675

WO 02/083139

WO 02/083140

WO 02/083138

WO 02/083064

U.S. S No. 60/370,833 filed on Apr. 8, 2002

U.S. S No. 60/370,842 filed on Apr. 8, 2002

U.S. S No. 60/370,847 filed on Apr. 8, 2002

U.S. S No. 60/370,827 filed on Apr. 8, 2002

U.S. S No. 60/370,846 filed on Apr. 8, 2002

Inhibitors of protein kinases useful in the instant invention include the following:

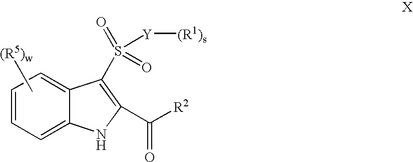

wherein

Y is selected from:

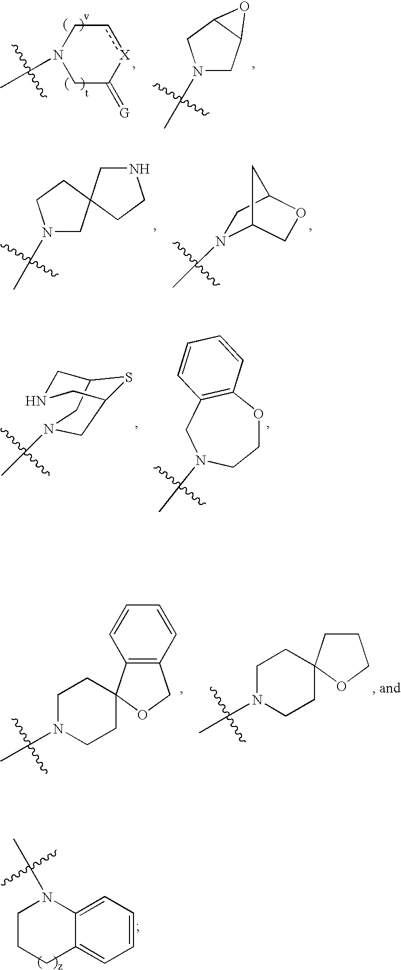

----- represents an optional double bond;

X is C, N, S(O)ₘ or O;

G is H₂ or O;

R^{a} is independently selected from:

1) H,

2) C₁-C₆ alkyl,

3) Halogen,

4) Aryl,

5) Heterocycle,

6) C₃-C₁₀ cycloalkyl, or

7) OR⁴;

said alkyl, aryl, heterocycle and cycloalkyl is optionally substituted with at least one substituent selected from R⁷;

R¹ is independently selected from:

1) H,

2) (CR^{a}₂)ₙR⁶,

3) (CR^{a}₂)ₙC(O)R⁴,

4) C(O)N(R⁴)₂,

5) (CR^{a}₂)ₙOR⁴,

6) (CR^{a}₂)ₙN(R⁴)₂,

7) S(O)ₘR⁶,

8) S(O)ₘR⁶OR⁴,

9) C(O)N(R⁴)(CR^{a}₂)ₙR⁶,

10) C(O)N(R⁴)(CR^{a}₂)ₙOR⁴,

11) C(O)R⁶(CR^{a}₂)ₙR⁶,

12) C(O)N(R⁴)(CR^{a}₂)ₙS(O)ₘ(CR^{a}₂)ₙR⁶,

13) C(O)N(R⁴)(CR^{a}₂)ₙC(O)R⁶,

14) C(O)N(R⁴)(CR^{a}₂)ₙN(R⁴)₂,

15) Halogen,

16) N(R⁴)S(O)ₘR⁶,

17) (CR^{a}₂)ₙC(O)OR⁴, and

18) R⁶C(O)OR;

R² is:

1) H,

2) unsubstituted or substituted C₁-C₁₀ alkyl,

3) N(R⁴)₂,

4) OR⁴,

5) unsubstituted or substituted aryl, and

6) unsubstituted or substituted C₃-C₁₀ cycloalkyl;

R⁴ is independently selected from:

1) H,

2) C₁-C₆ alkyl,

3) C₃-C₁₀ cycloalkyl,

4) Aryl,

5) Heterocycle,

6) CF₃,

7) C₂-C₆ alkenyl, and

8) C₂-C₆ alkynyl;

said alkyl, cycloalkyl, aryl, heterocycle, alkenyl and alkynyl is optionally substituted with at least one substituent selected from R⁷;

R⁵ is independently selected from:

1) H,

2) Halogen,

3) NO₂,

4) CN,

5) CR⁴═C(R⁴)₂,

6) C≡CR⁴,

7) (CR^{a}₂)ₙOR⁴,

8) (CR^{a}₂)ₙN(R⁴)₂,

9) C(O)R⁴,

10) C(O)OR⁴,

11) (CR^{a}₂)ₙR⁴,

12) S(O)ₘR⁶,

13) S(O)ₘN(R⁴)₂,

14) OS(O)ₘR⁶,

15) N(R⁴)C(O)R⁴,

16) N(R⁴)S(O)ₘR⁶,

17) (CR^{a}₂)ₙN(R⁴)R⁶,

18) (CR^{a}₂)ₙN(R⁴)R⁶OR⁴,

19) (CR^{a}₂)ₙN(R⁴)(CR^{a}₂)ₙC(O)N(R⁴)₂,

20) N(R⁴)(CR^{a}₂)ₙR⁶,

21) N(R⁴)(CR^{a}₂)ₙN(R⁴)₂,

22) (CR^{a}₂)ₙC(O)N(R⁴)₂,

23) O(CR^{a}₂)ₙC(O)OR⁴, and

24) O(CR^{a}₂)ₙC(O)N(R⁴)₂;

R⁶ is independently selected from:

1) C₁-C₆ alkyl,

2) Aryl,

3) Heterocycle, and

4) C₃-C₁₀ cycloalkyl;

said alkyl, aryl, heterocycle and cycloalkyl is optionally substituted with at least one substituent of R⁷;

R⁷ is independently selected from:

1) Unsubstituted or substituted C₁-C₆ alkyl,

2) Halogen,

3) OR⁴,

4) CF₃,

5) Unsubstituted or substituted aryl,

6) Unsubstituted or substituted C₃-C₁₀ cycloalkyl,

7) Unsubstituted or substituted heterocycle,

8) S(O)ₘN(R⁴)₂,

9) C(O)OR⁴,

10) C(O)R⁴,

11) CN,

12) C(O)N(R⁴)₂,

13) N(R⁴)C(O)R⁴,

14) NO₂; and

15) S(O)ₘR⁶;

m is independently 0, 1 or 2;

n is independently 0, 1, 2, 3, 4, 5 or 6;

s is 0 to 6;

t is 0, 1, or 2;

v is 0, 1 or 2;

w is 0, 1, 2, 3 or 4;

z is 1 or 2;

or a pharmaceutically acceptable salt or stereoisomer thereof.

A second embodiment of the instant invention is a compound of Formula XI:

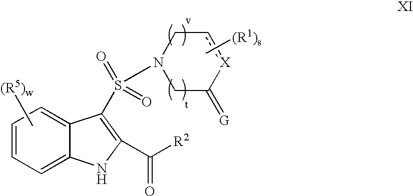

wherein:

----- represents an optional double bond;

X is C, N, S(O)ₘ or O;

G is H₂ or O;

R^{a} is independently selected from:

1) H,

2) C₁-C₆ alkyl,

3) Halogen,

4) Aryl,

5) Heterocycle,

6) C₃-C₁₀ cycloalkyl, and

7) OR⁴;

said alkyl, aryl, heterocycle and cycloalkyl is optionally substituted with at least one substituent selected from R⁷;

R¹ is independently selected from:

1) H,

2) (CR^{a}₂)ₙR⁶,

3) (CR^{a}₂)ₙC(O)R⁴,

4) C(O)N(R⁴)₂,

5) (CR^{a}₂)ₙOR⁴,

6) (CR^{a}₂)ₙN(R⁴)₂,

7) S(O)ₘR⁶,

8) S(O)ₘR⁶OR⁴,

9) C(O)N(R⁴)(CR^{a}₂)ₙR⁶,

10) C(O)N(R⁴)(CR^{a}₂)ₙOR⁴,

11) C(O)R⁶(CR^{a}₂)ₙR⁶,

12) C(O)N(R⁴)(CR^{a}₂)ₙS(O)ₘ(CR^{a}₂)ₙR⁶,

13) C(O)N(R⁴)(CR^{a}₂)ₙC(O)R⁶,

14) C(O)N(R⁴)(CR^{a}₂)ₙN(R⁴)₂,

15) Halogen,

16) N(R⁴)S(O)ₘR⁶,

17) (CR^{a}₂)ₙC(O)OR⁴, and

18) R⁶C(O)OR;

R² is:

1) H,

2) Unsubstituted or substituted C₁-C₁₀ alkyl,

3) N(R⁴)₂, or

4) OR⁴;

R⁴ is independently selected from:

1) H,

2) C₁-C₆ alkyl,

3) C₃-C₁₀ cycloalkyl,

4) Aryl,

5) Heterocycle,

6) CF₃,

7) C₂-C₆ alkenyl, and

8) C₂-C₆ alkynyl;

said alkyl, cycloalkyl, aryl, heterocycle, alkenyl and alkynyl is optionally substituted with at least one substituent selected from R⁷;

R⁵ is independently selected from:

1) H,

2) Halogen,

3) NO₂,

4) CN,

5) CR⁴═C(R⁴)₂,

6) C≡CR⁴,

7) (CR^{a}₂)ₙOR⁴,

8) (CR^{a}₂)ₙN(R⁴)₂,

9) C(O)R⁴,

10) C(O)OR⁴,

11) (CR^{a}₂)ₙR⁴,

12) S(O)ₘR⁶,

13) S(O)ₘN(R⁴)₂,

14) OS(O)ₘR⁶,

15) N(R⁴)C(O)R⁴,

16) N(R⁴)S(O)ₘR⁶,

17) (CR^{a}₂)ₙN(R⁴)R⁶,

18) (CR^{a}₂)ₙN(R⁴)R⁶OR⁴,

19) (CR^{a}₂)ₙN(R⁴)(CR^{a}₂)ₙC(O)N(R⁴)₂,

20) N(R⁴)(CR^{a}₂)ₙR⁶,

21) N(R⁴)(CR^{a}₂)ₙN(R⁴)₂, and

22) (CR^{a}₂)ₙC(O)N(R⁴)₂;

R⁶ is independently selected from:

1) C₁-C₆ alkyl,

2) Aryl,

3) Heterocycle, and

4) C₃-C₁₀ cycloalkyl;

said alkyl, aryl, heterocycle and cycloalkyl is optionally substituted with at least one substituent of R⁷;

R⁷ is independently selected from:

1) Unsubstituted or substituted C₁-C₆ alkyl,

2) Halogen,

3) OR⁴,

4) CF₃,

5) Unsubtituted or substituted aryl,

6) Unsubstituted or substituted C₃-C₁₀ cycloalkyl,

7) Unsubstituted or substituted heterocycle,

8) S(O)ₘN(R⁴)₂,

9) C(O)OR⁴,

10) C(O)R⁴,

11) CN,

12) C(O)N(R⁴)₂,

13) N(R⁴)C(O)R⁴,

14) S(O)ₘR⁶, and

15) NO₂;

m is independently 0, 1 or 2;

n is independently 0, 1, 2, 3, 4, 5 or 6;

s is 0 to 6;

t is 0, 1, or 2;

v is 0, 1 or 2;

w is 0, 1, 2, 3 or 4;

or a pharmaceutically acceptable salt or stereoisomer thereof.

A third embodiment of the instant invention is a compound of Formula XI, as described above, wherein:

R^{a} is independently selected from:

1) H,

2) C₁-C₆ alkyl,

3) Aryl, and

4) C₃-C₁₀ cycloalkyl;

said alkyl, aryl, and cycloalkyl is optionally substituted with at least one substituent selected from R⁷;

R¹ is independently selected from:

1) H,

2) (CR^{a}₂)ₙR⁶,

3) (CR^{a}₂)ₙC(O)R⁴,

4) C(O)N(R⁴)₂,

5) (CR^{a}₂)ₙOR⁴,

6) (CR^{a}₂)ₙN(R⁴)₂,

7) S(O)ₘR⁶,

8) S(O)ₘR⁶OR⁴,

9) C(O)N(R⁴)(CR^{a}₂)ₙR⁶,

10) C(O)N(R⁴)(CR^{a}₂)ₙOR⁴,

11) N(R⁴)S(O)ₘR⁶,

12) (CR^{a}₂)ₙC(O)OR⁴, and

13) R⁶C(O)OR;

R² is:

1) N(R⁴)₂, or

2) OR⁴;

s is 0 to 3;

and all other substituents and variables are as defined in the second embodiment;

or a pharmaceutically acceptable salt or stereoisomer thereof.

A further embodiment of the third embodiment is a compound of Formula XI, as described above, wherein:

R¹ is independently selected from:

1) H,

2) (CR^{a}₂)ₙR⁶,

3) (CR^{a}₂)ₙC(O)R⁴,

4) C(O)N(R⁴)₂,

5) (CR^{a}₂)ₙOR⁴,

6) (CR^{a}₂)ₙN(R⁴)₂,

7) S(O)ₘR⁶, and

8) S(O)ₘR⁶OR⁴;

and all other substituents and variables are as defined in the third embodiment;

or a pharmaceutically acceptable salt or stereoisomer thereof.

In another embodiment, compounds which inhibit protein kinases and are useful in the instant method of treating cancer are represented by compounds of the formula XI.

Examples of compounds which inhibit protein kinases include the following:

5-Chloro-3-(morpholin-4-ylsulfonyl)-1H-indole-2-carboxamide;

5-Bromo-3-(morpholin-4-ylsulfonyl)-1H-indole-2-carboxamide;

5-Iodo-3-(morpholin-4-ylsulfonyl)-1H-indole-2-carboxamide;

5-Methoxy-3-(morpholin-4-ylsulfonyl)-1H-indole-2-carboxamide;

6-Methoxy-3-(morpholin-4-ylsulfonyl)-1H-indole-2-carboxamide;

5-(Methylsulfonyl)-3-(morpholin-4-ylsulfonyl)-1H-indole-2-carboxamide;

7-Amino-3-(morpholin-4-ylsulfonyl)-1H-indole-2-carboxamide;

3-(Morpholin-4-ylsulfonyl)-5-nitro-1H-indole-2-carboxamide;

5-Chloro-3-(piperazin-1-ylsulfonyl)-1H-indole-2-carboxamide;

3-[(4-Benzylpiperazin-1-yl)sulfonyl]-5-chloro-1H-indole-2-carboxamide;

3-[(4-Acetylpiperazin-1-yl)sulfonyl]-5-chloro-1H-indole-2-carboxamide;

5-Chloro-3-(piperidin-1-ylsulfonyl)-1H-indole-2-carboxamide;

5-Chloro-3-(pyrrolidin-1-ylsulfonyl)-1H-indole-2-carboxamide;

5-Chloro-3-(thiomorpholin-4-ylsulfonyl)-1H-indole-2-carboxamide;

3-(Azetidin-1-ylsulfonyl)-5-chloro-1H-indole-2-carboxamide;

5-Chloro-3-[(oxidothiomorpholin-4-yl)sulfonyl]-1H-indole-2-carboxamide;

5-Chloro-3-[(1,1-dioxidothiomorpholin-4-yl)sulfonyl]-1H-indole-2-carboxamide;

cis-5-Chloro-3-(2,6-dimethylmorpholin-4-ylsulfonyl)-1H-indole-2-carboxamide;

trans-5-Chloro-3-(2,6-dimethylmorpholin-4-ylsulfonyl)-1H-indole-2-carboxamide;

5-Chloro-3-[(3-hydroxyazetidin-1-yl)sulfonyl]-1H-indole-2-carboxamide;

(±)-5-Chloro-3-{[2-(phenoxymethyl)morpholin-4-yl]sulfonyl}-1H-indole-2-carboxamide;

(S)-5-Chloro-3-{[2-(phenoxymethyl)morpholin-4-yl]sulfonyl}-1H-indole-2-carboxamide;

(R)-5-Chloro-3-{[2-(phenoxymethyl)morpholin-4-yl]sulfonyl}-1H-indole-2-carboxamide;

5-Bromo-3-({4-[2-(dimethylamino)ethyl]-5-oxo-1,4-diazepan-1-yl}sulfonyl)-1H-indole-2-carboxamide;

5-Bromo-3-({5-oxo-1,4-diazepan-1-yl}sulfonyl)-1H-indole-2-carboxamide;

5-Bromo-3-[(3-oxopiperazin-1-yl)sulfonyl]-1H-indole-2-carboxamide;

5-Bromo-3-[(3-hydroxyazetidin-1-yl)sulfonyl]-1H-indole-2-carboxamide;

(±)-5-Bromo-3-{[2-(aminocarbonyl)morpholin-4-yl]sulfonyl}-1H-indole-2-carboxamide;

3-(Azetidin-1-ylsulfonyl)-5-bromo-1H-indole-2-carboxamide;

5-Bromo-3-({4-[(4-methoxyphenyl)sulfonyl]piperazin-1-yl}sulfonyl)-1H-indole-2-carboxamide;

5-Bromo-3-({4-[(4-bromophenyl)sulfonyl]piperazin-1-yl}sulfonyl)-1H-indole-2-carboxamide;

5-Bromo-3-{[4-(3-morpholin-4-ylpropyl)-3-oxopiperazin-1-yl]sulfonyl}-1H-indole-2-carboxamide;

5-Bromo-3-({4-[3-(dimethylamino)propyl]-3-oxopiperazin-1-yl}sulfonyl)-1H-indole-2-carboxamide;

5-Bromo-3-(2,5-dihydroxy-1H-pyrrol-1-ylsulfonyl)-1H-indole-2-carboxamide;

5-Bromo-3-(6-oxa-3-azabicyclo[3.1.0]hex-3-ylsulfonyl)-1H-indole-2-carboxamide;

(±)-5-Bromo-3-{[2-(phenoxymethyl)morpholino-4-yl]sulfonyl}-1H-indole-2-carboxamide;

(S)-5-Bromo-3-{[2-(phenoxymethyl)morpholino-4-yl]sulfonyl}-1H-indole-2-carboxamide;

(R)-5-Bromo-3-{[2-(phenoxymethyl)morpholin-4-yl]sulfonyl}-1H-indole-2-carboxamide;

6-Hydroxy-3-(morpholin-4-ylsulfonyl)-1H-indole-2-carboxamide;

3-(Morpholin-4-ylsulfonyl)-1H-indole-2-carboxamide;

5-(2-Furyl)-3-(morpholin-4-ylsulfonyl)-1H-indole-2-carboxamide;

3-(Morpholin-4-ylsulfonyl)-5-(phenylethynyl)-1H-indole-2-carboxamide;

3-(Morpholin-4-ylsulfonyl)-5-(2-phenylethyl)-1H-indole-2-carboxamide;

5-Hex-1-ynyl-3-(morpholin-4-ylsulfonyl)-1H-indole-2-carboxamide;

5-Hexyl-3-(morpholin-4-ylsulfonyl)-1H-indole-2-carboxamide;

Methyl 2-(aminocarbonyl)-3-(morpholin-4-ylsulfonyl)-1H-indole-5-carboxylate;

3-(Morpholin-4-ylsulfonyl)-5-vinyl-1H-indole-2-carboxamide;

5-Hydroxy-3-(morpholin-4-ylsulfonyl)-1H-indole-2-carboxamide;

5-Ethoxy-3-(morpholin-4-ylsulfonyl)-1H-indole-2-carboxamide;

3-(Morpholin-4-ylsulfonyl)-5-propoxy-1H-indole-2-carboxamide;

5-Isopropoxy-3-(morpholin-4-ylsulfonyl)-1H-indole-2-carboxamide;

5-Ethyl-3-(morpholin-4-ylsulfonyl)-1H-indole-2-carboxamide;

2-(Aminocarbonyl)-3-(morpholin-4-ylsulfonyl)-1H-indol-5-yl methanesulfonate;

3-(Morpholin-4-ylsulfonyl)-5-prop-1-ynyl-1H-indole-2-carboxamide;

3-(Morpholin-4-ylsulfonyl)-5-thien-2-yl-1H-indole-2-carboxamide;

3-(Azetidin-1-ylsulfonyl)-5-methoxy-1H-indole-2-carboxamide;

5-Formyl-3-(morpholin-4-ylsulfonyl)-1H-indole-2-carboxamide;

5-Methyl-3-(morpholin-4-ylsulfonyl)-1H-indole-2-carboxamide;

7-(Acetylamino)-3-(morpholin-4-ylsulfonyl)-1H-indole-2-carboxamide;

7-[(Methylsulfonyl)amino]-3-(morpholin-4-ylsulfonyl)-1H-indole-2-carboxamide;

5-{[(4-Methoxyphenyl)amino]methyl}-3-morpholino-4-ylsulfonyl)-1H-indole-2-carboxamide;

5-{[(2-Acetamide)amino]methyl}-3-morpholino-4-ylsulfonyl)-1H-indole-2-carboxamide;

3-(Morpholino-4-ylsulfonyl)-5-phenyl-1H-indole-2-carboxamide;

3-(Morpholino-4-ylsulfonyl)-5-pyrazin-2-yl-1H-indole-2-carboxamide;

3-(Morpholino-4-ylsulfonyl)-5-pyridin-2-yl-1H-indole-2-carboxamide;

3-(Morpholino-4-ylsulfonyl)-5-pyridin-4-yl-1H-indole-2-carboxamide;

5-(1-Benzofuran-2-yl)-3-(morpholino-4-ylsulfonyl)-1H-indole-2-carboxamide;

5-(5-Methyl-2-furyl)-3-(morpholino-4-ylsulfonyl)-1H-indole-2-carboxamide;

5-(3,5-Dimethylisoxazole-4-yl)-3-(morpholino-4-ylsulfonyl)-1H-indole-2-carboxamide;

3-(Morpholin-4-ylsulfonyl)-5-(1H-pyrrol-2-yl)-1H-indole-2-carboxamide;

3-(Morpholin-4-ylsulfonyl)-5-pyridin-3-yl-1H-indole-2-carboxamide;

3-(Morpholin-4-ylsulfonyl)-5-(1,3-thiazol-2-yl)-1H-indole-2-carboxamide;

3-(Morpholin-4-ylsulfonyl)-5-thien-3-yl-1H-indole-2-carboxamide;

5-(1-Benzothien-3-yl)-3-(morpholin-4-ylsulfonyl)-1H-indole-2-carboxamide;

3-(Azetidin-1-yl}sulfonyl)-5-iodo-1H-indole-2-carboxamide;

3-[(3-Hydroxyazetidin-1-yl)sulfonyl]-5-iodo-1H-indole-2-carboxamide;

(±)-5-Iodo-3-{[2-(phenoxymethyl)morpholino-4-yl]sulfonyl}-1H-indole-2-carboxamide;

(S)-5-Iodo-3-{[2-(phenoxymethyl)morpholino-4-yl]sulfonyl}-1H-indole-2-carboxamide;

(R)-5-Iodo-3-{[2-(phenoxymethyl)morpholin-4-yl]sulfonyl}-1H-indole-2-carboxamide;

7-Amino-6-bromo-3-(morpholin-4-ylsulfonyl)-1H-indole-2-carboxamide;

7-Amino-4,6-dibromo-3-(morpholin-4-ylsulfonyl)-1H-indole-2-carboxamide;

6-Bromo-7-(dimethylamino)-3-(morpholin-4-ylsulfonyl)-1H-indole-2-carboxamide;

3-(Morpholin-4-ylsulfonyl)-7-[(pyridin-4-ylmethyl)amino]-1H-indole-2-carboxamide;

7-{[(2-Chloropyridin-4-yl)methyl]amino}-3-(morpholin-4-ylsulfonyl)-1H-indole-2-carboxamide;

7-Nitro-3-{[(2S)-2-(phenoxymethyl)morpholin-4-yl]sulfonyl}-1H-indole-2-carboxamide;

7-Amino-3-{[(2S)-2-(phenoxymethyl)morpholin-4-yl]sulfonyl}-1H-indole-2-carboxamide;

3-{[(2S)-2-(Phenoxymethyl)morpholin-4-yl]sulfonyl}-7-[(pyridin-4-ylmethyl)amino]-1H-indole-2-carboxamide;

7-(Benzylamino)-3-{[(2S)-2-(phenoxymethyl)morpholin-4-yl]sulfonyl}-1H-indole-2-carboxamide;

7-Chloro-3-(morpholin-4-ylsulfonyl)-1H-indole-2-carboxamide;

6-Bromo-3-(morpholin-4-ylsulfonyl)-1H-indole-2-carboxamide;

7-Bromo-3-(morpholin-4-ylsulfonyl)-1H-indole-2-carboxamide;

7-Cyano-3-(morpholin-4-ylsulfonyl)-1H-indole-2-carboxamide;

(±)-7-(Methylsulfinyl)-3-(morpholin-4-ylsulfonyl)-1H-indole-2-carboxamide;

7-Aminomethyl-3-(morpholin-4-ylsulfonyl)-1H-indole-2-carboxamide;

5-Amino-3-(morpholin-4-ylsulfonyl)-1H-indole-2-carboxamide;

(S)-5-Fluoro-3-{[2-(phenoxymethyl)morpholino-4-yl]sulfonyl}-1H-indole-2-carboxamide;

(R)-5-Fluoro-3-{[2-(phenoxymethyl)morpholin-4-yl]sulfonyl}-1H-indole-2-carboxamide;

5-Acetylamino-3-(morpholin-4-ylsulfonyl)-1H-indole-2-carboxamide;

5-[(Methylsulfonyl)amino]-3-(morpholin-4-ylsulfonyl)-1H-indole-2-carboxamide;

3-(Morpholin-4-ylsulfonyl)-5-[(trifluoroacetyl)amino]-1H-indole-2-carboxamide;

5-[(2-Aminoethyl)amino]-3-(morpholin-4-ylsulfonyl)-1H-indole-2-carboxamide;

5-(Dimethylamino)-3-(morpholin-4-ylsulfonyl)-1H-indole-2-carboxamide;

4,5-Dibromo-3-(morpholin-4-ylsulfonyl)-1H-indole-2-carboxamide;

5,6-Dibromo-3-(morpholin-4-ylsulfonyl)-1H-indole-2-carboxamide;

5-Bromo-4-nitro-3-(morpholin-4-ylsulfonyl)-1H-indole-2-carboxamide;

5-Bromo-6-nitro-3-(morpholin-4-ylsulfonyl)-1H-indole-2-carboxamide;

5-Bromo-6-amino-3-(morpholin-4-ylsulfonyl)-1H-indole-2-carboxamide;

5-Bromo-4-amino-3-(morpholin-4-ylsulfonyl)-1H-indole-2-carboxamide;

5-Bromo-3-({2-[(cyclohexylamino)carbonyl]morpholin-4-yl}sulfonyl)-1H-indole-2-carboxamide;

5-Bromo-3-({2-[(2,3-dihydro-1H-inden-1-ylamino)carbonyl]morpholin-4-yl}sulfonyl)-1H-indole-2-carboxamide;

5-Bromo-3-[(2-{[(2-phenylethyl)amino]carbonyl}morpholin-4-yl)sulfonyl]-1H-indole-2-carboxamide;

5-Bromo-3-[(2-{[(3-phenylpropyl)amino]carbonyl}morpholin-4-yl)sulfonyl]-1H-indole-2-carboxamide;

5-Bromo-3-[(2-{[(3,3-diphenylpropyl)amino]carbonyl}morpholin-4-yl)sulfonyl]-1H-indole-2-carboxamide;

5-Bromo-3-{[2-(3,4-dihydroisoquinolin-2(1H)-ylcarbonyl)morpholin-4-yl]sulfonyl}-1H-indole-2-carboxamide;

5-Bromo-3-[(2-{[(2-phenoxyethyl)amino]carbonyl}morpholin-4-yl)sulfonyl]-1H-indole-2-carboxamide;

3-({2-[(3-Benzylpyrrolidin-1-yl)carbonyl]morpholin-4-yl}sulfonyl)-5-bromo-1H-indole-2-carboxamide;

5-Bromo-3-[(2-{[(1,2,3,4-tetrahydronaphthalen-2-ylmethyl)amino]carbonyl}morpholin-4-yl)sulfonyl]-1H-indole-2-carboxamide;

3-({2-[(Benzylamino)carbonyl]morpholin-4-yl}sulfonyl)-5-bromo-1H-indole-2-carboxamide;

5-Bromo-3-{[2-({[3-(trifluoromethyl)benzyl]amino}carbonyl)morpholin-4-yl]sulfonyl}-1H-indole-2-carboxamide;

5-Bromo-3-[(2-{[(2,2-diphenylethyl)amino]carbonyl}morpholin-4-yl)sulfonyl]-1H-indole-2-carboxamide;

5-Bromo-3-({2-[(2,3-dihydro-1H-inden-2-ylamino)carbonyl]morpholin-4-yl}sulfonyl)-1H-indole-2-carboxamide;

7-{[2-(Aminocarbonyl)-5-bromo-1H-indol-3-yl]sulfonyl}-2-benzyl-7-aza-2-azoniaspiro[4.4]nonane;

5-Bromo-3-{[2-({[(5-methylpyrazin-2-yl)methyl]amino}carbonyl)morpholin-4-yl]sulfonyl}-1H-indole-2-carboxamide;

3-({[(4-{[2-(Aminocarbonyl)-5-bromo-1H-indol-3-yl]sulfonyl}morpholin-2-yl)carbonyl]amino}methyl)pyridine;

5-Bromo-3-[(2-{[(1-phenylethyl)amino]carbonyl}morpholin-4-yl)sulfonyl]-1H-indole-2-carboxamide;

1-(3-{[(4-{[2-(Aminocarbonyl)-5-bromo-1H-indol-3-yl]sulfonyl}morpholin-2-yl)carbonyl]amino}propyl)-1H-imidazole;

5-Bromo-3-{[2-({[(1R)-1-phenylethyl]amino}carbonyl)morpholin-4-yl]sulfonyl}-1H-indole-2-carboxamide;

5-Bromo-3-[(2-{[(2-phenylpropyl)amino]carbonyl}morpholin-4-yl)sulfonyl]-1H-indole-2-carboxamide;

3-[(2-{[Benzyl(methyl)amino]carbonyl}morpholin-4-yl)sulfonyl]-5-bromo-1H-indole-2-carboxamide;

1-[(4-{[2-(Aminocarbonyl)-5-bromo-1H-indol-3-yl]sulfonyl}morpholin-2-yl)carbonyl]-4-benzylpiperazine;

2-({[(4-{[2-(Aminocarbonyl)-5-bromo-1H-indol-3-yl]sulfonyl}morpholin-2-yl)carbonyl]amino}methyl)pyridine;

5-Bromo-3-{[2-({[2-(tert-butylthio)ethyl]amino}carbonyl)morpholin-4-yl]sulfonyl}-1H-indole-2-carboxamide;

3-({2-[(Benzhydrylamino)carbonyl]morpholin-4-yl}sulfonyl)-5-bromo-1H-indole-2-carboxamide;

5-Bromo-3-{[2-({[(2S)-2-phenylcyclopropyl]amino}carbonyl)morpholin-4-yl]sulfonyl}-1H-indole-2-carboxamide;

5-Bromo-3-({2-[(3-phenylpyrrolidin-1-yl)carbonyl]morpholin-4-yl}sulfonyl)-1H-indole-2-carboxamide;

5-Bromo-3-({2-[(4,4-diphenylpiperidin-1-yl)carbonyl]morpholin-4-yl}sulfonyl)-1H-indole-2-carboxamide;

5-Bromo-3-[(2-{[(2,3-dihydro-1H-inden-2-ylmethyl)amino]carbonyl}morpholin-4-yl)sulfonyl]-1H-indole-2-carboxamide;

5-Bromo-3-({2-[(2,3-dihydro-1H-inden-1-ylamino)carbonyl]morpholin-4-yl}sulfonyl)-1H-indole-2-carboxamide;

5-Bromo-3-({2-[(2,3-dihydro-1H-inden-1-ylamino)carbonyl]morpholin-4-yl}sulfonyl)-1H-indole-2-carboxamide;

5-Bromo-3-({2-[(3-pyridin-4-ylpyrrolidin-1-yl)carbonyl]morpholin-4-yl}sulfonyl)-1H-indole-2-carboxamide;

5-Bromo-3-[(2-{[(2-hydroxy-2,3-dihydro-1H-inden-1-yl)amino]carbonyl}morpholin-4-yl)sulfonyl]-1H-indole-2-carboxamide;

5-Bromo-3-({2-[(4-hydroxy-4-phenylpiperidin-1-yl)carbonyl]morpholin-4-yl}sulfonyl)-1H-indole-2-carboxamide;

3-{[2-(Anilinocarbonyl)morpholin-4-yl]sulfonyl}-5-bromo-1H-indole-2-carboxamide;

5-Bromo-3-[(2-{[(2-oxo-2-phenylethyl)amino]carbonyl}morpholin-4-yl)sulfonyl]-1H-indole-2-carboxamide;

5-Bromo-3-({2-[(neopentylamino)carbonyl]morpholin-4-yl}sulfonyl)-1H-indole-2-carboxamide;

5-Bromo-3-[(2-{[(1,2-diphenylethyl)amino]carbonyl}morpholin-4-yl)sulfonyl]-1H-indole-2-carboxamide;

5-Bromo-3-[(2-{[(4-chlorophenyl)amino]carbonyl}morpholin-4-yl)sulfonyl]-1H-indole-2-carboxamide;

5-Bromo-3-[(2-{[(4-phenoxyphenyl)amino]carbonyl}morpholin-4-yl)sulfonyl]-1H-indole-2-carboxamide;

5-Bromo-3-[(2-{[(4-tert-butylphenyl)amino]carbonyl}morpholin-4-yl)sulfonyl]-1H-indole-2-carboxamide;

5-Bromo-3-{[2-({[3-(2-oxopyrrolidin-1-yl)propyl]amino}carbonyl)morpholin-4-yl]sulfonyl}-1H-indole-2-carboxamide;

5-Bromo-3-[(2-{[(3-isopropoxypropyl)amino]carbonyl}morpholin-4-yl)sulfonyl]-1H-indole-2-carboxamide;

5-Bromo-3-[(2-{[(3-ethoxypropyl)amino]carbonyl}morpholin-4-yl)sulfonyl]-1H-indole-2-carboxamide;

5-Bromo-3-[(2-{[(2-cyclohex-1-en-1-ylethyl)amino]carbonyl}morpholin-4-yl)sulfonyl]-1H-indole-2-carboxamide;

5-Bromo-3-[(2-{[(2,2,3,3,4,4,4-heptafluorobutyl)amino]carbonyl}morpholin-4-yl)sulfonyl]-1H-indole-2-carboxamide;

5-Bromo-3-[(2-{[(3-isobutoxypropyl)amino]carbonyl}morpholin-4-yl)sulfonyl]-1H-indole-2-carboxamide;

5-Bromo-3-[(2-{[(3-butoxypropyl)amino]carbonyl}morpholin-4-yl)sulfonyl]-1H-indole-2-carboxamide;

5-Bromo-3-[(2-{[(2-thien-2-ylethyl)amino]carbonyl}morpholin-4-yl)sulfonyl]-1H-indole-2-carboxamide;

2-({[(4-{[2-(Aminocarbonyl)-5-bromo-1H-indol-3-yl]sulfonyl}morpholin-2-yl)carbonyl]amino}methyl)-1H-benzimidazole;

3-{[2-(Azepan-1-ylcarbonyl)morpholin-4-yl]sulfonyl}-5-bromo-1H-indole-2-carboxamide;

5-Bromo-3-({2-[({2-[(2,6-dichlorobenzyl)thio]ethyl}amino)carbonyl]morpholin-4-yl}sulfonyl)-1H-indole-2-carboxamide;

3-{[2-({[4-(Aminosulfonyl)benzyl]amino}carbonyl)morpholin-4-yl]sulfonyl}-5-bromo-1H-indole-2-carboxamide;

5-Bromo-3-{[2-(thiomorpholin-4-ylcarbonyl)morpholin-4-yl]sulfonyl}-1H-indole-2-carboxamide;

5-Bromo-3-[(2-{[(2-methoxyethyl)amino]carbonyl}morpholin-4-yl)sulfonyl]-1H-indole-2-carboxamide;

5-Bromo-3-[(2-{[(2-methoxy-1-methylethyl)amino]carbonyl}morpholin-4-yl)sulfonyl]-1H-indole-2-carboxamide;

5-Bromo-3-[(2-{[(1-ethylpropyl)amino]carbonyl}morpholin-4-yl)sulfonyl]-1H-indole-2-carboxamide;

5-Bromo-3-{[2-({[6-(dimethylamino)hexyl]amino}carbonyl)morpholin-4-yl]sulfonyl}-1H-indole-2-carboxamide;

5-Bromo-3-[(2-{[(tetrahydrofuran-2-ylmethyl)amino]carbonyl}morpholin-4-yl)sulfonyl]-1H-indole-2-carboxamide;

5-Bromo-3-[(2-{[(1-phenylcyclopropyl)amino]carbonyl}morpholin-4-yl)sulfonyl]-1H-indole-2-carboxamide;

5-Bromo-3-{[2-({[phenyl(pyridin-4-yl)methyl]amino}carbonyl)morpholin-4-yl]sulfonyl}-1H-indole-2-carboxamide;

5-Bromo-3-[(2-{[(dicyclopropylmethyl)amino]carbonyl}morpholin-4-yl)sulfonyl]-1H-indole-2-carboxamide;

5-Bromo-3-[(2-{[(1,4-dioxan-2-ylmethyl)amino]carbonyl}morpholin-4-yl)sulfonyl]-1H-indole-2-carboxamide;

5-Bromo-3-{[2-({methyl[2-(4-methylphenoxy)ethyl]amino}carbonyl)morpholin-4-yl]sulfonyl}-1H-indole-2-carboxamide;

5-Bromo-3-{[2-({[(1,1-dioxidotetrahydrothien-3-yl)methyl]amino}carbonyl)morpholin-4-yl]sulfonyl}-1H-indole-2-carboxamide;

5-Bromo-3-[(2-{[2-(2-phenylethyl)pyrrolidin-1-yl]carbonyl}morpholin-4-yl)sulfonyl]-1H-indole-2-carboxamide;

5-Bromo-3-[(2-{[(2-cyclohexylethyl)amino]carbonyl}morpholin-4-yl)sulfonyl]-1H-indole-2-carboxamide;

4-({[(4-{[2-(Aminocarbonyl)-5-bromo-1H-indol-3-yl]sulfonyl}morpholin-2-yl)carbonyl]amino}methyl)-1-methyl-1H-imidazole;

5-Bromo-3-[(2-{[(1,1-dioxidotetrahydrothien-3-yl)amino]carbonyl}morpholin-4-yl)sulfonyl]-1H-indole-2-carboxamide;

5-Bromo-3-[(2-{[(1-naphthylmethyl)amino]carbonyl}morpholin-4-yl)sulfonyl]-1H-indole-2-carboxamide;

5-Bromo-3-[(2-{[(imidazo[2,1-b][1,3]thiazol-6-ylmethyl)amino]carbonyl}morpholin-4-yl)sulfonyl]-1H-indole-2-carboxamide;

3-[(2-{[2-(1,3-Benzothiazol-2-yl)pyrrolidin-1-yl]carbonyl}morpholin-4-yl)sulfonyl]-5-bromo-1H-indole-2-carboxamide;

5-Chloro-3-({2-[(2-ethoxyphenoxy)methyl]morpholin-4-yl}sulfonyl)-1H-indole-2-carboxamide;

5-Chloro-3-[(1R,4R)-2-oxa-5-azabicyclo[2.2.1]hept-5-ylsulfonyl]-1H-indole-2-carboxamide;

7-{[2-(Aminocarbonyl)-5-chloro-1H-indol-3-yl]sulfonyl}-3-benzyl-9-thia-7-aza-3-azoniabicyclo[3.3.1]nonane;

5-Chloro-3-{[2-(1H-indol-4-yl)morpholin-4-yl]sulfonyl}-1H-indole-2-carboxamide;

5-Chloro-3-(2,3-dihydro-1,4-benzoxazepin-4(5H)-ylsulfonyl)-1H-indole-2-carboxamide;

3-[(Benzofuran-yl-1-oxa-8-azaspiro[4.5]dec-8-yl)sulfonyl]-5-chloro-1H-indole-2-carboxamide;

5-Chloro-3-{[4-fluoro-4-(3-phenylpropyl)piperidin-1-yl]sulfonyl}-1H-indole-2-carboxamide;

3-[(3-Benzyl-1-oxa-8-azaspiro[4.5]dec-8-yl)sulfonyl]-5-chloro-1H-indole-2-carboxamide;

3-({4-[(Benzyloxy)methyl]-4-phenylpiperidin-1-yl}sulfonyl)-5-chloro-1H-indole-2-carboxamide;

5-Chloro-3-{[4-hydroxy-4-(3-phenylpropyl)piperidin-1-yl]sulfonyl}-1H-indole-2-carboxamide;

7-{[2-(Aminocarbonyl)-5-chloro-1H-indol-3-yl]sulfonyl}-2-(4-chlorophenyl)-7-aza-2-azoniaspiro[4.4]nonane;

3-(1-{[2-(Aminocarbonyl)-5-chloro-1H-indol-3-yl]sulfonyl}piperidin-3-yl)-4-methyl-4H-1,2,4-triazole;

5-Chloro-3-{[3-(2-phenylethyl)piperidin-1-yl]sulfonyl}-1H-indole-2-carboxamide;

5-Chloro-3-{[3-(2-phenylethyl)pyrrolidin-1-yl]sulfonyl}-1H-indole-2-carboxamide;

5-Chloro-3-{[4-(cyclopropyl {[3-(trifluoromethyl)phenyl]sulfonyl}amino)piperidin-1-yl]sulfonyl}-1H-indole-2-carboxamide;

5-Chloro-3-({2-[(4-chlorophenoxy)methyl]morpholin-4-yl}sulfonyl)-1H-indole-2-carboxamide;

Tert-butyl (1-{[2-(aminocarbonyl)-5-chloro-1H-indol-3-yl]sulfonyl}piperidin-3-yl)acetate;

3-[(3-Benzylpiperidin-1-yl)sulfonyl]-5-chloro-1H-indole-2-carboxamide;

5-Chloro-3-{[3-(2-methylphenyl)piperidin-1-yl]sulfonyl}-1H-indole-2-carboxamide;

2-(1-{[2-(Aminocarbonyl)-5-chloro-1H-indol-3-yl]sulfonyl}piperidin-4-yl)-N,N-dimethylethanamine;

1-(1-{[2-(Aminocarbonyl)-5-chloro-1H-indol-3-yl]sulfonyl}piperidin-4-yl)-3-(ethoxycarbonyl)piperidine;

5-Bromo-3-{[3-(4-tert-butoxybenzyl)piperidin-1-yl]sulfonyl}-1H-indole-2-carboxamide;

5-Bromo-3-{[4-(3-phenylpropyl)piperidin-1-yl]sulfonyl}-1H-indole-2-carboxamide;

5-Bromo-N-methoxy-N-methyl-3-{[2-(phenoxymethyl)morpholin-4-yl]sulfonyl}-1H-indole-2-carboxamide; and

(S)-3-{[2-(phenoxymethyl)morpholino-4-yl]sulfonyl}-1H-indole-2-carboxamide;

or the pharmaceutically acceptable salts or stereoisomers thereof.

Specific examples of compounds of the instant invention include:

5-Bromo-3-(morpholin-4-ylsulfonyl)-1H-indole-2-carboxamide;

(S)-5-Chloro-3-{[2-(phenoxymethyl)morpholin-4-yl]sulfonyl}-1H-indole-2-carboxamide;

(S)-5-Bromo-3-{[2-(phenoxymethyl)morpholino-4-yl]sulfonyl}-1H-indole-2-carboxamide;

(S)-5-Iodo-3-{[2-(phenoxymethyl)morpholino-4-yl]sulfonyl}-1H-indole-2-carboxamide;

7-Amino-3-{[(2S)-2-(phenoxymethyl)morpholin-4-yl]sulfonyl}-1H-indole-2-carboxamide;

3-{[(2S)-2-(Phenoxymethyl)morpholin-4-yl]sulfonyl}-7-[(pyridin-4-ylmethyl)amino]-1H-indole-2-carboxamide;

5-bromo-3-({2-[(2,3-dihydro-1H-inden-2-ylamino)carbonyl]morpholin-4-yl}sulfonyl)-1H-indole-2-carboxamide;

5-bromo-3-[(2-{[(1-naphthylmethyl)amino]carbonyl}morpholin-4-yl)sulfonyl]-1H-indole-2-carboxamide;

5-chloro-3-({2-[(4-chlorophenoxy)methyl]morpholin-4-yl}sulfonyl)-1H-indole-2-carboxamide; and

(S)-3-{[2-(phenoxymethyl)morpholino-4-yl]sulfonyl}-1H-indole-2-carboxamide;

or a pharmaceutically acceptable salt or stereoisomer thereof.

Compounds which are inhibitors of protein kinases and are useful in the present invention, and methods of synthesis thereof, can be found in the following patents, pending applications and publications, which are herein incorporated by reference:

U.S. S No. 60/342,900 filed on Oct. 25, 2001

U.S. S No. 60/343,119 filed on Oct. 25, 2001

U.S. S No. 60/343,000 filed on Oct. 25, 2001

U.S. S No. 60/342,902 filed on Oct. 25, 2001

U.S. S No. 60/402,482 filed on Aug. 9, 2001

U.S. S No. 60/402,478 filed on Aug. 9, 2001

U.S. S No. 60/372,358 filed on Apr. 12, 2002

U.S. S No. 60/372,232 filed on Apr. 12, 2002

With respect to compounds of formulas I through V the following definitions apply:

As used herein, the expression “C₁₋₆ alkyl” includes methyl and ethyl groups, and straight-chained or branched propyl, butyl, pentyl and hexyl groups. Particular alkyl groups are methyl, ethyl, n-propyl, isopropyl, tert-butyl and 2,2-dimethylpropyl. Derived expressions such as “C₁₋₆ alkoxy” are to be construed accordingly.

As used herein, the expression “C₁₋₄ alkyl” includes methyl and ethyl groups, and straight-chained or branched propyl and butyl groups. Particular alkyl groups are methyl, ethyl, n-propyl, isopropyl and tert-butyl. Derived expressions such as “C₁₋₄ alkoxy” are to be construed accordingly.

Typical C₃₋₇ cycloalkyl groups include cyclopropyl, cyclobutyl, cyclopentyl and cyclohexyl.

The expression “C₃₋₇ cycloalkyl(C₁₋₆)alkyl” as used herein includes cyclopropylmethyl, cyclobutylmethyl, cyclopentylmethyl and cyclohexylmethyl.

Typical C₄₋₇ cycloalkenyl groups include cyclobutenyl, cyclopentenyl and cyclohexenyl.

Typical aryl groups include phenyl and naphthyl, preferably phenyl.

The expression “aryl(C₁₋₆)alkyl” as used herein includes benzyl, phenylethyl, phenylpropyl and naphthylmethyl.

The term “halogen” as used herein includes fluorine, chlorine, bromine and iodine, especially fluorine or chlorine.

The present invention includes within its scope prodrugs of the compounds of formulae I-V above. In general, such prodrugs will be functional derivatives of the compounds of formulae I-V which are readily convertible in vivo into the required compound of formulae IV. Conventional procedures for the selection and preparation of suitable prodrug derivatives are described, for example, in Design of Prodrugs, ed. H. Bundgaard, Elsevier, 1985.

Where the compounds useful in the instant methods of treatment have at least one asymmetric center, they may accordingly exist as enantiomers. Where such compounds possess two or more asymmetric centers, they may additionally exist as diastereoisomers. It is to be understood that all such isomers and mixtures thereof in any proportion are encompassed within the scope of the present invention.

Examples of suitable values for the substituent R⁴ include methyl, ethyl, isopropyl, tert-butyl, 1,1-dimethylpropyl, methyl-cyclopropyl, cyclobutyl, methyl-cyclobutyl, cyclopentyl, methyl-cyclopentyl, cyclohexyl, cyclobutenyl, phenyl, pyrrolidinyl, methyl-pyrrolidinyl, piperidinyl, morpholinyl, thiomorpholinyl, pyridinyl, furyl, thienyl, chloro-thienyl and diethylamino.

In a particular embodiment, the substituent R⁴ represents C₃₋₇ cycloalkyl or phenyl, either unsubstituted or substituted by C₁₋₆ alkyl, especially methyl. Favourably, Z represents cyclobutyl or phenyl.

Examples of typical optional substituents on the group R¹ include methyl, fluoro and methoxy.

Representative values of R¹ include cyclopropyl, phenyl, methylphenyl, fluorophenyl, difluorophenyl, methoxyphenyl, furyl, thienyl, methyl-thienyl and pyridinyl.

In a particular embodiment, R² represents amino-C₁₋₆ alkyl, C₁₋₄ alkylamino-(C₁₋₆)alkyl or di(C₁₋₄ alkyl)amino-(C₁₋₆)alkyl. Representative values of R² include but are not limited to dimethylaminomethyl, aminoethyl, dimethylaminoethyl, diethylaminoethyl, 3-dimethylaminopropyl, 3-methylaminopropyl, 3-dimethylamino-2,2-dimethylpropyl and, 3-dimethylamino-2-methylpropyl.

Suitably, R³ represents hydrogen or methyl.

With respect to compounds of formulas VI through IX the following definitions apply:

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention.

In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof. The two tautomeric forms of the benzimidazolonyl moiety are also within the scope of the instant ivention.

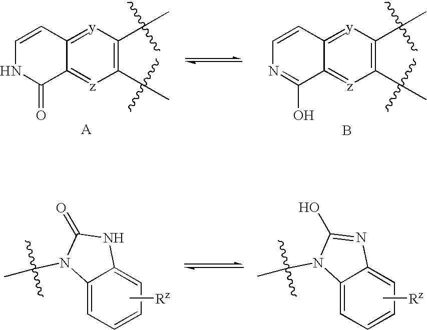

When any variable (e.g. R¹, R², R^{z}, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on.

“Alkoxy” represents either a cyclic or non-cyclic alkyl group of indicated number of carbon atoms attached through an oxygen bridge. “Alkoxy” therefore encompasses the definitions of alkyl and cycloalkyl above.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively. Such heteraoaryl moieties for substituent Q include but are not limited to: 2-benzimidazolyl, 2-quinolinyl, 3-quinolinyl, 4-quinolinyl, 1-isoquinolinyl, 3 isoquinolinyl and 4-isoquinolinyl.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzoimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

In another embodiment, heterocycle is selected from 2-azepinone, benzimidazolyl, 2-diazapinone, imidazolyl, 2-imidazolidinone, indolyl, isoquinolinyl, morpholinyl, piperidyl, piperazinyl, pyridyl, pyrrolidinyl, 2-piperidinone, 2-pyrimidinone, 2-pyrollidinone, quinolinyl, tetrahydrofuryl, tetrahydroisoquinolinyl, and thienyl.

As appreciated by those of skill in the art, “halo” or “halogen” as used herein is intended to include chloro, fluoro, bromo and iodo.

As used herein, unless otherwise specifically defined, substituted alkyl, substituted cycloalkyl, substituted aroyl, substituted aryl, substituted heteroaroyl, substituted heteroaryl, substituted arylsulfonyl, substituted heteroaryl-sulfonyl and substituted heterocycle include moieties containing from 1 to 3 substituents in addition to the point of attachment to the rest of the compound. Preferably, such substituents are selected from the group which includes but is not limited to F, Cl, Br, CF₃, NH₂, N(C₁-C₆ alkyl)₂, NO₂, CN, (C₁-C₆ alkyl)O—, (aryl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, (C₁-C₆ alkyl)OC(O)NH—, phenyl, pyridyl, imidazolyl, oxazolyl, isoxazolyl, thiazolyl, thienyl, furyl, isothiazolyl and C₁-C₂₀ alkyl. For example, a (C₁-C₆)alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition: C═O)CH₂CH(OH)CH₃, —(C═O)OH, —CH₂(OH)CH₂CH(O), and so on.

The moiety illustrated in formulas VI and VII by the structure:

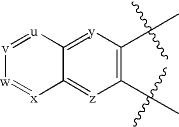

includes the following structures, which are meant to be merely illustrative and not limiting:

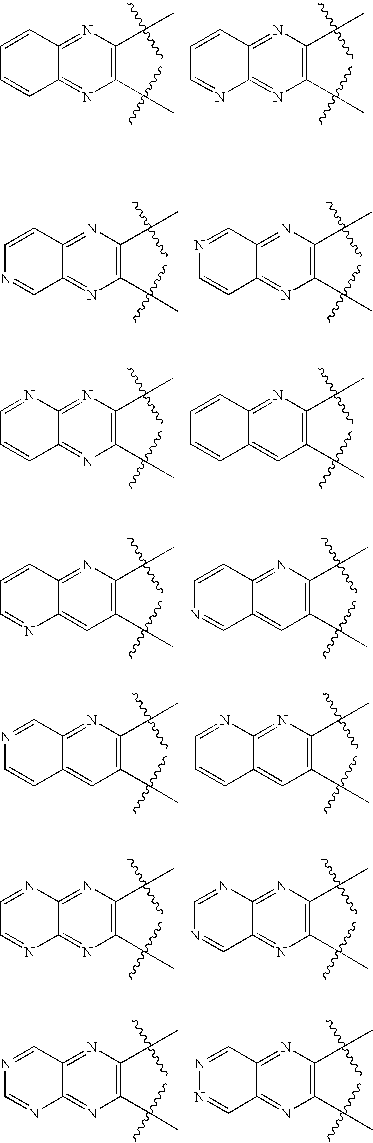

In another embodiment, the moiety illustrated by the formula:

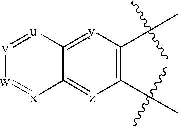

is selected from:

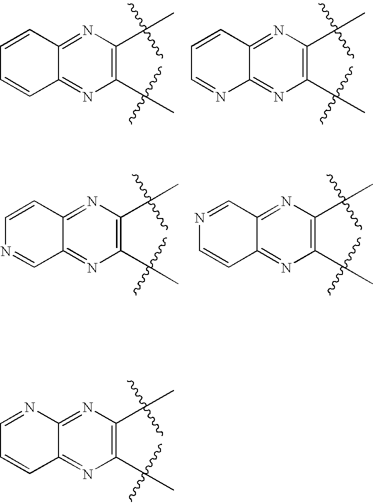

The moieties form when R¹ is oxo include the following structures, which are meant to be merely illustrative and not limiting:

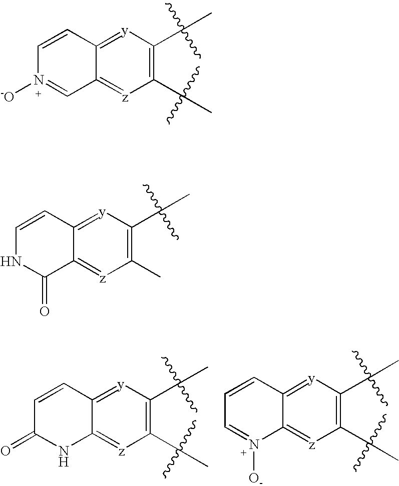

The moiety formed when, in the definition of R³ and R⁴ on the same carbon atom are combined to form —(CH₂)ₜ— is illustrated by the following:

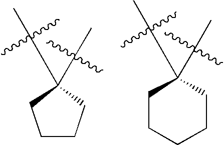

In addition, such cyclic moieties may optionally include a heteroatom(s). Examples of such heteroatom-containing cyclic moieties include, but are not limited to:

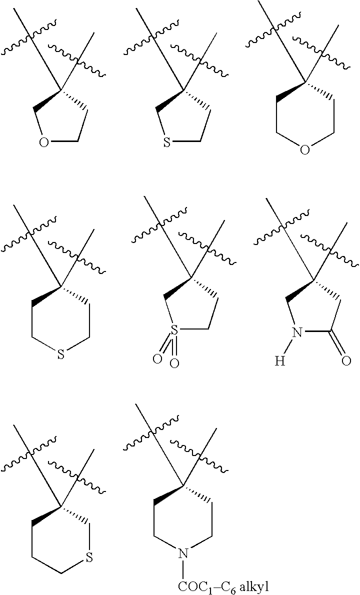

In certain instances, R⁷ and R⁸ are defined such that they can be taken together with the nitrogen to which they are attached to form a monocyclic or bicyclic heterocycle with 57 members in each ring and optionally containing, in addition to the nitrogen, one or two additional heteroatoms selected from N, O and S, said heterocycle optionally substituted with one or more substituents selected from R^{z}. Examples of the heterocycles that can thus be formed include, but are not limited to the following, keeping in mind that the heterocycle is optionally substituted with one or more (and preferably one, two or three) substituents chosen from R^{z}:

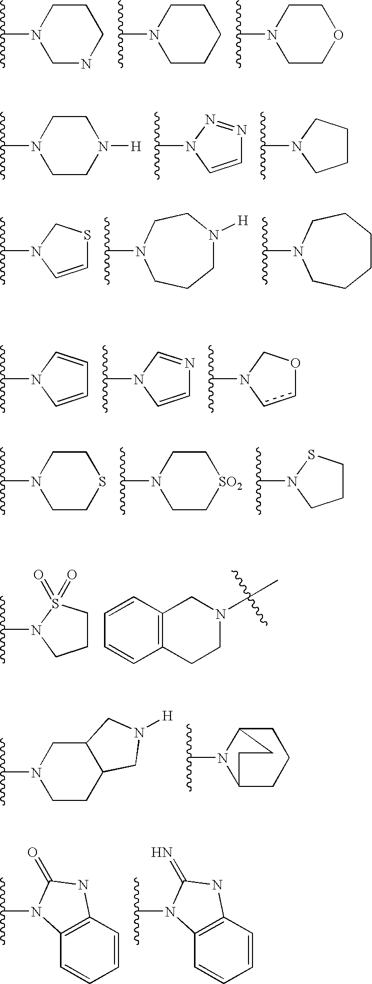

In another embodiment, y and z are N.

In another embodiment, R¹ is selected from: halogen, —OH, —CN, —NO₂, —CF₃, —OC₁-C₆alkyl, C₁-C₆alkyl, aryl, heterocyclyl, SO₂C₁-C₆ alkyl, —NR^{c}SO₂C₁-C₆ alkyl, —CO₂H, (C═O)OC₁-C₆alkyl, —(C═O)NR⁷R⁸, —(C═O)aryl, SO₂aryl and SO₂NR⁷R⁸, optionally substituted with one to three substituents selected from R^{z}. In another embodiment, R¹ is —OH, —OC₁-C₆alkyl, —CO₂H, —(C═O)NR⁷R⁸ and C₁-C₆alkyl.

In another embodiment, R² is selected from C₁-C₆alkyl, —OH, —OC₁-C₆alkyl, —CF₃, CN and halogen, optionally substituted with one substituent selected from R^{z}.

In another embodiment, is the definition of R³ and R⁴ selected from H and —CH₃. In another embodiment, R³ and R⁴ are H.

With respect to formula VI, and in another embodiment, R⁵ is selected from H and C₁-C₆ alkyl. In another embodiment, R⁵ is H. In another embodiment, R⁷ and R⁸ are selected from H, C₁-C₆ alkyl and aryl, optionally substituted with one to two substituents selected from R^{z}. In another embodiment, R⁷ and R⁸ are selected from H or C₁-C₆ alkyl.

With respect to formula VII, and in another embodiment, R⁵ and R⁶ are selected from H, C₁-C₆ alkyl and aryl, optionally substituted with one to two substituents selected from R^{z}, or R⁵ and R⁶ together with the nitrogen to which they are attached form a monocyclic or bicyclic heterocycle, optionally substituted with one to two substituents selected from R^{z}. In another embodiment, R⁵ and R⁶ are selected from H or C₁-C₆ alkyl, or R⁵ and R⁶ together with the nitrogen to which they are attached form a monocyclic or bicyclic heterocycle, optionally substituted with one to two substituents selected from R^{z}. In another embodiment, Q is selected from:

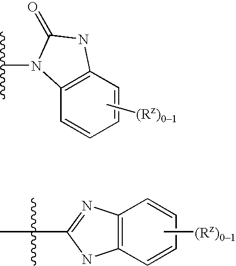

wherein R^{z} is selected from C₁-C₆ alkyl and halogen.

With respect to the compounds of formula VIII, the moieties formed when R¹ is oxo include the following structure, which are meant to be merely illustrative and not limiting:

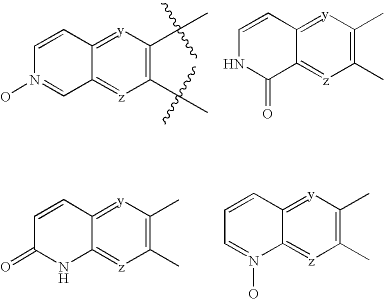

The moiety formed when, in the definition of R³ and R⁴ on the same carbon atom are combined to form —(CH₂)ₜ— is illustrated by the following:

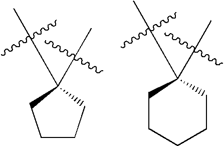

In addition, such cyclic moieties may optionally include a heteroatom(s). Examples of such heteroatom-containing cyclic moieties include, but are not limited to:

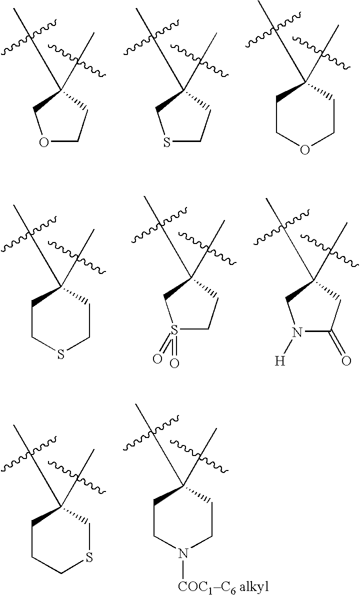

In certain instances, R⁵ and R⁶ or R⁷ and R⁸ are defined such that they can be taken together with the nitrogen to which they are attached to form a monocyclic or bicyclic heterocycle with 5-7 members in each ring and optionally containing, in addition to the nitrogen, one or two additional heteroatoms selected from N, O and S, said heterocycle optionally substituted with one or more substituents selected from R^{z}. Examples of the heterocycles that can thus be formed include, but are not limited to the following, keeping in mind that the heterocycle is optionally substituted with one or more (and preferably one, two or three) substituents chosen from R^{z}:

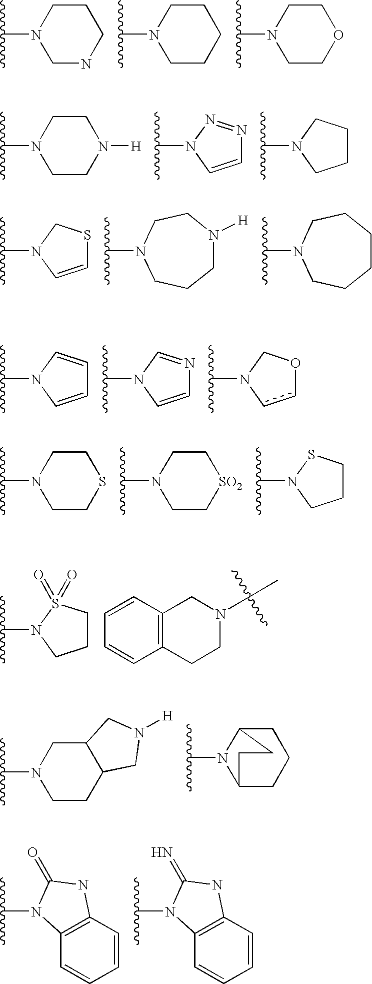

In another embodiment, R¹ is selected from: —OH, —OC₁-C₆alkyl, C₁-C₆alkyl, aryl, heterocyclyl, SO₂C₁-C₆ alkyl, —CO₂H, (C═O)OC₁-C₆alkyl, (C═O)NR⁷R⁸, SO₂aryl and SO₂NR⁷R⁸, optionally substituted with one to three substituents selected from R^{z}. In another embodiment, R¹ is selected from: —OH, —OC₁-C₆alkyl and C₁-C₆alkyl.

In another embodiment, R² is selected from C₁-C₆alkyl, —OH, —OC₁-C₆alkyl, —CF₃, CN and halogen, optionally substituted with one substituent selected from R^{z}.

In another embodiment is the definition of R³ and R⁴ selected from H and —CH₃. In another embodiment, R³ and R⁴ are H.

In another embodiment, R⁷ and R⁸ are selected from H, C₁-C₆ alkyl and aryl, optionally substituted with one to two substituents selected from R^{z}. In another embodiment, R⁷ and R⁸ are selected from H or C₁-C₆ alkyl.

In another embodiment, Q is selected from:

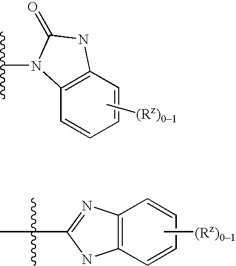

wherein R^{z} is selected from C₁-C₆ alkyl and halogen.

With respect to the compounds of the formula IX, the moiety illustrated by the formula:

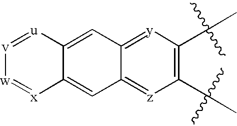

includes the following structures, which are meant to be merely illustrative and not limiting:

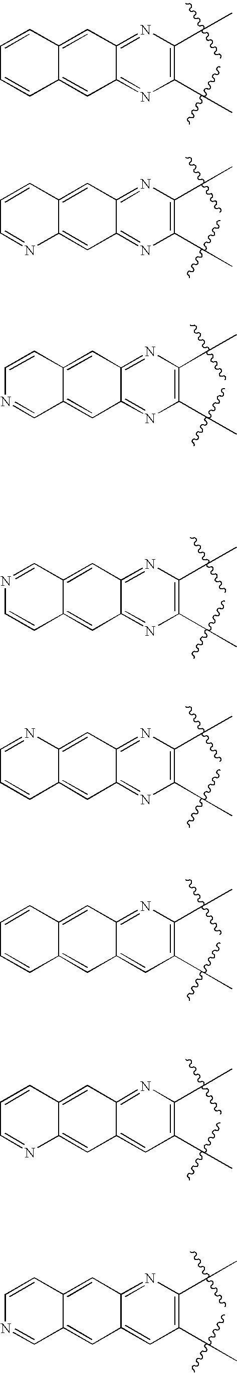

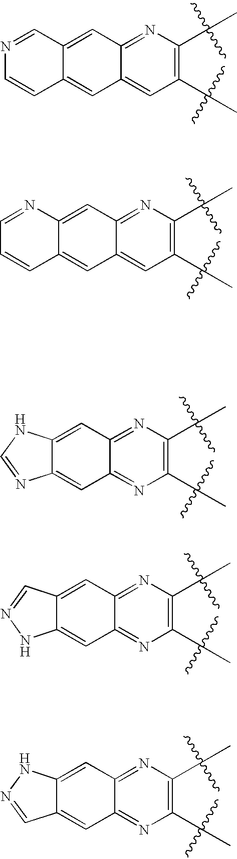

In another embodiment, the moiety illustrated by the formula:

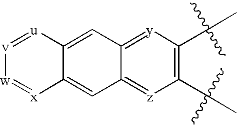

is selected from:

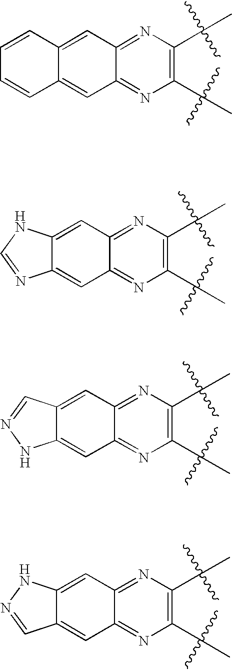

The moieties formed when R¹ is oxo include the following structures, which are meant to be merely illustrative and not limiting:

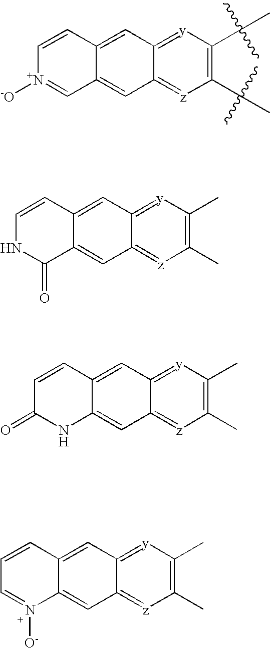

The moiety formed when, in the definition of R³ and R⁴ on the same carbon atom are combined to form —(CH₂)ₜ— is illustrated by the following:

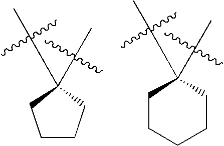

In addition, such cyclic moieties may optionally include a heteroatom(s). Examples of such heteroatom-containing cyclic moieties include, but are not limited to:

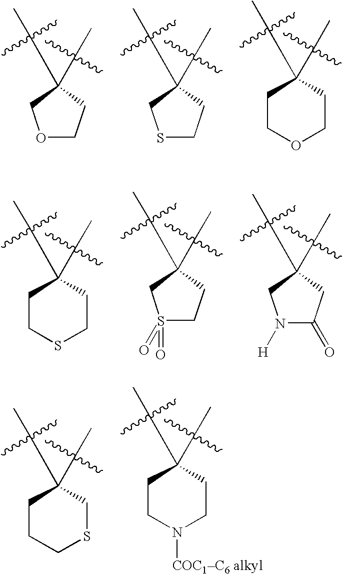

In certain instances, R⁵ and R⁶ or R⁷ and R⁸ are defined such that they can be taken together with the nitrogen to which they are attached to form a monocyclic or bicyclic heterocycle with 5-7 members in each ring and optionally containing, in addition to the nitrogen, one or two additional heteroatoms selected from N, O and S, said heterocycle optionally substituted with one or more substituents selected from R^{z}. Examples of the heterocycles that can thus be formed include, but are not limited to the following, keeping in mind that the heterocycle is optionally substituted with one or more (and preferably one, two or three) substituents chosen from R^{z}:

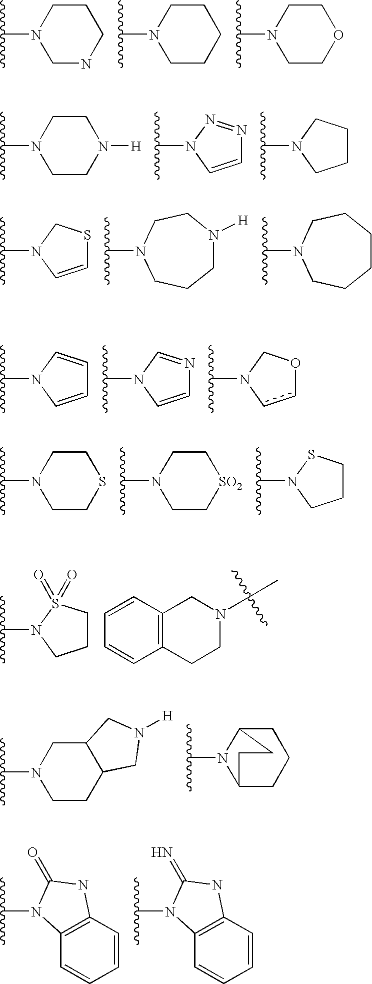

In another embodiment, y and z are N.

In another embodiment, when w is a bond, two of u, v and x are N.

In another embodiment, R¹ is selected from: —OH, —OC₁-C₆alkyl, C₁-C₆alkyl, aryl, heterocyclyl, SO₂C₁-C₆ alkyl, —CO₂H, (C═O)OC₁-C₆alkyl, (C═O)NR⁷R⁸, SO₂aryl and SO₂NR⁷R⁸, optionally substituted with one to three substituents selected from R^{z}. In another embodiment, R¹ is selected from —OH, —OC₁-C₆alkyl and C₁-C₆alkyl.

In another embodiment, R² is selected from C₁-C₆alkyl, —OH, —OC₁-C₆alkyl, —CF₃, CN and halogen, optionally substituted with one substituent selected from R^{z}.

In another embodiment is the definition of R³ and R⁴ selected from H and —CH₃. In another embodiment, R³ and R⁴ are H.

In another embodiment, R⁷ and R⁸ are selected from H, C₁-C₆ alkyl and aryl, optionally substituted with one to two substituents selected from R^{z}. In another embodiment, R⁷ and R⁸ are selected from H or C₁-C₆ alkyl.

In another embodiment, Q is selected from:

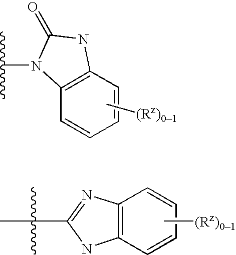

wherein R^{z} is selected from C₁-C₆ alkyl and halogen.

Included in the instant invention is the free form of compounds herein disclosed, as well as the pharmaceutically acceptable salts and stereoisomers thereof. Some of the isolated specific compounds exemplified herein are the protonated salts of amine compounds. The term “free form” refers to the amine compounds in non-salt form. The encompassed pharmaceutically acceptable salts not only include the isolated salts exemplified for the specific compounds described herein, but also all the typical pharmaceutically acceptable salts of the free form of compounds of Formulas I-IX. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

With respect to compounds of formulas X through XI the following definitions apply:

The compounds of formulas X through XI may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted or named.

When any variable (e.g. aryl, heterocycle, R¹, R^{a} etc.) occurs more than one time in any substituent, its definition on each occurrence is independent at every other occurrence. Also, combinations of substituents and variables are permissible only if such combinations result in stable compounds.

Lines drawn into the ring systems from substituents (such as from R¹, R⁵, etc.) indicate that the indicated bond may be attached to any of the substitutable ring carbon atoms or heteroatoms, including the carbon atom or heteroatom that is the point of attachment. If the ring system is polycyclic, such as

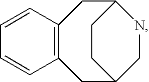

it is intended that the bond may be attached to any of the suitable carbon atoms or heteroatoms of any ring. It is also intended that a moiety such as

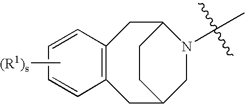

could also be represented as:

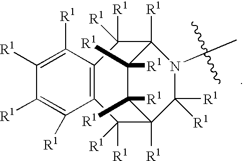

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

As used herein, “alkyl” is intended to include both branched and straight-chain aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, propyl, isopropyl, butyl, t-butyl, pentyl, hexyl, heptyl, octyl, nonyl, decyl, and so on.

“Cycloalkyl” as used herein is intended to include non-aromatic cyclic hydrocarbon groups, having the specified number of carbon atoms, which may or may not be bridged or structurally constrained. Examples of such cycloalkyls include, but are not limited to, cyclopropyl, cyclobutyl, cyclopentyl, cyclohexyl, adamantyl, cyclooctyl, cycloheptyl, tetrahydro-naphthalene, methylenecylohexyl, and the like. As used herein, examples of “C₃-C₁₀cycloalkyl” may include, but are not limited to:

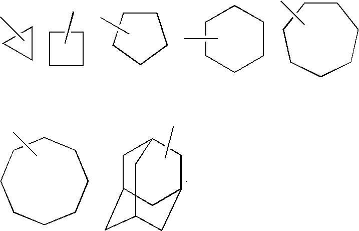

As used herein, the term “alkoxy” represents an alkyl group of indicated number of carbon atoms attached through an oxygen bridge.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 7 atoms in each ring, wherein at least one ring is aromatic. Examples of such aryl elements include phenyl, naphthyl, indanyl, indanonyl, indenyl, biphenyl, tetralinyl, tetralonyl, fluorenonyl, phenanthryl, anthryl, acenaphthyl, tetrahydronaphthyl, and the like.

As appreciated by those of skill in the art, “halo” or “halogen” as used herein is intended to include chloro, fluoro, bromo and iodo.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzimidazolyl, benzodioxolyl, benzotriazolyl, benzothiofuranyl, benzothiazolyl, furanyl, thienyl, benzothienyl, benzofuranyl, benzoquinolinyl, imidazolyl, isoquinolinyl, oxazolyl, isoxazolyl, indolyl, pyrazinyl, pyridazinyl, pyridinyl, pyrimidinyl, pyrrolyl, quinolinyl, tetrahydronaphthyl, tetrahydroquinoline, and the like.

The term heterocycle or heterocyclic or heterocyclyl, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. “Heterocycle” or “heterocyclyl” therefore includes the above mentioned heteroaryls, as well as dihydro and tetrathydro analogs thereof. Further examples of “heterocyclyl” include, but are not limited to the following: azepanyl, azetidinyl, benzimidazolyl, benzodioxolyl, benzofuranyl, benzofurazanyl, benzopyranyl, benzopyrazolyl, benzotriazolyl, benzothiazolyl, benzothienyl, benzothiofuranyl, benzothiophenyl, benzothiopyranyl, benzoxazepinyl, benzoxazolyl, carbazolyl, carbolinyl, chromanyl, cinnolinyl, diazepanyl, diazapinonyl, dihydrobenzofuranyl, dihydrobenzofuryl, dihydrobenzoimidazolyl, dihydrobenzothienyl, dihydrobenzothiopyranyl, dihydrobenzothiopyranyl sulfone, dihydrobenzothiophenyl, dihydrobenzoxazolyl, dihydrocyclopentapyridinyl, dihydrofuranyl, dihydroimidazolyl, dihydroindolyl, dihydroisooxazolyl, dihydroisoquinolinyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxanyl, dioxidotetrahydrothienyl, dioxidothiomorpholinyl, furyl, furanyl, imidazolyl, imidazolinyl, imidazolidinyl, imidazothiazolyl, imidazopyridinyl, indazolyl, indolazinyl, indolinyl, indolyl, isobenzofuranyl, isochromanyl, isoindolyl, isoindolinyl, isoquinolinone, isoquinolyl, isothiazolyl, isothiazolidinyl, isoxazolinyl, isoxazolyl, methylenedioxybenzoyl, morpholinyl, naphthpyridinyl, oxadiazolyl, oxazolyl, oxazolinyl, oxetanyl, oxoazepinyl, oxadiazolyl, oxidothiomorpholinyl, oxodihydrophthalazinyl, oxodihydroindolyl, oxoimidazolidinyl, oxopiperazinyl, oxopiperdinyl, oxopyrtolidinyl, oxopyrimidinyl, oxopyrtolyl, oxotriazolyl, piperidyl, piperidinyl, piperazinyl, pyranyl, pyrazinyl, pyrazolyl, pyridazinyl, pyridinonyl, pyridopyridinyl, pyridazinyl, pyridyl, pyrimidinyl, pyrrolyl, pyrrolidinyl, quinazolinyl, quinolinyl, quinolyl, quinolinonyl, quinoxalinyl, tetrahydrocycloheptapyridinyl, tetrahydrofuranyl, tetrahydrofuryl, tetrahydroisoquinolinyl, tetrahydropyranyl, tetrahydroquinolinyl, tetrazolyl, tetrazolopyridyl, thiadiazolyl, thiazolyl, thiazolinyl, thienofuryl, thienyl, thiomorpholinyl, triazolyl, azetidinyl, 1,4-dioxanyl, hexahydroazepinyl, and the like. In another embodiment, heterocycle is selected from oxoazepinyl, benzimidazolyl, diazepanyl, diazapinonyl, imidazolyl, oxoimidazolidinyl, indolyl, isoquinolinyl, morpholinyl, piperidyl, piperazinyl, pyridyl, pyrrolidinyl, oxopiperidinyl, oxopyrimidinyl, oxopyrrolidinyl, quinolinyl, tetrahydrofuryl, tetrahydrofuranyl, tetrahydroisoquinolinyl, thienyl, furyl, furanyl, pyrazinyl, benzofuranyl, isoxazolyl, pyrrolyl, thiazolyl, benzothienyl, dihydroisoquinolinyl, azepanyl, thiomorpholinyl, dioxanyl, dioxidotetrahydrothienyl, imidazothiazolyl, benzothiazolyl, and triazolyl.

As used herein, “aralkyl” is intended to mean an aryl moiety, as defined above, attached through a C₁-C₁₀ alkyl linker, where alkyl is defined above. Examples of aralkyls include, but are not limited to, benzyl, naphthylmethyl and phenylpropyl.

As used herein, “heterocyclylalkyl” is intended to mean a heterocyclic moiety, as defined below, attached through a C₁-C₁₀ alkyl linker, where alkyl is defined above. Examples of heterocyclylalkyls include, but are not limited to, pyridylmethyl, imidazolylethyl, pyrrolidinylmethyl, morpholinylethyl, quinolinylmethyl, imidazolylpropyl and the like.

As used herein, the terms “substituted C₁-C₁₀ alkyl” and “substituted C₁-C₆ alkoxy” are intended to include the branch or straight-chain alkyl group of the specified number of carbon atoms, wherein the carbon atoms may be substituted with 1 to 3 substituents selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆ alkyl) O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

As used herein, the terms “substituted C₃-C₁₀ cycloalkyl”, “substituted aryl”, “substituted heterocycle”, “substituted aralkyl” and “substituted heterocyclylalkyl” are intended to include the cyclic group containing from 1 to 3 substituents in addition to the point of attachment to the rest of the compound. Preferably, the substituents are selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (C₀-C₆ alkyl)S(O)₀₋₂(C₀-C₆ alkyl)-, (C₀-C₆ alkyl)C(O)NH—, H₂N—C(NH)—, O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heteroaryl, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

As used herein, the phrase “substituted with at least one substituent” is intended to mean that the substituted group being referenced has from 1 to 6 substituents. In another embodiment, the substituted group being referenced contains from 1 to 3 substituents, in addition to the point of attachment to the rest of the compound.

In another embodiment, R² is OR⁴ or NR⁴₂. In another embodiment, R² is N(R⁴)₂.

In another embodiment, G is H₂.

In another embodiment, X is O, N or C. In another embodiment, X is O or N. In another embodiment, X is O.

In another embodiment, n is independently 0, 1, 2, 3 or 4. In another embodiment, n is independently 0, 1 or 2.

In another embodiment, s and w are independently 0, 1, 2, 3 or 4. In another embodiment, s is 0, 1 or 2. In another embodiment, w is 0, 1, 2 or 3.

In another embodiment, t and v are independently 0 or 1.

It is intended that the definition of any substituent or variable (e.g., R¹, R^{a}, n, etc.) at a particular location in a molecule be independent of its definitions elsewhere in that molecule. Thus, —N(R⁴)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

For use in medicine, the salts of the compounds of Formulas X-XI will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, ptoluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

All patents, publications and pending patent applications identified are hereby incorporated by reference.

The compounds used in the present method may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration

The pharmaceutically acceptable salts of the compounds of this invention can be synthesized from the compounds of this invention which contain a basic moiety by conventional chemical methods. Generally, the salts are prepared by reacting the free base with stoichiometric amounts or with an excess of the desired salt-forming inorganic or organic acid in a suitable solvent or various combinations of solvents.

Abbreviations used in the description of the chemistry and in the Examples that follow are:

Ac₂O acetic anhydride;

Boc t-butoxycarbonyl;

DBU 1,8-diazabicyclo[5.4.0]undec-7-ene;

TFA: trifluoroacetic acid

AA: acetic acid

4-Hyp 4-hydroxyproline

Boc/BOC t-butoxycarbonyl;

Chg cyclohexylglycine

DMA dimethylacetamide

DMF dimethylformarnmide;

DMSO dimethyl sulfoxide;

EDC 1-(3-dimethylaminopropyl)-3-ethyl-carbodiimide hydrochloride;

EtOAc ethyl acetate;

EtOH ethanol;

FAB Fast atom bombardment;

HOAt 1-hydroxy-7-azabenzotriazole

HOBt 1-hydroxybenzotriazole hydrate;

HOPO 2-hydroxypyridine-N-oxide

HPLC High-performance liquid chromatography;

IPAc isopropylacetate

MeOH methanol

RPLC Reverse Phase Liquid Chromatography

THF tetrahydrofuran.

DCE dichloroethane

DCM dichloromethane

n-Pr n-propyl

PS-NMM polystyrene N-methylmorpholine

TFA trifluoroacetic acid

MP-CNBH₃ macroporous cyanoborohydride;

PS-DCC polystyrene-dicyclohexyl carbodiimide;

PS-DIEA polystyrene diisopropylethylamine;

Ac₂O Acetic anhydride;

AcOH Acetic acid;

AIBN 2,2′-Azobisisobutyronitrile;

Ar Aryl;

BINAP 2,2′-Bis(diphenylphosphino)-1,1′ binaphthyl;

Bn Benzyl;

BOC/Boc tert-Butoxycarbonyl;

BSA Bovine Serum Albumin;

CAN Ceric Ammonia Nitrate;

CBz Carbobenzyloxy;

CI Chemical Ionization;

DBAD Di-tert-butyl azodicarboxylate;

DBU 1,8-Diazabicyclo[5.4.0]undec-7-ene;

DCC 1,3-Dichlorohexylcarbodiimide;

DCE 1,2-Dichloroethane;

DCM Dichloromethane;

DIEA N,N-Diisopropylethylamine;

DMAP 4-Dimethylaminopyridine;

DME 1,2-Dimethoxyethane;

DMF N,N-Dimethylformamide;

DMSO Methyl sulfoxide;

DPPA Diphenylphosphoryl azide;

DTT Dithiothreitol;

EDC 1-(3-Dimethylaminopropyl)-3-ethyl-carbodiimide hydrochloride;

EDTA Ethylenediaminetetraacetic acid;

ELSD Evaporative Light Scattering Detector;

ES Electrospray;

ESI Electrospray ionization;

Et₂O Diethyl ether;

Et₃N Triethylamine;

EtOAc Ethyl acetate;

EtOH Ethanol;

FAB Fast Atom Bombardment;

HEPES 4-(2-Hydroxyethyl)-1-piperazineethanesulfonic acid;

HMPA Hexamethylphosphoramide;

HOAc Acetic acid;

HOBt 1-Hydroxybenzotriazole hydrate;

HOOBt 3-Hydroxy-1,2,2-benzotriazin-4(3H)-one;

HPLC High-performance liquid chromatography;

HRMS High Resolution Mass Spectroscopy;

KOtBu Potassium tert-butoxide;

LAH Lithium aluminum hydride;

LCMS Liquid Chromatography Mass Spectroscopy;

MCPBA m-Chloroperoxybenzoic acid;

Me Methyl;

MeOH Methanol;

MP-CarbonateMacroporous polystyrene carbonate;

Ms Methanesulfonyl;

MS Mass Spectroscopy;

MsCl Methanesulfonyl chloride;

n-Bu n-butyl;

n-Bu₃P Tri-n-butylphosphine;

NaHMDS Sodium bis(trimethylsilyl)ₐmide;

NBS N-Bromosuccinimide;

NMM N-methylmorpholine;

NMR Nuclear Magnetic Resonance;

Pd(PPh₃)₄ Palladium tetrakis(triphenylphosphine);

Pd₂(dba)₃ Tris(dibenzylideneacetone)dipalladium (0);

Ph phenyl;

PMSF α-Toluenesulfonyl fluoride;

PS-DCC Polystyrene dicyclohexylcarbodiimide;

PS-DMAP Polystyrene dimethylaminopyridine;

PS-NMM Polystyrene N-methylmorpholine;

Py or pyr Pyridine;

PYBOP Benzotriazol-1-yloxytripyrrolidinophosphonium

(or PyBOP) hexafluorophosphate;

RPLC Reverse Phase Liquid Chromatography;

RT Room Temperature;

SCX SPE Strong Cation Exchange Solid Phase Extraction;

t-Bu tert-Butyl;

TBAF Tetrabutylammonium fluoride;

TBSCl tert-Butyldimethylsilyl chloride;

TFA Trifluoroacetic acid;

THF Tetrahydrofuran;

TIPS Triisopropylsilyl;

TMS Tetramethylsilane; and

Tr Trityl.

Reactions used to generate the compounds which are selective inhibitors of Akt activity and are therefore useful in the methods of treatment of this invention are shown in the Reaction Schemes 1-10, in addition to other standard manipulations such as ester hydrolysis, cleavage of protecting groups, etc., as may be known in the literature or exemplified in the experimental procedures. Substituents R and R^{a}, as shown in the Reaction Schemes, represent the substituents R¹ and R²; however their point of attachment to the ring is illustrative only and is not meant to be limiting.

These reactions may be employed in a linear sequence to provide the compounds of the invention or they may be used to synthesize fragments that are subsequently joined by the alkylation reactions described in the Reaction Schemes.

Synopsis of Reaction Schemes 1-10:

The requisite intermediates are in some cases commercially available, or can be prepared according to literature procedures. As illustrated in Reaction Scheme 1, a suitably substituted phenylmaleic anhydride i is treated with hydrazine to form the dihydropyridazone dione ii. Subsequent oxidative chlorination and reaction with a suitably substituted benzoic hydrazide provide the 6-chloro triazolo [4,3-b]pyridazine iii. This intermediate can then be treated with a variety of alcohols and amines to provide the compound iv.

Reaction Scheme 2 illustrates preparation of compounds useful in the methods of the instant invention having a cycloalkyl substituent at the 7-position. While a cyclobutyl group is illustrated, the sequence of reactions is generally applicable to incorporation of a variety of unsubstituted or substituted cycloalkyl moieties. Thus, 3,6-dichloropyridazine is alkylated via silver catalyzed oxidative decarboxylation with cyclobutyl carboxylic acid to provide the cyclobutyl dichloropyridazine v, which then undergoes the reactions described above to provide the instant compound vi.

Reaction Scheme 3 illustrates the same reaction sequence used to prepare compounds of the Formula I Reaction Scheme 4 illustrates an alternative preparation of the instant compounds (Tetrahedron Letters 41:781-784 (2000)).

Reaction Scheme 5 illustrates a synthetic method of preparing the compounds of the Formula IV hereinabove.

Reaction Scheme 6 illustrates a synthetic method of preparing the compounds of the Formula III hereinabove.

Reaction Schemes 7-8 illustrates a synthetic method of preparing the compounds of the Formula VII hereinabove.

Reaction Schemes 9-10 illustrates a synthetic method of preparing the compounds of the Formula IX hereinabove.

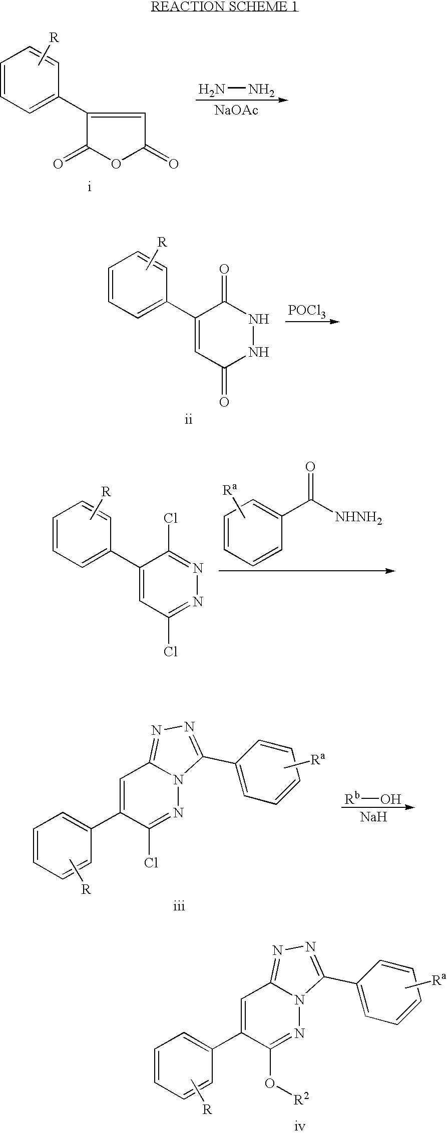

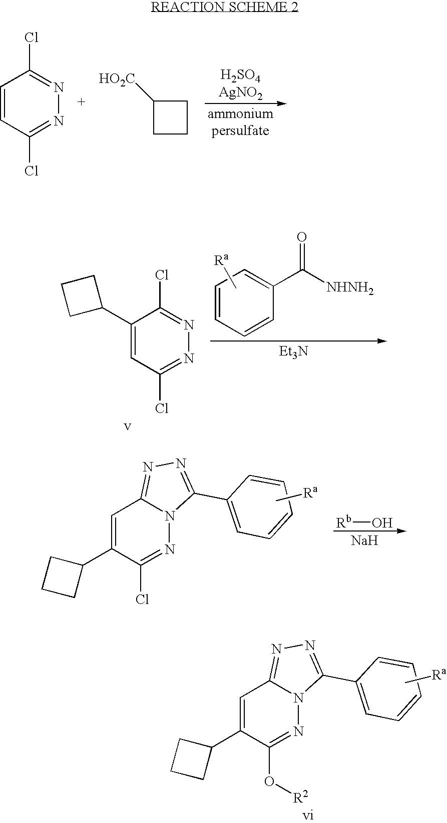

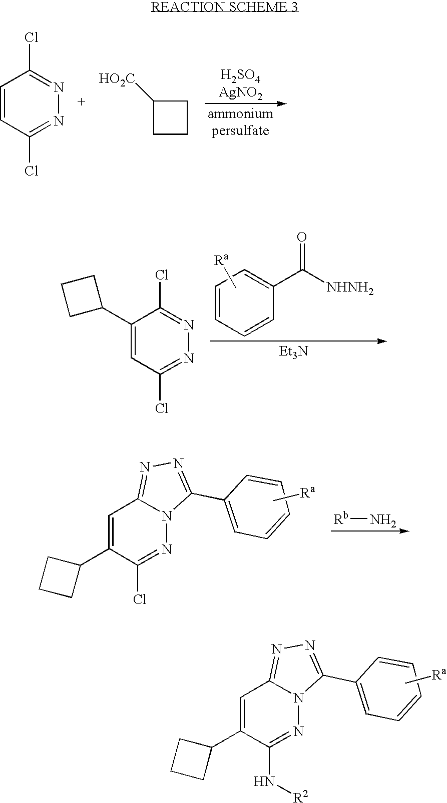

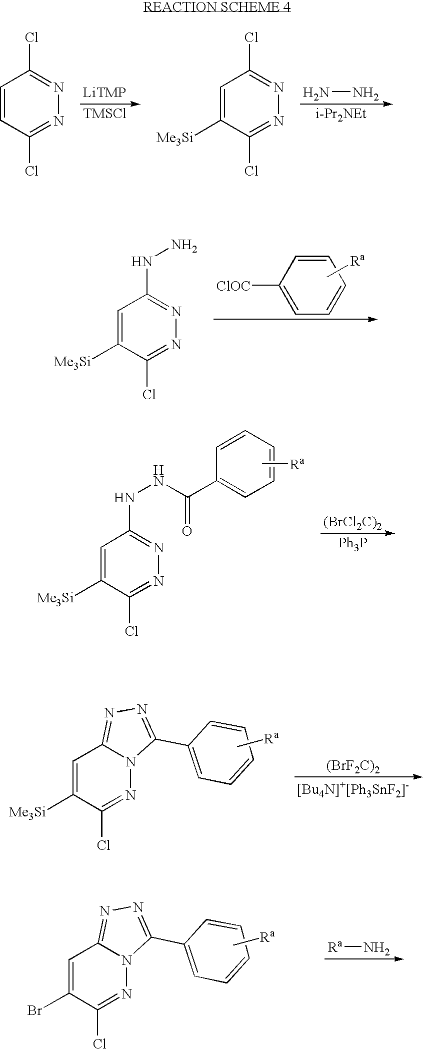

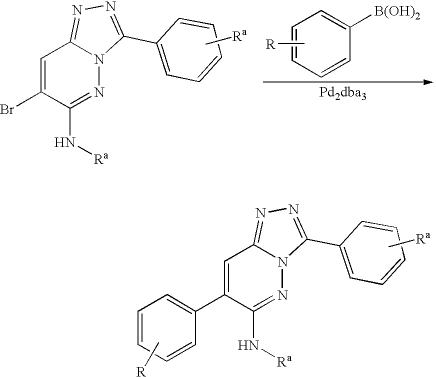

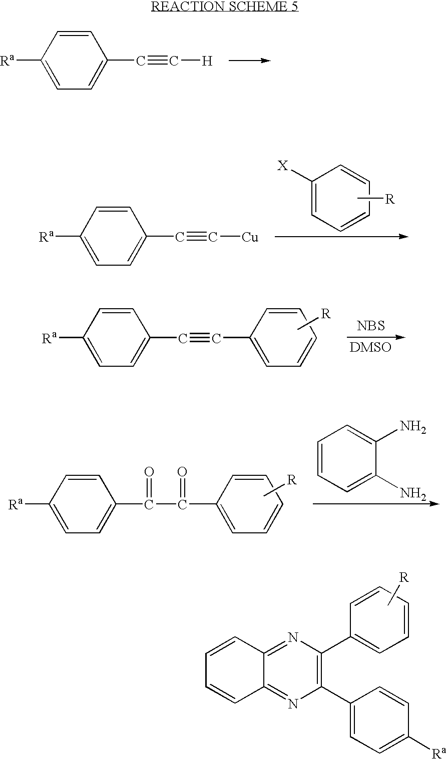

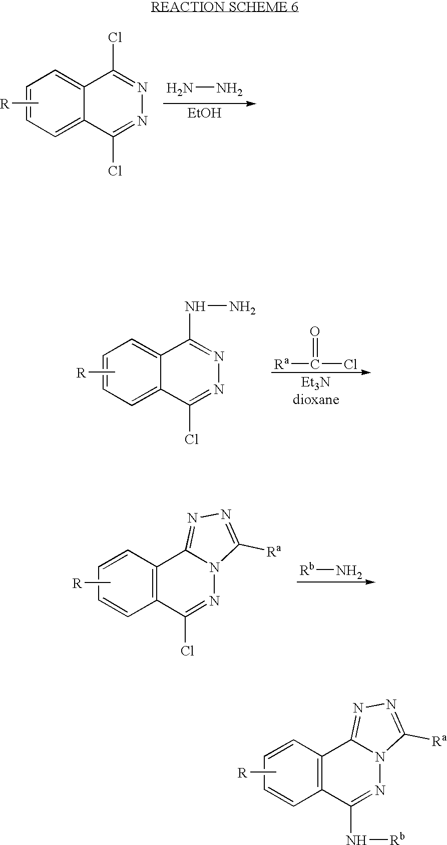

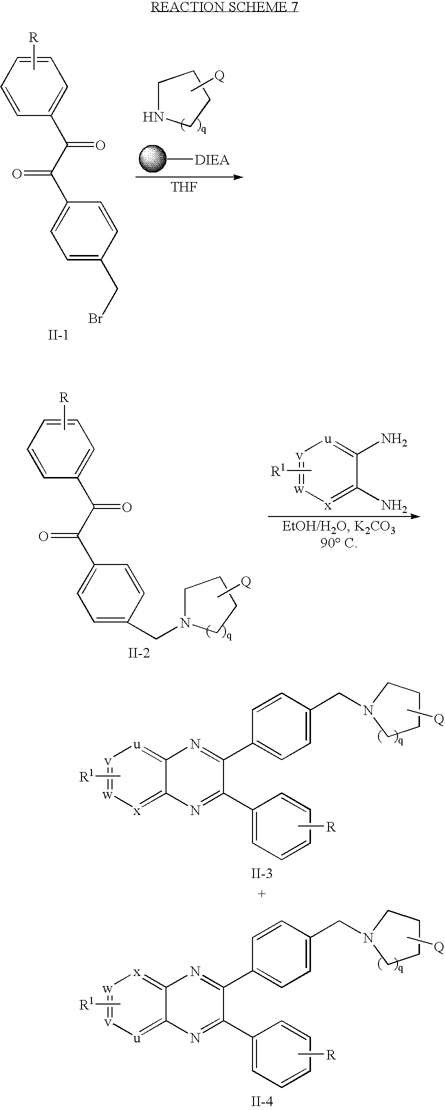

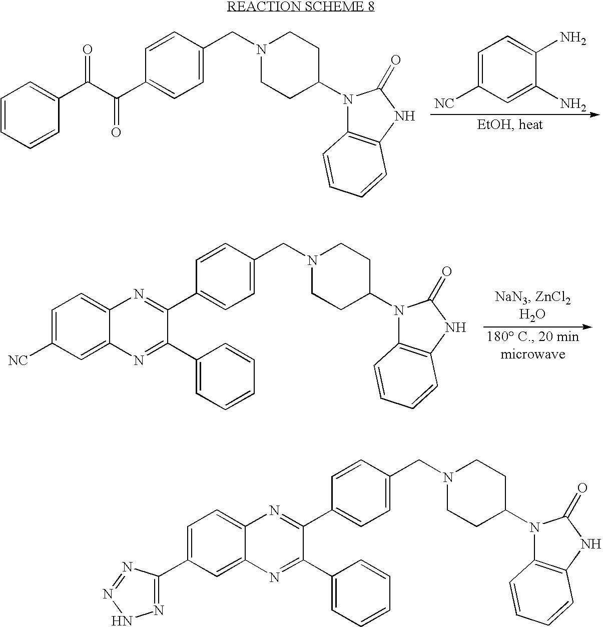

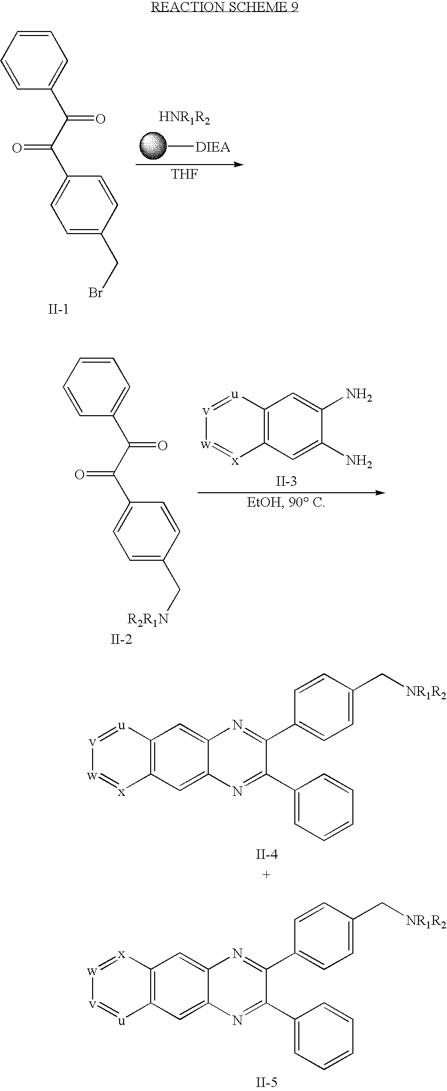

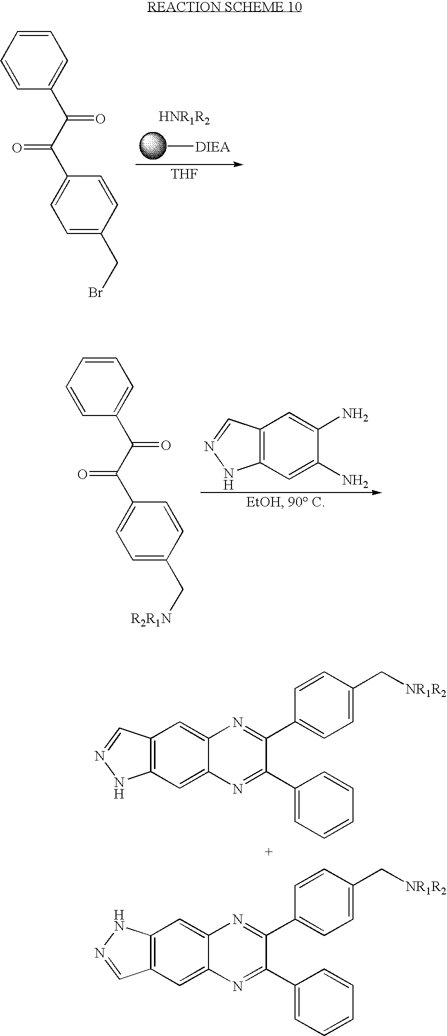

The compounds which are inhibitors of protein kinases may be prepared by employing reactions as shown in the following Reaction Schemes, in addition to other standard manipulations that are known in the literature or exemplified in the experimental procedures. These Reaction Schemes, therefore, are not limited by the compounds listed nor by any particular substituents employed for illustrative purposes. Substituent numbering, as shown in the schemes, does not necessarily correlate to that used in the claims.

As shown in the Reaction Schemes below, the term “phosphine” includes, but is not limited to, tri-substituted phosphines. Examples of tri-substituted phosphines include, but are not limited to, dppf, dppe, dppp, trialkyl phosphines (such as triphenyl phosphine, tributyl phosphine, triorthortolulyl phosphine, etc.) and the like.

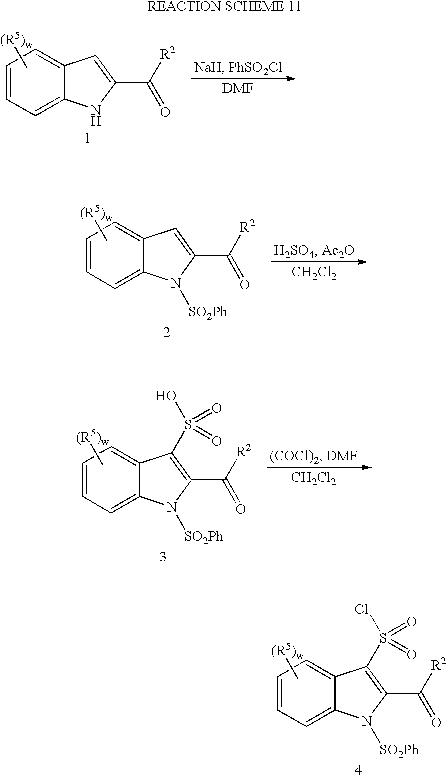

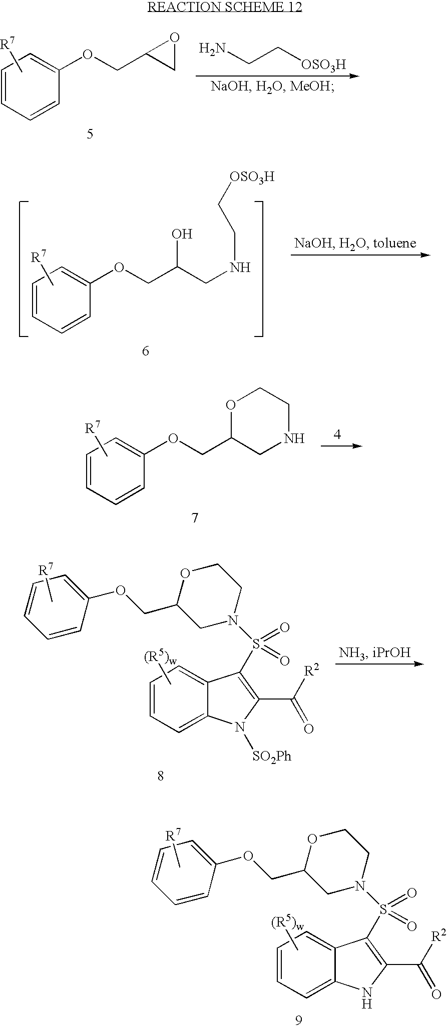

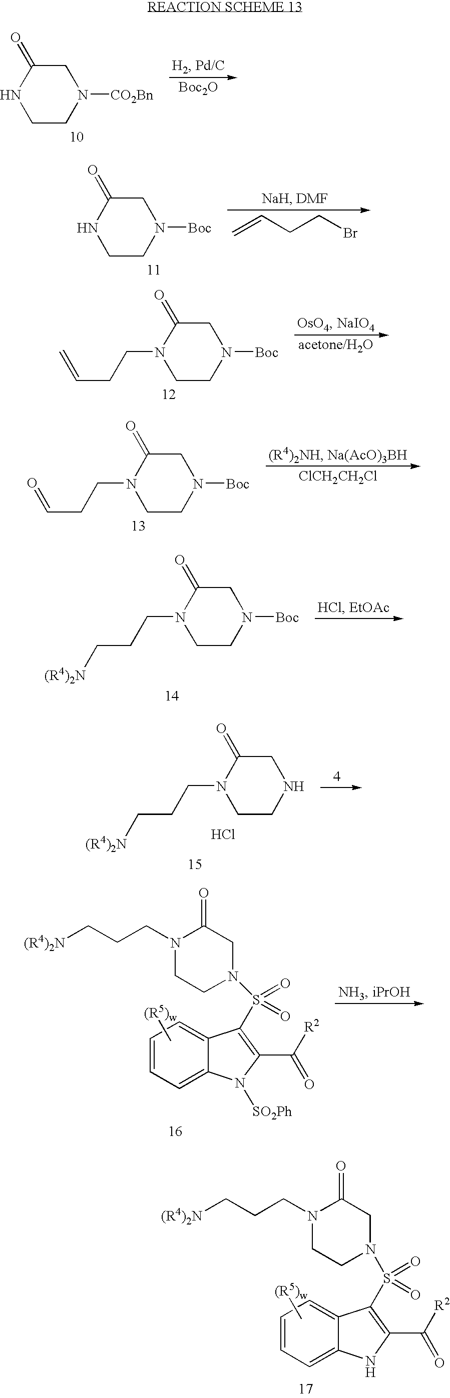

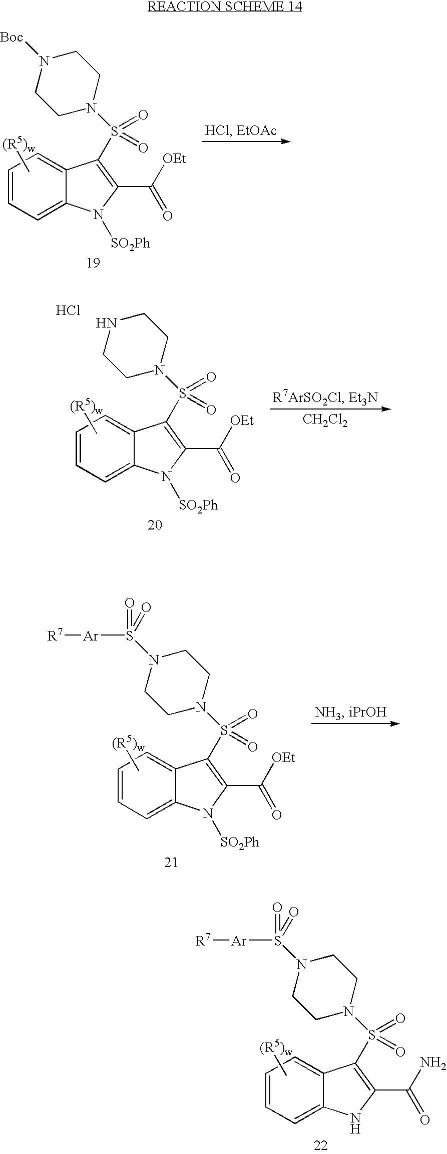

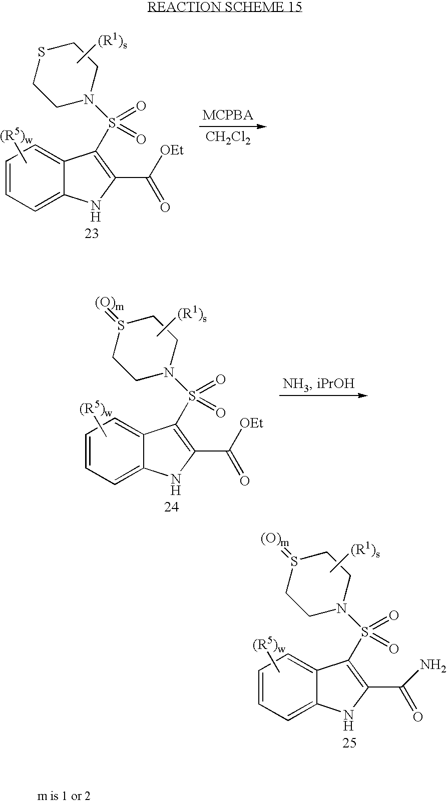

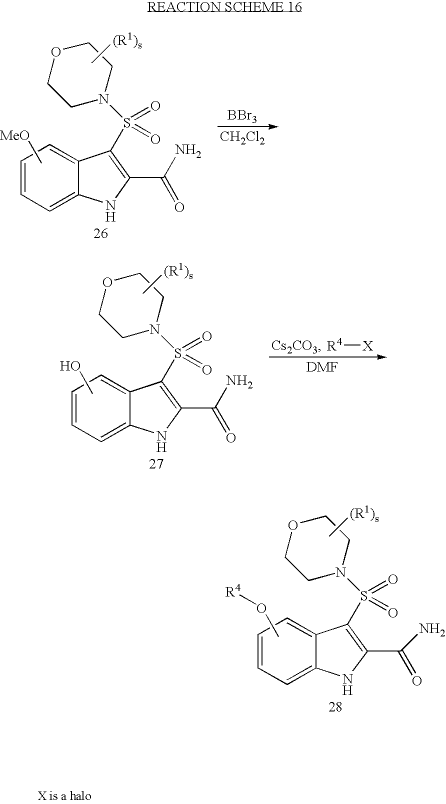

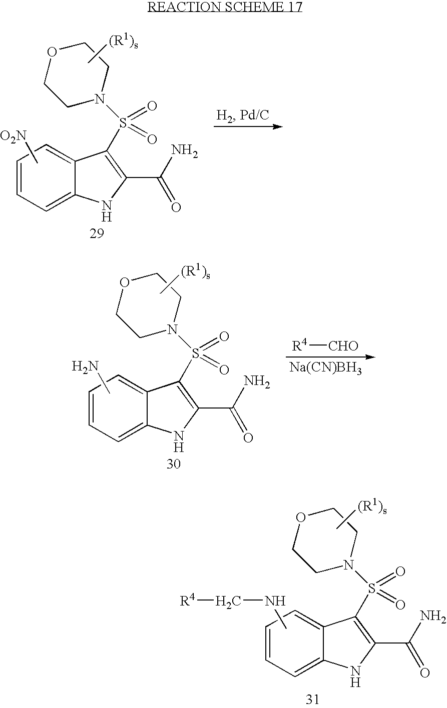

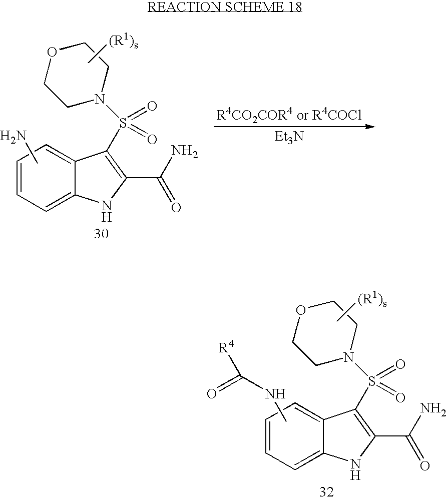

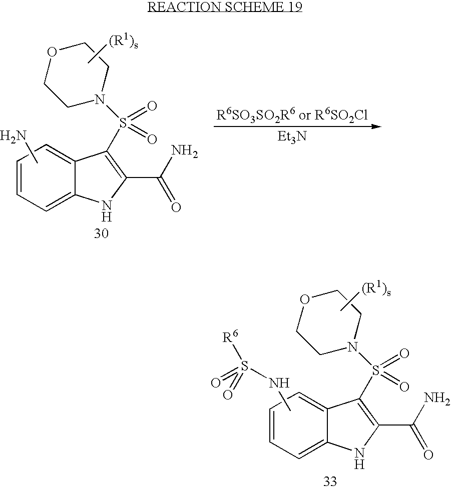

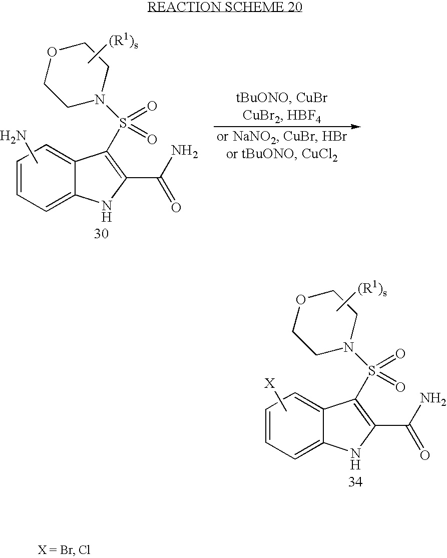

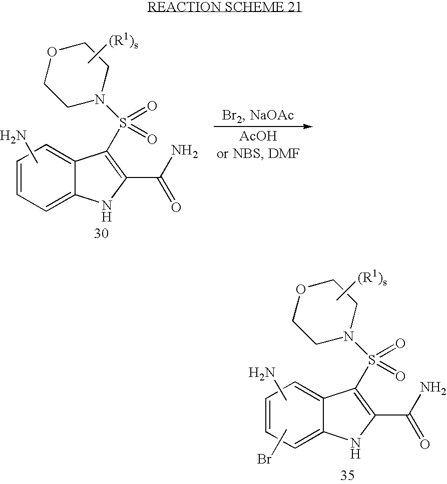

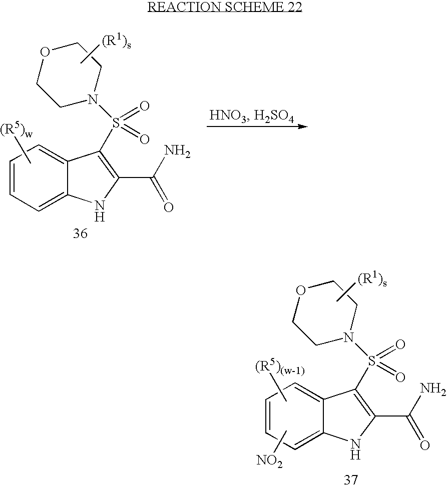

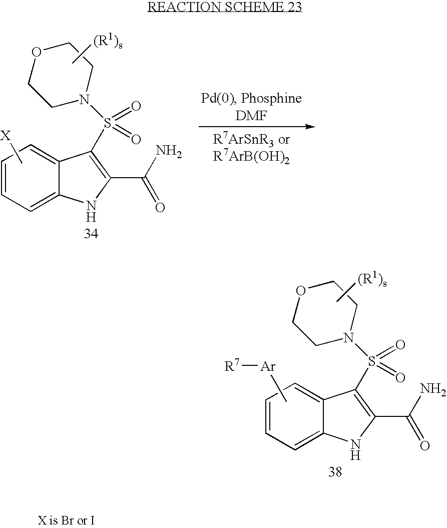

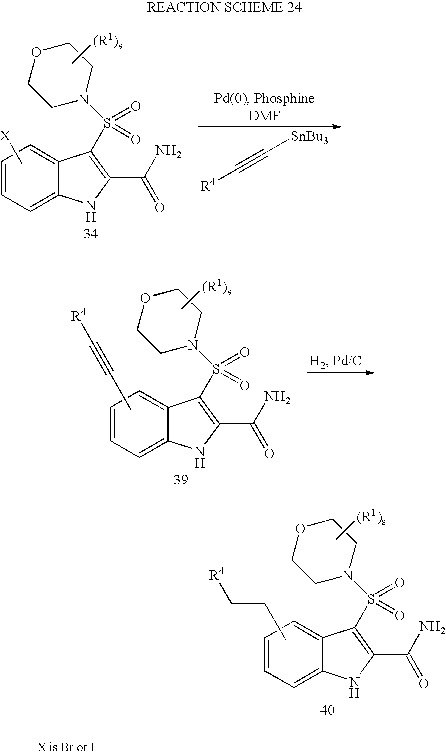

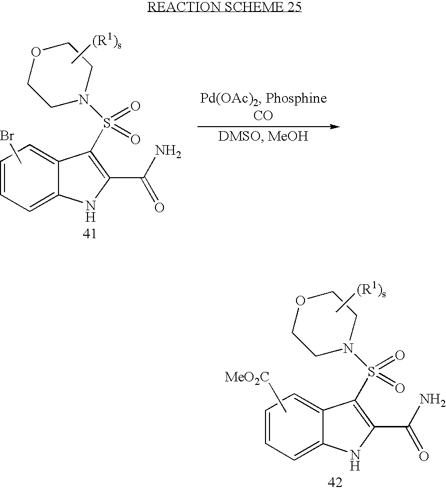

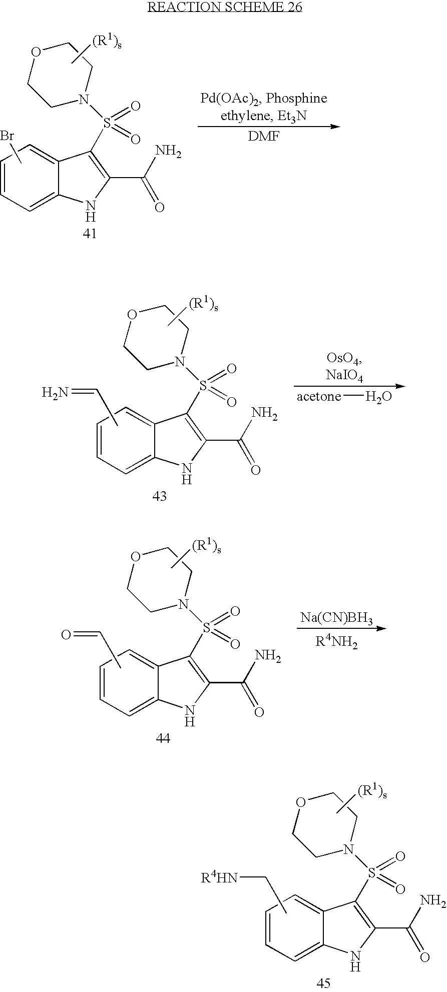

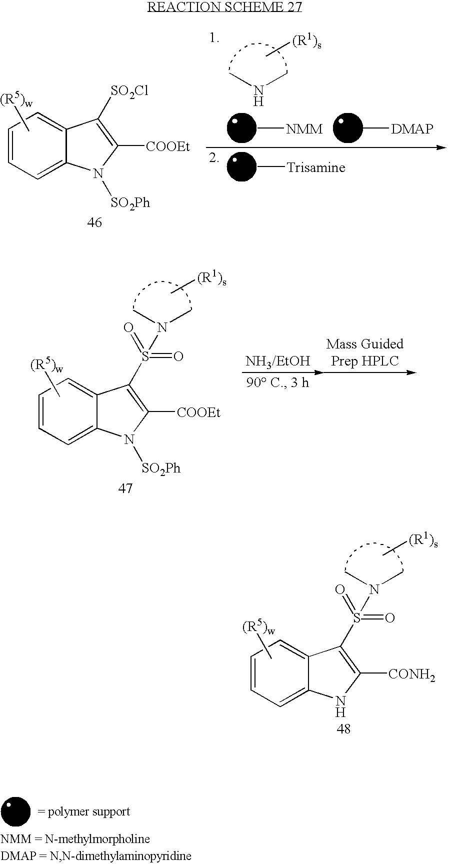

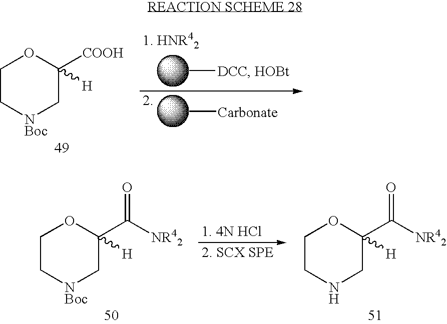

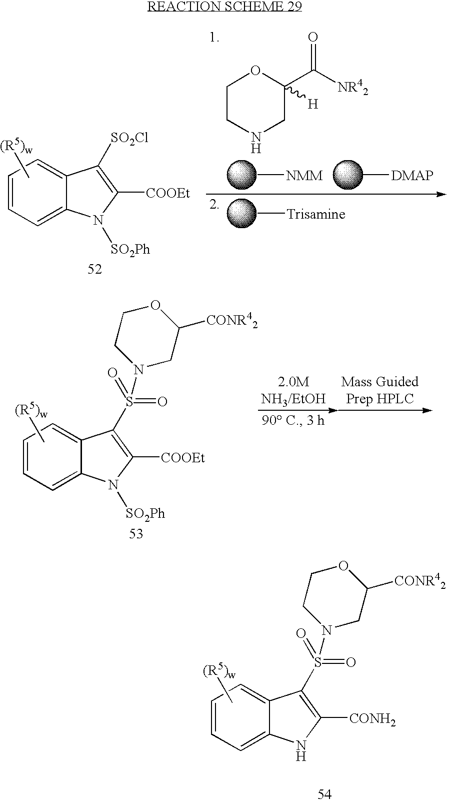

### EXAMPLES

Examples provided are intended to assist in a further understanding of the invention. Particular materials employed, species and conditions are intended to be further illustrative of the invention and not limitative of the reasonable scope thereof.

### Example 1

N′-(7-Cyclobutyl-3-phenyl-[1,2,4]triazolo[4,3-b]pyridazin-6-yl)-2,2,N,N-tetramethyl-propane-1,3-diamine (Compound 1)

Step 1: 3,6-Dichloro-4-cyclobutylpyridazine

Concentrated sulphuric acid (53.6 ml, 1.0 mol) was added carefully to a stirred suspension of 3,6-dichloropyridazine (50.0 g, 0.34 mol) in water (1.25 l). This mixture was then heated to 70° C. (internal temperature) before the addition of cyclobutane carboxylic acid (35.3 ml, 0.37 mol). A solution of silver nitrate (11.4 g, 0.07 mol) in water (20 ml) was then added over approximately one minute. This caused the reaction mixture to become milky in appearance. A solution of ammonium persulphate (230 g, 1.0 mol) in water (0.63 l) was then added over 20-30 minutes. The internal temperature rose to approximately 85° C. During the addition the product formed as a sticky precipitate. Upon complete addition the reaction was stirred for an additional 5 minutes, then allowed to cool to room temperature. The mixture was then poured onto ice and basified with concentrated aqueous ammonia, with the addition of more ice as required to keep the temperature below 10° C. The aqueous phase was extracted with dichloromethane (×3). The combined extracts were dried (MgSO₄), filtered and evaporated to give the title compound (55.7 g, 82%) as an oil. ¹H nmr (CDCl₃) indicated contamination with approximately 5% of the 4,5-dicyclobutyl compound. However, this material was used without further purification. Data for the title compound: ¹H NMR (360 MHz, d₆-DMSO) 81.79-1.90 (1H, m), 2.00-2.09 (1H, m), 2.18-2.30 (2H, m), 2.33-2.40 (2H, m), 3.63-3.72 (1H, m), 7.95 (1H, s); MS (ES⁺) m/e 203 [MH]⁺, 205 [MH]⁺, 207 [MH]⁺.

Step 2: 6-Chloro-7-cyclobutyl-3-phenyl-1,2,4-triazolo[4.3-b]pyridazine

A mixture of 3,6-dichloro-4-cyclobutylpyridazine from above (55.7 g, 0.27 mol), benzoic hydrazide (41.1 g, 0.30 mol) and triethylamine hydrochloride (41.5 g, 0.30 mol) in p-xylene (0.4 l) was stirred and heated at reflux under a stream of nitrogen for 24 hours. Upon cooling the volatiles were removed in vacuo. The residue was partitioned between dichloromethane and water. The aqueous layer was basified by the addition of solid potassium carbonate. Some dark insoluble material was removed by filtration at this stage. The aqueous phase was further extracted with dichloromethane (×2). The combined extracts were dried (MgSO₄), filtered and evaporated. The residue was purified by chromatography on silica gel eluting with 5%→10%→25% ethyl acetate/dichloromethane to give the title compound, (26.4 g, 34%) as an off-white solid. Data for the title compound: ¹H NMR (360 MHz, CDCl₃) δ 1.90-2.00 (1H, m), 2.12-2.28 (3H, m), 2.48-2.57 (2H, m), 3.69-3.78 (1H, m), 7.49-7.59 (3H, m), 7.97 (1H, s), 8.45-8.48 (2H, m); MS (ES⁺) m/e 285 [MH]⁺, 287 [MH]⁺.

Step 3: N′-(7-Cyclobutyl-3-phenyl-[1,2,4]triazolo[4,3-b]pyridazin-6-yl)-2,2,N,N-tetramethyl-propane-1,3-diamine

6-Chloro-7-cyclobutyl-3-phenyl-[1,2,4]triazolo[4,3-b]pyridazine (100 mg) and N,N,2,2-tetramethyl-1,3-propanediamine (2 ml) were heated together in a sealed tube at 70° C. for 16 hours. Cooled and water (5 ml) added. Precipitate filtered, washed (water, ether) and dried. ¹H NMR (250 MHz, DMSO)□ 1.20 (6H, s), 2.10 (1H, m), 2.24-2.65 (14H, m), 3.53-3.70 (2H, m), 7.69-7.82 (4H, m), 8.03 (1H, s), 8.70 (2H, m). MS (ES+) MH⁺=379

### Example 2

N′-(7-Cyclobutyl-3-(3,5-difluoro-phenyl)-[1,2,4]triazolo[4,3-b]pyridazin-6-yl)-2,2,N,N-tetramethyl-propane-1,3-diamine (Compound 2)

The title compound was prepared in an analogous fashion to Example 1, except substituting 3,5-difluorobenzoic hydrazine for the benzoic hydrazine in Step 2. ¹H NMR (360 MHz, CDCl₃)δ 1.07 (6H, s), 1.99 (1H, m), 2.10-2.50 (13H, m), 3.31-3.35 (3H, m), 6.84-6.89 (1H, m), 7.63 (1H, s), 7.90 (1H, vbs), 8.20-8.23 (2H, m). MS (ES+) MH⁺=415

### Example 3

N′-(7-Cyclobutyl-3-(3,4-difluoro-phenyl)-[1,2,4]triazolo[4,3-b]pyridazin-6-yl)-2,2,N,N-tetramethyl-propane-1,3-diamine (Compound 3)

The title compound was prepared in an analogous fashion to Example 1, except substituting 3,4-difluorobenzoic hydrazine for the benzoic hydrazine in Step 2. ¹H NMR (360 MHz, CDCl₃)δ 1.07 (6H, s), 1.99-2.49 (14H, m), 3.30-3.33 (3H, m), 7.25-7.30 (1H, m), 7.62 (1H, s), 7.87 (1H, vbs), 8.32-8.34 (1H, m), 8.51-8.57 (1H, m). MS (ES+) MH⁺=415

### Example 4

N′-(7-Cyclobutyl-3-(4-fluoro-phenyl)-[1,2,4]triazolo[4,3-b]pyridazin-6-yl)-2,2,N,N-tet 1,3-diamine (Compound 4)

The title compound was prepared in an analogous fashion to Example 1, except substituting 4-fluorobenzoic hydrazine for the benzoic hydrazine in Step 2. ¹H NMR (360 MHz, CDCl₃)δ 1.06 (6H, s), 1.98-2.49 (14H, m), 3.31-3.32 (3H, m), 7.18-7.26 (2H, m), 7.61 (1H, s), 7.80 (1H, vbs), 8.55-8.59 (2H, m). MS (ES+) MH⁺=397

### Example 5

N′-(7-Cyclobutyl-3-(3-fluoro-phenyl)-[1,2,4]triazolo[4,3-b]pyridazin-6-yl)-2,2N,N-tetramethyl-propane-1,3-diamine (Compound 5)

The title compound was prepared in an analogous fashion to Example 1, except substituting 3-fluorobenzoic hydrazine for the benzoic hydrazine in Step 2.

¹H NMR (360 MHz, CDCl₃)δ 1.07 (6H, s), 1.96-2.50 (14H, m), 3.31-3.35 (3H, m), 7.10-7.15 (1H, m), 7.44-7.50 (1H, m), 7.63 (1H, m) 7.81 (1H, vbs), 8.35-8.42 (2H, m). MS (ES+) MH⁺=397

### Example 6

2,2,N,N-tetramethyl-N-(3-phenyl-[1,2,4]triazolo[3,4-a]phthalazin-6-yl)-propane-1,3-diamine (Compound 6)

Step 1:1-Chloro-4-hydrazinophthalazine Hydrochloride

To a stirred solution of hydrazine hydrate (40 ml) in ethanol (120 Ml) at 80° C. was added 1,4-dichlorophthalazine (20 g). This reaction mixture was stirred at 80° C. for 0.5 hours, then left to cool and the product was collected by filtration and dried under vacuum to give 1-chloro-4-hydrazinophthalazinehydrochloride (14.6 g). ¹H NMR (250 MHz, DMSO) δ 4.64 (2H, vbs), 7.2 (1H, vbs), 7.92 (4H, bm).

Step 2: 6-Chloro-3-phenyl-1,2,4-triazolo[3,4-a]phthalazine

To a solution of 1-chloro-4-hydrazinophthalazine hydrochloride (10 g) in dioxan (220 ml) was added triethylamine (7.24 ml) and benzoyl chloride (6.04 ml). This mixture was heated at reflux for 8 hours under nitrogen. After cooling the reaction mixture was concentrated under vacuum and the solid obtained was collected by filtration, washed with water and diethyl ether and dried under vacuum, to yield the title compound (12.0 g). ¹H NMR (250 MHz, DMSO) δ 7.60 (3H, m), 8.00 (1H, t, J=8.4 Hz), 8.19 (1H, t, J=8.4 Hz), 8.31 (3H, m), 8.61 (1H, d, J=6.3 Hz).

Step 3: 2,2,N,N-tetramethyl-N-(3-phenyl-[1,2,4]triazolo[3,4-a]phthalazin-6-yl)-propane-1,3-diamine

The title compound was prepared as described in Example 1, Step 3, but replacing the 6-Chloro-7-cyclobutyl-3-phenyl-[1,2,4]triazolo[4,3-b]pyridazine with the 6-Chloro-3phenyl-1,2,4-triazolo[3,4-a]phthalazinefrom Step 2. ¹H NMR (360 MHz, CDCl₃)δ 1.13 (6H, s), 2.35 (2H, s), 2.46-2.50 (8H, m), 3.47 (2H, vbs), 7.16-7.27 (2H, m), 7.44-7.86 (5H, m), 8.55-8.57 (2H, m), 8.68 (1H, m). MS (ES+) MH⁺=375

### Example 7

N′-[3-(4-Methoxy-phenyl)-[1,2,4]triazolo[4,3-a]phthalazin-6-yl)-2,2,N,N-tetramethyl-propane-1,3-diamine (Compound 7)

The title compound was prepared in an analogous fashion to Example 1, except substituting 3-fluorobenzoic hydrazine for the benzoic hydrazine in Step 2. ¹H NMR (360 MHz, CDCl₃)δ 1.13 (6H, s), 2.45 (6H, s), 2.49 (2H, s), 3.45-3.46 (2H, m), 3.90 (3H, s) 7.04-7.07 (2H, m), 7.65-7.70 (2H, m), 7.80-7.84 (1H, m), 8.51 (2H, m), 8.66 (1H, m). MS (ES+) MH⁺=405

### Example 8

6-(2-Hydroxyethyl)oxy-3,7-diphenyl-[1,2,4]triazolo[4,3-b]pyridazine (Compound 8)

Step 1: 4-Phenyl-1,2-dihydropyridazine-3,6-dione

Phenylmaleic anhydride (30 g, 0.17 mol), sodium acetate trihydrate (28 g, 0.21 mol) and hydrazine monohydrate (10 ml, 0.21 mol) were heated together at reflux in 40% acetic acid (600 ml) for 18 hours. The mixture was cooled at 7° C. for 2 hours, then filtered. The solid was washed with diethyl ether and dried in vacuo to give 11 g (34%) of the title compound: ¹H NMR (250 MHz, DMSO-d₆) δ 7.16 (1H, br s), 7.44 (5H, m), 7.80 (2H, br s); MS (ES⁺) m/e 189 [MH⁺].

Step 2: 3,6 Dichloro-4-phenylpyridazine

4-Phenyl-1,2-dihydropyridazine-3,6-dinoe (3.4 g, 18 mmol) was heated at reflux in phosphorus oxychloride (70 ml) for 6 hours. The solution was concentrated in vacuo, then the residue was dissolved in dichloromethane (100 ml) and was neutralized by the addition of cold 10% aqueous sodium hydrogen carbonate (150 ml). The aqueous phase was washed with dichloromethane (2×50 ml), then the combined organic layers were washed with saturated aqueous sodium chloride (50 ml), dried (Na₂SO₄), and concentrated in vacuo to yield 3.9 g (97%) of the title compound: ¹H NMR (250 MHz, DMSO-d₆) δ 7.54-7.66 (5H, m) 8.14 (1H, s); MS (ES⁺) m/e 225/227/229 [MH⁺].

Step 3: 6-Chloro-3,7-diphenyl-1,2,3-trizolo[4,3-b]pyridazine

3,6-Dichloro-4-phenylpyridazine (2.9 g, 13 mmol), benzoic hydrazide (1.9 g, 21 mmol) and triethylammonium chloride (2.0 g, 14 mmol) were heated together at reflux in xylene (150 ml) for three days. More benzoic hydrazide (0.88 g, 6.5 mmol) was added and the mixture was heated as before for another day. The solvent was removed in vacuo, and the residue was purified by flash chromatography (silica gel, 0-50% EtAOc/CH₂Cl₂) to afford 1.4 g (36%) of the title compound as a solid: ¹H NMR (250 MHz, CDCl₃) δ 7.55 (8H, m), 8.12 (1H, s), 8.50 (2H, m); MS (ES⁺) m/e 307/309 [MH⁺].

Step 4: 6-(2-Hydroxyethyl)oxy-3.7-diphenyl-1,2,3-trizolo[4,3-b]pyridazine

Anhydrous DMF (1.5 ml) was added to a test tube containing NaH (13 mg) under nitrogen. Ethylene glycol (2 ml) was added and the mixture stirred at room temperature for 1 hour. The 6-chloro-3,7-diphenyl-1,2,3-trizolo[4,3-b]pyridazine (50 mg) (prepared as described in Step 3) was added as a solid and the reaction stirred at room temperature for 30 minutes and then heated at 60° C. for 8 hours and then stirred 10 hours at room temperature. The reaction mixture was then poured over 20 ml of hot water, the mixture cooled and the aqueous mixture extracted with ether. The organic phases were combined, washed with water, dried over MgSO₄, filtered and concentrated under vacuum to provide the title compound. ¹H NMR (CDCl₃, 500 MHz at 20° C.) δ 8.48 (d, 2H, J=8.3), 8.04 (d, 1H, J=0.7), 7.61 (m, 2H), 7.57 (dd, 2H, J=7.6 and 8.1), 7.52 (m, 4H), 4.62 (dd, 2H, J=3.9 and 5.1), 4.04 (d, 2H, J=3.7). LC/MS (ES+) [M+1]=333.2.

### Example 9

6-(2-Hydroxybutyl)oxy-3,7-diphenyl-[1,2,4]triazolo[4,3-b]pyridazine (Compound 9)

The title compound was prepared by the procedure described in Example 1, but replacing ethylene glycol with 1,4-butanediol in Step 4. ¹H NMR (CDCl₃, 500 MHz at 20° C.) δ 8.52 (dd, 2H, J=7.8 and 1.5), 8.02 (d, 1H, J=0.5), 7.58 (m, 4H), 7.51 (m, 4H), 4.53 (t, 2H, J=6.4), 3.69 (app. t, 2H, J=5.5), 1.97 (m 2H), 1.72 (m, 2H). LC/MS (ES+) [M+1]=361.3.

### Example 10

Preparation of 2-(2-aminoprop-2-ylphenyl)-3-phenylquinazoline (Compound 10)

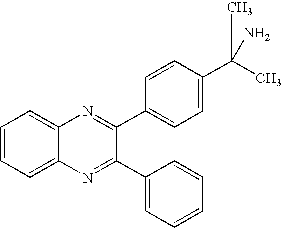

Step 1: Preparation of Ethyl 4-iodobenzoate

A mixture of 21.0 g of 4-iodobenzoic acid, 100 ml of absolute EtOH and 6 ml of concentrated sulfuric acid was refluxed with stirring for 6 days. At the end of this time the reaction mixture was concentrated by boiling and an additional 4 ml of concentrated sulfuric acid added. The mixture was then refluxed for an additional 11 days, after which the mixture was cooled and 50 g of ice and 150 ml Et₂O were added. The phases were separated and the aqueous layer was extracted with Et₂O. The combined organic phases were washed with water, sat. aqueous NaHCO₃ and water. The organic phase was then dried over MgSO₄ and concentrated under vacuum to provide the title compound as a clear brownish liquid.

Step 2: Preparation of α,α-dimethyl-4-iodobenzyl Alcohol

To a cooled (ice/H₂O) solution of 2.76 g of ethyl 4-iodobenzoate (prepared as described in Step 1) in 10 ml of anhyd. Et₂O was added, over a 5 minute period, 26.5 ml of 1.52M CH₃MgBr/Et₂O solution. The mixture was stirred at ice bath temperature for 2.5 hours and then quenched by slow addition of 6 ml of H₂O. The reaction mixture was filtered and the solid residue rinsed with ether. The combined filtrates were dried over MgSO₄ and concentrated under vacuum to provide the title compound as a clear yellowish liquid.

Step 3: Preparation of α,α-dimethyl-4-iodo-N-formamido-benzyl Amine

19 ml of glacial acetic acid was cooled in an ice bath until a slurry formed. 4.18 g of sodium cyanide was added over a 30 minute period. A cooled (ice/H₂O) solution of 10.3 ml conc. sulfuric acid in 95 ml glacial acetic acid was added to the cyanide solution over a 15 min. period. The ice bath was removed and 19.92 g of the α,α-dimethyl-4-iodobenzyl alcohol (prepared as described in Step 2) was added over a 10 minute period. The resulting white suspension was stirred 90 minutes. And left standing overnight at room temperature. The reaction mixture was poured over ice and water and ether added. This mixture was neutralized with solid Na₂CO₃.

Step 4: Preparation of Copper (I) Phenylacetylide

To a solution of 10.7 g of phenylacetylene in 500 ml of absolute ethanol was added a solution of 20 g of copper iodide in 250 ml of conc. NH₄OH and 100 ml of water. The solution was stirred 30 minutes and then filtered. The solid that was collected was washed with water, 95% aq. Ethanol and then ether. The solid was then collected and dried under vacuum to provide the title compound as a bright yellow solid.

Step 5: Preparation of 1-(2-formamidoprop-2-ylphenyl)-2-phenylacetylene

A mixture of 11.83 g of the iodophenyl compound described in Step 3, 6.74 g of Copper (I) phenylacetylide and 165 ml of dry pyridine was stirred at 120° C. for 72 hours. The reaction was then allowed to cool and the mixture was poured over approximately 300 g of ice and water with vigorous stirring. The mixture was then extracted with 1:1 benzene:diethylether. The organic solution was washed with 3N hydrochloric acid, dried over MgSO₄, filtered and concentrated to provide a solid, that was recrystallized from benzene/cyclohexane to provide the title compound.

Step 6: Preparation of 4-(2-formamidoprop-2-yl)-benzil

1-(2-formamidoprop-2-ylphenyl)-2-phenylacetylene from Step 5 (4.81 g) was dissolved in 30 ml of dried DMSO. N-Bromosuccinamide (NBS) (5.65 g) was added and the reaction stirred at room temperature for 96 hours. At this time 500 mg of NBS was added and the reaction stirred an additional 24 hours. The reaction mixture was then poured over water and the aqueous mixture extracted with benzene. The combined organic phases were washed with water and dried over MgSO₄. The organic slurry was then filtered and concentrated in vacuo to provide the title compound

Step 7: Preparation of 4-(2-aminoprop-2-yl)-benzil

4-(2-formamidoprop-2-yl)-benzil, prepared as described in Step 6 (6.17 g) was dissolved in 100 ml of glacial acetic acid, 84 ml of water and 6 ml of concentrated HCl. The mixture was stirred at reflux for 3 hours and then the solvent removed under vacuum at 60° C. The residue was converted to the free based form, extracted with organic solvent, washed with water, dried and concentrated to provide the title compound as an oil.

Step 8: Preparation of 2-(2-aminoprop-2-ylphenyl)-3-phenylquinazoline

A mixture of 1.0 g of 4-(2-aminoprop-2-yl)-benzil from Step 7, 0.406 g of o-phenylenediamine, 25 ml of glacial acetic acid and 15 ml of water was refluxed for 4.5 hours. The mixture was then allowed to stand overnight at room temperature. Most of the solvent was then removed under vacuum and the residue was taken up in 30 ml of water and 50 ml of 6 N aq. NaOH was added. The gum that precipitated was extracted with chloroform. The organic solution was washed with water, dried over MgSO₄ and concentrated under vacuum.

The residue was redissolved in chloroform and ethanolic HCl was added, precipitating out the hydrochloride salt. The salt was recrystallized from i-PrOH to provide the title compound as the hydrochloride salt—i-PrOH solvate (pale yellow plates). Mp 269° C.-271° C. (melted/resolidified at 250° C.).

Anal. Calc. for C₂₃H₂₁N₃.HCl.i-PrOH: C, 71.62; H, 6.94; N, 9.64. Found: C, 71.93; H, 6.97; N, 9.72

¹H NMR (CDCl₃, 500 MHz at 20° C.) δ 9.04 (broad s, 2.4H), 8.10 (d, 1H, J=7.8), 8.02 (d, 1H, J=7.8), 7.72 (dd, 1H, J=7.0 and 8.2), 7.66 (dd, 1H, J=7.0 and 8.2), 7.56 (m, 4H), 7.46 (dd, 2H, J=1.2 and 8.5), 7.31 (m, 3H), 1.81 (s, 6H). LC/MS (ES+) [M+1]=340.3.

### Example 11

Preparation of 2,3-bis(4-aminophenyl)-quinoxaline (Compound 11)

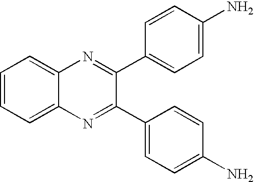

Step 1: Preparation of Meso (d,l) Hydrobenzoin

To a slurry of 97.0 g of benzil in 1 liter of 95% EtOH was added 20 g of sodium borohydride. After stirring 10 minutes, the mixture was diluted with 1 liter of water and the mixture was treated with activated carbon. The mixture was then filtered trough supercel and the filtrate heated and diluted with an additional 2 liters of water until it became slightly cloudy. The mixture was then cooled to 0 to 5° C. and the resulting crytals were collected and washed with cold water. The crystals were then dried in vacuo.

Step 2: Preparation of 4,4′-dinitrobenzil

150 ml of fuming nitric acid was cooled to −10° C. and 25 g of the hydrobenzoin (prepared as described in Step 1) was added slowly portionwise while maintaining the temperature between −10° C. to −5° C. The reaction mixture was maintained at 0° C. for an additional 2 hours. 70 ml of water was added and the mixture was refluxed for 30 minutes and then poured onto 500 g of cracked ice. The residue was separated from the mixture by decantation and the residue was then boiled with 500 ml of water. The water layer was removed.

The remaining gum was dissolved in boiling acetone and the solution treated with decolorizing carbon and filtered. The filtrated was then cooled to −5° C. and the resulting crystals were collected and washed with cold acetone and dried in vacuo. An additional crop of crystalline title compound was obtained from recrystallization of the mother liquor residue.

Step 2: Preparation of 4,4′-diaminobenzil

3.8 g of 4,4′-dinitrobenzil was reduced under hydrogen with 3.8 g 10% R^{u} on C in EtOH. The mixture was filtered through Supracel and the filtrate concentrated under vacuum to dryness. The residue was dissolved in 50% denatured ethanol in water, treated with Darco and filtered. The filtrate was cooled to 0° C. and the resulting crystals were collected and washed with 50% denatured ethanol in water. The crystals were then dried under a heat lamp to give the title compound as a yellow powder.

Step 3: Preparation of 2,3-bis(4-aminophenyl)-quinoxaline

A mixture of 1.0 g (4.17 mmole) of 4,4′-diaminobenzil and 0.45 g of o-phenylenediamine in 250 ml glacial acetic acid was heated at 50° C. for 15 minutes, then stirred for 16 hours at room temperature. The mixture was then heated to 80° C. and allowed to cool slowly. The solvent was removed under vacuum and the residue was redissolved in ethanol and that was removed under vacuum.

The solid residue was recrystalized from boiling acetone, and the solid collected. The residue from the mother liquors was recrystalized form 95% EtOH and the resulting crystals combined with the crystals from the acetone crystalization and all were recrystalized from 1:1 abs. EtOH:95% EtOH to provide crystalline material. The crystals were dried for over 5 hours at 110° C. under vacuum to provide the title compound.

Anal. Calc. for C₂₀H₁₆N₄: C, 76.90; H, 5.16; N, 17.94. Found: C, 76.83; H, 4.88; N, 18.16

¹H NMR (CDCl₃, 500 MHz at 20° C.) δ 8.08 (m, 2H), 7.67 (m, 2H), 7.39 (m, 4H), 6.64 (m, 4H), 3.80 (broad s, 4H).

LC/MS (ES+) [M+1]=313.3.

### Example 12

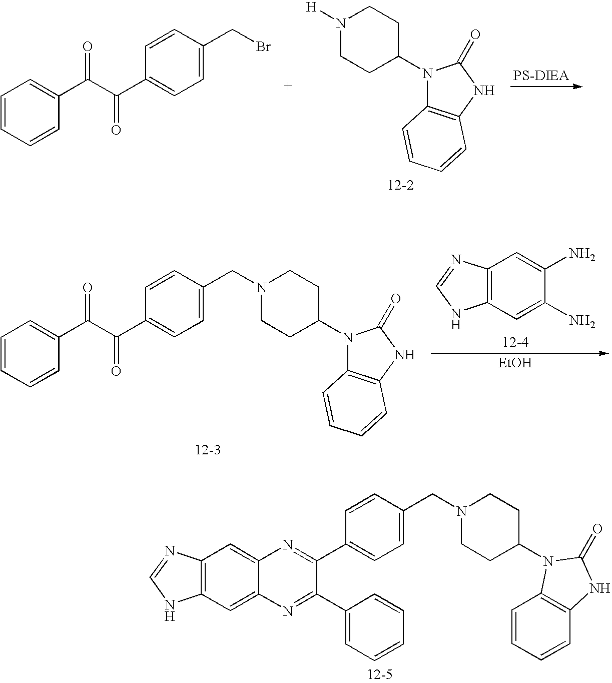

Step 1:1-(4-{[4-(2-Oxo-2,3-dihydro-1H-benzimidazol-1-yl)pipepridin-1-yl]methyl}phenyl)-2-phenylethane-1,2-dione (12-3)

To an 8 mL vial was placed bromomethyl benzil (12-2) (Toronto Research chemicals, 500 mg, 1.65 mmol), 4-(2-keto-1-benzimidazolinyl)piperidine (Aldrich, 358 mg, 1.65 mol), PS-DIEA (887 mg, 3.3 mmol, 3.72 mml/g) and dry THF (6 mL, 0.3 M). The vial was placed on a GlasCol rotator and allowed to rotate for 2 hours. After this time, the contents of the vial were filtered through a 10 mL BioRad tube, washed with THF and concentrated in vaccuo. The crude material was then purified on an Agilent 1100 series Mass Guided HPLC purification system to afford the TFA salt of (12-3) as a pale yellow solid. Analytical LCMS: single peak (214 nm) at 2.487 min (CH₃CN/H₂O/1% TFA, 4 min gradient). ¹H NMR (500 MHz, DMSO-d₆): δ 10.9 (s, 1H), 8.05 (m, 2H), 7.93 (m, 2H), 7.79 (m, 2H), 7.63 (m, 2H), 7.24 (s, 1H), 6.98 (s, 4H), 4.47 ((s, 2H), 3.5 (m, 2H), 3.2 (m, 3H), 2.61 (q, J=11 Hz, 2H), 1.9 (d, J=11 Hz, 2H). HRMS, calc'd for C₂₇H₂₆N₃O₃ (M+H), 440.1965; found 440.1968.

Step 2: 1-{1-[4-(7-Phenyl-1H-imidazo[4,5-g]quinoxalin-6-yl)benzyl]piperidin-4-yl}-1,3-dihydro-2H-benzimidazol-2-one (12-5)

To an 8 mL vial was placed 1-(4-{[4-(2-oxo-2,3-dihydro-1H-benzimidazol-1-yl)pipepridin-1-yl]methyl}phenyl)-2-phenylethane-1,2-dione (12-3) (56 mg, 0.10 mmol), 5,6-diaminobenzimidazole, trihydrochloride 12-4 (25 mg, 0.10 mol) and dissolved in EtOH (2 mL). The vial was placed in a J-KEM heater/shaker block and warmed to 90 degrees for 9 hours. After this time, the vials were cooled and concentrated in vaccuo. The crude material was then purified on an Agilent 1100 series Mass Guided HPLC purification system to afford of the TFA salt of (12-5) as a brown solid. Analytical LCMS: single peak (214 nm) at 2.066 min (CH₃CN/H₂O/1% TFA, 4 min gradient). ¹H NMR (600 MHz, CD₃OD): δ 9.32 (s, 1H), 8.52 (s, 2H), 7.71 (d, J=8.1 Hz, 1H), 7.58 (d, J=8.1 Hz, 2H), 7.55 (d, J=7.7 Hz, 2H), 7.43 (t, J=7.0 Hz, 1H) 7.38 (t, J=7.0 Hz, 2H), 7.28 (m, 1H), 7.07 (m, 3H), 4.59 (m, 1H), 4.43 (s, 2H), 3.66 (d, J=12.1 Hz, 2H), 3.28 (t, J=12.0 Hz, 2H), 2.82 (q, J=11.8 Hz, 2H), 2.08 (d, J=13.9 Hz, 2H). HRMS, calc'd for C₃₄H₂₉N₇O(M+H), 552.2503; found 552.2503.

Compounds in Table 1 were synthesized as shown in Example, but substituting the appropriately substituted cyclic amine for compound (12-2) in the example: The TFA salt of the compound shown was isolated by Mass Guided HPLC purification.

TABLE 1

| # | Compound | MS M + 1 |
|---|---|---|
| 12-6 | 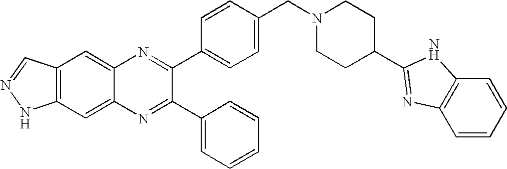 | 536.6 |
| 12-7 | 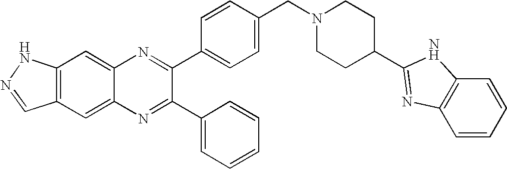 | 536.6 |

### Example 13

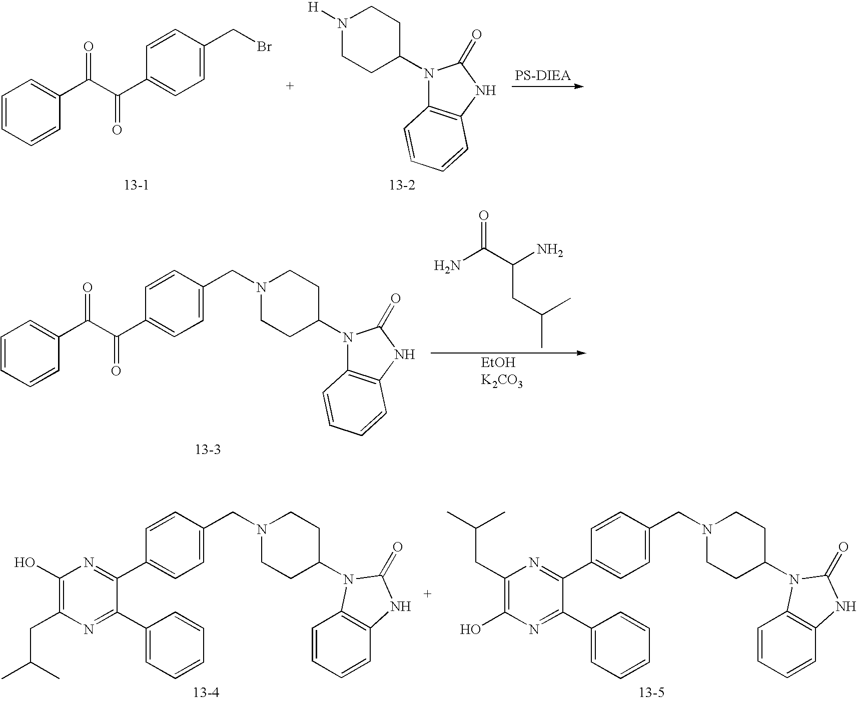

Step 1:1-(4-{[4-(2-Oxo-2,3-dihydro-1H-benzimidazol-1-yl)pipepridin-1-yl]methyl}phenyl)-2-phenylethane-1,2-dione (13-3)

To an 8 mL vial was placed bromomethyl benzil (13-1) (Toronto Research Chemicals, 500 mg, 1.65 mmol), 4-(2-keto-1-benzimidazolinyl)piperidine (13-2) (Aldrich, 358 mg, 1.65 mol), PS-DIEA (887 mg, 3.3 mmol, 3.72 mml/g) and dry THF (6 mL, 0.3 M). The vial was placed on a GlasCol rotator and allowed to rotate for 2 hours. After this time, the contents of the vial were filtered through a 10 mL BioRad tube, washed with THF and concentrated in vaccuo. The crude material was then purified on an Agilent 1100 series Mass Guided HPLC purification system to afford the TFA salt of (13-3) as a pale yellow solid. Analytical LCMS: single peak (214 nm) at 2.487 min (CH₃CN/H₂O/1% TFA, 4 min gradient). ¹H NMR (500 MHz, DMSO-d₆): δ 10.9 (s, 1H), 8.05 (m, 2H), 7.93 (m, 2H), 7.79 (m, 2H), 7.63 (m, 2H), 7.24 (s, 1H), 6.98 (s, 4H), 4.47 ((s, 2H), 3.5 (m, 2H), 3.2 (m, 3H), 2.61 (q, J=11 Hz, 2H), 1.9 (d, J=11 Hz, 2H). HRMS, calc'd for C₂₇H₂₆N₃O₃ (M+H), 440.1965; found 440.1968.

Step 2: 1-{1-[4-(6-Hydroxy-5-isobutyl-3-phenylpyrazin-2-yl)benzyl]piperidin-4-yl}-1,3-dihydro-2H-benzimidazol-2-one 13-4 and 1-{1-[4-(5-Hydroxy-6-isobutyl-3-phenylpyrazin-2-yl)benzyl]piperidin-4-yl}-1,3-dihydro-2H-benzimidazol-2-one (13-5)

1-(4-{[4-(2-Oxo-2,3-dihydro-1H-benzimidazol-1-yl)pipepridin-1-yl]methyl}phenyl)-2-phenylethane-1,2-dione (13-3) (1.661 g, 30 mmol), leucine carboxamide HCl (0.501 g, 3.0 mmol), and K₂CO₃ (0.829 g, 6.0 mmol) were dissolved in 30 mL of EtOH/H₂O (5/1) in a one-necked, 100 ML flask. The mixture solution is heated at 90° C. for 16 hours. After this time, the reaction were cooled and concentrated in vaccuo. The crude material was then purified on an Agilent 1100 series Mass Guided HPLC purification system to afford the TFA salts of 13-4 and 13-5 as slightly yellow solids.

(13-4): Analytical LCMS: single peak (214 nm) at 2.655 min (CH₃CN/H₂O/1% TFA, 4 min gradient). ¹H NMR (500 MHz, CD₃OD): δ 7.54 (d, J=7.9 Hz, 2H), 7.47 (d, J=7.7 Hz, 2H), 7.24 (m, 6H) 7.08 (d, J=2.4 Hz, 3H), 4.57 (m, 1H), 4.40 (s, 2H), 3.63 (d, J=11.5 Hz, 2H), 3.26 (t, J=12.6 Hz, 2H), 2.78 (m, 4H), 2.29 (m, 2H) 2.09 (d, J=12.8 Hz, 2H) 1.02 (d, J=6.8 Hz, 6H). HRMS, calc'd for C₃₃H₃₅N₅O₂(M+H), 534.2846; found 534.2864.

(13-5): Analytical LCMS: single peak (214 nm) at 2.343 min (CH₃CN/H₂O/1% TFA, 4 min gradient). ¹H NMR (500 MHz, CD₃OD): δ 7.39 (m, 9H), 7.24 (m, 1H), 7.07 (m, 3H), 4.54 (m, 1H), 4.33 (s, 2H), 3.63 (d, J=12.1 Hz, 2H), 3.21 (t, J=12.6 Hz, 2H), 2.77 (q, J=12.5, 2H), 2.74 (d, J=7.0, 2H) 2.29 (m, 1H) 2.07 (d, J=13.9 Hz, 2H) 1.02 (d, J=6.8 Hz, 6H); HRMS, calc'd for C₃₃H₃₅N₅O₂(M+H), 534.2846; found 534.2864. HRMS, calc'd for C₃₃H₃₅N₅O₂ (M+H), 534.2846; found 534.2850.

### Example 14

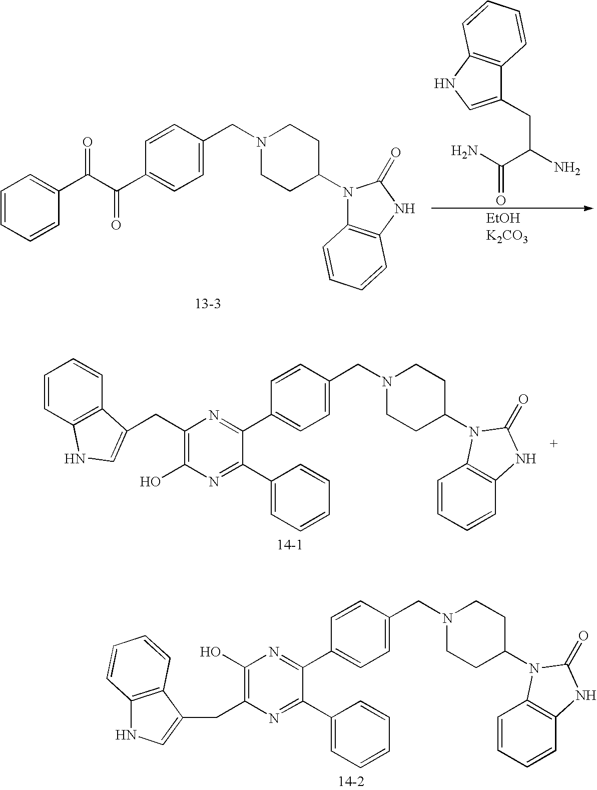

1-(1-{4-[5-Hydroxy-6-(1H-indol-3-ylmethyl)-3-phenylpyrazin-2-yl]benzyl}piperidin-4-yl)-1,3-dihydro-2H-benzimidazol-2-one (14-1) and 1-(1-{4-[6-Hydroxy-5-(1H-indol-3-ylmethyl)-3-phenylpyrazin-2-yl]benzyl}piperidin-4-yl)-1.3-dihydro-2H-benzimidazol-2-one (14-2)

1-(4-{[4-(2-oxo-2,3-dihydro-1H-benzimidazol-1-yl)pipepridin-1-yl]methyl}phenyl)-2-phenylethane-1,2-dione (13-3) (56 mg, 0.1 mmol), L-tryptophan carboxamide (HCl) (24 mg, 0.1 mmol), and K₂CO₃ (28 mg, 0.2 mmol) were dissolved in 2 mL of EtOH/H₂O (5/1) in an 8 mL vial. The mixture solution is heated at 90° C. for 16 hours. After this time, the reaction were cooled and concentrated in vaccuo. The crude material was then purified on an Agilent 1100 series Mass Guided HPLC purification system to afford the TFA salts of (141) and (14-2) as brown solids.

(14-1): Analytical LCMS: single peak (214 nm) at 2.381 min (CH₃CN/H₂O/1% TFA, 4 min gradient). ¹H NMR (600 MHz, CD₃OD): δ 7.76 (d, J=7.9 Hz 1H), 7.48 (d, J=8.6 Hz, 2H), 7.42 (d, J=8.6 Hz, 2H) 7.32 (d, J=8.0 Hz, 1H), 7.20(m, 6H), 7.07 (m, 5H), 6.99(t, J=7.0 Hz, 1H), 4.53 (m, 1H), 4.34 (s, 2H), 4.30 (s, 2H), 3.57 (d, J=10.5 Hz, 2H), 3.19 (t, J=12.9 Hz, 2H), 2.75 (q, J=12.9, 2H), 2.04 (d, J=14.1 2H). HRMS, calc'd for C₃₈H₃₄N₆O₂ (M+H), 607.2816; found 607.2790.

(14-2): TFA salt as a brown solid. Analytical LCMS: single peak (214 nm) at 2.558 min (CH₃CN/H₂O/1% TFA, 4 min gradient). ¹H NMR (500 MHz, CD₃OD): δ 7.76 (d, J=7.9 Hz 1H), 7.48 (d, J=7.7 Hz, 2H), 7.42 (d, J=8.0 Hz, 2H), 7.36 (m, 1H), 7.32 (d, J=8.0 Hz, 1H), 7.23(m, 6H), 7.07 (m, 4H), 6.99(t, J=7.5 Hz, 1H), 4.53 (m, 1H), 4.32 (m, 4H), 3.58 (d, J=11.0 Hz, 2H), 3.19 (t, J=12.9 Hz, 2H), 2.75 (q, J=6.7 Hz, 2H), 2.07 (d, J=13.9 Hz, 2H). HRMS, calc'd for C₃₈H₃₄N₆O₂ (M+H), 607.2816; found 607.2790.

Compounds in Table 2 were synthesized as shown in Examples 13 and 14. The TFA salt of the compound shown was isolated by Mass Guided HPLC purification.

TABLE 2

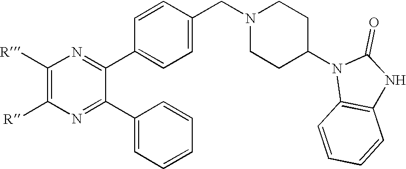

| # | R″ | R′′′ | MS M + 1 |
|---|---|---|---|
| 13-6 | —CH₂Ph | —OH | 568.6 |
| 13-7 | 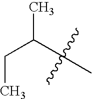 | —OH | 534.6 |
| 13-8 | —OH | 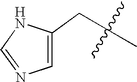 | 558.6 |
| 13-9 | —OH | —CH₃ | 492.5 |

### Example 15

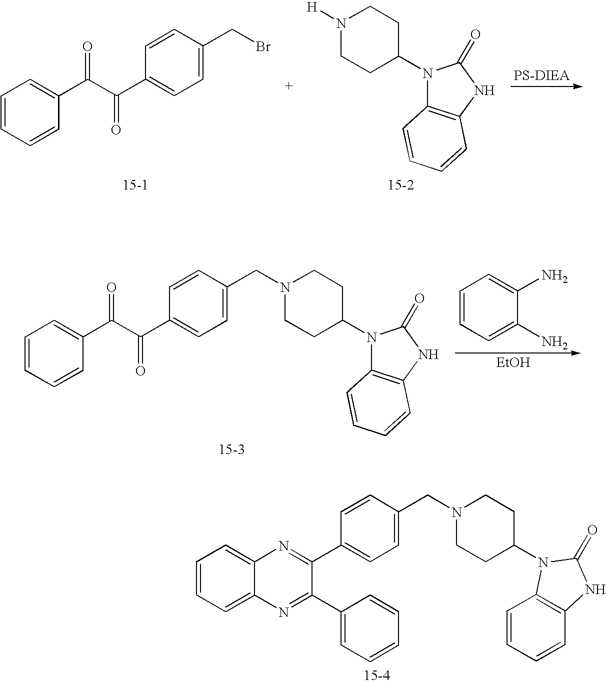

Step 1:1-(4-{[4-(2-Oxo-2,3-dihydro-1H-benzimidazol-1-yl)pipepridin-1-yl]methyl}phenyl)-2-phenylethane-1,2-dione (15-3)

To an 8 mL vial was placed bromomethyl benzil (15-1) (Toronto Research Chemicals, 500 mg, 1.65 mmol), 4-(2-keto-1-benzimidazolinyl)piperidine (15-2) (Aldrich, 358 mg, 1.65 mol), PS-DIEA (887 mg, 3.3 mmol, 3.72 mml/g) and dry THF (6 mL, 0.3 M). The vial was placed on a GlasCol rotator and allowed to rotate for 2 hours. After this time, the contents of the vial were filtered through a 10 mL BioRad tube, washed with THF and concentrated in vaccuo. The crude material was then purified on an Agilent 1100 series Mass Guided HPLC purification system to afford 775 mg of the TFA salt of (15-3) as a pale yellow solid. Analytical LCMS: single peak (214 nm) at 2.487 min (CH₃CN/H₂O/1% TFA, 4 min gradient). ¹H NMR (500 MHz, DMSO-d₆): δ 10.9 (s, 1H), 8.05 (m, 2H), 7.93 (m, 2H), 7.79 (m, 2H), 7.63 (m, 2H), 7.24 (s, 1H), 6.98 (s, 4H), 4.47 ((s, 2H), 3.5 (m, 2H), 3.2 (m, 3H), 2.61 (q, J=11 Hz, 2H), 1.9 (d, J=11 Hz, 2H). HRMS, calc'd for C₂₇H₂₆N₃O₃ (M+H), 440.1965; found 440.1968.

Step 2: 1-{1-[4-(3-Phenylquinoxalin-2-yl)benzyl]piperidin-4-yl}-1,3-dihydro-2H-benzimidazol-2-one (15-4)

To an 8 mL vial was placed 1-(4-{[4-(2-oxo-2,3-dihydro-1H-benzimidazol-1-yl)pipepridin-1-yl]methyl phenyl)-2-phenylethane-1,2-dione (15-3) (88 mg, 0.16 mmol), 1,2-diaminobenzene (17 mg, 0.16 mol) and dissolved in EtOH (3 mL). The vial was placed in a J-KEM heater/shaker block and warmed to 90 degrees for 9 hours. After this time, the vials were cooled and concentrated in vaccuo. The crude material was then purified on an Agilent 1100 series Mass Guided HPLC purification system to afford 80 mg of the TFA salt of (15-4) as a brown solid. Analytical LCMS: single peak (214 nm) at 2.625 min (CH₃CN/H₂O/1% TFA, 4 min gradient). ¹H NMR (400 MHz, DMSO-d₆): δ 10.9 (s, 1H), 8.18 (m, 2H), 7.92 (m, 2H), 7.6 (m, 2H), 7.52 (m, 4H), 7.4 (m, 3H), 7.28 (m, 1H), 7.0 (s, 3H), 4.50 (m, 1H), 4.4 (s, 2H), 3.5 (d, J=12 Hz, 2H), 3.2 (t, J=12 Hz, 2H), 2.6 (q, J=11.8 Hz, 2H), 1.94 (d, J=12 Hz, 2H). HRMS, calc'd for C₃₃H₃₀N₅O(M+H), 512.2445; found 512.2443.

### Example 16

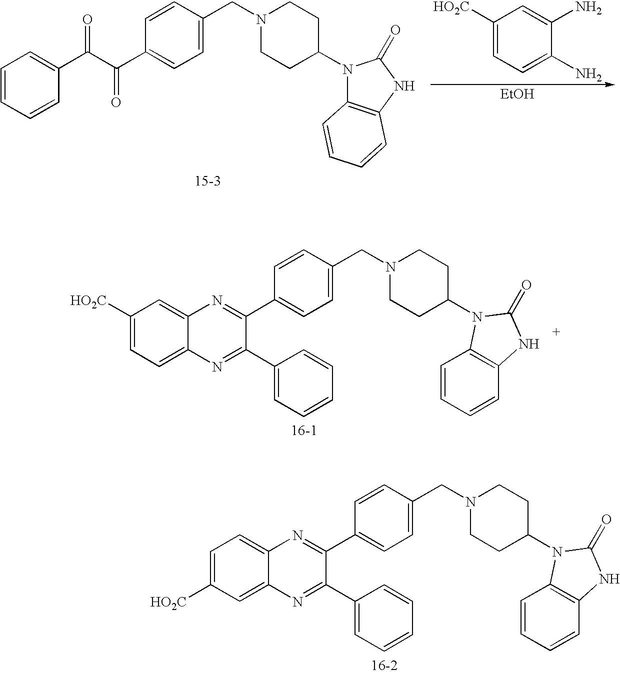

3-(4-{[4-(2-Oxo-2,3-dihydro-1H-benzamidazol-1-yl)piperdin-1-yl]methyl}phenyl)-2-phenylquinaxoline-6-carboxylic Acid (16-1) and 2-(4-{[4-(2-Oxo-2,3-dihydro-1H-benzamidazol-1-yl)piperdin-1-yl]methyl}phenyl)-2-phenylquinaxoline-6-carboxylic Acid (16-2)

To an 8 mL vial was placed 1-(4-{[4-(2-oxo-2,3-dihydro-1H-benzimidazol-1-yl)pipepridin-1-yl]methyl}phenyl)-2-phenylethane-1,2-dione (15-3) (500 mg, 1.1 mmol), 4-carboxy-1,2-diaminobenzene (170 mg, 1.1 mol) and dissolved in EtOH (10 mL). The vial was placed in a J-KEM heater/shaker block and warmed to 90 degrees for 9 hours. After this time, the vials were cooled and concentrated in vaccuo. The crude material was then purified on an Agilent 1100 series Mass Guided HPLC purification system to afford the TFA salt as a white solid. This protocol afforded a 1:1 mixture of regioisomers (16-1) and (16-2) which were separated by prep HPLC.

(16-1): Analytical LCMS: single peak (214 nm) at 2.430 min (CH₃CN/H₂O/1% TFA, 4 min gradient). ¹H NMR for 2-1 (400 MHz, DMSO-d₆): δ 13.1 (s, 1H), 10.8 (s, 1H), 8.66 (s, 1H), 8.32 (m, 1H), 8.23 (m, 1H), 7.52 (m, 2H), 7.49 (m, 2H), 7.42 (m, 1H), 7.38 (m, 4H), 7.24 (m, 1H), 6.97 (s, 3H), 4.17 (m, 1H), 3.61 (s, 2H), 2.97 (d, J=11.4 Hz, 2H), 2.38 (q, J=10 Hz, 2H), 2.17 (t, J=11.4 Hz, 2H), 1.66 (d, J=10 Hz, 2H). HRMS calc'd for C₃₄H₃₀N₅O₃ (M+H), 556.2343; found 556.2352.

(16-2): Analytical LCMS: single peak (214 nm) at 2.620 min (CH₃CN/H₂O/1% TFA, 4 min gradient). ¹H NMR for 2-1 (400 MHz, DMSO-d₆): δ 12.9 (s, 1H), 10.6 (s, 1H), 8.60 (s, 1H), 8.30 (m, 1H), 8.27 (m, 1H), 7.55 (m, 2H), 7.49 (m, 2H), 7.42 (m, 1H), 7.38 (m, 4H), 7.24 (m, 1H), 6.97 (s, 3H), 4.17 (m, 1H), 3.61 (s, 2H), 2.97 (d, J=11.4 Hz, 2H), 2.38 (q, J=10 Hz, 2H), 2.17 (t, J=11.4 Hz, 2H), 1.66 (d, J=10 Hz, 2H). HRMS calc'd for C₃₄H₃₀N₅O₃ (M+H), 556.2343; found 556.2350.

### Example 17

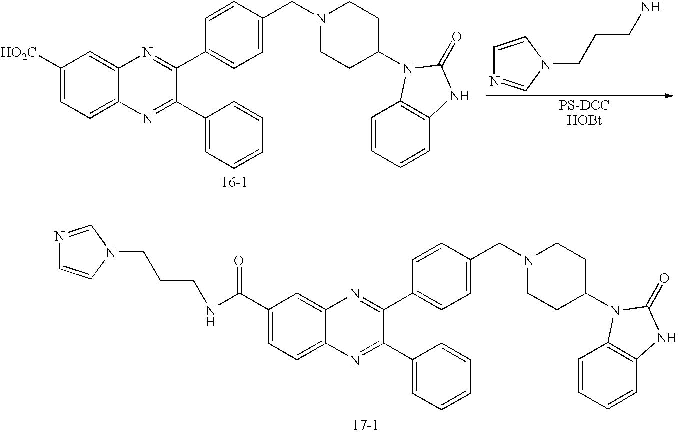

N-[3-(1H-Imidazol-1-yl)propyl]-3-(4-{[4-(2-oxo-2,3-dihydro-1H-benzamidazol-1-yl)piperdin-1-yl]methyl}phenyl)-2-phenylquinaxoline-6-carboxamide (17-1)

To an 8 mL vial was placed 3-(4-{[4-(2-oxo-2,3-dihydro-1H-benzamidazol-1-yl)piperdin-1-yl]methyl}phenyl)-2-phenylquinaxoline-6-carboxylic acid (16-1) (35 mg, 0.08 mol), 3-imidazoylpropylamine (10 μL, 0.08 mol), PS-DCC (110 mg, 0.15 mmol, 1.38 mmol/g), HOBt (15 mg, 0.11 mmol) and DCM (4 mL). The vial was placed on a GlasCol rotator and allowed to rotate overnight. In the morning, MP-carbonate (90 mg, 0.32 mmol, 3.38 mmol/g) was added, and the vial allowed to rotate for another 3 hours. After this time, the vial's contents were filtered through a BioRad tube, washed with DCM and concentrated. The crude material was then purified on an Agilent 1100 series Mass Guided HPLC purification system to afford the bis TFA salt of (17-1) as a brown solid. Analytical LCMS: single peak (214 nm) at 2.090 min (CH₃CN/H₂O/1% TFA, 4 min gradient). ¹H NMR (400 MHz, DMSO-d₆): δ 10.9 (s, 1H), 9.1 (s, 1H), 9.0 (t, J=4.8 Hz, 1H), 8.71 (s, 1H), 8.29 (s, 2H), 7.84 (s, 1H), 7.69 (2, 1H), 7.55 (m, 7H), 7.3 (s, 1H), 7.0 (s, 3H), 4.51 (m, 1H), 4.39 (s, 2H), 4.31 (t, J=6.8 Hz, 2H), 3.47 (m, 2H), 3.19 (m, 2H), 2.66 (q, J=11.2 Hz, 2H), 2.16 (quint, J=6.8 Hz, 2H), 1.94 (d, J=12.4 Hz, 2H). HRMS calc'd for C₄₀H₃₉N₈O₂ (M+H), 663.3190; found 663.3191.

### Example 18

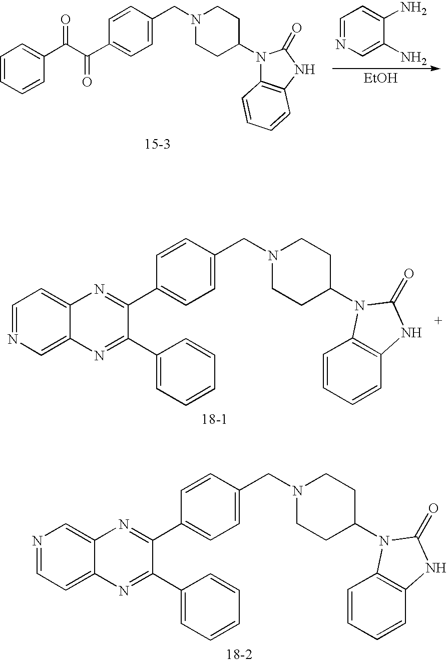

1-{1-[4-(3-phenylpyrido[3,4-b]pyrazin-2-yl)benzyl]piperidin-4-yl}-1,3-dihydro-2H-benzimidazol-2-one (18-1) and 1-{1-[4-(2-phenylpyrido[3,4-b]pyrazin-3-yl)benzyl]piperidin-4-yl}-1.3-dihydro-2H-benzimidazol-2-one (18-2)

To an 8 mL vial was placed 1-(4-{[4-(2-oxo-2,3-dihydro-1H-benzimidazol-1-yl)pipepridin-1-yl]methyl}phenyl)-2-phenylethane-1,2-dione (15-3) (59 mg, 0.10 mmol), 3,4-diaminopyridine (11.1 mg, 0.10 mol) and dissolved in EtOH (3 mL). The vial was placed in a J-KEM heater/shaker block and warmed to 90 degrees for 9 hours. After this time, the vials were cooled and concentrated in vaccuo. The crude material was then purified on an Agilent 1100 series Mass Guided HPLC purification system to afford the TFA salts of (18-1) and (18-2) as brown solids.

(18-1): Analytical LCMS: single peak (214 nm) at 2.220 min (CH₃CN/H₂O/1% TFA, 4 min gradient). ¹H NMR (400 MHz, CD₃OD): δ 9.64 (d, J=4 Hz, 1H), 8.85 (dd, J=6.2, 0.9 Hz, 1H), 8.22 (dd, J=6.1, 2.0 Hz, 1H), 7.58 (m, 4H), 7.46 (m, 1H), 7.38 (m, 2H), 7.28 (m, 1H), 7.07 (d, J=2.6 Hz, 3H), 4.59 (m, 1H), 4.43 (s, 2H), 3.60 (d, J=12.5 Hz, 2H), 3.28 (t, J=11.1 Hz, 2H), 2.82 (q, J=12.5 Hz, 2H), 2.08 (d, J=13.4 Hz, 2H). HRMS, calc'd for C₃₂H₂₉N₆O(M+H), 513.2393; found 512.2393.

(18-2): Analytical LCMS: single peak (214 nm) at 2.410 min (CH₃CN/H₂O/1% TFA, 4 min gradient). ¹H NMR (400 MHz, CD₃OD): δ 9.60 (d, J=4 Hz, 1H), 8.81 (dd, J=6.2, 0.9 Hz, 1H), 8.20 (dd, J=6.1, 2.0 Hz, 1H), 7.58 (m, 4H), 7.46 (m, 1H), 7.38 (m, 2H), 7.28 (m, 1H), 7.07 (d, J=2.6 Hz, 3H), 4.59 (m, 1H), 4.43 (s, 2H), 3.60 (d, J=12.5 Hz, 2H), 3.28 (t, J=1.1 Hz, 2H), 2.82 (q, J=12.5 Hz, 2H), 2.08 (d, J=13.4 Hz, 2H). HRMS, calc'd for C₃₂H₂₉N₆O(M+H), 513.2393; found 512.2391.

### Example 19

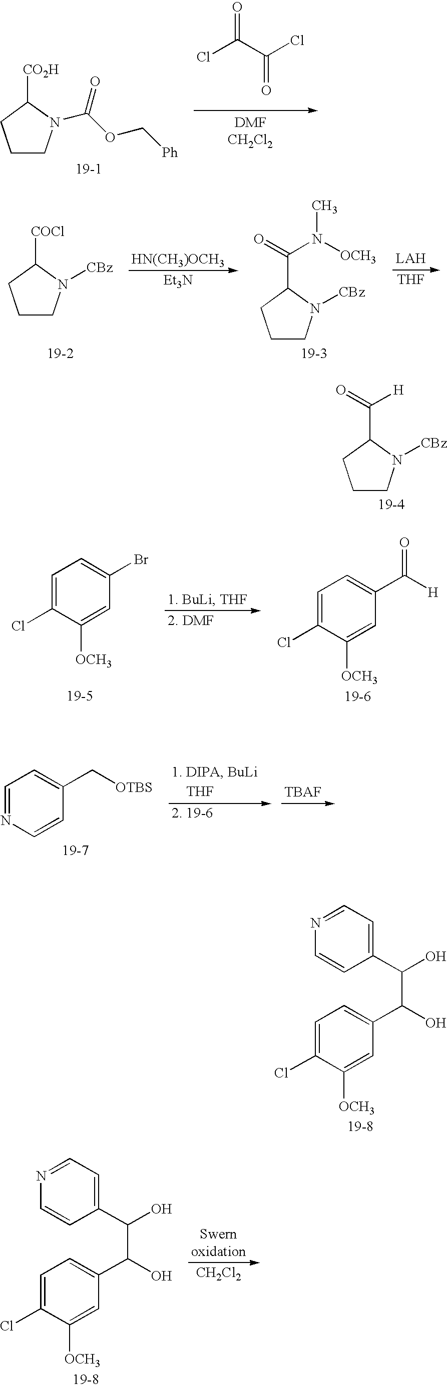

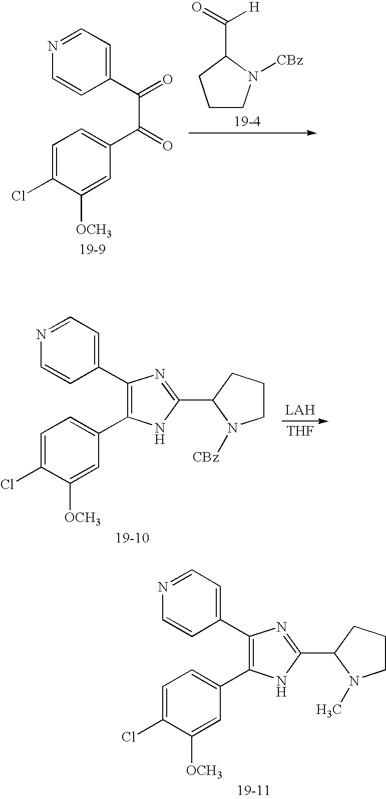

Step 1: N-benzyloxyvcarbonyl-2-pyrrolidine-N-methoxy-N-methylcarboxamide (19-3)

N-benzyloxycarbonylproline (25 g, 0.116 moles) and oxalyl chloride (10.12 mL) was dissolved in 310 mL of CH₂Cl₂ and DMF (0.8 mL) and the mixture stirred at room temperature for 2 hours. At the end of this time the solvent was evaporated and the residue was dissolved in 400 mL of CH₂Cl₂ and the solution cooled to 0° C. N,O-dimethylhydroxylamine hydrochloride (11.32 g, 0.116 moles) was added, followed by dropwise addition of Et₃N (35.8 mL). The solution was allowed to warm to room temperature and stirred for 2 hours. The reaction mixture was further diluted with 300 mL of CH₂Cl₂ and poured into a bicarbonate solution. The aqueous layer was extracted with CH₂Cl₂ and the combined organic layers were dried over Na₂SO₄ and filtered. The organic solvents were evaporated and the residue suspended in a EtOAc/CH₂Cl₂/MeOH mixture. The mixture was filtered and the filtrate concentrated under vacuum and redissolved/filtered. The resulting organic soluble residue was purified on a silica gel column (70% EtOAc in hexane) to provide compound (19-3).

Step 2: N-benzyloxycarbonyl-2-pyrrolidine Carboxaldehyde (19-4)

Compound (19-3) (25 g) was dissolved in 200 mL of THF and the solution cooled to 0° C. The solution was flushed with Ar and LiAlH₄ (49 mL of 1 M solution) was added and the reaction mixture was stirred for 12 hours. An additional 0.25 eq. of the LiAlH₄ solution was added and the reaction mixture was stirred an additional 20 minutes. At the end of this time the reaction was quenched by the addition of 2 mL of water and diluted with EtOAc. The aluminum salts were removed by filtration and the filtrate was ashed with potassium sulfate solution, brine and then dried over Mg₂SO₄. The mixture was then filtered and concentrated under vacuum. The residue was purified on a silica gel column (20% EtOAc in hexane) to provide compound (19-4).

Step 3: 4-chloro-3-methoxybenzaldehyde (19-6)

5-Bromo-2-chloroanisole (19-5) (2.2 g) was dissolved in 200 mL of THF and the solution cooled to −78° C. Butyl lithium (4.4 mL of 2.5M solution) was added slowly, the reaction solution was stirred 5 minutes and DMF (0.93 mL) was added slowly. The reaction mixture was stirred briefly and then poured over sodium bicarbonate and ice. The aqueous mixture was extracted with EtOAc, the organic layer was washed with brine, dried over MgSO4 and filtered. The filtrate was concentrated under vacuum and the residue then purified by silica gel chromatography 1:9 EtOAc:hexane to provide the aldehyde 19-6 as a white solid.

Step 4: 1-(4-Chloro-3-methoxyphenyl)-2-pyridin-4-yl-ethane-1,2-diol (19-8)

To a stirring solution of diisopropylamine (14.4 mL, 110 mmol) in tetrahydrofuran (400 mL) at −78° C. was added, dropwise, n-butyllithium (44 mL of a 2.5 M solution in tetrahydrofuran). After ten minutes, a solution of 4-pyridylcarbinol t-butyldimethylsilyl ether (22.3 g, 100 mmol) in tetrahydrofuran (80 mL) was added dropwise and the temperature allowed to rise to −15° C. The solution was again cooled to −78° C. and a solution of 4-chloro-3-methoxybenzaldehyde (19-6) (17 g, 100 mmol) in tetrahydrofuran (60 mL) added dropwise. After the solution was allowed to warm to room temperature, it was poured into saturated aqueous sodium hydrogen carbonate (2 L). The aqueous layer was extracted with ethyl acetate (3×400 mL), the combined organic layers dried over anhydrous magnesium sulfate, filtered and concentrated at reduced pressure. The resulting oil was dissolved in tetrahydrofuran and to this solution was added tetrabutylammonium fluoride (120 mL of a 1.0 M solution in tetrahydrofuran) dropwise. After ten minutes, the reaction mixture was concentrated at reduced pressure and the resulting oil chromatographed on silica gel, eluting with 95:5 to 90:10 dichloromethane:methanol to give the title compound as a mixture of diastereomeric diols (19-8) which was used without further purification.

Step 5: 1-(3,4-Dichlorophenyl)-2-pyridin-4-yl-ethane-1.2-dione (19-9)

To a stirring solution of methyl sulfoxide (28.7 mL, 403 mmol) in dichloromethane (600 mL) at −78° C. was added trifluoroacetic anhydride (42.7 mL, 302 mmol) dropwise. After ten minutes, 1-(4-Chloro-3-methoxyphenyl)-2-pyridin-4-yl-ethane-1,2-diol (19-8) (25.6 g, 91.5 mmol) in dichloromethane (200 mL) was added dropwise. After another ten minutes, triethylamine (79 mL, 567 mmol) was added dropwise and the reaction mixture immediately warmed to −10° C. and poured into water. The aqueous layer was extracted with methylene chloride and the organic layers were combined, dried over anhydrous magnesium sulfate, filtered and concentrated at reduced pressure. The resulting solid was triturated with ether to give the dione (19-9) as a yellow solid.

Step 6: 2-[5-(4-chloro-3-methoxyphenyl)-4-pyridin-4-yl-1H-imidazol-2-yl]-pyrrolidine-1-benzyloxycarbonyl Ester (19-10)

Compound (194) (2.0 g) and the dione (19-9) (2.76 g) were dissolved in 20 mL of acetic acid and the mixture was heated to 100° C. Ammonium acetate (15.48 g) was added slowly and the reaction mixture stirred for 2 hours. The mixture was then poured into ice and the ice slurry was extracted with 2:1 EtOAc:aqueous NH₄OH. The aqueous layer was extracted 4 times with EtOAc and the combined organic layers were washed with brine and dried over Mg₂SO₄. The mixture was filtered and concentrated under vacuum to provide a brown foam. The residue was purified on a silica gel column (3% MeOH in CH₂Cl₂) and the main fractions were repurified under the same silica gel conditions to provide compound (19-10).

Step 7: 1-methyl-2-[5-(4-chloro-3-methoxyphenyl)-4-pyridin-4-yl-1H-imidazol-2-yl]-pyrrolidine (19-11)

Compound (19-10) (580 mg, 1.19 mmol) was dissolved in 10 mL THF and the solution flushed with Ar. A 1.0 M LiAlH₄ solution (1.79 mL, 1.79 mmol) was added and the reaction mixture was heated to 70° C. After stirring the reaction at 70° C. for 2.5 hours an additional 1 equiv. (1.19 mL) of the LiAlH₄ solution was added. The reaction was then quenched with of water and the mixture diluted with EtOAc. The mixture was then poured into a saturated sodium bicarbonate solution and the separated aqueous layer was extracted 3 times with EtOAc. The combined organic layers were washed with brine, dried over Mg₂SO₄, filtered and concentrated under vacuum. The residue was purified by silica gel chromatography (6% to 10% MeOH in CH₂Cl₂ gradient) to provide the titled compound (19-11).

### Example 20

Other compounds shown in Table 3 were synthesized as shown in Schemes 7-8 above. Unless otherwise stated, the TFA salt of the compound shown was isolated by Mass Guided HPLC purification.

TABLE 3

| Compound | MS M + 1 |
|---|---|
| 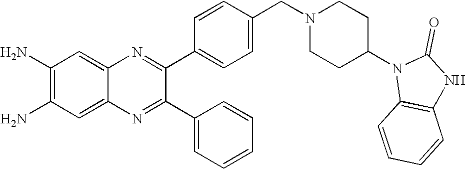 | 541.2590 |
| 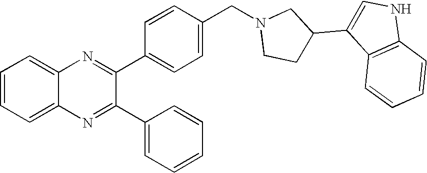 | 480.2314 |
| 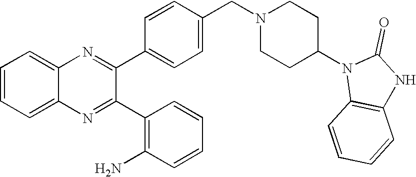 | 526.2481 |
| 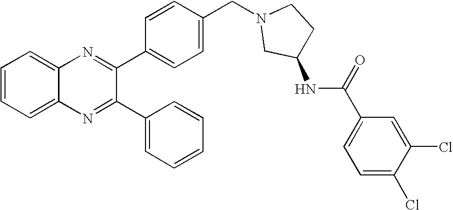 | 552.1483 |
| 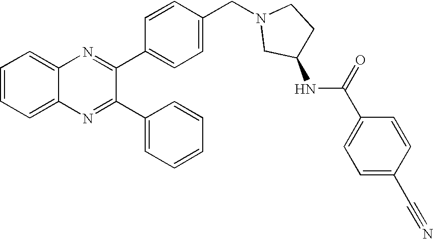 | 509.2215 |
| 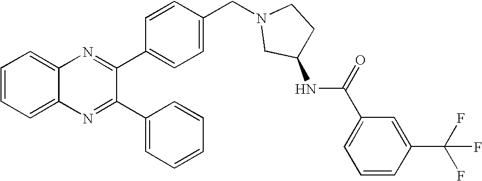 | 552.2185 |
| 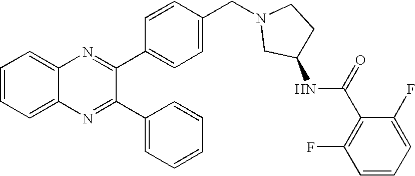 | 520.2106 |
| 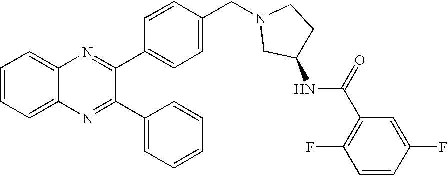 | 520.2106 |
| 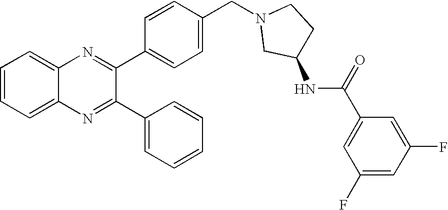 | 520.2106 |
| 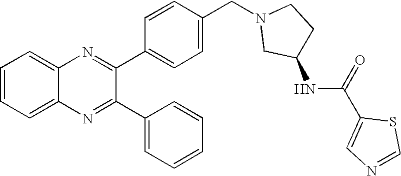 | 491.1779 |
| 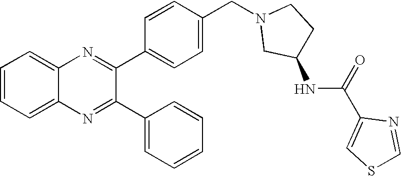 | 491.1779 |
| 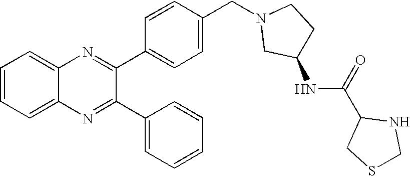 | 495.2092 |
| 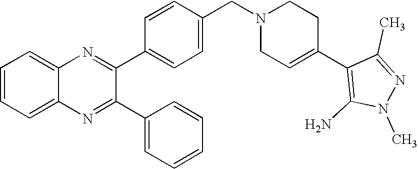 | 486.2532 |
| 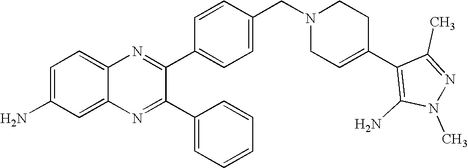 | 501.2641 |
| 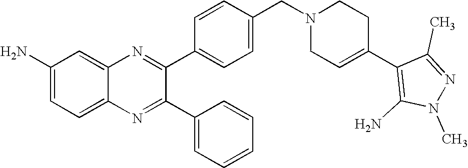 | 501.2641 |
| 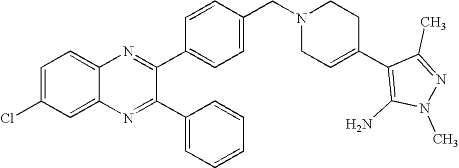 | 521.3 |
| 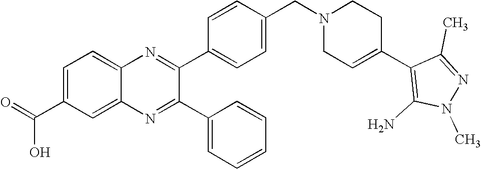 | 530.2430 |
| 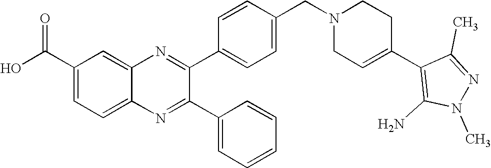 | 530.2430 |
| 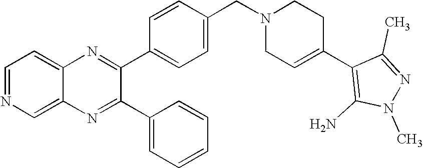 | 487.2484 |
| 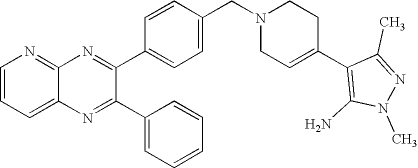 | 487.2484 |
| 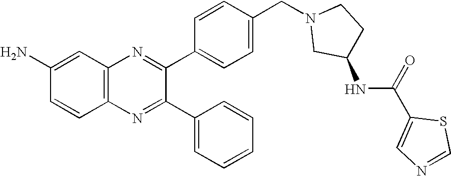 | 506.1888 |
| 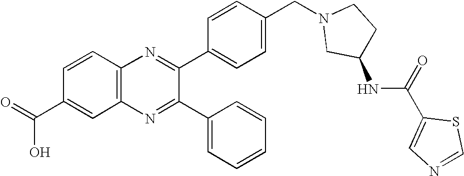 | 535.1678 |
| 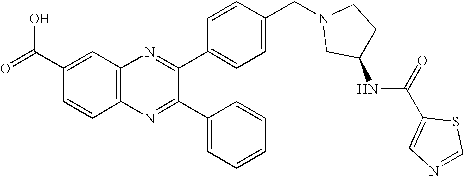 | 535.1678 |
| 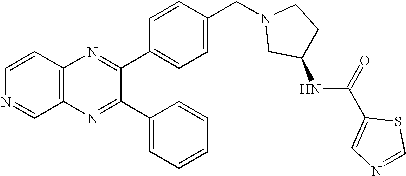 | 492.1732 |
| 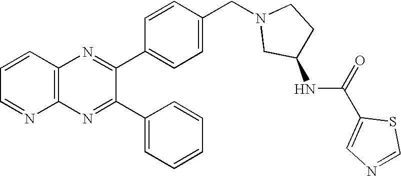 | 492.1732 |
| 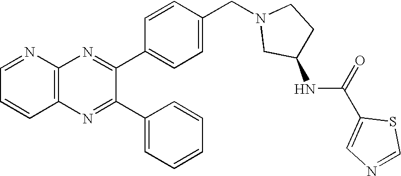 | 492.1732 |
| 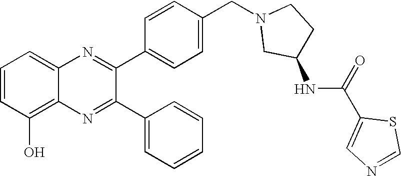 | 507.1729 |
| 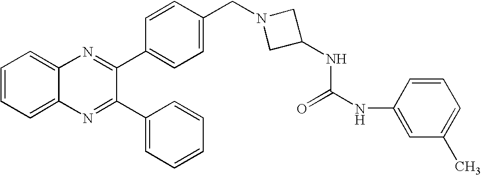 | 499.2372 |
| 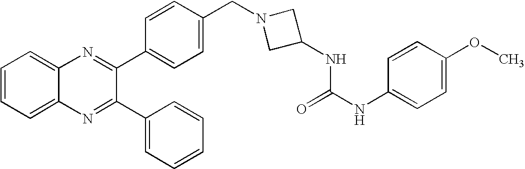 | 515.2321 |
| 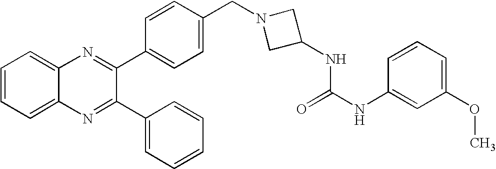 | 515.2321 |
| 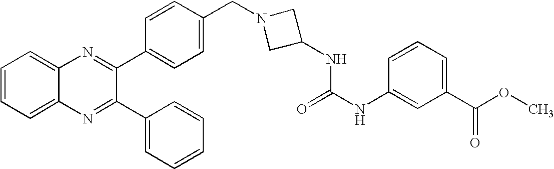 | 543.2270 |
| 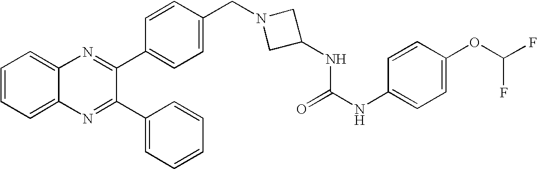 | 551.2164 |
| 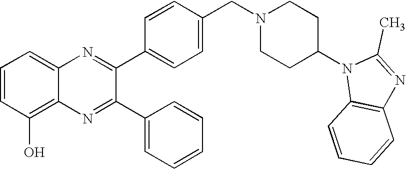 | 525.2528 |
| 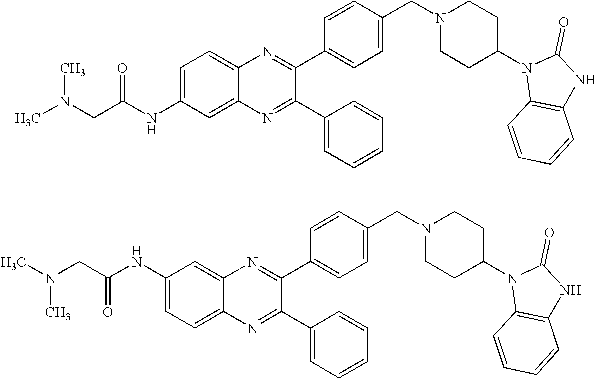 | 611.3008 |
| 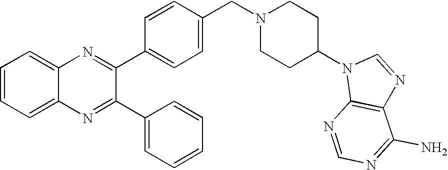 | 512.2436 |
| 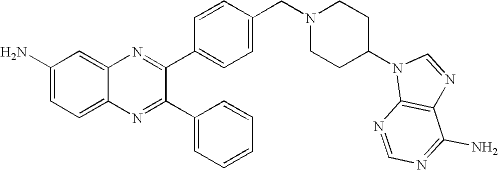 | 527.2545 |
| 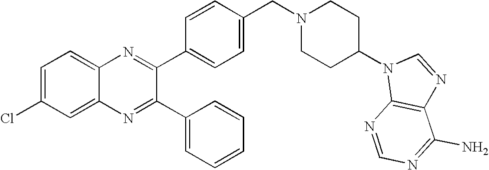 | 547.2 |
| 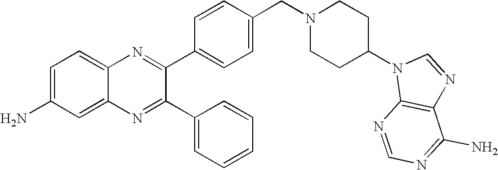 | 527.2545 |
| 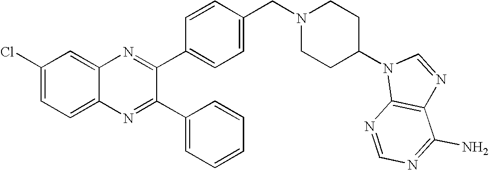 | 547.2 |
| 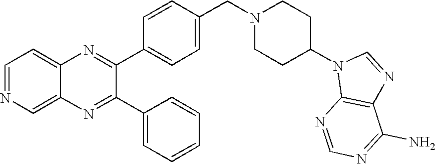 | 513.2389 |
| 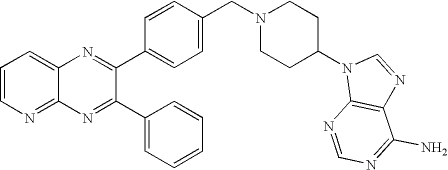 | 513.2389 |
| 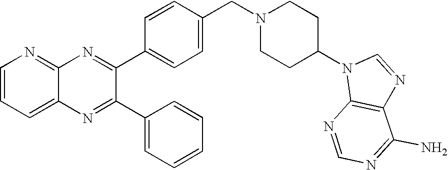 | 513.2389 |
| 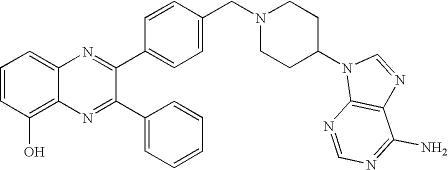 | 528.2386 |
| 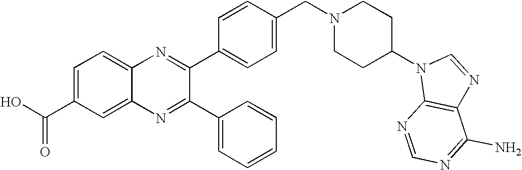 | 556.2335 |
| 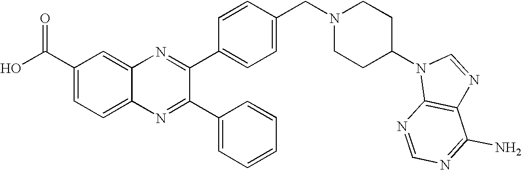 | 556.2335 |
| 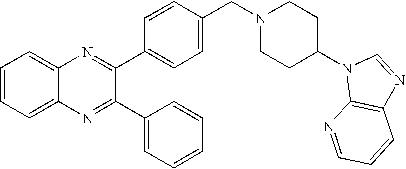 | 496.2375 |
| 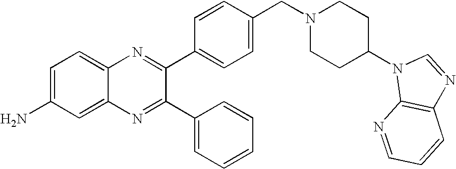 | 511.2484 |
| 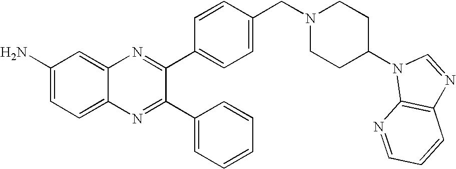 | 511.2484 |
| 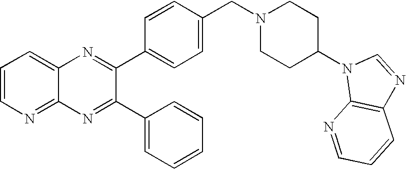 | 497.2328 |
| 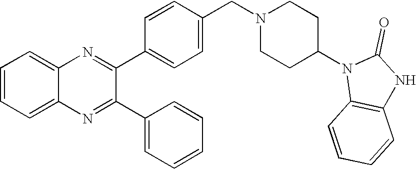 | 510.2419 |
| 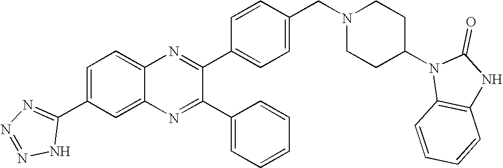 | 579.2495 |
| 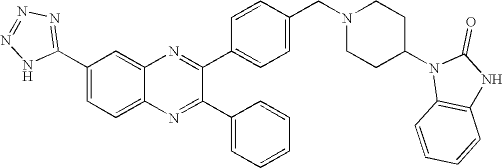 | 579.2495 |
| 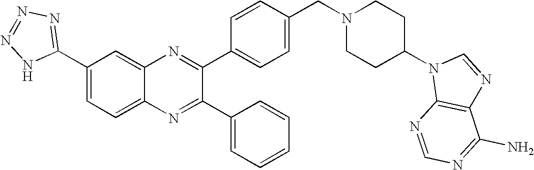 | 580.2559 |
| 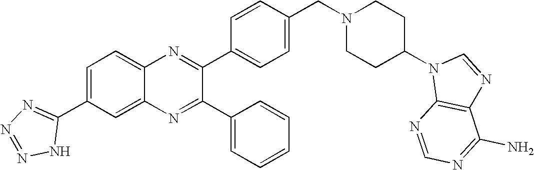 | 581.3 |
| 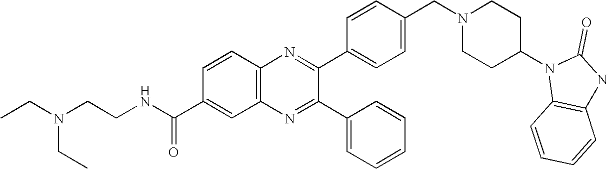 | 611.3008 |
| 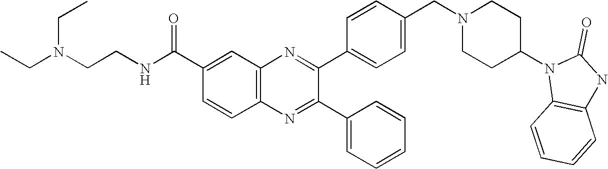 | 611.3008 |
| 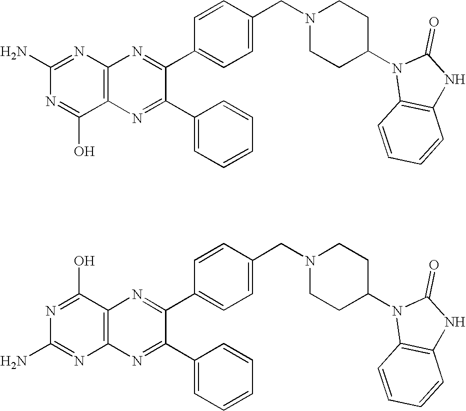 | 544.2335 |
| 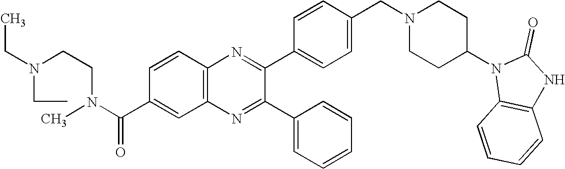 | 653.3478 |
| 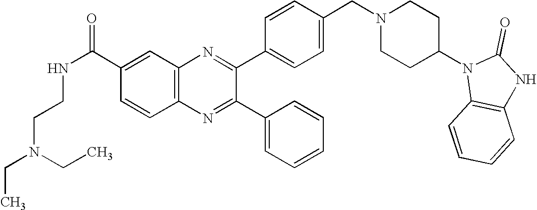 | 653.3478 |
| 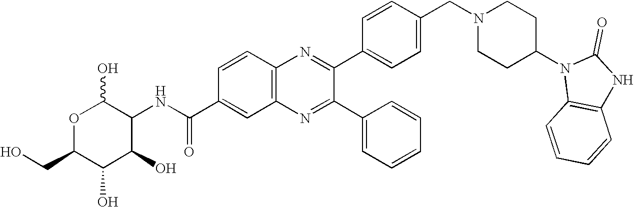 | 716.2958 |
| 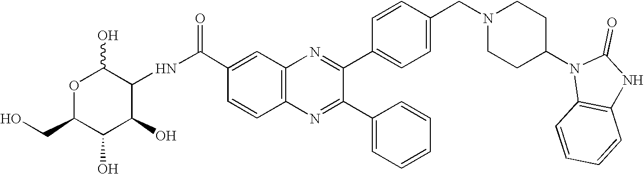 | 716.2958 |
| 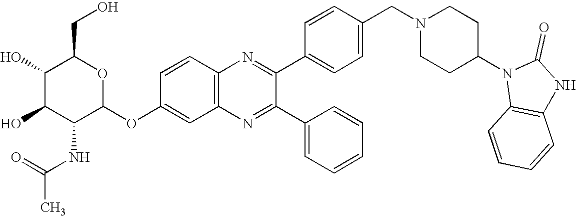 | 730.3115 |
| 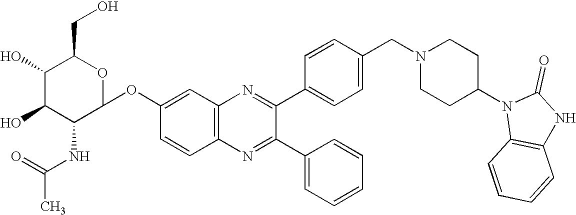 | 730.3115 |

### Example 21

Compounds in Table 4 were synthesized as shown in Schemes 9-10 above. Unless otherwise stated, the TFA salt of the compound shown was isolated by Mass Guided HPLC purification.

TABLE 4

| Compound | MS M + 1 |
|---|---|
| 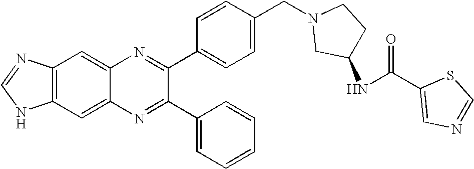 | 531.1841 |
| 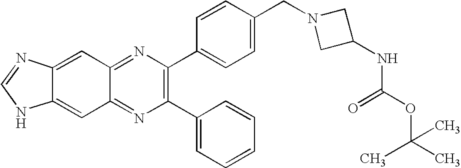 | 506.2430 |
| 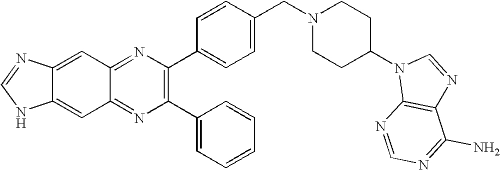 | 552.2498 |
| 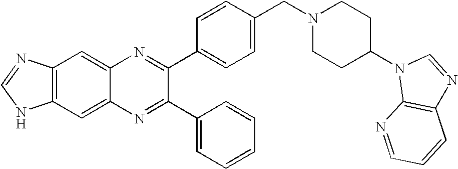 | 536.2436 |

### Example 22

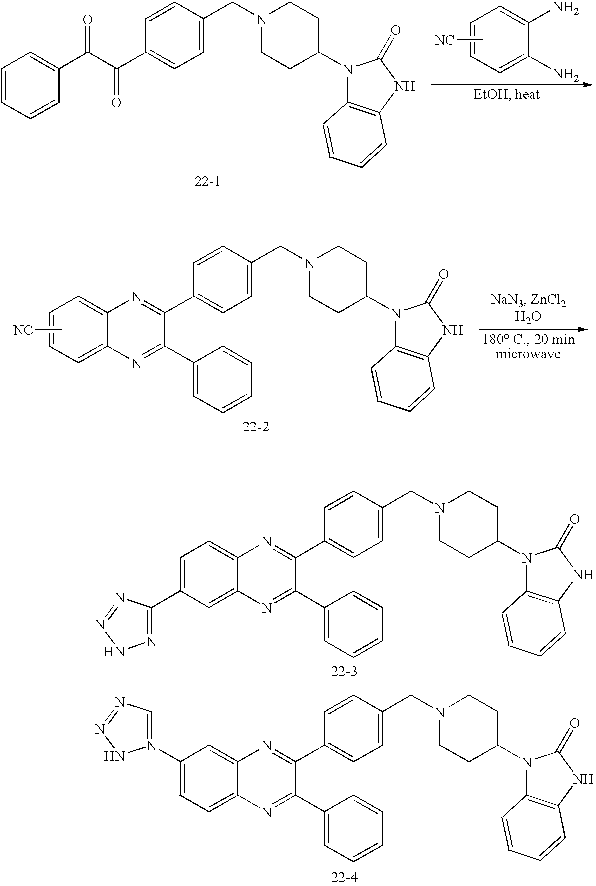

3-(4-{[4-(2-oxo-2,3-dihydro-1H-benzimidazol-1-yl)piperidin-1-yl]methyl}phenyl)-2-phenylquinoxaline-6-carbonitrile (22-2) and 3-(4-{[4-(2-oxo-2,3-dihydro-1H-benzimidazol-1-yl)piperidin-1-yl]methyl}phenyl)-2-phenylquinoxaline-7-carbonitrile (22-2)

1-(4-{[4-(2-oxo-2,3-dihydro-1H-benzimidazol-1-yl)pipepridin-1-yl]methyl}phenyl)-2-phenylethane-1,2-dione (22-1) (176 mg, 0.4 mmol), and 3,4-diaminobenzonitrile (81 mg, 0.6 mmol) were dissolved in 2 mL of MeOH/HOAc (9/1) in an 8 mL vial. The mixture solution is stirred at rt for 3 hours. After this time, the reaction was concentrated in vaccuo. The crude material was then purified on an Agilent 1100 series Mass Guided HPLC purification system to afford 149.1 mg of the TFA salt of the un-separatable mixture of 6- and 7-carbonitriles (22-2) as a brown solid. Analytical LCMS: single peak (214 nm) at 2.634 min (CH₃CN/H₂O/1% TFA, 4 min gradient), M+1 peak m/e 537.3.

1-(1-{4-[3-phenyl-6-(1H-tetrazol-5-yl)quinoxalin-2-yl]benzyl}piperidin-4-yl)-1,3-dihydro-2H-benzimidazol-2-one (22-3)

A mixture of 3-(4-{[4-(2-oxo-2,3-dihydro-1H-benzimidazol-1-yl)piperidin-1-yl]methyl}phenyl)-2-phenylquinoxaline-6-carbonitrile (22-2) and 3-(4-{[4-(2-oxo-2,3-dihydro-1H-benzimidazol-1-yl)piperidin-1-yl]methyl}phenyl)-2-phenylquinoxaline-7-carbonitrile (22-2) (53.6 mg, 0.08 mmol), 2 M NaN₃ (0.5 mL, 1.0 mmol), and 2 M ZnBr₂ (0.5 mL, 1.0 mmol) was charged in a microwave tube and microwaved at 180° C. for 20 min. After this time, the reaction was cooled to rt and the precipitate is collected by centrifuge. The crude material (precipitate) was then purified on an Agilent 1100 series Mass Guided HPLC purification system to afford 27 mg of the TFA salt of the pure 1-(1-{4-[3-phenyl-6-(1H-tetraazol-5-yl)quinoxalin-2-yl]benzyl}piperidin-4-yl)-1,3-dihydro-2H-benzimidazol-2-one (22-3) as a yellow-brown solid.

Analytical LCMS: single peak (214 nm) at 2.320 min (CH₃CN/H₂O/1% TFA, 4 min gradient), M+1 peak m/e 580.3. ¹H NMR (500 MHz, DMSO-d₆): δ 10.93 (s, 1H), δ 9.71 (s, 1H), 8.84 (d, J=2.0 Hz, 1H), 8.53 (dd, J=8.5, 1.9 Hz, 1H), 8.37 (d, J=8.5 Hz, 1H), 7.65(d, J=8.8 Hz, 2H), 7.54-7.58(m, 4H), 7.42-7.46 (m, 3H), 7.28(d, J=7.9 Hz, 1H), 7.01-7.04 (m, 3H), 4.48-4.52 (m, 1H), 4.40 (s, 2H), 3.52 (d, J=12.8 Hz, 2H), 3.22 (t, J=13.8 Hz, 2H), 2.67 (q, J=13.9 Hz, 2H), 1.96 (d, J=13.8 Hz, 2H).

1-(1-{4-[3-phenyl-7-(1H-tetrazol-5-yl)quinoxalin-2-yl]benzyl}piperidin-4-yl)-1,3-dihydro-2H-benzimidazol-2-one (22-4)

A mixture of 3-(4-{[4-(2-oxo-2,3-dihydro-1H-benzimidazol-1-yl)piperidin-1-yl]methyl}phenyl)-2-phenylquinoxaline-6-carbonitrile (22-2) and 3-(4-{[4-(2-oxo-2,3-dihydro-1H-benzimidazol-1-yl)piperidin-1-yl]methyl}phenyl)-2-phenylquinoxaline-7-carbonitrile (22-2) (53.6 mg, 0.08 mmol), 2 M NaN₃ (0.5 mL, 1.0 mmol), and 2 M ZnBr₂ (0.5 mL, 1.0 mmol) was charged in a microwave tube and microwaved at 180° C. for 20 min. After this time, the reaction was cooled to rt and the precipitate is collected by centrifuge. The crude material (precipitate) was then purified on an Agilent 1100 series Mass Guided HPLC purification system to afford 30 mg of the TFA salt of the pure 1-(1-{4-[3-phenyl-7-(1H-tetraazol-5-yl)quinoxalin-2-yl]benzyl}piperidin-4-yl)-1,3-dihydro-2H-benzimidazol-2-one (22-4) as a yellow-brown solid.

Analytical LCMS: single peak (214 nm) at 2.381 min (CH₃CN/H₂O/1% TFA, 4 min gradient), M+1 peak m/e 580.3. ¹H NMR (500 MHz, DMSO-d₆): δ 10.92 (s, 1H), δ 9.81 (s, 1H), 8.82 (d, J=1.9 Hz, 1H), 8.53 (dd, J=8.7, 1.9 Hz, 1H), 8.40 (d, J=8.7 Hz, 1H), 7.66(d, J=7.9 Hz, 2H), 7.58(d, J=8.4 Hz, 2H), 7.53(d, J=7.9 Hz, 2H), 7.45 (t, J=7.4 Hz, 1H), 7.40 (t, J=7.8 Hz, 2H), 7.29(d, J=6.7 Hz, 1H), 7.00-7.03 (m, 3H), 4.49-4.52 (m, 1H), 4.40 (s, 2H), 3.52 (d, J=11.5 Hz, 2H), 3.22 (t, J=13.2 Hz, 2H), 2.68 (q, J=13.1 Hz, 2H), 1.96 (d, J=13.3 Hz, 2H).

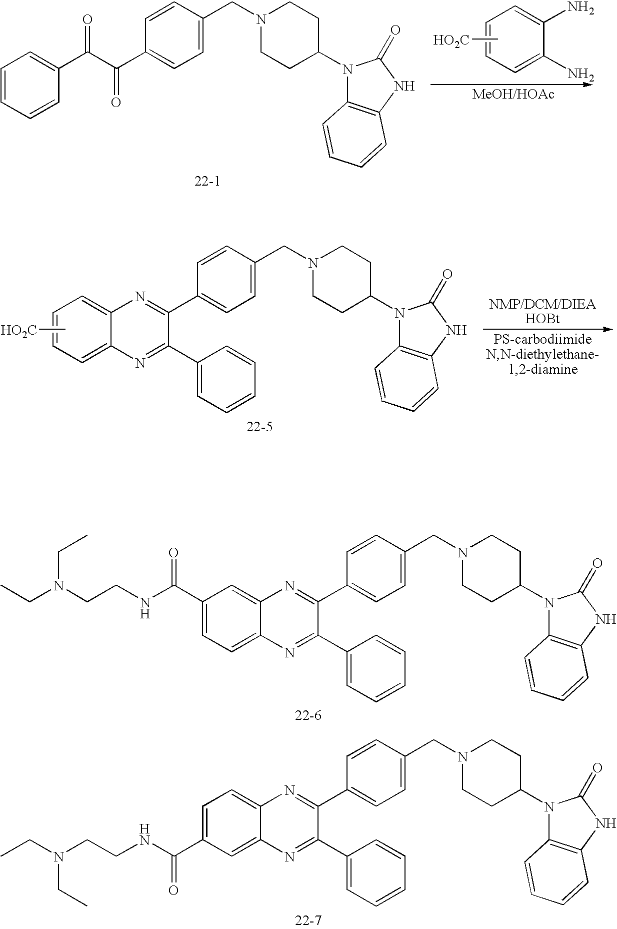

3-(4-{[4-(2-oxo-2,3-dihydro-1H-benzimidazol-1-yl)piperidin-1-yl]methyl}phenyl)-2-phenylquinoxaline-6-carboxylic Acid (22-5) and 2-(4-{[4-(2-oxo-2,3-dihydro-1H-benzimidazol-1-yl)piperidin-1-yl]methyl}phenyl)-3-phenylquinoxaline-6-carboxylic Acid (22-5)

To 1-(4-{[4-(2-oxo-2,3-dihydro-1H-benzimidazol-1-yl)pipepridin-1-yl]methyl}phenyl)-2-phenylethane-1,2-dione (22-1) (4.39 g, 10 mmol, in 40 mL of MeOH/HOAc (9/1)) was added 4-diaminobenzoic acid (1.55 g, 10.2 mmol, in 20 mL of DMSO/MeOH (3/1)) dropwise with stirring. After addition of 4-diaminobenzoic acid, the reaction was stirred for 2 h at room temperature. The solution precipitated and LCMS indicated that the reaction was completed. The reaction mixture was poured into water (150 mL). The precipitate was collected by centrifuge and washed with water (3×). LCMS indicated that the precipitate was the desired product of the two regioisomers (22-5). Analytical LCMS: double peaks (214 nm) at 2.317 and 2.388 min (CH₃CN/H₂O/1% TFA, 4 min gradient), M+1 peak at 2.353 min (m/e 556.3) and 2.428 min (m/e 556.3). The product was frozen dry (5.2 g) and used in the next step without further purification.

N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide (22-6) and N-[2-(diethylamino)ethyl]-2-(4-{[4-(2-oxo-2,3-dihydro-1H-benzimidazol-1-yl)piperidin-1-yl]methyl}phenyl)-3-phenylquinoxaline-6-carboxamide (22-7)

The mixture of 3-(4-{[4-(2-oxo-2,3-dihydro-1H-benzimidazol-1-yl)piperidin-1-yl]methyl}phenyl)-2-phenylquinoxaline-6-carboxylic acid (22-5) and 2-(4-{[4-(2-oxo-2,3-dihydro-1H-benzimidazol-1-yl)piperidin-1-yl]methyl}phenyl)-3-phenylquinoxaline-6-carboxylic acid (22-5), 222 mg (0.4 mmol) was dissolved in NMP/DCM/DIEA (10 mL, 9/1). To this solution was added HOBt (152 mg, 1.0 mmol), PS-carbodiimide (1.1 g, 1.3 mmol), and DCM (5 mL). The resultant mixture was shaken 0.5 h. After this time, N,N-diethylethane-1,2-diamine (232 mg, 2.0 mmol) was added to the NMP solution and the reaction was shaken over weekend. After this time, LCMS indicated that the coupling reaction was complete. The resin was filtered and washed with MeOH (3×15 mL). The combined solution was dried to give a brown residue. This residue was then purified on an Agilent 1100 series Mass Guided HPLC purification system to afford the two pure regioisomers N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide (22-6) (50.7 mg) and N-[2-(diethylamino)ethyl]-2-(4-{[4-(2-oxo-2,3-dihydro-1H-benzimidazol-1-yl)piperidin-1-yl]methyl}phenyl)-3-phenylquinoxaline-6-carboxamide (22-7) (119 mg). Analytical data for N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide (22-6): Analytical LCMS: single peak (214 nm) at 2.084 min (CH₃CN/H₂O/1% TFA, 4 min gradient). ¹H NMR (500 MHz, DMSO-d₆): δ 10.96 (s, 1H), 10.10 (s, 1H), 9.50 (s, 1H), 9.20 (t, J=5.9 Hz, 1H), 8.69 (s, 1H), 8.26-8.34 (m, 2H), 7.49-7.66 (m, 6H), 7.35-7.47 (m, 3H), 7.26-7.32 (m, 1H), 6.97-7.04 (m, 3H), 4.48-4.58 (m, 1H), 4.40 (s, 2H), 3.72 (q, J=6.1 Hz, 2H), 3.50 (d, J=11.7 Hz, 2H), 3.34(q, J=5.4 Hz, 2H), 3.20-3.30 (m, 6H), 2.67 (q, J=14.5 Hz, 2H), 1.96 (d, J=13.0 Hz, 2H), 1.25 (t, J=7.5 Hz, 6H). HRMS, calc'd for C₄₀H₄₄N₇O₂ (M+H), 654.3551; found 654.3573.

### Example 23

### Assays

Selective Akt inhibitor compounds useful in the methods of treatment of the instant invention may be tested by the assays described below to determine Akt inhibitory activity. Specific compounds of the instant invention were tested in the assay described herein and were found to have IC₅₀ of ≦20 μM against one or more of Akt1, Akt2 and Akt3.

Cloning of Human Akt1, Akt2, Akt3, ΔPH-Akt1, ΔPH-Akt2, ΔPH-Akt3 and Minimal ΔPH Akt1

The pS2neo vector (deposited in the ATCC on Apr. 3, 2001 as PTA-3253) was prepared as follows: The pRmHA3 vector (prepared as described in Nucl. Acid Res. 16:1043-1061 (1988)) was cut with BglII and a 2734 bp fragment was isolated. The pUChsneo vector (prepared as described in EMBO J. 4:167-171 (1985)) was also cut with BglII and a 4029 bp band was isolated. These two isolated fragments were ligated together to generate a vector termed pS2neo-1. This plasmid contains a polylinker between a metallothionein promoter and an alcohol dehydrogenase poly A addition site. It also has a neomycin resistance gene driven by a heat shock promoter. The pS2neo-1 vector was cut with Psp5II and BsiWI. Two complementary oligonucleotides were synthesized and then annealed (CTGCGGCCGC (SEQ.ID.NO.: 1) and GTACGCGGCCGCAG (SEQ.ID.NO.: 2)). The cut pS2neo-1 and the annealed oligonucleotides were ligated together to generate a second vector, pS2neo. Added in this conversion was a NotI site to aid in the linearization prior to transfection into S2 cells.

Human Akt1 gene was amplified by PCR (Clontech) out of a human spleen cDNA (Clontech) using the 5′ primer: 5′CGCGAATTCAGATCTACCATGAGCGACGTGGCTATTGTG 3′ (SEQ.ID.NO.: 3), and the 3′ primer: 5′CGCTCTAGAGGATCCTCAGGCCGTGCTGCTGGC3′ (SEQ.ID.NO.: 4). The 5′ primer included an EcoRI and BglII site. The 3′ primer included an XbaI and BamHI site for cloning purposes. The resultant PCR product was subcloned into pGEM3Z (Promega) as an EcoRI/Xba I fragment. For expression/purification purposes, a middle T tag was added to the 5′ end of the full length Akt1 gene using the PCR primer: 5′GTACGATGCTGAACGATATCTTCG 3′ (SEQ.ID.NO.: 5). The resulting PCR product encompassed a 5′ KpnI site and a 3′ BamHI site which were used to subclone the fragment in frame with a biotin tag containing insect cell expression vector, pS2neo.

For the expression of a pleckstrin homology domain (PH) deleted (Δaa 4-129, which includes deletion of a portion of the Akt1 hinge region) version of Akt1 (termed ΔPH-Akt1), PCR deletion mutagenesis was done using the full length Akt1 gene in the pS2neo vector as template. The PCR was carried out in 2 steps using overlapping internal primers (5′GAATACATGCCGATGGAAAGCGACGGGGCTGAAGAGATGGAGGTG 3′ (SEQ.ID.NO.: 6), and 5′CCCCTCCATCTCTTCAGCCCCGTCGCTTTCCATCGGCATG TATTC 3′ (SEQ.ID.NO.: 7)) which encompassed the deletion and 5′ and 3′ flanking primers which encompassed the KpnI site and middle T tag on the 5′ end. The final PCR product was digested with KpnI and SmaI and ligated into the pS2neo full length Akt1 KpnI/Sma I cut vector, effectively replacing the 5′ end of the clone with the deleted version.

For expression of a minimal ΔPH (Δaa 1-110) version of Akt1, PCR was performed using full length Akt1 as template and the following PCR oligo primers; 5′ PCR oligo=5′CGCGGCGCGCCAGGTACCATGGAATACATGCCGATGGAAAAGAAGCAGGAGGAG GAGGAG 3′ (SEQ.ID.NO.: 8) which encompassed a KpnI cloning site, the middle T antigen tag and the PH domain deletion. The 3′ PCR oligo=5′CGGAGAACACACGCTCCCGGG 3′ (SEQ.ID.NO.: 9). The resultant PCR product was digested with KpnI and SmaI and ligated into the pPS2neo full length Akt1 KpnI/SmaI cut vector, effectively replacing the 5′ end of the clone with the deleted version.

Human Akt3 gene was amplified by PCR of adult brain cDNA (Clontech) using the amino terminal oligo primer: 5′ GAATTCAGATCTACCATGAGCGATGTTACCATTGTG 3′ (SEQ.ID.NO.: 10); and the carboxy terminal oligo primer: 5′ TCTAGATCTTATTCTCGTCCACTTGCAGAG 3′(SEQ.ID.NO.: 11).

These primers included a 5′ EcoRI/BglII site and a 3′ XbaI/BglII site for cloning purposes. The resultant PCR product was cloned into the EcoRI and XbaI sites of pGEM4Z (Promega). For expression/purification purposes, a middle T tag was added to the 5′ end of the full length Akt3 clone using the PCR primer: 5′ GGTACCATGGAATACATGCCGATGGAAAGCGATGTTACCATTGTGAAG 3′(SEQ.ID.NO.: 12). The resultant PCR product encompassed a 5′ KpnI site which allowed in frame cloning with the biotin tag containing insect cell expression vector, pS2neo.

For expression of a PH domain deleted (Δaa 4-128, which includes deletion of a portion of the Akt3 hinge region) version of Akt 3 (termed ΔPH-Akt 3), PCR was performed using the full length Akt 3 as template and the following oligo primers; 5′PCR oligo=5′CGCAGGTACCATGGAATACATGCCGATGGAAAGCGATGGAGAGGAAGAGATGGA TGCC 3′ (SEQ.ID.NO.: 13) which encompassed a KpnI cloning site, the middle T antigen tag and the deleted PH domain. The 3′ PCR oligo=5′CGCTCTAGATCTTATTCTCGTCCACTTGCAGAG 3′ (SEQ.ID.NO.: 14). The resultant PCR product was digested with KpnI and BamHI and ligated into the pS2neo full length Akt 3 KpnI/BamHI cut vector, effectively replacing the 5′ end of the clone with the deleted version.

Human Akt2 gene was amplified by PCR from human thymus cDNA (Clontech) using the amino terminal oligo primer: 5′ AAGCTTAGATCTACCATGAATGAGGTGTCTGTC 3′ (SEQ.ID.NO.: 15); and the carboxy terminal oligo primer: 5′GAATTCGGATCCTCACTCGCGGATGCTGGC 3′ (SEQ.ID.NO.:

16). These primers included a 5′ HindIII/BglII site and a 3′ EcoRI/BamHI site for cloning purposes. The resultant PCR product was subcloned into the HindIII/EcoRI sites of pGem3Z (Promega). For expression/purification purposes, a middle T tag was added to the 5′ end of the full length Akt2 using the PCR primer: 5′GGTACCATGGAATACATGCCGATGGAAAATGAGGTGTCTGTCATCAAAG 3′ (SEQ.ID.NO.: 17). The resultant PCR product was subcloned into the pS2neo vector as described above.

For expression of a PH domain deleted (Δaa 4-131, which includes deletion of a portion of the Akt2 hinge region) version of Akt 2 (termed ΔPH-Akt2), PCR was performed using the full length Akt 2 gene as template and the following oligo primers; 5′ PCR oligo=5′CGCAGGTACCATGGAATACATGCCGATGGAAAATGAGACGACTGAGGAGATGGA AGTGGC 3′ (SEQ.ID.NO.: 18), which encompassed a KpnI cloning site, the middle T antigen tag and the deletion. The 3′ PCR oligo=5′CGCGAATTCGGATCCTCACTCGCGGATGCTGGC 3′ (SEQ.ID.NO.: 19). The resultant PCR product was digested with KpnI and SmaI and ligated into the pS2neo full length Akt 2 KpnI/SmaI cut vector, effectively replacing the 5′ end of the clone with the deleted version.

Expression of Human Akt1, Akt2, Akt3, ΔPH-Akt1, ΔPH-Akt2, ΔPH-Akt3 and Minimal ΔPH Akt1

The DNA containing the cloned Akt1, Akt2, Akt3, ΔPH-Akt1, ΔPH-Akt2, ΔPH-Akt3 and ΔPH domain specific-Akt1 genes in the pS2neo expression vector was purified and used to transfect Drosophila S2 cells (ATCC) by the calcium phosphate method. Pools of antibiotic (G418, 500 μg/ml) resistant cells were selected. Cell were expanded to a 1.0 L volume (˜7.0×10⁶/ml), biotin and CuSO₄ were added to a final concentration of 50 μM and 50 mM respectively. Cells were grown for 72 h at 27° C. and harvested by centrifugation. The cell paste was frozen at −70° C. until needed.

Purification of Human Akt1, Akt2, Akt3, ΔPH-Akt1, ΔPH-Akt2, ΔPH-Akt3 and Minimal ΔPH Akt1

Cell paste from one liter of S2 cells, described in above, was lysed by sonication with 50 mls 1% CHAPS in buffer A: (50 mM Tris pH 7.4, 1 mM EDTA, 1 mM EGTA, 0.2 mM AEBSF, 10 μg/ml benzamidine, 5 μg/ml of leupeptin, aprotinin and pepstatin each, 10% glycerol and 1 mM DTT). The soluble fraction was purified on a Protein G Sepharose fast flow (Pharmacia) column loaded with 9 mg/ml anti-middle T monoclonal antibody and eluted with 75 μM EYMPME (SEQ.ID.NO.: 20) peptide in buffer A containing 25% glycerol. Akt/PKB containing fractions were pooled and the protein purity evaluated by SDS-PAGE. The purified protein was quantitated using a standard Bradford protocol. Purified protein was flash frozen on liquid nitrogen and stored at −70° C.

Akt and Akt pleckstrin homology domain deletions purified from S2 cells required activation. Akt and Akt pleckstrin homology domain deletions were activated (Alessi et al. Current Biology 7:261-269) in a reaction containing 10 nM PDK1 (Upstate Biotechnology, Inc.), lipid vesicles (10 μM phosphatidylinositol-3,4,5-trisphosphate—Metreya, Inc, 100 μM phosphatidylcholine and 100 μM phosphatidylserine—Avanti Polar lipids, Inc.) and activation buffer (50 mM Tris pH7.4, 1.0 mM DTT, 0.1 mM EGTA, 1.0 μM Microcystin-LR, 0.1 mM ATP, 10 mM MgCl₂, 333 μg/ml BSA and 0.1 mM EDTA). The reaction was incubated at 22° C. for 4 hours. Aliquots were flash frozen in liquid nitrogen.

Akt Kinase Assays

Activated AKT isoforms and pleckstrin homology domain deletion constructs were assayed utilizing a GSK-derived biotinylated peptide substrate. The extent of peptide phosphorylation was determined by Homogeneous Time Resolved Fluorescence (HTRF) using a lanthamide chelate (Lance)-coupled monoclonal antibody specific for the phosphopeptide in combination with a streptavidin-linked allophycocyanin (SA-APC) fluorophore which will bind to the biotin moiety on the peptide. When the Lance and APC are in proximity (i.e. bound to the same phosphopeptide molecule), a non-radiative energy transfer takes place from the Lance to the APC, followed by emission of light from APC at 665 nm.

Materials required for the assay:

A. Activated AKT isozyme or pleckstrin homology domain deleted construct

B. AKT peptide substrate: GSK3a (S21) Peptide #3928 biotin-GGRARTSSFAEPG (SEQ.ID.NO.:21), Macromolecular Resources.

C. Lance labeled anti-phospho GSK3α monoclonal antibody (Cell Signaling Technology, clone # 27).

D. SA-APC (Prozyme catalog no. PJ25S lot # 896067).

E. Microfluor®B U Bottom Microtiter Plates (Dynex Technologies, Catalog no. 7205).

F. Discovery®HTRF Microplate Analyzer, Packard Instrument Company.

G. 100× Protease Inhibitor Cocktail (PIC): 1 mg/ml benzamidine, 0.5 mg/ml pepstatin, 0.5 mg/ml leupeptin, 0.5 mg/ml aprotinin.

H. 10× Assay Buffer: 500 mM HEPES, pH 7.5, 1% PEG, mM EDTA, 1 mM EGTA, 1% BSA, 20 mM [symbol] -Glycerol phosphate.

I. Quench Buffer: 50 mM HEPES pH 7.3, 16.6 mM EDTA, 0.1% BSA, 0.1% Triton X-100, 0.17 nM Lance labeled monoclonal antibody clone # 27, 0.0067 mg/ml SA-APC

J. ATP/MgCl₂ working solution: 1× Assay buffer, 1 mM DTT, 1×PIC, 125 mM KCl, 5% Glycerol, 25 mM MgCl₂, 375 TM ATP

K. Enzyme working solution: 1× Assay buffer, 1 mM DTT, 1×PIC, 5% Glycerol, active Akt. The final enzyme concentrations were selected so that the assay was in a linear response range.

L. Peptide working solution: 1× Assay buffer, 1 mM DTT, 1×PIC, 5% Glycerol, 2 TM GSK3 biotinylated peptide # 3928

The reaction is assembled by adding 16 TL of the ATP/MgCl₂ working solution to the appropriate wells of a 96-well microtiter plate. Inhibitor or vehicle (1.0 Tl) is added followed by 10 Tl of peptide working solution. The reaction is started by adding 13 Tl of the enzyme working solution and mixing. The reaction is allowed to proceed for 50 min and then stopped by the addition of 60 Tl HTRF quench buffer. The stopped reactions were incubated at room temperature for at least 30 min and then read on the Discovery instrument.

Procedure for Streptavidin Flash Plate Assay:

Step 1:

A 1 μl solution of the test compound in 100% DMSO was added to 20 μl of 2× substrate solution (20 uM GSK3 Peptide, 300 μM ATP, 20 mM MgCl₂, 20 μCi/ml [γ³³P] ATP, 1× Assay Buffer, 5% glycerol, 1 mM DTT, 1×PIC, 0.1% BSA and 100 mM KCl). Phosphorylation reactions were initiated by adding 19 μl of 2× Enzyme solution (6.4 nM active Akt/PKB, 1× Assay Buffer, 5% glycerol, 1 mM DTT, 1×PIC and 0.1% BSA). The reactions were then incubated at room temperature for 45 minutes.

Step 2:

The reaction was stopped by adding 170 μl of 125 mM EDTA. 200 μl of stopped reaction was transferred to a Streptavidin Flashplate® PLUS (NEN Life Sciences, catalog no. SMP103). The plate was incubated for >10 minutes at room temperature on a plate shaker. The contents of each well was aspirated, and the wells rinsed 2 times with 200 μl TBS per well. The wells were then washed 3 times for 5 minutes with 200 μl TBS per well with the plates incubated at room temperature on a platform shaker during wash steps.

The plates were covered with sealing tape and counted using the Packard TopCount with the appropriate settings for counting [³³P] in Flashplates.

Procedure for Streptavidin Filter Plate Assay:

Step 1:

The enzymatic reactions as described in Step 1 of the Streptavidin Flash Plate Assay above were performed.

Step 2:

The reaction was stopped by adding 20 μl of 7.5M Guanidine Hydrochloride. 50 μl of the stopped reaction was transferred to the Streptavidin filter plate (SAM²™ Biotin Capture Plate, Promega, catalog no. V7542) and the reaction was incubated on the filter for 1-2 minutes before applying vacuum.

The plate was then washed using a vacuum manifold as follows: 1) 4×200 μl/well of 2M NaCl; 2) 6×200 μl/well of 2M NaCl with 1% H₃PO₄; 3) 2×200 μl/well of diH₂O; and 4) 2×100 μl/well of 95% Ethanol. The membranes were then allowed to air dry completely before adding scintillant.

The bottom of the plate was sealed with white backing tape, 30 μl/well of Microscint 20 (Packard Instruments, catalog no. 6013621) was added. The top of the plate was sealed with clear sealing tape, and the plate then counted using the Packard TopCount with the appropriate settings for [³³P] with liquid scintillant.

Procedure for Phosphocellulose Filter Plate Assay:

Step 1:

The enzymatic reactions were performed as described in Step 1 of the Streptavidin Flash Plate Assay (above) utilizing KKGGRARTSSFAEPG (SEQ.ID.NO.: 22) as the substrate in place of biotin-GGRARTSSFAEPG.

Step 2:

The reaction was stopped by adding 20 μl of 0.75% H₃PO₄. 50 μl of stopped reaction was transferred to the filter plate (UNIFILTER™, Whatman P81 Strong Cation Exchanger, White Polystyrene 96 Well Plates, Polyfiltronics, catalog no. 7700-3312) and the reaction incubated on the filter for 1-2 minutes before applying vacuum.

The plate was then washed using a vacuum manifold as follows: 1) 9×200 μl/well of 0.75% H₃PO₄; and 2) 2×200 μl/well of diH₂O. The bottom of the plate was sealed with white backing tape, then 30 μl/well of Microscint 20 was added. The top of the plate was sealed with clear sealing tape, and the plate counted using the Packard TopCount with the appropriate settings for [³³P] and liquid scintillant.

PKA Assay:

Each individual PKA assay consists of the following components:

A. 5×PKA assay buffer (200 mM Tris pH7.5, 100 mM MgCl₂, 5 mM [symbol] -mercaptoethanol, 0.5 mM EDTA)

B. 50 μM stock of Kemptide (Sigma) diluted in water

C. ³³P-ATP prepared by diluting 1.0 μl, ³³P-ATP [10 mCi/ml] into 200 Tl of a 50 μM stock of unlabeled ATP

D. 10 μl of a 70 nM stock of PKA catalytic subunit (UBI catalog # 14-114) diluted in 0.5 mg/ml BSA

E. PKA/Kemptide working solution: equal volumes of 5×PKA assay buffer, Kemptide solution and PKA catalytic subunit.

The reaction is assembled in a 96 deep-well assay plate. The inhibitor or vehicle (10 Tl) is added to 10 Tl of the ³³P-ATP solution. The reaction is initiated by adding 30 Tl of the PKA/Kemptide working solution to each well. The reactions were mixed and incubated at room temperature for 20 min. The reactions were stopped by adding 50 Tl of 100 mM EDTA and 100 mM sodium pyrophosphate and mixing.

The enzyme reaction product (phosphorylated Kemptide) was collected on p81 phosphocellulose 96 well filter plates (Millipore). To prepare the plate, each well of a p81 filter plate was filled with 75 mM phosphoric acid. The wells were emptied through the filter by applying a vacuum to the bottom of the plate. Phosphoric acid (75 mM, 170 μl) was added to each well. A 30 μl aliquot from each stopped PKA reaction was added to corresponding wells on the filter plate containing the phosphoric acid. The peptide was trapped on the filter following the application of a vacuum and the filters were washed 5 times with 75 mM phosphoric acid. After the final wash, the filters were allowed to air dry. Scintillation fluid (30 μl) was added to each well and the filters counted on a TopCount (Packard).

PKC Assay:

Each PKC assay consists of the following components:

A. 10×PKC co-activation buffer: 2.5 mM EGTA, 4 mM CaCl₂

B. 5×PKC activation buffer: 1.6 mg/ml phosphatidylserine, 0.16 mg/ml diacylglycerol, 100 mM Tris pH 7.5, 50 mM MgCl₂, 5 mM [symbol] -mercaptoethanol

C. ³³P-ATP prepared by diluting 1.0 μl ³³P-ATP [10 mCi/ml] into 100 μl of a 100 μM stock of unlabeled ATP

D. Myelin basic protein (350 μg/ml, UBI) diluted in water

E. PKC (50 ng/ml, UBI catalog # 14-115) diluted into 0.5 mg/ml BSA

F. PKC/Myelin Basic Protein working solution: Prepared by mixing 5 volumes each of PKC co-activation buffer and Myelin Basic protein with 10 volumes each of PKC activation buffer and PKC.

The assays were assembled in 96 deep-well assay plates. Inhibitor or vehicle (10 Tl) was added to 5.0 μl of ³³P-ATP. Reactions were initiated with the addition of the PKC/Myelin Basic Protein working solution and mixing. Reactions were incubated at 30° C. for 20 min. The reactions were stopped by adding 50 Tl of 100 mM EDTA and 100 mM sodium pyrophosphate and mixing. Phosphorylated Mylein Basic Protein was collected on PVDF membranes in 96 well filter plates and quantitated by scintillation counting.

### Example 24

Cell Based Assays to Determine Inhibition of Akt/PKB

Cells (for example LnCaP or a PTEN^{(-/-)}tumor cell line with activated Akt/PKB) were plated in 100 mm dishes. When the cells were approximately 70 to 80% confluent, the cells were refed with 5 mls of fresh media and the test compound added in solution. Controls included untreated cells, vehicle treated cells and cells treated with either LY294002 (Sigma) or wortmannin (Sigma) at 20 μM or 200 nM, respectively. The cells were incubated for 2, 4 or 6 hrs, and the media removed. The cells were washed with PBS, scraped and transferred to a centrifuge tube. They were pelleted and washed again with PBS. Finally, the cell pellet was resuspended in lysis buffer (20 mM Tris pH8, 140 mM NaCl, 2 mM EDTA, 1% Triton, 1 mM Na Pyrophosphate, 10 mM β-Glycerol Phosphate, 10 mM NaF, 0.5 mN Na₃VO₄, 1 μM Microcystine, and 1× Protease Inhibitor Cocktail), placed on ice for 15 minutes and gently vortexed to lyse the cells. The lysate was spun in a Beckman tabletop ultra centrifuge at 100,000×g at 4° C. for 20 min. The supernatant protein was quantitated by a standard Bradford protocol (BioRad) and stored at −70° C. until needed.

Proteins were immunoprecipitated (IP) from cleared lysates as follows: For Akt1/PKBI, lysates are mixed with Santa Cruz sc-7126 (D-17) in NETN (100 mM NaCl, 20 mM Tris pH 8.0, 1 mM EDTA, 0.5% NP-40) and Protein A/G Agarose (Santa Cruz sc-2003) was added. For Akt2/PKBβ, lysates were mixed in NETN with anti-Akt 2 agarose (Upstate Biotechnology #16-174) and for Akt3/PKBγ, lysates were mixed in NETN with anti-Akt 3 agarose (Upstate Biotechnology #16-175). The IPs were incubated overnight at 4° C., washed and separated by SDS-PAGE.

Western blots were used to analyze total Akt, pThr308 Akt1, pSer473 Akt1, and corresponding phosphorylation sites on Akt2 and Akt3, and downstream targets of Akt using specific antibodies (Cell Signaling Technology): Anti-Total Akt (cat. no. 9272), Anti-Phospho Akt Serine 473 (cat. no. 9271), and Anti-Phospho Akt Threonine 308 (cat. no. 9275). After incubating with the appropriate primary antibody diluted in PBS+0.5% non-fat dry milk (NFDM) at 4° C. overnight, blots were washed, incubated with Horseradish peroxidase (HRP)-tagged secondary antibody in PBS+0.5% NFDM for 1 hour at room temperature. Proteins were detected with ECL Reagents (Amersham/Pharmacia Biotech RPN2134).

Heregulin Stimulated Akt Activation

MCF7 cells (a human breast cancer line that is PTEN^{+/+}) were plated at 1×10⁶ cells per 100 mm plate. When the cells were 70-80% confluent, they were refed with 5 ml of serum free media and incubated overnight. The following morning, compound was added and the cells were incubated for 1-2 hrs, after which time heregulin was added (to induce the activation of Akt) for 30 minutes and the cells were analyzed as described above.

### Example 25

Inhibition of Tumor Growth

In vivo efficacy as an inhibitor of the growth of cancer cells may be confirmed by several protocols well known in the art.

Human tumor cells from cell lines which exhibit a deregulation of the PI3K pathway (such as LnCaP, PC3, C33a, OVCAR-3, MDA-MB-468 or the like) are injected subcutaneously into the left flank of 6-10 week old female nude mice (Harlan) on day 0. The mice are randomly assigned to a vehicle, compound or combination treatment group. Daily subcutaneous administration begins on day 1 and continues for the duration of the experiment. Alternatively, the inhibitor test compound may be administered by a continuous infusion pump. Compound, compound combination or vehicle is delivered in a total volume of 0.2 ml. Tumors are excised and weighed when all of the vehicle-treated animals exhibited lesions of 0.5-1.0 cm in diameter, typically 4 to 5.5 weeks after the cells were injected. The average weight of the tumors in each treatment group for each cell line is calculated.

### Example 26

5-Chloro-3-(morpholin-4-ylsulfonyl)-1H-indole-2-carboxamide

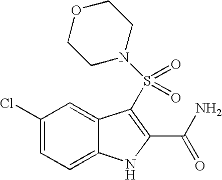

Step A: Ethyl 5-chloro-1-(phenylsulfonyl)-1H-indole-2-carboxylate

A 60% dispersion of NaH in mineral oil (1.07 g, 26.9 mmol) was washed with hexane, and the resulting powder was suspended in 40 mL of DMF. After cooling the stirred mixture to 0° C., ethyl 5-chloro-1H-indole-2-carboxylate (5.00 g, 22.4 mmol) was added in portions. The solution was warmed to room temperature, during which gas was released. After 15 minutes, the mixture was cooled again to 0° C., and benzenesulfonyl chloride was added dropwise (3.14 mL, 24.6 mmol). After warming to room temperature, the reaction was stirred for 1.5 hours, then poured into a mixture of EtOAc and saturated aqueous NaHCO₃ solution. The organic phase was washed with water and brine, dried with Na₂SO₄, filtered, and concentrated in vacuo. The resulting solid was stirred in 50 mL of a 10% EtOAc/hexane solution for 30 minutes, then filtered to provide the titled product as a white powder. Proton NMR for the product was consistent with the titled compound. ESI+MS: 364.1 [M+H]⁺.

Step B: 5-Chloro-2-(ethoxycarbonyl)-1-(phenylsulfonyl)-1H-indole-3-sulfonic Acid

To a solution of ethyl 5-chloro-1-(phenylsulfonyl)-1H-indole-2-carboxylate (5.56 g, 15.3 mmol) in 50 mL of dichloromethane at 0° C. was added acetic anhydride (7.23 mL, 76.6 mmol), followed by dropwise addition of concentrated sulfuric acid. The solution was warmed to room temperature, stirred for 3 hours, and partitioned between 0.5 L of EtOAc and 0.5 L of 3N HCl solution. The organic phase was washed with brine, dried with Na₂SO₄, filtered, and concentrated in vacuo. The product was reconcentrated from benzene in vacuo to give the titled product as a yellow solid. Proton NMR for the product was consistent with the titled compound of the formula C₁₇H₁₄ClNO₇S₂.0.5 CH₃CO₂H. ESI+MS: 444.0 [M+H]⁺, 466.0 [M+Na]⁺.

Step C: Ethyl 5-chloro-3-(chlorosulfonyl)-1-(phenylsulfonyl)-1H-indole-2-carboxylate

To a solution of the 5-chloro-2-(ethoxycarbonyl)-1-(phenylsulfonyl)-1H-indole-3-sulfonic acid (9.52 g, 21.4 mmol) in 100 mL of dichloromethane at 0° C. was added oxalyl chloride (5.61 mL, 64.3 mmol). Dimethylformamide (0.2 mL) was added, and the reaction was allowed to warm to room temperature. After 24 hours, another portion of oxalyl chloride (3.0 mL) was added, and the reaction was stirred for an additional 16 hours. The mixture was concentrated in vacuo to provide a yellow foam. Proton NMR for the product was consistent with the titled compound. ESI+MS: 426.2 [M−Cl]⁺.

Step D: Ethyl 5-chloro-3-(morpholin-4-ylsulfonyl)-1-(phenylsulfonyl)-1H-indole-2-carboxylate

To a solution of ethyl 5-chloro-3-(chlorosulfonyl)-1-(phenylsulfonyl)-1H-indole-2-carboxylate (149 mg, 0.322 mmol) in 5 mL of dichloromethane at 0° C. was added triethylamine (0.050 mL, 0.39 mmol), followed by morpholine (0.040 mL, 0.48 mmol). After four hours, the mixture was concentrated in vacuo to give the crude titled product. ESI+MS: 513.1 [M+H]⁺.

Step E: 5-chloro-3-[(methylamino)sulfonyl]-1H-indole-2-carboxamide

A sealed tube was charged with ethyl 5-chloro-3-(morpholin-4-ylsulfonyl)-1-(phenylsulfonyl)-1H-indole-2-carboxylate (ca. 0.32 mmol) and 5 mL of isopropanol. The solution was cooled in an ice bath, and ammonia gas was bubbled through the solution for 5 minutes. The tube was sealed, and heated at 100° C. for 18 hours. The mixture was concentrated in vacuo, taken up in 0.5 mL of 80% DMF/water solution, filtered, and purified by preparative reverse phase HPLC to afford the titled product. Proton NMR for the product was consistent with the titled compound. ESI+MS: 344.0 [M+H]⁺.

### Example 27

5-Bromo-3-(morpholin-4-ylsulfonyl)-1H-indole-2-carboxamide

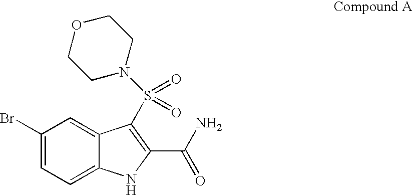

Step A: Ethyl 5-bromo-3-(chlorosulfonyl)-1-(phenylsulfonyl)-1H-indole-2-carboxylate

Following the procedures described in Steps A-C of Example 26, replacing ethyl 5-chloro-1H-indole-2-carboxylate with ethyl 5-bromo-1H-indole-2-carboxylate in Step A, the title compound was obtained. ESI+MS: 505.0 [M+H]⁺.

Step B: Ethyl 5-bromo-3-(morpholin-4-ylsulfonyl)-1-(phenylsulfonyl)-1H-indole-2-carboxylate

To a solution of ethyl 5-bromo-3-(chlorosulfonyl)-1-(phenylsulfonyl)-1H-indole-2-carboxylate (10.9 mmol) in 150 mL of dichloromethane at 0° C. was added triethylamine (1.53 mL, 10.9 mmol), followed by morpholine (1.34 mL, 15.3 mmol). After 30 minutes, the mixture was poured into ethyl acetate and saturated NaHCO₃ solution, and the aqueous phase was extracted three times with ethyl acetate. The combined organic layers were dried (Na₂SO₄), filtered, and concentrated in vacuo to give the titled product. ESI+MS: 557.0 [M+H]⁺.

Step C: Ethyl 5-bromo-3-(morpholin-4-ylsulfonyl)-1H-indole-2-carboxylate

To a solution of ethyl 5-bromo-3-(morpholin-4-ylsulfonyl)-1-(phenylsulfonyl)-1H-indole-2-carboxylate (10.9 mmol) in 75 mL of THF was added NaOH (482 mg, 12.0 mmol) dissolved in 2 mL of water. After one hour, the reaction was poured into ethyl acetate and water, and the aqueous phase was extracted three times with ethyl acetate. The combined organic layers were dried (Na₂SO₄), filtered, and concentrated in vacuo to give the titled product, which was recrystallized from EtOAc/hexane. ESI+MS: 417.1 [M+H]⁺.

Step D: 5-Bromo-3-(morpholin-4-ylsulfonyl)-1H-indole-2-carboxamide

Following the procedure described in Step E of Example 26, replacing ethyl 5-chloro-3-(morpholin-4-ylsulfonyl)-1-(phenylsulfonyl)-1H-indole-2-carboxylate with ethyl 5-bromo-3-(morpholin-4-ylsulfonyl)-1H-indole-2-carboxylate, the title compound was obtained after crystallization from EtOAc/hexane. Proton NMR for the product was consistent with the titled compound. ESI+MS: 388.0 [M+H]⁺.

### Example 28

5-Iodo-3-(morpholin-4-ylsulfonyl)-1H-indole-2-carboxamide

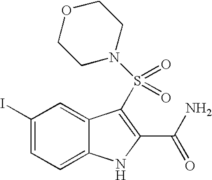

Step A: Ethyl 3,5-diiodo-1H-indole-2-carboxylate

Ethyl indole-2-carboxylate (5.00 g, 26.4 mmol), iodine (6.71 g, 26.4 mmol), sodium periodate (2.82 g, 13.2 mmol) and concentrated sulfuric acid (2.94 mL, 52.8 mmol) were combined in 50 mL of absolute ethanol and heated to reflux for 1.5 hours. The vessel was cooled to ambient temperature and poured into a biphasic mixture of ethyl acetate (100 mL) and saturated aqueous sodium sulfite (100 mL) solution. The organic layer was removed and the aqueous layer was further extracted twice with ethyl acetate. The combined organic extracts were washed once with aqueous saturated NaCl, dried with Na₂SO₄, filtered and concentrated in vacuo to provide the title product. ESI+MS: 441.8 [M+H]⁺.

Step B: Ethyl 5-iodo-1H-indole-2-carboxylate

Ethyl 3,5-diiodo-1H-indole-2-carboxylate (12.1 g, 26.4 mmol) was suspended in 250 mL of absolute ethanol, to which concentrated aqueous hydrogen chloride (22.0 mL, 264 mmol) was added. Zinc dust (17.3 g, 264 mmol) was added portionwise over 30 minutes. After stirring for 45 minutes, two additional portions of zinc were added slowly (5.2 and 4.4 g, 146 mmol). After stirring for 30 minutes, the mixture was poured into water and extracted four times with ethyl acetate. The combined organic extracts were washed once with aqueous saturated NaHCO₃ and once with aqueous saturated NaCl. The organic extract was dried with Na₂SO₄, filtered and concentrated in vacuo. The residue was crystallized three times from hexanes and ethyl acetate, providing the title compound. The mother liquor was columned by flash chromatography (0 to 8% ethyl acetate in hexanes) to provide an additional amount of the title compound. HRMS (ES) exact mass calculated for C₁₁H₁₀INO₂ (M+Na⁺): 377.9648. Found 377.9649.

Step C: Ethyl 5-iodo-3-(chlorosulfonyl)-1-(phenylsulfonyl)-1H-indole-2-carboxylate

Following the procedures described in Steps A-C of Example 26, replacing ethyl 5-chloro-1H-indole-2-carboxylate with ethyl 5-iodo-1H-indole-2-carboxylate in Step A, the title compound was obtained. ESI+MS: 518.07 [M−Cl]⁺.

Step D: 5-Iodo-3-(morpholin-4-ylsulfonyl)-1H-indole-2-carboxamide

Following the procedures described in Steps D and E of Example 26, replacing in

Step D ethyl 5-chloro-3-(chlorosulfonyl)-1-(phenylsulfonyl)-1H-indole-2-carboxylate with ethyl 5-iodo-3-(chlorosulfonyl)-1-(phenylsulfonyl)-1H-indole-2-carboxylate, the title compound was obtained. Proton NMR for the product was consistent with the titled compound. ESI+MS: 436.0 [M+H]⁺.

### Example 29

7-Amino-3-(morpholin-4-ylsulfonyl)-1H-indole-2-carboxamide

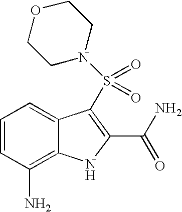

Step A: Ethyl 3-(chlorosulfonyl)-7-nitro-1-(phenylsulfonyl)-1H-indole-2-carboxylate

Following the procedures described in Steps A-C of Example 26, replacing ethyl 5-chloro-1H-indole-2-carboxylate with ethyl 7-nitro-1H-indole-2-carboxylate in Step A, the title compound was obtained.

Step B: 3-(Morpholin-4-ylsulfonyl)-7-nitro-1H-indole-2-carboxamide

Following the procedures described in Steps B and C of Example 27 and E of Example 26, replacing in Step B of Example 2 ethyl 5-bromo-3-(chlorosulfonyl)-1-(phenylsulfonyl)-1H-indole-2-carboxylate with ethyl 3-(chlorosulfonyl)-7-nitro-1-(phenylsulfonyl)-1H-indole-2-carboxylate, and replacing in Step E of Example 26 ethyl 5-chloro-3-(morpholin-4-ylsulfonyl)-1-(phenylsulfonyl)-1H-indole-2-carboxylate with ethyl 3-(morpholin-4-ylsulfonyl)-7-nitro-1H-indole-2-carboxylate, the title compound was obtained. HRMS (ES) exact mass calculated for C₁₃H₁₅N₄O₆S (M+H⁺): 355.0707. Found 355.0713.

Step C: 7-Amino-3-(morpholin-4-ylsulfonyl)-1H-indole-2-carboxamide

To a solution of 3-(morpholin-4-ylsulfonyl)-7-nitro-1H-indole-2-carboxamide (708 mg, 2.00 mmol) in 30 mL of methanol was added 10% palladium on carbon (200 mg), and the reaction was equipped with a balloon of hydrogen gas. After 2 hours, the reaction was filtered through celite, the filter pad was rinsed with ethyl acetate, and the resulting solution was concentrated in vacuo to provide the titled compound as a yellow solid. A portion of this was taken up in dichloromethane, treated with excess ethereal HCl, and concentrated in vacuo to give an off-white solid, used for biological testing. HRMS (ES) exact mass calculated for C₁₃H₁₇N₄O₄S (M+H⁺): 325.0965. Found 325.0971.

### Example 30

(±)-5-Chloro-3-{[2-(phenoxymethyl)morpholin-4-yl]sulfonyl}-1H-indole-2-carboxamide

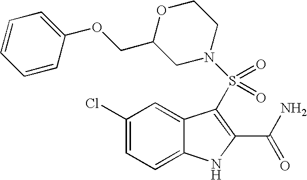

Step A: Ethyl (±)-5-chloro-3-{[2-(phenoxymethyl)morpholin-4-yl]sulfonyl}-1-(phenylsulfonyl)-1H-indole-2-carboxylate

Following the procedure described in Step D of Example 26, replacing morpholine with (±)-2-(phenoxymethyl)morpholine (G. A. Showell et al., Bioorg. Med. Chem. Lett. 1998, 6, 1-8.), the title compound was obtained after purification by flash chromatography on silica gel (ethyl acetate/hexanes). Proton NMR for the product was consistent with the titled compound. ESI+MS: 619.2 [M+H]⁺.

Step B: (±)-5-Chloro-3-{[2-(phenoxymethyl)morpholin-4-yl]sulfonyl}-1H-indole-2-carboxamide

Following the procedure described in Step E of Example 26, ethyl (±)-5-chloro-3-{[2-(phenoxymethyl)morpholin-4-yl]sulfonyl}-1-(phenylsulfonyl)-1H-indole-2-carboxylate was converted to the title compound. Proton NMR for the product was consistent with the titled compound. ESI+MS: 450.0 [M+H]⁺.

### Example 31

(S)-5-Chloro-3-{[2-(phenoxymethyl)morpholin-4-yl]sulfonyl}-1H-indole-2-carboxamide

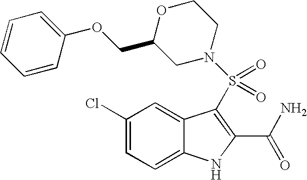

Step A: Ethyl (S)-5-chloro-3-{[2-(phenoxymethyl)morpholin-4-yl]sulfonyl}-1-(phenylsulfonyl)-1H-indole-2-carboxylate

The product from Step A of Example 30 was resolved by preparative chiral HPLC (ChiralPak AD) to produce a first-eluting enantiomer and a second-eluting enantiomer. To assign the (S)-configuration to the first-eluting enantiomer, (S)-2-(phenoxymethyl)morpholine (from resolution of the racemate by preparative ChiralPak AD HPLC) was converted to the titled product using the procedure described in Step A of Example 30. The configuration of (S)-2-(phenoxymethyl)morpholine was assigned by ¹H NMR analysis of the derived Mosher's amide (cf. J. Org. Chem. 1996, 61, 2056-2064).

Step B: (S)-5-Chloro-3-{[2-(phenoxymethyl)morpholin-4-yl]sulfonyl}-1H-indole-2-carboxamide

Following the procedure described in Step E of Example 26, ethyl (S)-5-chloro-3-{[2-(phenoxymethyl)morpholin-4-yl]sulfonyl}-1-(phenylsulfonyl)-1H-indole-2-carboxylate was converted to the title compound. Proton NMR for the product was consistent with the titled compound. ESI+MS: 450.0 [M+H]⁺.

### Example 32

(S)-5-Bromo-3-{[2-(phenoxymethyl)morpholino-4-yl]sulfonyl}-1H-indole-2-carboxamide

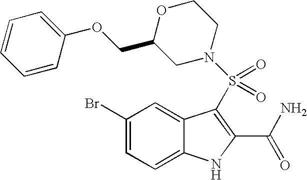

Following the procedures described in Steps A and B of Example 31, replacing the product from Step A of Example 30, ethyl (±)-5-chloro-3-{[2-(phenoxymethyl)morpholin-4-yl]sulfonyl}-1-(phenylsulfonyl)-1H-indole-2-carboxylate, with ethyl (±)-5-bromo-3-{[2-(phenoxymethyl)morpholin-4-yl]sulfonyl}-1-(phenylsulfonyl)-1H-indole-2-carboxylate, the title compound was obtained after purification by preparative reversed phase HPLC. Proton NMR for the product was consistent with the titled compound. ESI+MS: 493.95 [M+H]⁺.

### Example 33

(S)-5-Iodo-3-{[2-(phenoxymethyl)morpholino-4-yl]sulfonyl}-1H-indole-2-carboxamide

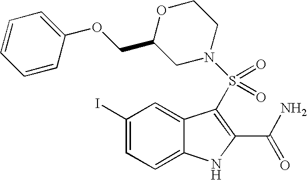

Following the procedures described in Steps A and B of Example 31, replacing the product from Step A of Example 30, ethyl (±)-5-chloro-3-{[2-(phenoxymethyl)morpholin-4-yl]sulfonyl}-1-(phenylsulfonyl)-1H-indole-2-carboxylate, with ethyl (±)-5-iodo-3-{[2-(phenoxymethyl)morpholin-4-yl]sulfonyl}-1-(phenylsulfonyl)-1H-indole-2-carboxylate, the title compound was obtained after purification by preparative reversed phase HPLC. Proton NMR for the product was consistent with the titled compound. ESI+MS: 541.82 [M+H]⁺.

### Example 34

3-(Morpholin-4-ylsulfonyl)-7-[(pyridin-4-ylmethyl)amino]-1H-indole-2-carboxamide

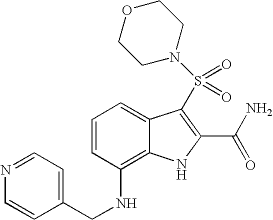

To a solution of 7-amino-3-(morpholin-4-ylsulfonyl)-1H-indole-2-carboxamide from Example 29 (36 mg, 0.11 mmol) in 1 mL of methanol was added 4-formylpyridine (24 mg, 0.22 mmol). After stirring overnight, sodium borohydride was added (13 mg, 0.33 mmol). After 2 hours, the reaction was quenched with 3N HCl, concentrated in vacuo, taken up in DMF, and purified by preparative reversed phase HPLC. The titled product was obtained as a yellow solid. Proton NMR for the product was consistent with the titled compound. ESI+MS: 416.4 [M+H]⁺.

### Example 35

7-Amino-3-{[(2S)-2-(phenoxymethyl)morpholin-4-yl]sulfonyl}-1H-indole-2-carboxamide

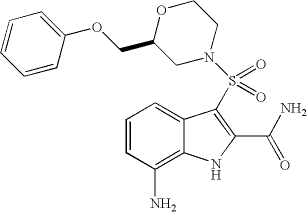

Using the method described in Step C of Example 29, 7-nitro-3-{[(2S)-2-(phenoxymethyl)morpholin-4-yl]sulfonyl}-1H-indole-2-carboxamide was converted to the titled product. Proton NMR for the product was consistent with the titled compound. HRMS (ES) exact mass calculated for C₂₀H₂₃N₄O₅S (M+H⁺): 431.1384. Found 431.1385.

### Example 36

3-{[(2S)-2-(Phenoxymethyl)morpholin-4-yl]sulfonyl}-7-[(pyridin-4-ylmethyl)amino]-1H-indole-2-carboxamide

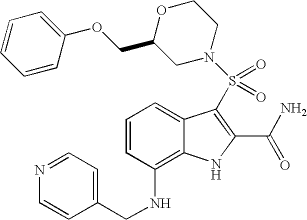

Using the method described in Example 34, 7-Amino-3-{[(2S)-2-(phenoxymethyl)morpholin-4-yl]sulfonyl}-1H-indole-2-carboxamide was converted to the titled product. Proton NMR for the product was consistent with the titled compound. HRMS (ES) exact mass calculated for C₂₆H₂₈N₅O₅S (M+H⁺): 522.1806. Found 522.1808.

### Example 37

(R,S)-5-Bromo-3-[(2-{[(cyclohexylmethly)amino]carbonyl}morpholin-4-yl)sulfonyl]-1H-indole-2-carboxamide

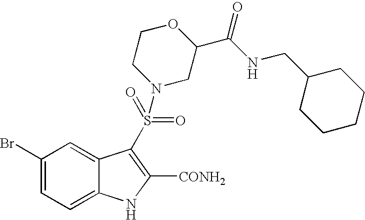

To a 20 mL tube was placed PS-DCC (832 mg, 0.018 mmol, 1.38 mmol/g), HOBt (109 mg, 0.81 mmol), and a 5:1:1 mixture of CHCl₃:CH₃CN:tBuOH. Then, cyclohexylmethyl amine (65 μL, 0.5 mmol) and (R,S)-Boc-2-carboxymorpholine (125 mg, 0.54 mmol) were added, and the vial was placed on a GlasCol orbital rotator for 16 hours. After this time, MP-carbonate (480 mg, 1.62 mmol, 3.38 mmol/g) was added to scavenge the HOBt and excess (R,S)-Boc-2-carboxymorpholine.

Three hours later, the vial's contents were filtered through an Applied Separations filter tube, washed with DCM (3×3 mL) and concentrated in an HTII-12 Genevac unit to afford an yellow oil. This material was then dissolved in DCM (2 mL) and 4 N HCl/dioxane (2 mL) added. After 10 minutes, the solvent was evapoarted under a stream of nitrogen gas and purified/free-based by standard SCX SPE.

Concentration provided a colorless oil, N-(cyclohexylmethyl)morpholine-2-carboxamide, 102 mg (90%)—a single peak (214 nm and ELSD) at 2.06 min (CH₃CN/H₂O/1% TFA, 4 min gradient). To an 8 mL vial was placed ethyl 5-bromo-3-(chlorosulfonyl)-1-(phenylsulfonyl)-1H-indole-2-carboxylate (50 mg, 0.099 mmol), PS-NMM (58 mg, 0.216 mmol, 3.72 mmol/g), PS-DMAP (37 mg, 0.05 mmol, 1.48 mmol/g) and DCM. Then, N-(cyclohexylmethyl)morpholine-2-carboxamide (18 mg, 0.08 mmol) was added, and the vial placed on a GlasCol orbital rotator for 16 hours. After this time, PS-trisamine resin (75 mg, 0.108 mmol, 1.44 mmol/g) was added to the vial to scavenge excess sulfonyl chloride.

Three hours later, the vial's contents were filtered through an Applied Separations filter tube, washed with DCM (3×3 mL) and concentrated in an HTII-12 Genevac unit to afford an yellow solid. This material was then dissolved in 2 M NH₃/EtOH, sealed in a scintillation vial and heated to 90 degrees on a J-KEM heater/shaker block for 3 hours. The vial was then dried in an HTII-12 Genevac unit to afford a brown solid. This material was then purified by Mass Guided HPLC on an Agilent 1100 Purification unit to afford a white crystalline solid.

Analytical LCMS: single peak (214 nm and ELSD) at 3.35 min (CH₃CN/H₂O/1% TFA, 4 min gradient).

¹H NMR (300 MHz, DMSO-d₆): δ 8.2 (s, 2H), 7.99 (s, 1H), 7.39 (m, 2H), 7.48 (m, 2H), 3.95 (t, J=11 Hz, 2H), 3.63 (m, 2H), 3.44 (d, J=11.4 Hz, 1H), 2.48 (m, 2H), 2.3 (t, J=18 Hz, 1H), 1.56 (m, 6H), 1.34 (m, 2H), 1.08 (m, 2H), 0.77 (m, 2H).

HRMS calc'd for C₂₁H₂₇BrN₄O₅S, 527.0958; found, 527.0940.

The analogs illustrated below were made utilizing the above-described procedure:

| Name | Structure | ESI + MS |
|---|---|---|
| 5-bromo-3-({2-[(2,3- dihydro-1H-inden-1- ylamino)carbonyl]morpholin-4-yl}sulfonyl)- 1H-indole-2-carboxamide | 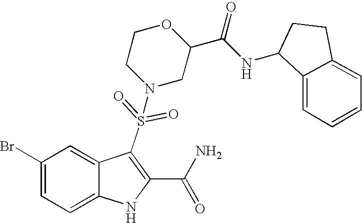 | 548.4 |
| 5-bromo-3-({2-[(2,3- dihydro-1H-inden-1- ylamino)carbonyl]morpholin-4-yl}sulfonyl)- 1H-indole-2-carboxamide | 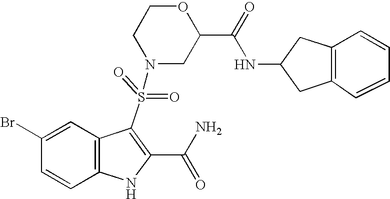 | 548.4 |
| 5-bromo-3-({2-[(2,3- dihydro-1H-inden-1- ylamino)carbonyl]morpholin-4-yl}sulfonyl)- 1H-indole-2-carboxamide | 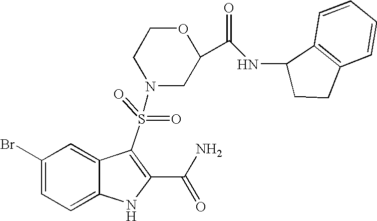 | 548.4 |
| 5-bromo-3-[(2-{[(1- naphthylmethyl)amino]carbonyl}morpholin-4- yl)sulfonyl]-1H-indole-2- carboxamide | 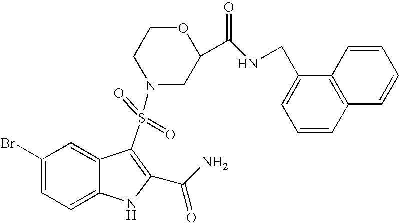 | 572.4 |

### Example 38

5-Bromo-3-{[4-(3-phenylpropyl)piperidin-1-yl]sulfonyl}-1H-indole Carboxamide

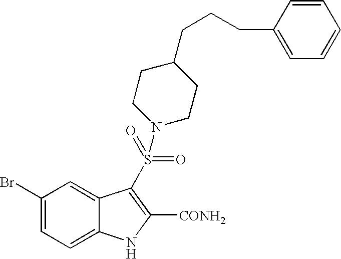

To an 8 mL vial was placed ethyl 5-bromo-3-(chlorosulfonyl)-1-(phenylsulfonyl)-1H-indole-2-carboxylate (50 mg, 0.099 mmol), PS-NMM (58 mg, 0.216 mmol, 3.72 mmol/g), PS-DMAP (37 mg, 0.05 mmol, 1.48 mmol/g) and DCM. Then, 4-(3-phenylpropyl)piperidine (26 mg, 0.08 mmol) was added, and the vial placed on a GlasCol orbital rotator for 16 hours. After this time, PS-trisamine resin (75 mg, 0.108 mmol, 1.44 mmol/g) was added to the vial to scavenge excess sulfonyl chloride.

Three hours later, the vial's contents were filtered through an Applied Separations filter tube, washed with DCM (3×3 mL) and concentrated in an HTII-12 Genevac unit to afford an yellow solid. This material was then dissolved in 2 M NH₃/EtOH, sealed in a scintillation vial and heated to 90 degrees on a J-KEM heater/shaker block for 3 hours. The vial was then dried in an HTII-12 Genevac unit to afford a brown solid. This material was then purified by Mass Guided HPLC on an Agilent 1100 Purification unit to afford a white crystalline solid.

Analytical LCMS: single peak (214 nm and ELSD) at 3.94 min (CH₃CN/H₂O/1% TFA, 4 min gradient).

¹H NMR (300 MHz, DMSO-d₆): δ 8.26 (s, 1H), 8.19 (s, 1H), 8.03 (s, 1H), 7.69 (m, 1H), 7.63 (m, 1H), 7.47 (m, 1H), 7.2 (m, 2H), 7.13 (m, 3H), 3.59 (m, 2H), 3.2 (m, 2H), 2.18 (m, 3H), 1.66 (d, J=11.7 Hz, 2H), 1.46 (m, 2H), 1.13 (m, 2H).

HRMS calc'd for C₂₃H₂₆BrN₃O₃S, 504.0951; found, 504.0944.

The analog illustrated in the table below was prepared utilizing the procedures described above.

| | | ESI + |
|---|---|---|
| Name | Structure | MS |
| 5-chloro-3-({2-[(4- chlorophenoxy)methyl]morpholin- 4-yl}sulfonyl)-1H-indole-2- carboxamide | 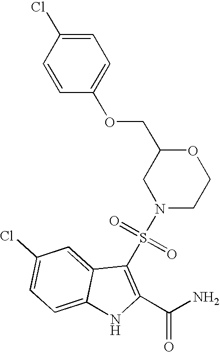 | 485.4 |

### Example 39

### Assays

The protein kinase inhibitor compounds of the instant invention described in Examples 26-38 were tested by the assays described below and were found to have kinase inhibitory activity. In particular, the compounds of the instant invention inhibited IGF-1R or insulin receptor kinase activity with an IC₅₀ of less than or equal to about 100 μM. Other assays are known in the literature and could be readily performed by those with skill in the art (see for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

IGF-1R Kinase Assay

IGF-1R receptor kinase activity is measured by incorporation of phosphate into a peptide substrate containing a tyrosine residue. Phosphorylation of the peptide substrate is quantitated using anti-IGF-1R and anti-phosphotyrosine antibodies in an HTRF (Homogeneous Time Resolved Fluorescence) detection system. (Park, Y-W., et al. Anal. Biochem., (1999) 269, 94-104)

Materials

IGF-1R Receptor Kinase Domain

The intracellular kinase domain of human IGF-1R was cloned as a glutathione S-transferase fusion protein. IGF-1R β-subunit amino acid residues 930 to 1337 (numbering system as per Ullrich et al., EMBO J. (1986) 5, 2503-2512) were cloned into the baculovirus transfer vector pAcGHLT-A (BD-Pharmingen) such that the N-terminus of the IGF-1R residues are fused to the C-terminus of the GST domain encoded in the transfer vector pAcGHLT-A. Recombinant virus was generated and the fusion protein expressed in SF-9 insect cells (BD-Pharmingen). Enzyme was purified by means of a glutathione sepharose column.

Insulin Receptor Kinase Domain

The intracellular kinase domain of human insulin receptor was cloned as a glutathione S-transferase fusion protein. Insulin receptor β-subunit amino acid residues 941 to 1343 (numbering system as per Ullrich et al., Nature, (1985) 313, 756-761) were cloned into the baculovirus transfer vector pAcGHLT-A (BD-Pharmingen) such that the N-terminus of the IGF-1R residues are fused to the C-terminus of the GST domain encoded in the transfer vector pAcGHLT-A. Recombinant virus was generated and the fusion protein expressed in SF-9 insect cells (BD-Pharmingen) Enzyme was purified by means of a glutathione sepharose column.

Insect Cell Lysis Buffer

10 mM Tris pH 7.5; 130 mM NaCl; 2 mM DTT; 1% Triton X-100; 10 mM NaF;

10 mM NaPi; 10 mM NaPPi; 1× protease inhibitor cocktail (Pharmingen).

Wash Buffer

Phosphate Buffered Saline (PBS): 137 Mm NaCl, 2.6 mM KCl, 10 mM Na₂HPO₄, 1.8 mM KH₂PO₄, pH 7.4; 1 mM DTT; 1× protease inhibitor cocktail

Dialysis Buffer

20 mM Tris pH 7.5; 1 mM DTT; 200 mM NaCl; 0.05% Triton X-100 and 50% glycerol

Enzyme Dilution Buffer

50 mM Tris pH 7.5; 1 mM DTT; 100 mM NaCl; 10% glycerol; 1 mg/ml BSA

Enzyme Reaction Buffer

20 mM Tris pH 7.4; 100 mM NaCl; 1 mg/ml BSA; 5 mM MgCl₂; 2 mM DTT

Quench Buffer

125 mM Tris pH 7.8; 75 mM EDTA; 500 mM KF; 0.125% Triton X-100; 1.25% BSA; 60 nM SA-XL665 (Packard); 300 pM europium cryptate labeled anti-phosphotyrosine antibody (Eu-PY20)

Peptide Substrate

Sequence LCB-EQEDEPEGDYFEWLE-NH₂; stock solution is 1 mM disolved in DMSO; diluted to 1 μM in 1× enzyme reaction buffer for 10× working stock. (LCB=aminohexanoylbiotin)

ATP

Stock solution is 0.5 M ATP (Boehringer) pH 7.4; stock solution is diluted to 40 mM ATP in enzyme reaction buffer to give 20×working stock solution

HEK-21 Cell Line

Human embryonic kidney cells (HEK-293) (ATCC) were transfected with an expression plasmid containing the entire IGF-1R coding sequence. After antibiotic selection, colonies were screened for IGF-1R overexpression by western blot analysis. One clone, designated HEK-21 was selected for cell based IGF-1R autophosphorylation assays.

HEK Cell Growth Media

Dulbecco's Modified Eagle's Media (DMEM), 10% Fetal Calf Serum, 1×Penn/Strep, 1×Glutamine, 1× Non-essential amino acids (all from Life Technologies)

Cell Lysis Buffer

50 mM Tris-HCl pH 7.4; 150 mM NaCl; 1% Triton X-100 (Sigma); 1×Mammalian protease inhibitors (Sigma); 10 mM NaF; 1 mM NaVanadate

Western Blocking Buffer

20 mM Tris-HCl pH 8.0; 150 mM NaCl; 5% BSA (Sigma); 0.1% Tween 20 (Biorad)

Methods

Protein Purifications

Spodoptera frugiperda SF9 cells were transfected with recombinant virus encoding either the GST-IGF-1R β-subunit or GST-InsR fusion protein at an MOI of 4 virus particles/cell. Cells are grown for 48 hours at 27° C., harvested by centrifugation and washed once with PBS. The cell pellet is frozen at −70° C. after the final centrifugation. All subsequent purification steps are performed at 4° C. 10 grams of frozen cell paste is thawed in a 90 ml volume of insect cell lysis buffer (BD-Pharmingen) and held on ice with occasional agitation for 20 minutes. The lysate is centrifuged at 12000 g to remove cellular debris. Lysis supernatant was mixed with 45 ml of glutathione agarose beads (BD-Pharmingen) and agitated slowly at 4° C. for one hour after which the beads were centrifuged and washed 3× with wash buffer. The beads are resuspended in 45 ml of wash buffer and poured as a slurry into a chromatography column. The column is washed with 5 volumes of wash buffer and the GST-IGF-1R is eluted from the column with 5 mM Glutathione in wash buffer. Pooled fractions are dialyzed vs. dialysis buffer and stored at −20° C.

IGF-1R Kinase Assay

The IGF-1R enzyme reaction is run in a 96 well plate format. The enzyme reaction consists of enzyme reaction buffer plus 0.1 nM GST-IGF-1R, 100 nM peptide substrate and 2 mM ATP in a final volume of 60 microliters. Inhibitor, in DMSO, is added in a volume 1 microliter and preincubated for 10 minutes at 22° C. Final inhibitor concentration can range from 100 μM to 1 nM. The kinase reaction is initiated with 3 microliters of 40 mM ATP. After 20 minutes at 22° C., the reaction is stopped with 40 microliters of quench buffer and allowed to equilibrate for 2 hours at 22° C. Relative fluorescent units are read on a Discovery plate reader (Packard). IC50s for compounds are determined by 4 point sigmoidal curve fit.

Insulin Receptor Kinase Assay

The kinase reaction for insulin receptor is identical to that used to assay IGF-1R (above), except that GST-InsR is substituted at a final concentration of 0.1 nM.

Cell Based IGF-1R Autophosphorylation Assay

IGF-1R inhibitor compounds are tested for their ability to block IGF-I induced IGF-1R autophosphorylation in a IGF-1R transfected human embryonic kidney cell line (HEK-21). HEK-21 cells over-expressing the human IGF-1R receptor are cultured in 6-well plates (37° C. in a 5% CO₂ atmosphere) in HEK cell growth media to 80% of confluence. Cells are serum starved for four hours in HEK growth media with 0.5% fetal calf serum. A 10× concentration of inhibitor in growth media is added to the cells in one-tenth the final media volume and allowed to preincubate for one hour at 37° C. Inhibitor concentration can range from 10 nM to 100 μM. IGF-I (Sigma) is added to the serum starved cells to a final concentration of 30 ng/ml. After a 10 minute incubation in the presence of IGF-I at 37° C., the media is removed, the cells washed once with PBS and 0.5 mls of cold cell lysis buffer added. After 5 minutes incubation on ice, cells are scraped from the wells and lysis buffer plus cells are transferred to a 1.5 ml microfuge tube. The total lysate is held at 4° C. for twenty minutes and then centrifuged at top speed in a microfuge. The supernatant is removed and saved for analysis. Phosphorylation status of the receptor is assessed by Western blot. Lysates are electrophoresed on 8% denaturing Tris-Glycine polyacryl-amide gels and the proteins transferred to nitrocellulose filters by electro-blotting. The blots are blocked with blocking reagent for 10 minutes after which anti-phosphotyrosine antibody (4G10, Upstate Biotechnology) is added to a final dilution of 1:1500. Blots and primary antibody are incubated at 4° C. overnight. After washing with PBS plus 0.2% Tween 20 (Biorad), an HRP conjugated anti-mouse secondary antibody (Jackson Labs) is added at a dilution of 1:15000 and incubated at 4° C. for 2 hours. Blots are then washed with PBS-Tween and developed using ECL (Amersham) luminescent reagent. Phosphorylated IGF-1R on the blots is visualized by autoradiography or imaging using a Kodak Image Station 440. IC50s are determined through densitometric scanning or quantitation using the Kodak Digital Science software.

### Example 40

### Combination Assay

The herein disclosed inhibitors were evaluated as to the ability to increase cell death. Inhibitors of Akt and inhibitors of Akt and a protein kinase were assessed alone and in combination to determine the effect on caspase 3 activation.

Apoptosis Assay: Caspase 3 Activation

Cells were seeded in 96 well plates at concentrations ranging from 2×10³ to 1×10⁴ cells per well in their respective growth media. For anchorage independent assays (spheroids) cells were plated in 96 well plates that had been coated with poly-heme(poly 2-hydroxyethylmethacrylate [Sigma] 10 mg/ml in 95% ethanol). 72 hours later caspase 3 assays were set up as follows:

a. For standard combination assays, compounds were added at respective concentrations indicated and plates incubated at 37° C. in a CO₂ incubator for 18 hours. For standard combination assays utilizing treatment with camptothecin, compounds and camptothecin were added at the same time at the respective concentrations indicated.

b. For combinations involving addition of the death receptor ligand TRAIL/Apo2L (Research Diagnostics) compounds were added for 1.5 hours prior to addition of TRAIL and plates incubated an additional 3 to 4 hours post TRAIL addition. In the case of the time course, plates were incubated for 2, 3, 4, 5, 6 and 7 hrs with Trail ligand before ending the assay.

For both set ups, total final volumes did not exceed 250 μl. At the end of the incubation period, the plates are spun at 13,000 rpm for 10 minutes at 4° C. (Beckman Model J-6M centrifuge). Using a multichannel pipet, the media is carefully removed from the wells and 50 μl of cell lysis buffer (Clontech ApoAlert™ Caspase 3 fluorescent assay kit) added to each well. The plates are put at 4° C. for 20 minutes and then at −70° C. for 1 hour (or till actual running of caspase enzyme assay). The plates are thawed to room temperature and caspase 3 substrate (AcDEVD-AFC, BSI/QCB) and DTT are added in 50 μl of a 2× reaction buffer (200 mM Hepes pH 7.6, 1 mM EDTA) to a final concentration of 50 μM substrate, 5 mM DTT. The plates are placed at 37° C. for 6 to 18 hours before reading on a Spectra Max Gemini fluorescence plate reader (Molecular Devices) at 400/505 nM wavelength. All data is plotted as percent activity of control.

What is claimed is:

1. A method for treating cancer in a mammal in need thereof which comprises administering to said mammal amounts of at least one inhibitor of Akt and at least one inhibitor of a protein kinase.

2. The method according to claim 1 wherein an amount of an inhibitor of Akt and an amount of an inhibitor of a protein kinase are administered consecutively.

3. The method according to claim 1 wherein an amount of an inhibitor of Akt and an amount of an inhibitor of a protein kinase are administered simultaneously.

4. The method according to claim 1 wherein the method of treating cancer is selected from inhibition of cancerous tumor growth and regression of cancerous tumors.

5. The method according to claim 1 wherein the method of treating cancer is selected from cancer comprising breast cancer, prostate cancer, pancreatic cancer, colorectal cancer, lung cancer, ovarian cancer, renal cell carcinoma, endometrial carcinoma, glioblastoma, colon cancer and bladder cancer.

6. The method according to claim 5 wherein the cancer is selected from breast cancer, prostate cancer, pancreatic cancer and ovarian cancer.

7. The method according to claim 1 wherein the inhibitor of Akt inhibits the activity of one or more of the isoforms of Akt.

8. The method according to claim 1 wherein the inhibitor of Akt is a small organic molecule.

9. The method according to claim 1 wherein the inhibitor of Akt inhibits the phosphorylation of one or more of the isoforms of Akt by upstream kinases and inhibits the phosphorylation of protein targets of an isoform or isoforms of Akt by the activated isoform or isoforms of Akt.

10. The method according to claim 1 wherein the inhibitor of Akt inhibits the phosphorylation of one or more of the isoforms of Akt by upstream kinases or inhibits the phosphorylation of protein targets of an isoform or isoforms of Akt by the activated isoform or isoforms of Akt.

11. The method according to claim 1 wherein the inhibitor of Akt is a selective inhibitor of the activity of Akt1.

12. The method according to claim 1 wherein the inhibitor of Akt is a selective inhibitor of the activity of Akt2.

13. The method according to claim 1 wherein the inhibitor of Akt is a selective inhibitor of the activity of Akt1 and Akt2.

14. The method according to claim 1 wherein the inhibitor of Akt is a selective inhibitor of the activity of Akt1 and Akt3.

15. The method according to claim 1 wherein the inhibitor of Akt is a selective inhibitor of the activity of Akt2 and Akt3.

16. The method according to claim 1 wherein the inhibitor of Akt is a selective inhibitor of the activity of Akt3.

17. The method according to claim 1 wherein the inhibitor of Akt inhibits the activity of one or more of the isoforms of Akt wherein the inhibition by the inhibitor is dependent on the presence of the region of the isoform of Akt selected from:
a) the pleckstrin homology domain,
b) the hinge region, and
c) the pleckstrin homology domain and the hinge region.

18. The method according to claim 17 wherein the region of the isoform of Akt is the pleckstrin homology domain.

19. The method according to claim 17 wherein the region of the isoform of Akt is the hinge region.

20. The method according to claim 17 wherein the region of the isoform of Akt is the pleckstrin homology domain and the hinge region.

21. The method according to claim 17 wherein the inhibitor of Akt is a selective inhibitor of the activity of Akt1.

22. The method according to claim 17 wherein the inhibitor of Akt is a selective inhibitor of the activity of Akt2.

23. The method according to claim 17 wherein the inhibitor of Akt is a selective inhibitor of the activity of Akt3.

24. The method according to claim 17 wherein the inhibitor of Akt is a selective inhibitor of Akt1 and Akt2.

25. The method according to claim 17 wherein the inhibitor of Akt is a selective inhibitor of Akt1 and Akt3.

26. The method according to claim 17 wherein the inhibitor of Akt is a selective inhibitor of Akt2 and Akt3.

27. The method according to claim 17 wherein the inhibitor of Akt is a selective inhibitor of Akt1, Akt2 and Akt3.

28. The method according to claim 1 wherein the inhibitor of Akt is a selective inhibitor of the activity of Akt1, but is not an inhibitor of the activity of a modified Akt1 that lacks the pleckstrin homology domain.

29. The method according to claim 1 wherein the inhibitor of Akt is a selective inhibitor of the activity of Akt2, but is not an inhibitor of the activity of a modified Akt2 that lacks the pleckstrin homology domain.

30. The method according to claim 1 wherein the inhibitor of Akt is a selective inhibitor of the activity of Akt3, but is not an inhibitor of the activity of a modified Akt3 that lacks the pleckstrin homology domain.

31. The method according to claim 1 wherein the inhibitor of Akt is a selective inhibitor of the activity of Akt1 and Akt2, but is not an inhibitor of the activity of a modified Akt1 that lacks the pleckstrin homology domain, a modified Akt2 that lacks the pleckstrin homology domain or both a modified Akt1 and a modified Akt2 protein that lack their pleckstrin homology domains.

32. The method according to claim 1 wherein the inhibitor of Akt is a selective inhibitor of the activity of Akt1 and Akt3, but is not an inhibitor of the activity of a modified Akt 1 that lacks the pleckstrin homology domain, a modified Akt3 that lacks the pleckstrin homology domain or both a modified Akt1 and a modified Akt3 protein that lack their pleckstrin homology domains.

33. The method according to claim 1 wherein the inhibitor of Akt is a selective inhibitor of the activity of Akt2 and Akt3, but is not an inhibitor of the activity of a modified Akt2 that lacks the pleckstrin homology domain, a modified Akt3 that lacks the pleckstrin homology domain or both a modified Akt2 and a modified Akt3 protein that lack their pleckstrin homology domains.

34. The method according to claim 1 wherein the inhibitor of Akt is a selective inhibitor of the activity of Akt1, Akt2 and Akt3, but is not an inhibitor of the activity of a modified Akt1 that lacks the pleckstrin homology domain, a modified Akt2 that lacks the pleckstrin homology domain, a modified Akt3 that lacks the pleckstrin homology domain or two or three modified Akt isoforms that lack their pleckstrin homology domains.

35. A method for treating cancer in a mammal in need thereof which comprises administering to said mammal amounts of at least two selective inhibitors of Akt.

36. The method of claim 35 wherein the activity of Akt1 and the activity of Akt2 is inhibited.

37. The method of claim 35 wherein the activity of Akt1 and the activity of Akt2 is inhibited but the activity of Akt3 is not inhibited.

38. The method according to claim 35 wherein the selective inhibitors of Akt are selected from:
a) an Akt1 selective inhibitor,
b) an Akt2 selective inhibitor,
c) an Akt3 selective inhibitor,
d) a selective inhibitor of both Akt1 and Akt2,
e) a selective inhibitor of both Akt1 and Akt3,
f) a selective inhibitor of both Akt2 and Akt3, and
g) a selective inhibitor of Akt1, Akt2 and Akt3.

39. The method according to claim 35 wherein the inhibitors of Akt are small organic molecules.

40. The method according to claim 38 wherein the selective inhibitors are selected from an Akt1 selective inhibitor, an Akt2 selective inhibitor and a selective inhibitor of both Akt1 and Akt2.

41. The method according to claim 35 wherein the selective inhibitors do not inhibit Akt3.

42. The method according to claim 40 wherein the selective inhibitors do not inhibit Akt3.

43. A method for selectively inhibiting Akt activity in a cell which comprises the administration of one or more selective Akt inhibitors.

44. The method of claim 43 wherein the activity of Akt1 and the activity of Akt2 is inhibited.

45. The method of claim 43 wherein the activity of Akt1 and the activity of Akt2 is inhibited but the activity of Akt3 is not inhibited.

46. The method of claim 43 wherein the selective inhibitors of Akt are selected from:
a) an Akt1 selective inhibitor,
b) an Akt2 selective inhibitor,
c) an Akt3 selective inhibitor,
d) a selective inhibitor of both Akt1 and Akt2,
e) a selective inhibitor of both Akt1 and Akt3,
f) a selective inhibitor of both Akt2 and Akt3, and
g) a selective inhibitor of Akt1, Akt2 and Akt3.

47. The method of claim 46 wherein the selective inhibitors are selected from an Akt1 selective inhibitor, an Akt2 selective inhibitor and a selective inhibitor of both Akt1 and Akt2.

48. The method of claim 43 wherein the selective inhibitors do not inhibit Akt3.

49 The method of claim 47 wherein the selective inhibitors do not inhibit Akt3.

50. The method of claim 43 wherein the selective inhibitor is a small molecule.

51. The method of claim 43 which is useful for the treatment of cancer.

52. The method according to claim 1 wherein the inhibitor of Akt is selected from a compound of the formula VII: 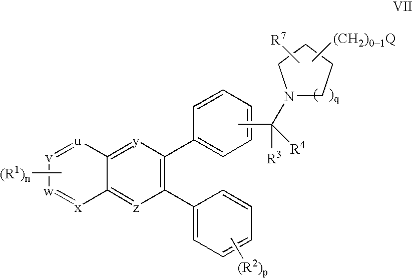
wherein:
a is 0 or 1;
b is 0 or 1;
m is 0, 1 or 2;
n is 0, 1, 2 or 3;
p is 0, 1 or 2;
q is 0, 1, 2, 3 or 4;
r is 0 or 1;
s is 0 or 1;
t is 2, 3, 4, 5 or 6;
u, v, w and x are independently selected from: CH and N;
y and z are independently selected from: CH and N, provided that at least one of y and z is N;
Q is selected from: —NR⁵R⁶, aryl and heterocyclyl, said aryl and heterocycle which is optionally substituted with one to three R^{z};
R¹ is independently selected from:
1) (C═O)ₐO_{b}C₁-C₁₀ alkyl,
2) (C═O)ₐO_{b}aryl,
3) C₂-C₁₀ alkenyl,
4) C₂-C₁₀ alkynyl,
5) (C═O)ₐO_{b} heterocyclyl,
6) (C═O)ₐO_{b}C₃-C₈ cycloalkyl,
7) CO₂H,
8) halo,
9) CN,
10) OH,
11) O_{b}C₁-C₆ perfluoroalkyl,
12) Oₐ(C═O)_{b}NR⁵R⁶,
13) NR^{c}(C═O)NR⁵R⁶,
14) S(O)ₘR^{a},
15) S(O)₂NR⁵R⁶,
16) NR^{c}S(O)ₘR^{a},
17) oxo,
18) CHO,
19) NO₂,
20) NR^{c}(C═O)O_{b}R^{a},
21) O(C═O)O_{b}C₁-C₁₀ alkyl,
22) O(C═O)O_{b}C₃-C₈ cycloalkyl,
23) O(C═O)O_{b}aryl, and
24) O(C═O)O_{b}-heterocycle,
said alkyl, aryl, alkenyl, alkynyl, heterocyclyl, and cycloalkyl optionally substituted with one or more substituents selected from R^{z};
R² is independently selected from:
1) (C═O)ₐO_{b}C₁-C₁₀ alkyl,
2) (C═O)ₐO_{b}aryl,
3) C₂-C₁₀ alkenyl,
4) C₂-C₁₀ alkynyl,
5) (C═O)ₐO_{b} heterocyclyl,
6) (C═O)ₐO_{b}C₃-C₈ cycloalkyl,
7) CO₂H,
8) halo,
9) CN,
10) OH,
11) O_{b}C₁-C₆ perfluoroalkyl,
12) Oₐ(C═O)_{b}NR⁵R⁶,
13) NR^{c}(C═O)NR⁵R⁶,
14) S(O)ₘR^{a},
15) S(O)₂NR⁵R⁶,
16) NR^{c}S(O)ₘR^{a},
17) CHO,
18) NO₂,
19) NR^{c}(C═O)O_{b}R^{a},
20) O(C═O)O_{b}C₁-C₁₀ alkyl,
21) O(C═O)O_{b}C₃-C₈ cycloalkyl,
22) O(C═O)O_{b}aryl, and
23) O(C═O)O_{b}-heterocycle,
said alkyl, aryl, alkenyl, alkynyl, heterocyclyl, and cycloalkyl optionally substituted with one, two or three substituents selected from R^{z};
R³ and R⁴ are independently selected from: H, C₁-C₆-alkyl and C₁-C₆-perfluoroalkyl, or
R³ and R⁴ are combined to form —(CH₂)ₜ— wherein one of the carbon atoms is optionally replaced by a moiety selected from O, S(O)ₘ, —N(R^{b})C(O)—, and —N(COR^{a})—;
R⁵ and R⁶ are independently selected from:
1) H,
2) (C═O)O_{b}R^{a},
3) C₁-C₁₀ alkyl,
4) aryl,
5) C₂-C₁₀ alkenyl,
6) C₂-C₁₀ alkynyl,
7) heterocyclyl,
8) C₃-C₈ cycloalkyl,
9) SO₂R^{a}, and
10) (C═O)NR^{b}₂,
said alkyl, cycloalkyl, aryl, heterocylyl, alkenyl, and alkynyl is optionally substituted with one or more substituents selected from R^{z}, or
R⁵ and R⁶ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};
R⁷ is independently selected from:
1) (C═O)ₐO_{b}C₁-C₁₀ alkyl,
2) (C═O)ₐO_{b}aryl,
3) C₂-C₁₀ alkenyl,
4) C₂-C₁₀ alkynyl,
5) (C═O)ₐO_{b} heterocyclyl,
6) (C═O)ₐO_{b}C₃-C₈ cycloalkyl,
7) CO₂H,
8) halo,
9) CN,
10) OH,
11) O_{b}C₁-C₆ perfluoroalkyl,
12) Oₐ(C═O)_{b}NR⁵R⁶,
13) NR⁵(C═O)NR⁵R⁶,
14) S(O)ₘR^{a},
15) S(O)₂NR⁵R⁶,
16) NR⁵S(O)ₘR^{a},
17) oxo,
18) CHO,
19) NO₂,
20) O(C═O)O_{b}C₁-C₁₀ alkyl, and
21) O(C═O)O_{b}C₃-C₈ cycloalkyl,
said alkyl, aryl, alkenyl, alkynyl, heterocyclyl, and cycloalkyl optionally substituted with one or more substituents selected from R^{z};
R^{z} is selected from:
1) (C═O)ᵣOₛ(C₁-C₁₀)alkyl,
2) Oᵣ(C₁-C₃)perfluoroalkyl,
3) (C₀-C₆)alkylene-S(O)ₘR^{a},
4) oxo,
5) OH,
6) halo,
7) CN,
8) (C═O)ᵣOₛ(C₂-C₁₀)alkenyl,
9) (C═O)ᵣOₛ(C₂-C₁₀)alkynyl,
10) (C═O)ᵣOₛ(C₃-C₆)cycloalkyl,
11) (C═O)ᵣOₛ(C₀-C₆)alkylene-aryl,
12) (C═O)ᵣOₛ(C₀-C₆)alkylene-heterocyclyl,
13) (C═O)ᵣOₛ(C₀-C₆)alkylene-N(R^{b})₂,
14) C(O)R^{a},
15) (C₀-C₆)alkylene-CO₂R^{a},
16) C(O)H,
17) (C₀-C₆)alkylene-CO₂H,
21) C(O)N(R^{b})₂,
22) S(O)ₘR^{a},
23) S(O)₂N(R^{b})₂
21) NR^{c}(C═O)O_{b}R^{a},
22) O(C═O)O_{b}C₁-C₁₀ alkyl,
23) O(C═O)O_{b}C₃-C₈ cycloalkyl,
24) O(C═O)O_{b}aryl, and
25) O(C═O)O_{b}-heterocycle,
said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;
R^{a} is substituted or unsubstituted (C₁-C₆)alkyl, substituted or unsubstituted (C₂-C₆)alkenyl, substituted or unsubstituted (C₂-C₆)alkynyl, substituted or unsubstituted (C₃-C₆)cycloalkyl, substituted or unsubstituted aryl, (C₁-C₆)perfluoroalkyl, 2,2,2-trifluoroethyl, or substituted or unsubstituted heterocyclyl; and
R^{b} is H, (C₁-C₆)alkyl, substituted or unsubstituted aryl, substituted or unsubstituted benzyl, substituted or unsubstituted heterocyclyl, (C₃-C₆)cycloalkyl, (C═O)OC₁-C₆ alkyl, (C═O)C₁-C₆ alkyl or S(O)₂R^{a};
R^{c} is selected from:
1) H,
2) C₁-C₁₀ alkyl,
3) aryl,
4) C₂-C₁₀ alkenyl,
5) C₂-C₁₀ alkynyl,
6) heterocyclyl,
7) C₃-C₈ cycloalkyl,
8) C₁-C₆ perfluoroalkyl,
said alkyl, cycloalkyl, aryl, heterocylyl, alkenyl, and alkynyl is optionally substituted with one or more substituents selected from R^{z},
or a pharmaceutically acceptable salt or a stereoisomer thereof.

53. The method according to claim 1 wherein the inhibitor of Akt is selected from:
N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide;
N-[2-(diethylamino)ethyl]-2-(4-{[4-(2-oxo-2,3-dihydro-1H-benzimidazol-1-yl)piperidin-1-yl]methyl}phenyl)-3-phenylquinoxaline-6-carboxamide;
N′-(7-Cyclobutyl-3-phenyl-[1,2,4]triazolo[4,3-b]pyridazin-6-yl)-2,2,N,N-tetramethyl-propane-1,3-diamine;
N′-(7-Cyclobutyl-3-(3,5-difluoro-phenyl)-[1,2,4]triazolo[4,3-b]pyridazin-6-yl)-2,2,N,N-tetramethyl-propane-1,3-diamine;
N′-(7-Cyclobutyl-3-(3,4-difluoro-phenyl)-[1,2,4]triazolo[4,3-b]pyridazin-6-yl)-2,2,N,N-tetramethyl-propane-1,3-diamine;
N′-(7-Cyclobutyl-3-(4-fluoro-phenyl)-[1,2,4]triazolo[4,3-b]pyridazin-6-yl)-2,2,N,N-tetramethyl-propane-1,3-diamine;
N′-(7-Cyclobutyl-3-(3-fluoro-phenyl)-[1,2,4]triazolo[4,3-b]pyridazin-6-yl)-2,2,N,N-tetramethyl-propane-1,3-diamine;
2,2,N,N-tetramethyl-N-(3-phenyl-[1,2,4]triazolo[3,4-a]phthalazin-6-yl)-propane-1,3-diamine;
N′-[3-(4-Methoxy-phenyl)-[1,2,4]triazolo[4,3-a]phthalazin-6-yl)-2,2,N,N-tetramethyl-propane-1,3-diamine;
6-(2-hydroxyethyl)oxy-3,7-diphenyl-[1,2,4]triazolo[4,3-b]pyridazine;
6-(4-hydroxybutyl)oxy-3,7-diphenyl-[1,2,4]triazolo[4,3-b]pyridazine;
2-(2-aminoprop-2-ylphenyl)-3-phenylquinazoline;
1-{1-[4-(7-Phenyl-1H-imidazo[4,5-g]quinoxalin-6-yl)benzyl]piperidin-4-yl}-1,3-dihydro-2H-benzimidazol-2-one;
1-{1-[4-(6-Hydroxy-5-isobutyl-3-phenylpyrazin-2-yl)benzyl]piperidin-4-yl}-1,3-dihydro-2H-benzimidazol-2-one;
1-{1-[4-(5-Hydroxy-6-isobutyl-3-phenylpyrazin-2-yl)benzyl]piperidin-4-yl}-1,3-dihydro-2H-benzimidazol-2-one;
1-(1-{4-[5-Hydroxy-6-(1H-indol-3-ylmethyl)-3-phenylpyrazin-2-yl]benzyl}piperidin-4-yl)-1,3-dihydro-2H-benzimidazol-2-one;
1-(1-{4-[6-Hydroxy-5-(1H-indol-3-ylmethyl)-3-phenylpyrazin-2-yl]benzyl}piperidin-4-yl)-1,3-dihydro-2H-benzimidazol-2-one;
1-{1-[4-(3-Phenylquinoxalin-2-yl)benzyl]piperidin-4-yl}-1,3-dihydro-2H-benzimidazol-2-one;
3-(4-{[4-(2-Oxo-2,3-dihydro-1H-benzamidazol-1-yl)piperdin-1-yl]methyl}phenyl)-2-phenylquinaxoline-6-carboxylic acid;
2-(4-{[4-(2-Oxo-2,3-dihydro-1H-benzamidazol-1-yl)piperdin-1-yl]methyl}phenyl)-2-phenylquinaxoline-6-carboxylic acid;
N-[3-(1H-Imidazol-1-yl)propyl]-3-(4-{[4-(2-oxo-2,3-dihydro-1H-benzamidazol-1-yl)piperdin-1-yl]methyl}phenyl)-2-phenylquinaxoline-6-carboxamide;
1-{1-[4-(3-phenylpyrido[3,4-b]pyrazin-2-yl)benzyl]piperidin-4-yl}-1,3-dihydro-2H-benzimidazol-2-one;
1-{1-[4-(2-phenylpyrido[3,4-b]pyrazin-3-yl)benzyl]piperidin-4-yl}-1,3-dihydro-2H-benzimidazol-2-one;
4-cyano-N-{(3R)-1-[4-(3-phenylquinoxalin-2-yl)benzyl]pyrrolidin-3-yl}benzamide;
N-{(3R)-1-[4-(3-phenylquinoxalin-2-yl)benzyl]pyrrolidin-3-yl}-1,3-thiazole-5-carboxamide;
2-(4-{[4-(6-amino-9H-purin-9-yl)piperidin-1-yl]methyl}phenyl)-3-phenylquinoxalin-6-amine;
9-{1-[4-(3-phenylpyrido[3,4-b]pyrazin-2-yl)benzyl]piperidin-4-yl}-9H-purin-6-amine;
9-{1-[4-(3-phenylpyrido[2,3-b]pyrazin-2-yl)benzyl]piperidin-4-yl}-9H-purin-6-amine;
2-(4-{[4-(6-amino-9H-purin-9-yl)piperidin-1-yl]methyl}phenyl)-3-phenylquinoxaline-6-carboxylic acid;
1-{1-[4-(3-phenylquinolin-2-yl)benzyl]piperidin-4-yl}-1,3-dihydro-2H-benzimidazol-2-one;
1-(1-{4-[3-phenyl-6-(1H-tetrazol-5-yl)quinoxalin-2-yl]benzyl}piperidin-4-yl)-1,3-dihydro-2H-benzimidazol-2-one;
1-(1-{4-[3-phenyl-7-(1H-tetrazol-5-yl)quinoxalin-2-yl]benzyl}piperidin-4-yl)-1,3-dihydro-2H-benzimidazol-2-one;
9-(1-{4-[3-phenyl-7-(1H-tetrazol-5-yl)quinoxalin-2-yl]benzyl}piperidin-4-yl)-9H-purin-6-amine; and
9-(1-{4-[3-phenyl-6-(1H-tetrazol-5-yl)quinoxalin-2-yl]benzyl}piperidin-4-yl)-9H-purin-6-amine;
or a pharmaceutically acceptable salt or a stereoisomer thereof.

54. The method according to claim 53 wherein the inhibitor of Akt is selected from:
N-[2-(diethylamino)ethyl]-3-(4-{[4-(2-oxo-2,3-dihydro-1H-benzimidazol-1-yl)piperidin-1-yl]methyl}phenyl)-2-phenylquinoxaline-6-carboxamide;
N-[2-(diethylamino)ethyl]-2-(4-{[4-(2-oxo-2,3-dihydro-1H-benzimidazol-1-yl)piperidin-1-yl]methyl}phenyl)-3-phenylquinoxaline-6-carboxamide;
4-cyano-N-{(3R)-1-[4-(3-phenylquinoxalin-2-yl)benzyl]pyrrolidin-3-yl}benzamide;
N-{(3R)-1-[4-(3-phenylquinoxalin-2-yl)benzyl]pyrrolidin-3-yl}-1,3-thiazole-5-carboxamide;
2-(4-{[4-(6-amino-9H-purin-9-yl)piperidin-1-yl]methyl}phenyl)-3-phenylquinoxalin-6-amine;
9-{1-[4-(3-phenylpyrido[3,4-b]pyrazin-2-yl)benzyl]piperidin-4-yl}-9H-purin-6-amine;
9-{1-[4-(3-phenylpyrido[2,3-b]pyrazin-2-yl)benzyl]piperidin-4-yl}-9H-purin-6-amine;
2-(4-{[4-(6-amino-9H-purin-9-yl)piperidin-1-yl]methyl}phenyl)-3-phenylquinoxaline-6-carboxylic acid;
1-{1-[4-(3-phenylquinolin-2-yl)benzyl]piperidin-4-yl}-1,3-dihydro-2H-benzimidazol-2-one;
1-(1-{4-[3-phenyl-6-(1H-tetrazol-5-yl)quinoxalin-2-yl]benzyl}piperidin-4-yl)-1,3-dihydro-2H-benzimidazol-2-one;
1-(1-{4-[3-phenyl-7-(1H-tetrazol-5-yl)quinoxalin-2-yl]benzyl}piperidin-4-yl)-1,3-dihydro-2H-benzimidazol-2-one;
9-(1-{4-[3-phenyl-7-(1H-tetrazol-5-yl)quinoxalin-2-yl]benzyl}piperidin-4-yl)-9H-purin-6-amine; and
9-(1-{4-[3-phenyl-6-(1H-tetrazol-5-yl)quinoxalin-2-yl]benzyl}piperidin-4-yl)-9H-purin-6-amine;
or a pharmaceutically acceptable salt or a stereoisomer thereof.

55. The method according to claim 1 wherein the inhibitor of a protein kinase inhibits a protein kinase selected from the group comprising an RTK, a CTK or an STK.

56. The method according to claim 55 wherein the protein kinase is an RTK.

57. The method according to claim 56 wherein the RTK is selected from the group comprising EGF, HER2, HER3, HER4, IR, IGF-1R, IRR, PDGFRα, PDGFRβ, TrkA, TrkB, TrkC, HGF, CSFIR, C-Kit, C-fms, Flk-1R, Flk4, KDR/Flk-1, Flt-1, FGFR-1R, FGFR-1R, FGFR-3R and FGFR-4R.

58. The method according to claim 57 wherein the RTK is selected from IR, IGF-1R, and IRR.

59. The method according to claim 1 wherein the inhibitor of a protein kinase is selected from a small molecule compound, an antibody and an antisense molecule.

60. The method according to claim 59 wherein the inhibitor of a protein kinase is selected from a small molecule compound and an antibody.

61. The method according to claim 59 wherein the inhibitor of a protein kinase is a small molecule compound.

62. The method according to claim 59 wherein the inhibitor of a protein kinase is a herceptin antibody.

63. The method according to claim 1 wherein the inhibitor of a protein kinase is selected from a compound of the formula XI: 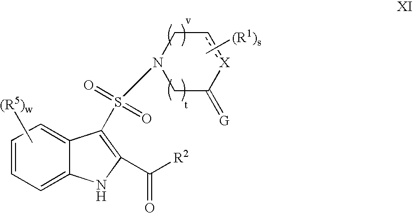
wherein:
----- represents an optional double bond;
X is C, N, S(O)ₘ or O;
G is H₂ or O;
R^{a} is independently selected from:
1) H,
2) C₁-C₆ alkyl,
3) Halogen,
4) Aryl,
5) Heterocycle,
6) C₃-C₁₀ cycloalkyl, and
7) OR⁴;
said alkyl, aryl, heterocycle and cycloalkyl is optionally substituted with at least one substituent selected from R⁷;
R¹ is independently selected from:
1) H,
2) (CR^{a}₂)ₙR⁶,
3) (CR^{a}₂)ₙC(O)R⁴,
4) C(O)N(R⁴)₂,
5) (CR^{a}₂)ₙOR⁴,
6) (CR^{a}₂)ₙN(R⁴)₂,
7) S(O)ₘR⁶,
8) S(O)ₘR⁶OR⁴,
9) C(O)N(R⁴)(CR^{a}₂)ₙR⁶,
10) C(O)N(R⁴)(CR^{a}₂)ₙOR⁴,
11) C(O)R⁶(CR^{a}₂)ₙR⁶,
12) C(O)N(R⁴)(CR^{a}₂)ₙS(O)ₘ(CR^{a}₂)ₙR⁶,
13) C(O)N(R⁴)(CR^{a}₂)ₙC(O)R⁶,
14) C(O)N(R⁴)(CR^{a}₂)ₙN(R⁴)₂,
15) Halogen,
16) N(R⁴)S(O)ₘR⁶,
17) (CR^{a}₂)ₙC(O)OR⁴, and
18) R⁶C(O)OR;
R² is:
1) H,
2) Unsubstituted or substituted C₁-C₁₀ alkyl,
3) N(R⁴)₂, or
4) OR⁴;
R⁴ is independently selected from:
1) H,
2) C₁-C₆ alkyl,
3) C₃-C₁₀ cycloalkyl,
4) Aryl,
5) Heterocycle,
6) CF₃,
7) C₂-C₆ alkenyl, and
8) C₂-C₆ alkynyl;
said alkyl, cycloalkyl, aryl, heterocycle, alkenyl and alkynyl is optionally substituted with at least one substituent selected from R⁷;
R⁵ is independently selected from:
1) H,
2) Halogen,
3) NO₂,
4) CN,
5) CR⁴═C(R⁴)₂,
6) C≡CR⁴,
7) (CR^{a}₂)ₙOR⁴,
8) (CR^{a}₂)ₙN(R⁴)₂,
9) C(O)R⁴,
10) C(O)OR⁴,
11) (CR^{a}₂)ₙR⁴,
12) S(O)ₘR⁶,
13) S(O)ₘN(R⁴)₂,
14) OS(O)ₘR⁶,
15) N(R⁴)C(O)R⁴,
16) N(R⁴)S(O)ₘR⁶,
17) (CR^{a}₂)ₙN(R⁴)R⁶,
18) (CR^{a}₂)ₙN(R⁴)R⁶OR⁴,
19) (CR^{a}₂)ₙN(R⁴)(CR^{a}₂)ₙC(O)N(R⁴)₂,
20) N(R⁴)(CR^{a}₂)ₙR⁶,
21) N(R⁴)(CR^{a}₂)ₙN(R⁴)₂, and
22) (CR^{a}₂)ₙC(O)N(R⁴)₂;
R⁶ is independently selected from:
1) C₁-C₆ alkyl,
2) Aryl,
3) Heterocycle, and
4) C₃-C₁₀ cycloalkyl;
said alkyl, aryl, heterocycle and cycloalkyl is optionally substituted with at least one substituent of R⁷;
R⁷ is independently selected from:
1) Unsubstituted or substituted C₁-C₆ alkyl,
2) Halogen,
3) OR⁴,
4) CF₃,
5) Unsubtituted or substituted aryl,
6) Unsubstituted or substituted C₃-C₁₀ cycloalkyl,
7) Unsubstituted or substituted heterocycle,
8) S(O)ₘN(R⁴)₂,
9) C(O)OR⁴,
10) C(O)R⁴,
11) CN,
12) C(O)N(R⁴)₂,
13) N(R⁴)C(O)R⁴,
14) S(O)ₘR⁶, and
15) NO₂;
m is independently 0, 1 or 2;
n is independently 0, 1, 2, 3, 4, 5 or 6;
s is 0 to 6;
t is 0, 1, or 2;
v is 0, 1 or 2;
w is 0, 1, 2, 3 or 4;
or a pharmaceutically acceptable salt or stereoisomer thereof.

64. The method according to claim 1 wherein the inhibitor of a protein kinase selected from the formula XI is:
5-Chloro-3-(morpholin-4-ylsulfonyl)-1H-indole-2-carboxamide;
5-Bromo-3-(morpholin-4-ylsulfonyl)-1H-indole-2-carboxamide;
5-Iodo-3-(morpholin-4-ylsulfonyl)-1H-indole-2-carboxamide;
5-Methoxy-3-(morpholin-4-ylsulfonyl)-1H-indole-2-carboxamide;
6-Methoxy-3-(morpholin-4-ylsulfonyl)-1H-indole-2-carboxamide;
5-(Methylsulfonyl)-3-(morpholin-4-ylsulfonyl)-1H-indole-2-carboxamide;
7-Amino-3-(morpholin-4-ylsulfonyl)-1H-indole-2-carboxamide;
3-(Morpholin-4-ylsulfonyl)-5-nitro-1H-indole-2-carboxamide;
5-Chloro-3-(piperazin-1-ylsulfonyl)-1H-indole-2-carboxamide;
3-[(4-Benzylpiperazin-1-yl)sulfonyl]-5-chloro-1H-indole-2-carboxamide;
3-[(4-Acetylpiperazin-1-yl)sulfonyl]-5-chloro-1H-indole-2-carboxamide;
5-Chloro-3-(piperidin-1-ylsulfonyl)-1H-indole-2-carboxamide;
5-Chloro-3-(pyrrolidin-1-ylsulfonyl)-1H-indole-2-carboxamide;
5-Chloro-3-(thiomorpholin-4-ylsulfonyl)-1H-indole-2-carboxamide;
3-(Azetidin-1-ylsulfonyl)-5-chloro-1H-indole-2-carboxamide;
5-Chloro-3-[(oxidothiomorpholin-4-yl)sulfonyl]-1H-indole-2-carboxamide;
5-Chloro-3-[(1,1-dioxidothiomorpholin-4-yl)sulfonyl]-1H-indole-2-carboxamide;
cis-5-Chloro-3-(2,6-dimethylmorpholin-4-ylsulfonyl)-1H-indole-2-carboxamide;
trans-5-Chloro-3-(2,6-dimethylmorpholin-4-ylsulfonyl)-1H-indole-2-carboxamide;
5-Chloro-3-[(3-hydroxyazetidin-1-yl)sulfonyl]-1H-indole-2-carboxamide;
(±)-5-Chloro-3-{[2-(phenoxymethyl)morpholin-4-yl]sulfonyl}-1H-indole-2-carboxamide;
(S)-5-Chloro-3-{[2-(phenoxymethyl)morpholin-4-yl]sulfonyl}-1H-indole-2-carboxamide;
(R)-5-Chloro-3-{[2-(phenoxymethyl)morpholin-4-yl]sulfonyl}-1H-indole-2-carboxamide;
5-Bromo-3-({4-[2-(dimethylamino)ethyl]-5-oxo-1,4-diazepan-1-yl}sulfonyl)-1H-indole-2-carboxamide;
5-Bromo-3-({5-oxo-1,4-diazepan-1-yl}sulfonyl)-1H-indole-2-carboxamide;
5-Bromo-3-[(3-oxopiperazin-1-yl)sulfonyl]-1H-indole-2-carboxamide;
5-Bromo-3-[(3-hydroxyazetidin-1-yl)sulfonyl]-1H-indole-2-carboxamide;
(±)-5-Bromo-3-{[2-(aminocarbonyl)morpholin-4-yl]sulfonyl}-1H-indole-2-carboxamide;
3-(Azetidin-1-ylsulfonyl)-5-bromo-1H-indole-2-carboxamide;
5-Bromo-3-({4-[(4-methoxyphenyl)sulfonyl]piperazin-1-yl}sulfonyl)-1H-indole-2-carboxamide;
5-Bromo-3-({4-[(4-bromophenyl)sulfonyl]piperazin-1-yl}sulfonyl)-1H-indole-2-carboxamide;
5-Bromo-3-{[4-(3-morpholin-4-ylpropyl)-3-oxopiperazin-1-yl]sulfonyl}-1H-indole-2-carboxamide;
5-Bromo-3-({4-[3-(dimethylamino)propyl]-3-oxopiperazin-1-yl}sulfonyl)-1H-indole-2-carboxamide;
5-Bromo-3-(2,5-dihydroxy-1H-pyrrol-1-ylsulfonyl)-1H-indole-2-carboxamide;
5-Bromo-3-(6-oxa-3-azabicyclo[3.1.0]hex-3-ylsulfonyl)-1H-indole-2-carboxamide;
(±)-5-Bromo-3-{[2-(phenoxymethyl)morpholino-4-yl]sulfonyl}-1H-indole-2-carboxamide;
(S)-5-Bromo-3-{[2-(phenoxymethyl)morpholino-4-yl]sulfonyl}-1H-indole-2-carboxamide;
(R)-5-Bromo-3-{[2-(phenoxymethyl)morpholin-4-yl]sulfonyl}-1H-indole-2-carboxamide;
6-Hydroxy-3-(morpholin-4-ylsulfonyl)-1H-indole-2-carboxamide;
3-(Morpholin-4-ylsulfonyl)-1H-indole-2-carboxamide;
5-(2-Furyl)-3-(morpholin-4-ylsulfonyl)-1H-indole-2-carboxamide;
3-(Morpholin-4-ylsulfonyl)-5-(phenylethynyl)-1H-indole-2-carboxamide;
3-(Morpholin-4-ylsulfonyl)-5-(2-phenylethyl)-1H-indole-2-carboxamide;
5-Hex-1-ynyl-3-(morpholin-4-ylsulfonyl)-1H-indole-2-carboxamide;
5-Hexyl-3-(morpholin-4-ylsulfonyl)-1H-indole-2-carboxamide;
Methyl 2-(aminocarbonyl)-3-(morpholin-4-ylsulfonyl)-1H-indole-5-carboxylate;
3-(Morpholin-4-ylsulfonyl)-5-vinyl-1H-indole-2-carboxamide;
5-Hydroxy-3-(morpholin-4-ylsulfonyl)-1H-indole-2-carboxamide;
5-Ethoxy-3-(morpholin-4-ylsulfonyl)-1H-indole-2-carboxamide;
3-(Morpholin-4-ylsulfonyl)-5-propoxy-1H-indole-2-carboxamide;
5-Isopropoxy-3-(morpholin-4-ylsulfonyl)-1H-indole-2-carboxamide;
5-Ethyl-3-(morpholin-4-ylsulfonyl)-1H-indole-2-carboxamide;
2-(Aminocarbonyl)-3-(morpholin-4-ylsulfonyl)-1H-indol-5-yl methanesulfonate;
3-(Morpholin-4-ylsulfonyl)-5-prop-1-ynyl-1H-indole-2-carboxamide;
3-(Morpholin-4-ylsulfonyl)-5-thien-2-yl-1H-indole-2-carboxamide;
3-(Azetidin-1-ylsulfonyl)-5-methoxy-1H-indole-2-carboxamide;
5-Formyl-3-(morpholin-4-ylsulfonyl)-1H-indole-2-carboxamide;
5-Methyl-3-(morpholin-4-ylsulfonyl)-1H-indole-2-carboxamide;
7-(Acetylamino)-3-(morpholin-4-ylsulfonyl)-1H-indole-2-carboxamide;
7-[(Methylsulfonyl)amino]-3-(morpholin-4-ylsulfonyl)-1H-indole-2-carboxamide;
5-{[(4-Methoxyphenyl)amino]methyl}-3-morpholino-4-ylsulfonyl)-1H-indole-2-carboxamide;
5-{[(2-Acetamide)amino]methyl}-3-morpholino-4-ylsulfonyl)-1H-indole-2-carboxamide;
3-(Morpholino-4-ylsulfonyl)-5-phenyl-1H-indole-2-carboxamide;
3-(Morpholino-4-ylsulfonyl)-5-pyrazin-2-yl-1H-indole-2-carboxamide;
3-(Morpholino-4-ylsulfonyl)-5-pyridin-2-yl-1H-indole-2-carboxamide;
3-(Morpholino-4-ylsulfonyl)-5-pyridin-4-yl-1H-indole-2-carboxamide;
5-(1-Benzofuran-2-yl)-3-(morpholino-4-ylsulfonyl)-1H-indole-2-carboxamide;
5-(5-Methyl-2-furyl)-3-(morpholino-4-ylsulfonyl)-1H-indole-2-carboxamide;
5-(3,5-Dimethylisoxazole-4-yl)-3-(morpholino-4-ylsulfonyl)-1H-indole-2-carboxamide;
3-(Morpholin-4-ylsulfonyl)-5-(1H-pyrrol-2-yl)-1H-indole-2-carboxamide;
3-(Morpholin-4-ylsulfonyl)-5-pyridin-3-yl-1H-indole-2-carboxamide;
3-(Morpholin-4-ylsulfonyl)-5-(1,3-thiazol-2-yl)-1H-indole-2-carboxamide;
3-(Morpholin-4-ylsulfonyl)-5-thien-3-yl-1H-indole-2-carboxamide;
5-(1-Benzothien-3-yl)-3-(morpholin-4-ylsulfonyl)-1H-indole-2-carboxamide;
3-(Azetidin-1-yl}sulfonyl)-5-iodo-1H-indole-2-carboxamide;
3-[(3-Hydroxyazetidin-1-yl)sulfonyl]-5-iodo-1H-indole-2-carboxamide;
(±)-5-Iodo-3-{[2-(phenoxymethyl)morpholino-4-yl]sulfonyl}-1H-indole-2-carboxamide;
(S)-5-Iodo-3-{[2-(phenoxymethyl)morpholino-4-yl]sulfonyl}-1H-indole-2-carboxamide;
(R)-5-Iodo-3-{[2-(phenoxymethyl)morpholin-4-yl]sulfonyl}-1H-indole-2-carboxamide;
7-Amino-6-bromo-3-(morpholin-4-ylsulfonyl)-1H-indole-2-carboxamide;
7-Amino-4,6-dibromo-3-(morpholin-4-ylsulfonyl)-1H-indole-2-carboxamide;
6-Bromo-7-(dimethylamino)-3-(morpholin-4-ylsulfonyl)-1H-indole-2-carboxamide;
3-(Morpholin-4-ylsulfonyl)-7-[(pyridin-4-ylmethyl)amino]-1H-indole-2-carboxamide;
7-{[(2-Chloropyridin-4-yl)methyl]amino}-3-(morpholin-4-ylsulfonyl)-1H-indole-2-carboxamide;
7-Nitro-3-{[(2S)-2-(phenoxymethyl)morpholin-4-yl]sulfonyl 1-1H-indole-2-carboxamide;
7-Amino-3-{[(2S)-2-(phenoxymethyl)morpholin-4-yl]sulfonyl}-1H-indole-2-carboxamide;
3-{[(2S)-2-(Phenoxymethyl)morpholin-4-yl]sulfonyl}-7-[(pyridin-4-ylmethyl)amino]-1H-indole-2-carboxamide;
7-(Benzylamino)-3-{[(2S)-2-(phenoxymethyl)morpholin-4-yl]sulfonyl}-1H-indole-2-carboxamide;
7-Chloro-3-(morpholin-4-ylsulfonyl)-1H-indole-2-carboxamide;
6-Bromo-3-(morpholin-4-ylsulfonyl)-1H-indole-2-carboxamide;
7-Bromo-3-(morpholin-4-ylsulfonyl)-1H-indole-2-carboxamide;
7-Cyano-3-(morpholin-4-ylsulfonyl)-1H-indole-2-carboxamide;
(±)-7-(Methylsulfinyl)-3-(morpholin-4-ylsulfonyl)-1H-indole-2-carboxamide;
7-Aminomethyl-3-(morpholin-4-ylsulfonyl)-1H-indole-2-carboxamide;
5-Amino-3-(morpholin-4-ylsulfonyl)-1H-indole-2-carboxamide;
(S)-5-Fluoro-3-{[2-(phenoxymethyl)morpholino-4-yl]sulfonyl}-1H-indole-2-carboxamide;
(R)-5-Fluoro-3-{[2-(phenoxymethyl)morpholin-4-yl]sulfonyl}-1H-indole-2-carboxamide;
5-Acetylamino-3-(morpholin-4-ylsulfonyl)-1H-indole-2-carboxamide;
5-[(Methylsulfonyl)amino]-3-(morpholin-4-ylsulfonyl)-1H-indole-2-carboxamide;
3-(Morpholin-4-ylsulfonyl)-5-[(trifluoroacetyl)amino]-1H-indole-2-carboxamide;
5-[(2-Aminoethyl)amino]-3-(morpholin-4-ylsulfonyl)-1H-indole-2-carboxamide;
5-(Dimethylamino)-3-(morpholin-4-ylsulfonyl)-1H-indole-2-carboxamide;
4,5-Dibromo-3-(morpholin-4-ylsulfonyl)-1H-indole-2-carboxamide;
5,6-Dibromo-3-(morpholin-4-ylsulfonyl)-1H-indole-2-carboxamide;
5-Bromo-4-nitro-3-(morpholin-4-ylsulfonyl)-1H-indole-2-carboxamide;
5-Bromo-6-nitro-3-(morpholin-4-ylsulfonyl)-1H-indole-2-carboxamide;
5-Bromo-6-amino-3-(morpholin-4-ylsulfonyl)-1H-indole-2-carboxamide;
5-Bromo-4-amino-3-(morpholin-4-ylsulfonyl)-1H-indole-2-carboxamide;
5-Bromo-3-({2-[(cyclohexylamino)carbonyl]morpholin-4-yl}sulfonyl)-1H-indole-2-carboxamide;
5-Bromo-3-({2-[(2,3-dihydro-1H-inden-1-ylamino)carbonyl]morpholin-4-yl}sulfonyl)-1H-indole-2-carboxamide;
5-Bromo-3-[(2-{[(2-phenylethyl)amino]carbonyl}morpholin-4-yl)sulfonyl]-1H-indole-2-carboxamide;
5-Bromo-3-[(2-{[(3-phenylpropyl)amino]carbonyl}morpholin-4-yl)sulfonyl]-1H-indole-2-carboxamide;
5-Bromo-3-[(2-{[(3,3-diphenylpropyl)amino]carbonyl}morpholin-4-yl)sulfonyl]-1H-indole-2-carboxamide;
5-Bromo-3-{[2-(3,4-dihydroisoquinolin-2(1H)-ylcarbonyl)morpholin-4-yl]sulfonyl}-1H-indole-2-carboxamide;
5-Bromo-3-[(2-{[(2-phenoxyethyl)amino]carbonyl}morpholin-4-yl)sulfonyl]-1H-indole-2-carboxamide;
3-({2-[(3-Benzylpyrrolidin-1-yl)carbonyl]morpholin-4-yl}sulfonyl)-5-bromo-1H-indole-2-carboxamide;
5-Bromo-3-[(2-{[(1,2,3,4-tetrahydronaphthalen-2-ylmethyl)amino]carbonyl}morpholin-4-yl)sulfonyl]-1H-indole-2-carboxamide;
3-({2-[(Benzylamino)carbonyl]morpholin-4-yl}sulfonyl)-5-bromo-1H-indole-2-carboxamide;
5-Bromo-3-{[2-({[3-(trifluoromethyl)benzyl]amino}carbonyl)morpholin-4-yl]sulfonyl}-1H-indole-2-carboxamide;
5-Bromo-3-[(2-{[(2,2-diphenylethyl)amino]carbonyl}morpholin-4-yl)sulfonyl]-1H-indole-2-carboxamide;
5-Bromo-3-({2-[(2,3-dihydro-1H-inden-2-ylamino)carbonyl]morpholin-4-yl}sulfonyl)-1H-indole-2-carboxamide;
7-{[2-(Aminocarbonyl)-5-bromo-1H-indol-3-yl]sulfonyl}-2-benzyl-7-aza-2-azoniaspiro[4.4]nonane;
5-Bromo-3-{[2-({[(5-methylpyrazin-2-yl)methyl]amino}carbonyl)morpholin-4-yl]sulfonyl}-1H-indole-2-carboxamide;
3-({[(4-{[2-(Aminocarbonyl)-5-bromo-1H-indol-3-yl]sulfonyl}morpholin-2-yl)carbonyl]amino}methyl)pyridine;
5-Bromo-3-[(2-{[(1-phenylethyl)amino]carbonyl}morpholin-4-yl)sulfonyl]-1H-indole-2-carboxamide;
1-(3-{[(4-{[2-(Aminocarbonyl)-5-bromo-1H-indol-3-yl]sulfonyl}morpholin-2-yl)carbonyl]amino}propyl)-1H-imidazole;
5-Bromo-3-{[2-({[(1R)-1-phenylethyl]amino}carbonyl)morpholin-4-yl]sulfonyl}-1H-indole-2-carboxamide;
5-Bromo-3-[(2-{[(2-phenylpropyl)amino]carbonyl}morpholin-4-yl)sulfonyl]-1H-indole-2-carboxamide;
3-[(2-{[Benzyl(methyl)amino]carbonyl}morpholin-4-yl)sulfonyl]-5-bromo-1H-indole-2-carboxamide;
1-[(4-{[2-(Aminocarbonyl)-5-bromo-1H-indol-3-yl]sulfonyl}morpholin-2-yl)carbonyl]-4-benzylpiperazine;
2-({[(4-{[2-(Aminocarbonyl)-5-bromo-1H-indol-3-yl]sulfonyl}morpholin-2-yl)carbonyl]amino}methyl)pyridine;
5-Bromo-3-{[2-({[2-(tert-butylthio)ethyl]amino}carbonyl)morpholin-4-yl]sulfonyl}-1H-indole-2-carboxamide;
3-({2-[(Benzhydrylamino)carbonyl]morpholin-4-yl}sulfonyl)-5-bromo-1H-indole-2-carboxamide;
5-Bromo-3-{[2-({[(2S)-2-phenylcyclopropyl]amino}carbonyl)morpholin-4-yl]sulfonyl}-1H-indole-2-carboxamide;
5-Bromo-3-({2-[(3-phenylpyrrolidin-1-yl)carbonyl]morpholin-4-yl}sulfonyl)-1H-indole-2-carboxamide;
5-Bromo-3-({2-[(4,4-diphenylpiperidin-1-yl)carbonyl]morpholin-4-yl}sulfonyl)-1H-indole-2-carboxamide;
5-Bromo-3-[(2-{[(2,3-dihydro-1H-inden-2-ylmethyl)amino]carbonyl}morpholin-4-yl)sulfonyl]-1H-indole-2-carboxamide;
5-Bromo-3-({2-[(2,3-dihydro-1H-inden-1-ylamino)carbonyl]morpholin-4-yl}sulfonyl)-1H-indole-2-carboxamide;
5-Bromo-3-({2-[(2,3-dihydro-1H-inden-1-ylamino)carbonyl]morpholin-4-yl}sulfonyl)-1H-indole-2-carboxamide;
5-Bromo-3-({2-[(3-pyridin-4-ylpyrrolidin-1-yl)carbonyl]morpholin-4-yl}sulfonyl)-1H-indole-2-carboxamide;
5-Bromo-3-[(2-{[(2-hydroxy-2,3-dihydro-1H-inden-1-yl)amino]carbonyl}morpholin-4-yl)sulfonyl]-1H-indole-2-carboxamide;
5-Bromo-3-({2-[(4-hydroxy-4-phenylpiperidin-1-yl)carbonyl]morpholin-4-yl}sulfonyl)-1H-indole-2-carboxamide;
3-{[2-(Anilinocarbonyl)morpholin-4-yl]sulfonyl}-5-bromo-1H-indole-2-carboxamide;
5-Bromo-3-[(2-{[(2-oxo-2-phenylethyl)amino]carbonyl}morpholin-4-yl)sulfonyl]-1H-indole-2-carboxamide;
5-Bromo-3-({2-[(neopentylamino)carbonyl]morpholin-4-yl}sulfonyl)-1H-indole-2-carboxamide;
5-Bromo-3-[(2-{[(1,2-diphenylethyl)amino]carbonyl}morpholin-4-yl)sulfonyl]-1H-indole-2-carboxamide;
5-Bromo-3-[(2-{[(4-chlorophenyl)amino]carbonyl}morpholin-4-yl)sulfonyl]-1H-indole-2-carboxamide;
5-Bromo-3-[(2-{[(4-phenoxyphenyl)amino]carbonyl}morpholin-4-yl)sulfonyl]-1H-indole-2-carboxamide;
5-Bromo-3-[(2-{[(4-tert-butylphenyl)amino]carbonyl}morpholin-4-yl)sulfonyl]-1H-indole-2-carboxamide;
5-Bromo-3-{[2-({[3-(2-oxopyrrolidin-1-yl)propyl]amino}carbonyl)morpholin-4-yl]sulfonyl}-1H-indole-2-carboxamide;
5-Bromo-3-[(2-{[(3-isopropoxypropyl)amino]carbonyl}morpholin-4-yl)sulfonyl]-1H-indole-2-carboxamide;
5-Bromo-3-[(2-{[(3-ethoxypropyl)amino]carbonyl}morpholin-4-yl)sulfonyl]-1H-indole-2-carboxamide;
5-Bromo-3-[(2-{[(2-cyclohex-1-en-1-ylethyl)amino]carbonyl}morpholin-4-yl)sulfonyl]-1H-indole-2-carboxamide;
5-Bromo-3-[(2-{[(2,2,3,3,4,4,4-heptafluorobutyl)amino]carbonyl}morpholin-4-yl)sulfonyl]-1H-indole-2-carboxamide;
5-Bromo-3-[(2-{[(3-isobutoxypropyl)amino]carbonyl}morpholin-4-yl)sulfonyl]-1H-indole-2-carboxamide;
5-Bromo-3-[(2-{[(3-butoxypropyl)amino]carbonyl}morpholin-4-yl)sulfonyl]-1H-indole-2-carboxamide;
5-Bromo-3-[(2-{[(2-thien-2-ylethyl)amino]carbonyl}morpholin-4-yl)sulfonyl]-1H-indole-2-carboxamide;
2-({[(4-{[2-(Aminocarbonyl)-5-bromo-1H-indol-3-yl]sulfonyl}morpholin-2-yl)carbonyl]amino}methyl)-1H-benzimidazole;
3-{[2-(Azepan-1-ylcarbonyl)morpholin-4-yl]sulfonyl}-5-bromo-1H-indole-2-carboxamide;
5-Bromo-3-({2-[({2-[(2,6-dichlorobenzyl)thio]ethyl}amino)carbonyl]morpholin-4-yl}sulfonyl)-1H-indole-2-carboxamide;
3-{[2-({[4-(Aminosulfonyl)benzyl]amino}carbonyl)morpholin-4-yl]sulfonyl}-5-bromo-1H-indole-2-carboxamide;
5-Bromo-3-{[2-(thiomorpholin-4-ylcarbonyl)morpholin-4-yl]sulfonyl}-1H-indole-2-carboxamide;
5-Bromo-3-[(2-{[(2-methoxyethyl)amino]carbonyl}morpholin-4-yl)sulfonyl]-1H-indole-2-carboxamide;
5-Bromo-3-[(2-{[(2-methoxy-1-methylethyl)amino]carbonyl}morpholin-4-yl)sulfonyl]-1H-indole-2-carboxamide;
5-Bromo-3-[(2-{[(1-ethylpropyl)amino]carbonyl}morpholin-4-yl)sulfonyl]-1H-indole-2-carboxamide;
5-Bromo-3-{[2-({[6-(dimethylamino)hexyl]amino}carbonyl)morpholin-4-yl]sulfonyl}-1H-indole-2-carboxamide;
5-Bromo-3-[(2-{[(tetrahydrofuran-2-ylmethyl)amino]carbonyl}morpholin-4-yl)sulfonyl]-1H-indole-2-carboxamide;
5-Bromo-3-[(2-{[(1-phenylcyclopropyl)amino]carbonyl}morpholin-4-yl)sulfonyl]-1H-indole-2-carboxamide;
5-Bromo-3-{[2-({[phenyl(pyridin-4-yl)methyl]amino}carbonyl)morpholin-4-yl]sulfonyl}-1H-indole-2-carboxamide;
5-Bromo-3-[(2-{[(dicyclopropylmethyl)amino]carbonyl}morpholin-4-yl)sulfonyl]-1H-indole-2-carboxamide;
5-Bromo-3-[(2-{[(1,4-dioxan-2-ylmethyl)amino]carbonyl}morpholin-4-yl)sulfonyl]-1H-indole-2-carboxamide;
5-Bromo-3-{[2-({methyl[2-(4-methylphenoxy)ethyl]amino}carbonyl)morpholin-4-yl]sulfonyl}-1H-indole-2-carboxamide;
5-Bromo-3-{[2-({[(1,1-dioxidotetrahydrothien-3-yl)methyl]amino}carbonyl)morpholin-4-yl]sulfonyl}-1H-indole-2-carboxamide;
5-Bromo-3-[(2-{[2-(2-phenylethyl)pyrrolidin-1-yl]carbonyl}morpholin-4-yl)sulfonyl]-1H-indole-2-carboxamide;
5-Bromo-3-[(2-{[(2-cyclohexylethyl)amino]carbonyl}morpholin-4-yl)sulfonyl]-1H-indole-2-carboxamide;
4-({[(4-{[2-(Aminocarbonyl)-5-bromo-1H-indol-3-yl]sulfonyl}morpholin-2-yl)carbonyl]amino}methyl)-1-methyl-1H-imidazole;
5-Bromo-3-[(2-{[(1,1-dioxidotetrahydrothien-3-yl)amino]carbonyl}morpholin-4-yl)sulfonyl]-1H-indole-2-carboxamide;
5-Bromo-3-[(2-{[(1-naphthylmethyl)amino]carbonyl}morpholin-4-yl)sulfonyl]-1H-indole-2-carboxamide;
5-Bromo-3-[(2-{[(imidazo[2,1-b][1,3]thiazol-6-ylmethyl)amino]carbonyl}morpholin-4-yl)sulfonyl]-1H-indole-2-carboxamide;
3-[(2-{[2-(1,3-Benzothiazol-2-yl)pyrrolidin-1-yl]carbonyl}morpholin-4-yl)sulfonyl]-5-bromo-1H-indole-2-carboxamide;
5-Chloro-3-({2-[(2-ethoxyphenoxy)methyl]morpholin-4-yl}sulfonyl)-1H-indole-2-carboxamide;
5-Chloro-3-[(1R,4R)-2-oxa-5-azabicyclo[2.2.1]hept-5-ylsulfonyl]-1H-indole-2-carboxamide;
7-{[2-(Aminocarbonyl)-5-chloro-1H-indol-3-yl]sulfonyl}-3-benzyl-9-thia-7-aza-3-azoniabicyclo[3.3.1]nonane;
5-Chloro-3-{[2-(1H-indol-4-yl)morpholin-4-yl]sulfonyl}-1H-indole-2-carboxamide;
5-Chloro-3-(2,3-dihydro-1,4-benzoxazepin-4(5H)-ylsulfonyl)-1H-indole-2-carboxamide;
3-[(Benzofuran-yl-1-oxa-8-azaspiro[4.5]dec-8-yl)sulfonyl]-5-chloro-1H-indole-2-carboxamide;
5-Chloro-3-{[4-fluoro-4-(3-phenylpropyl)piperidin-1-yl]sulfonyl}-1H-indole-2-carboxamide;
3-[(3-Benzyl-1-oxa-8-azaspiro[4.5]dec-8-yl)sulfonyl]-5-chloro-1H-indole-2-carboxamide;
3-({4-[(Benzyloxy)methyl]-4-phenylpiperidin-1-yl}sulfonyl)-5-chloro-1H-indole-2-carboxamide;
5-Chloro-3-{[4-hydroxy-4-(3-phenylpropyl)piperidin-1-yl]sulfonyl}-1H-indole-2-carboxamide;
7-{[2-(Aminocarbonyl)-5-chloro-1H-indol-3-yl]sulfonyl}-2-(4-chlorophenyl)-7-aza-2-azoniaspiro[4.4]nonane;
3-(1-{[2-(Aminocarbonyl)-5-chloro-1H-indol-3-yl]sulfonyl}piperidin-3-yl)-4-methyl-4H-1,2,4-triazole;
5-Chloro-3-{[3-(2-phenylethyl)piperidin-1-yl]sulfonyl}-1H-indole-2-carboxamide;
5-Chloro-3-{[3-(2-phenylethyl)pyrrolidin-1-yl]sulfonyl}-1H-indole-2-carboxamide;
5-Chloro-3-{[4-(cyclopropyl {[3-(trifluoromethyl)phenyl]sulfonyl}amino)piperidin-1-yl]sulfonyl}-1H-indole-2-carboxamide;
5-Chloro-3-({2-[(4-chlorophenoxy)methyl]morpholin-4-yl}sulfonyl)-1H-indole-2-carboxamide;
Tert-butyl (1-1-{[2-(aminocarbonyl)-5-chloro-1H-indol-3-yl]sulfonyl}piperidin-3-yl)acetate;
3-[(3-Benzylpiperidin-1-yl)sulfonyl]-5-chloro-1H-indole-2-carboxamide;
5-Chloro-3-{[3-(2-methylphenyl)piperidin-1-yl]sulfonyl}-1H-indole-2-carboxamide;
2-(1-{[2-(Aminocarbonyl)-5-chloro-1H-indol-3-yl]sulfonyl}piperidin-4-yl)-N,N-dimethylethanamine;
1-(1-{[2-(Aminocarbonyl)-5-chloro-1H-indol-3-yl]sulfonyl}piperidin-4-yl)-3-(ethoxycarbonyl)piperidine;
5-Bromo-3-{[3-(4-tert-butoxybenzyl)piperidin-1-yl]sulfonyl}-1H-indole-2-carboxamide;
5-Bromo-3-{[4-(3-phenylpropyl)piperidin-1-yl]sulfonyl}-1H-indole-2-carboxamide;
5-Bromo-N-methoxy-N-methyl-3-{[2-(phenoxymethyl)morpholin-4-yl]sulfonyl}-1H-indole-2-carboxamide; and
(S)-3-{[2-(phenoxymethyl)morpholino-4-yl]sulfonyl}-1H-indole-2-carboxamide;
or the pharmaceutically acceptable salts or stereoisomers thereof.

65. The method according to claim 64 wherein the inhibitor of a protein kinase is selected from:
5-Bromo-3-(morpholin-4-ylsulfonyl)-1H-indole-2-carboxamide;
(S)-5-Chloro-3-{[2-(phenoxymethyl)morpholin-4-yl]sulfonyl}-1H-indole-2-carboxamide;
(S)-5-Bromo-3-{[2-(phenoxymethyl)morpholino-4-yl]sulfonyl}-1H-indole-2-carboxamide;
(S)-5-Iodo-3-{[2-(phenoxymethyl)morpholino-4-yl]sulfonyl}-1H-indole-2-carboxamide;
7-Amino-3-{[(2S)-2-(phenoxymethyl)morpholin-4-yl]sulfonyl}-1H-indole-2-carboxamide;
3-{[(2S)-2-(Phenoxymethyl)morpholin-4-yl]sulfonyl}-7-[(pyridin-4-ylmethyl)amino]-1H-indole-2-carboxamide;
5-bromo-3-({2-[(2,3-dihydro-1H-inden-2-ylamino)carbonyl]morpholin-4-yl}sulfonyl)-1H-indole-2-carboxamide;
5-bromo-3-[(2-{[(1-naphthylmethyl)amino]carbonyl}morpholin-4-yl)sulfonyl]-1H-indole-2-carboxamide;
5-chloro-3-({2-[(4-chlorophenoxy)methyl]morpholin-4-yl}sulfonyl)-1H-indole-2-carboxamide; and
(S)-3-{[2-(phenoxymethyl)morpholino-4-yl]sulfonyl}-1H-indole-2-carboxamide;
or a pharmaceutically acceptable salt or stereoisomer thereof.

66. A pharmaceutical composition for treating cancer in a mammal in need thereof which comprises amounts of at least one inhibitor of Akt and one inhibitor of a protein kinase.

67. A pharmaceutical composition for treating cancer in a mammal in need thereof which comprises amounts of at least two inhibitors of Akt.

68. The pharmaceutical composition according to claim 66 comprising an amount of an inhibitor of Akt and an inhibitor of a protein kinase.

69. The pharmaceutical composition according to claim 67 comprising an amount of two inhibitors of Akt.

70. The pharmaceutical composition according to claim 66 wherein the treatment of cancer is selected from inhibition of cancerous tumor growth and the regression of cancerous tumors.

71. The pharmaceutical composition according to claim 67 wherein the treatment of cancer is selected from inhibition of cancerous tumor growth and the regression of cancerous tumors.

72. The pharmaceutical composition according to claim 66 wherein the treatment of cancer is selected from cancer comprising breast cancer, prostate cancer, pancreatic cancer, colorectal cancer, lung cancer, ovarian cancer, renal cell carcinoma, endometrial carcinoma, glioblastoma, colon cancer and bladder cancer.

73. The pharmaceutical composition according to claim 67 wherein the treatment of cancer is selected from cancer comprising breast cancer, prostate cancer, pancreatic cancer, colorectal cancer, lung cancer, ovarian cancer, renal cell carcinoma, endometrial carcinoma, glioblastoma, colon cancer and bladder cancer.

74. The pharmaceutical composition according to claim 72 wherein the cancer is selected from breast cancer, prostate cancer, pancreatic cancer and ovarian cancer.

75. The pharmaceutical composition according to claim 73 wherein the cancer is selected from breast cancer, prostate cancer, pancreatic cancer and ovarian cancer.

76. A method of preparing a pharmaceutical composition for treating cancer in a mammal in need thereof which comprises mixing amounts of at least one inhibitor of Akt and at least one inhibitor of a protein kinase.

77. A method of preparing a pharmaceutical composition for treating cancer in a mammal in need thereof which comprises mixing amounts of at least two selective inhibitors of Akt.

78. The method of preparing a pharmaceutical composition according to claim 75 comprising mixing an amount of an inhibitor of Akt and an amount of an inhibitor of a protein kinase.

79. The method of preparing a pharmaceutical composition according to claim 77 comprising mixing an amount of two selective inhibitors of Akt.

80. A method of treating cancer in a mammal in need thereof which comprises administering to said mammal amounts of at least one inhibitor of Akt and at least one inhibitor of a protein kinase and applying to the mammal radiation therapy.

81. A method of treating cancer in a mammal in need thereof which comprises administering to said mammal amounts of at least two inhibitors of Akt and applying to the mammal radiation therapy.

82. The method according to claim 80 wherein an amount of an inhibitor of Akt and an amount of an inhibitor of a protein kinase are administered simultaneously.

83. The method according to claim 81 wherein the amount of at least two inhibitors of Akt are administered simultaneously.

84. The method according to claim 80 wherein an amount of an inhibitor of Akt and an amount of an inhibitor of a protein kinase are administered consecutively.

85. The method according to claim 81 wherein the amounts of at least two inhibitors of Akt are administered consecutively.

86. A method of treating cancer in a mammal in need thereof which comprises administering to said mammal amounts of at least one inhibitor of Akt and at least one inhibitor of a protein kinase in combination with a third compound selected from:
1) an estrogen receptor modulator,
2) an androgen receptor modulator,
3) a retinoid receptor modulator,
4) a cytotoxic/cytostatic agent,
5) an antiproliferative agent,
6) a prenyl-protein transferase inhibitor,
7) an HMG-CoA reductase inhibitor,
8) an HIV protease inhibitor,
9) a reverse transcriptase inhibitor,
10) an angiogenesis inhibitor,
11) PPAR-γ agonists,
12) PPAR-δ agonists,
13) an inhibitor of inherent multidrug resistance,
14) an anti-emetic agent,
15) an agent useful in the treatment of anemia,
16) an agent useful in the treatment of neutropenia,
17) an immunologic-enhancing drug,
18) an inhibitor of cell proliferation and survival signaling, and
19) an agent that interferes with a cell cycle checkpoint.

87. A method of treating cancer in a mammal in need thereof which comprises administering to said mammal amounts of at least two inhibitors of Akt in combination with a third compound selected from:
1) an estrogen receptor modulator,
2) an androgen receptor modulator,
3) a retinoid receptor modulator,
4) a cytotoxic/cytostatic agent,
5) an antiproliferative agent,
6) a prenyl-protein transferase inhibitor,
7) an HMG-CoA reductase inhibitor,
8) an HIV protease inhibitor,
9) a reverse transcriptase inhibitor,
10) an angiogenesis inhibitor,
11) PPAR-γ agonists,
12) PPAR-δ agonists,
13) an inhibitor of inherent multidrug resistance,
14) an anti-emetic agent,
15) an agent useful in the treatment of anemia,
16) an agent useful in the treatment of neutropenia,
17) an immunologic-enhancing drug,
18) an inhibitor of cell proliferation and survival signaling, and
19) an agent that interferes with a cell cycle checkpoint.

88. The method of claim 86 wherein the third compound is an estrogen receptor modulator selected from tamoxifen and raloxifene.

89. The method of claim 87 wherein the third compound is an estrogen receptor modulator selected from tamoxifen and raloxifene.

90. The method of claim 86 wherein the third compound is paclitaxel or trastuzumab.

91. The method of claim 87 wherein the third compound is paclitaxel or trastuzumab.

92. The method of claim 86 wherein the third compound is a GPIIb/IIIa antagonist.

93. The method of claim 87 wherein the third compound is a GPIIb/IIIa antagonist.

94. The method of claim 92 wherein the GPIIb/IIIa antagonist is tirofiban.

95. The method of claim 93 wherein the GPIIb/IIIa antagonist is tirofiban.

96. The method of claim 86 wherein the third compound is a COX-2 inhibitor.

97. The method of claim 87 wherein the third compound is a COX-2 inhibitor.


SEQUENCE LISTING (22 sequences)

SEQ ID NO: 1 - DNA, 10 bp, Artificial Sequence
  Completely synthetic DNA Sequence
ctgcggccgc

SEQ ID NO: 2 - DNA, 14 bp, Artificial Sequence
  Completely synthetic DNA Sequence
gtacgcggccgcag

SEQ ID NO: 3 - DNA, 39 bp, Artificial Sequence
  Completely synthetic DNA Sequence
cgcgaattcagatctaccatgagcgacgtggctattgtg

SEQ ID NO: 4 - DNA, 33 bp, Artificial Sequence
  Completely synthetic DNA Sequence
cgctctagaggatcctcaggccgtgctgctggc

SEQ ID NO: 5 - DNA, 24 bp, Artificial Sequence
  Completely synthetic DNA Sequence
gtacgatgctgaacgatatcttcg

SEQ ID NO: 6 - DNA, 45 bp, Artificial Sequence
  Completely synthetic DNA Sequence
gaatacatgccgatggaaagcgacggggctgaagagatggaggtg

SEQ ID NO: 7 - DNA, 40 bp, Artificial Sequence
  Completely synthetic DNA Sequence
cccctccatctcttcagccccgtcgctttccatcggcatg

SEQ ID NO: 8 - DNA, 60 bp, Artificial Sequence
  Completely synthetic DNA Sequence
cgcggcgcgccaggtaccatggaatacatgccgatggaaaagaagcaggaggaggaggag

SEQ ID NO: 9 - DNA, 21 bp, Artificial Sequence
  Completely synthetic DNA Sequence
cggagaacacacgctcccggg

SEQ ID NO: 10 - DNA, 36 bp, Artificial Sequence
  Completely synthetic DNA Sequence
gaattcagatctaccatgagcgatgttaccattgtg

SEQ ID NO: 11 - DNA, 30 bp, Artificial Sequence
  Completely synthetic DNA Sequence
tctagatcttattctcgtccacttgcagag

SEQ ID NO: 12 - DNA, 48 bp, Artificial Sequence
  Completely synthetic DNA Sequence
ggtaccatggaatacatgccgatggaaagcgatgttaccattgtgaag

SEQ ID NO: 13 - DNA, 58 bp, Artificial Sequence
  Completely synthetic DNA Sequence
cgcaggtaccatggaatacatgccgatggaaagcgatggagaggaagagatggatgcc

SEQ ID NO: 14 - DNA, 33 bp, Artificial Sequence
  Completely synthetic DNA Sequence
cgctctagatcttattctcgtccacttgcagag

SEQ ID NO: 15 - DNA, 33 bp, Artificial Sequence
  Completely synthetic DNA Sequence
aagcttagatctaccatgaatgaggtgtctgtc

SEQ ID NO: 16 - DNA, 30 bp, Artificial Sequence
  Completely synthetic DNA Sequence
gaattcggatcctcactcgcggatgctggc

SEQ ID NO: 17 - DNA, 49 bp, Artificial Sequence
  Completely synthetic DNA Sequence
ggtaccatggaatacatgccgatggaaaatgaggtgtctgtcatcaaag

SEQ ID NO: 18 - DNA, 60 bp, Artificial Sequence
  Completely synthetic DNA Sequence
cgcaggtaccatggaatacatgccgatggaaaatgagacgactgaggagatggaagtggc

SEQ ID NO: 19 - DNA, 33 bp, Artificial Sequence
  Completely synthetic DNA Sequence
cgcgaattcggatcctcactcgcggatgctggc

SEQ ID NO: 20 - protein, 6 aa, Artificial Sequence
  Completely synthetic Amino Acid Sequence
EYMPME

SEQ ID NO: 21 - protein, 13 aa, Artificial Sequence
  Completely synthetic Amino Acid Sequence
GGRARTSSFAEPG

SEQ ID NO: 22 - protein, 15 aa, Artificial Sequence
  Completely synthetic Amino Acid Sequence
KKGGRARTSSFAEPG